(19) United States
(12) Patent Application Publication
(10) Pub. No.: US 2014/0288097 A1
(43) Pub. Date: Sep. 25, 2014

(54) USE OF TRANSLATIONAL PROFILING TO IDENTIFY TARGET MOLECULES FOR THERAPEUTIC TREATMENT

(71) Applicant: The Regents of the University of California, Oakland, CA (US)

(72) Inventors: Davide Ruggero, San Francisco, CA (US); Andrew Hsieh, San Francisco, CA (US); Merritt Edlind, Berkeley, CA (US); Kevan M. Shokat, San Francisco, CA (US); James Appleman, San Diego, CA (US); Steve Worland, Del Mar, CA (US)

(73) Assignee: The Regents of The University of California, Oakland, CA (US)

(21) Appl. No.: 14/176,018

(22) Filed: Feb. 7, 2014

Related U.S. Application Data
(60) Provisional application No. 61/762,115, filed on Feb. 7, 2013.

(51) Int. Cl. C12Q 1/68

(52) U.S. Cl. CPC C12Q 1/6883
USPC 514/262.1; 506/9; 514/291

(57) ABSTRACT

The present invention provides methods of identifying an agent or drug candidate molecule, validating a target, and identifying normalizing therapeutics that modulates translation, such as in an oncogenic signaling pathway, in a biological sample as determined by translational profiling of one or more genes in the biological sample. The present invention also provides diagnostic and therapeutic methods using the translational profiling methods described herein.

### CROSS-REFERENCE TO RELATED APPLICATIONS

The present application claims priority to U.S. Provisional Application No. 61/762,115, filed Feb. 7, 2013, the entire content of which is incorporated by reference herein for all purposes.

### STATEMENT AS TO RIGHTS TO INVENTIONS MADE UNDER FEDERALLY SPONSORED RESEARCH AND DEVELOPMENT

This invention was made with government support under Grant No. RO1 CA154916 awarded by the National Institutes of Health. The government has certain rights in the invention.

### BACKGROUND OF THE INVENTION

Gene expression studies have been used to examine mRNA in cell populations under different conditions, e.g., for comparing gene expression under different drug treatments or in different cell types. For example, Cheok et al. (Nat. Genet. 34:85-90 (2003)) demonstrated that lymphoid leukemia cells of different molecular subtypes share common pathways of genomic response to the same treatment, and that changes in gene expression are treatment-specific and that gene expression can illuminate differences in cellular response to drug combinations versus single agents. However, these types of gene expression studies have many drawbacks. For example, genome-scale predictions of synthesis rates of mRNAs and proteins have been used to demonstrate that cellular abundance of proteins is predominantly controlled at the level of translation. Schwanhausser et al. (Nature 473:337-342 (2011)).

The mammalian target of rapamycin (mTOR) kinase is a master regulator of protein synthesis that couples nutrient sensing to cell growth and cancer. However, the downstream translationally regulated nodes of gene expression that may direct cancer development have not been well characterized. Thus, there remains a need for methods of characterizing the translational control of mRNAs in oncogenic mTOR signaling and in cell populations generally. The present invention addresses this need and others.

### BRIEF SUMMARY OF THE INVENTION

In one aspect, the present invention relates to methods for identifying an agent that modulates an oncogenic signaling pathway (e.g., an agent that inhibits an oncogenic signaling pathway) in a biological sample. In some embodiments, the method comprises:
(a) contacting the biological sample with an agent;
(b) determining a first translational profile for the contacted biological sample, wherein the translational profile comprises translational levels for one or more genes having a 5′ terminal oligopyrimidine tract (5′ TOP) and/or a pyrimidine-rich translational element (PRTE); and
(c) comparing the first translational profile to a second translational profile comprising translational levels for the one or more genes in a control sample that has not been contacted with the agent;
   wherein a difference in the translational levels of the one or more genes in the first translation profile as compared to the second translation profile identifies the agent as a modulator of the oncogenic signaling pathway.

In some embodiments, the method comprises:
(a) contacting the biological sample with an agent;
(b) determining a first translational profile for the contacted biological sample, wherein the translational profile comprises translational levels for one or more genes selected from the group consisting of SEQ ID NOs:1-144; and
(c) comparing the first translational profile to a second translational profile comprising translational levels for the one or more genes in a control sample that has not been contacted with the agent;
   wherein a difference in the translational levels of the one or more genes in the first translation profile as compared to the second translation profile identifies the agent as a modulator of the oncogenic signaling pathway.

In some embodiments, the method comprises:
(a) contacting the biological sample with an agent;
(b) determining a first translational profile for the contacted biological sample, wherein the translational profile comprises a measurement of gene translational levels for a substantial portion of the genome;
(c) comparing the first translational profile to a second translational profile comprising a measurement of gene translational levels for the substantial portion of the genome translational levels for the one or more genes in a control sample that has not been contacted with the agent;
(d) identifying in the first translational profile a plurality of genes having decreased translational levels as compared to the translational levels of the plurality of genes in the second translational profile; and
(e) determining whether, for the plurality of genes identified in step (d), there is a common consensus sequence and/or regulatory element in the untranslated regions (UTRs) of the genes that is shared by at least 10% of the plurality of genes identified in step (d);
   wherein a decrease in the translational levels of at least 10% of the genes sharing the common consensus sequence and/or UTR regulatory element in the first translational profile as compared to the second translational profile identifies the agent as an inhibitor of an oncogenic signaling pathway.

In some embodiments, the one or more genes are selected from the genes listed in Table 1, Table 2, and/or Table 3. In some embodiments, the one or more genes are cell invasion and/or metastasis genes. In some embodiments, the one or more genes are selected from Y-box binding protein 1 (YB1), vimentin, metastasis associated 1 (MTA1), and CD44.

In some embodiments, the oncogenic signaling pathway is the mammalian target of rapamycin (mTOR) pathway, the PI3K pathway, the AKT pathway, the Ras pathway, the Myc pathway, the Wnt pathway, or the BRAF pathway. In some embodiments, the oncogenic signaling pathway is the mTOR pathway.

In some embodiments, the translational level for the one or more genes is decreased for the first translational profile as compared to the second translational profile, thereby identifying the agent as an inhibitor of the oncogenic signaling pathway. In some embodiments, the translational level of the one or more genes in the first translational profile is decreased by at least three-fold as compared to the second translational profile. In some embodiments, the translational level for the one or more genes is increased for the first translational profile as compared to the second translational profile, thereby identifying the agent as a potentiator of the oncogenic signaling pathway. In some embodiments, the translational level of the one or more genes in the first translational profile is increased by at least three-fold as compared to the second translational profile.

In some embodiments, the first and/or second translational profiles are generated using ribosomal profiling. In some embodiments, the first and/or second translational profiles are generated using polysome microarray. In some embodiments, the first and/or second translational profiles are generated using immunoassay. In some embodiments, the first and/or second translational profiles are generated using mass spectrometry analysis.

In some embodiments, the first and/or second translation profile comprises measuring the translational levels of at least 500 genes in the sample (e.g., at least 500, 600, 700, 800, 900, 1000, 2000, 3000, 4000, 5000, 6000, 7000, 8000, 9000, 10,000, 11,000, 12,000, 13,000, 14,000, or 15,000 genes or more). In some embodiments, the first and/or second translational profile comprises a genome-wide measurement of gene translational levels.

In some embodiments, the biological sample comprises a cell. In some embodiments, the cell is a human cell. In some embodiments, the cell is a cancer cell. In some embodiments, the cancer is prostate cancer, breast cancer, bladder cancer, lung cancer, renal cell carcinoma, endometrial cancer, melanoma, ovarian cancer, thyroid cancer, or brain cancer.

In some embodiments, the identified agent binds to a 5′ TOP or PRTE sequence in the one or more genes having a different translational level in the first translational profile as compared to the second translational profile. In some embodiments, the identified agent inhibits the activity of a downstream effector of the oncogenic signaling pathway, wherein the effector is 4EBP1, p70S6K1/2, or AKT.

In some embodiments, the method further comprises chemically synthesizing a structurally related agent derived from the identified agent. In some embodiments, the method further comprises administering the structurally related agent to an animal and determining the oral bioavailability of the structurally related agent. In some embodiments, the method further comprises administering the structurally related agent to an animal and determining the potency of the structurally related agent.

In another aspect, the present invention relates to a structurally related agent to an agent identified as described herein.

In still another aspect, the present invention relates to methods of validating a target for therapeutic intervention. In some embodiments, the method comprises:
(a) contacting a biological sample with an agent that modulates the target;
(b) determining a first translational profile for the contacted biological sample, wherein the first translational profile comprises translational levels for a plurality of genes; and
(c) comparing the first translational profile to a second translational profile comprising translational levels for the plurality of genes in a control sample that has not been contacted with the agent;
   wherein identifying one or more genes of a biological pathway as differentially translated in the first translational profile as compared to the second translational profile validates the target for therapeutic intervention, wherein said biological pathway is selected from a protein synthesis pathway, a cell invasion/metastasis pathway, a cellular metabolism pathway, a cell division pathway, an apoptosis pathway, a signal transduction pathway, a cellular transport pathway, a post-translational protein modification pathway, a DNA repair pathway, and a DNA methylation pathway.

In some embodiments, the one or more genes have a 5′ terminal oligopyrimidine tract (5′ TOP) and/or a pyrimidine-rich translational element (PRTE). In some embodiments, the one or more genes are selected from the group consisting of SEQ ID NOs:1-144.

In some embodiments, the target for therapeutic intervention is part of an oncogenic signaling pathway. In some embodiments, the oncogenic signaling pathway is the mammalian target of rapamycin (mTOR) pathway. In some embodiments, the target for therapeutic intervention is a protein. In some embodiments, the target for therapeutic intervention is a nucleic acid.

In some embodiments, one or more genes from each of at least two of the biological pathways is differentially translated in the first translational profile as compared to the second translational profile. In some embodiments, one or more genes from each of at least three of the biological pathways is differentially translated in the first translational profile as compared to the second translational profile. In some embodiments, there is at least a two-fold difference in translational level for the one or more genes in the first translational profile as compared to the second translational profile.

In some embodiments, the first and/or second translational profile comprises a genome-wide measurement of gene translational levels. In some embodiments, less than 20% of the genes in the genome are differentially translated by at least two-fold in the first translational profile as compared to the second translational profile. In some embodiments, less than 5% of the genes in the genome are differentially translated by at least two-fold in the first translational profile as compared to the second translational profile. In some embodiments, less than 1% of the genes in the genome are differentially translated by at least two-fold in the first translational profile as compared to the second translational profile.

In some embodiments, the first and/or second translational profiles are generated using ribosomal profiling. In some embodiments, the first and/or second translational profiles are generated using polysome microarray. In some embodiments, the first and/or second translational profiles are generated using immunoassay. In some embodiments, the first and/or second translational profiles are generated using mass spectrometry analysis.

In some embodiments, the biological sample comprises a cell. In some embodiments, the cell is a human cell. In some embodiments, the cell is a cancer cell. In some embodiments, the cancer is prostate cancer, breast cancer, bladder cancer, lung cancer, renal cell carcinoma, endometrial cancer, melanoma, ovarian cancer, thyroid cancer, or brain cancer.

In some embodiments, the therapeutic intervention is an anti-cancer therapy.

In some embodiments, the agent is a peptide, protein, RNA, or small organic molecule. In some embodiments, the agent is an inhibitory RNA.

In yet another aspect, the present invention relates to methods of identifying a drug candidate molecule. In some embodiments, the method comprises:
(a) contacting a biological sample with the drug candidate molecule;
(b) determining a translational profile for the contacted biological sample, wherein the translational profile comprises translational levels for a plurality of genes; and
(c) comparing the first translational profile to a second translational profile comprising translational levels for the plurality of genes in a control sample that has not been contacted with the drug candidate molecule,
   wherein the drug candidate molecule is identified as suitable for use in a therapeutic intervention when one or more genes of a biological pathway is differentially translated in the first translational profile as compared to the second translational profile, wherein the biological pathway is selected from a protein synthesis pathway, a cell invasion/metastasis pathway, a cellular metabolism pathway, a cell division pathway, an apoptosis pathway, a signal transduction pathway, a cellular transport pathway, a post-translational protein modification pathway, a DNA repair pathway, and DNA methylation pathway.

In some embodiments, the one or more genes have a 5′ terminal oligopyrimidine tract (5′ TOP) and/or a pyrimidine-rich translational element (PRTE). In some embodiments, the one or more genes are selected from the group consisting of SEQ ID NOs:1-144.

In some embodiments, one or more genes from each of at least two of the biological pathways is differentially translated in the first translational profile as compared to the second translational profile. In some embodiments, one or more genes from each of at least three of the biological pathways is differentially translated in the first translational profile as compared to the second translational profile. In some embodiments, there is at least a two-fold difference in translational level for the one or more genes in the first translational profile as compared to the second translational profile.

In some embodiments, the first and/or second translational profile comprises a genome-wide measurement of gene translational levels. In some embodiments, less than 20% of the genes in the genome are differentially translated by at least two-fold in the first translational profile as compared to the second translational profile. In some embodiments, less than 5% of the genes in the genome are differentially translated by at least two-fold in the first translational profile as compared to the second translational profile. In some embodiments, less than 1% of the genes in the genome are differentially translated by at least two-fold in the first translational profile as compared to the second translational profile.

In some embodiments, the first and/or second translational profiles are generated using ribosomal profiling. In some embodiments, In some embodiments, the first and/or second translational profiles are generated using polysome microarray. In some embodiments, the first and/or second translational profiles are generated using immunoassay. In some embodiments, the first and/or second translational profiles are generated using mass spectrometry analysis.

In some embodiments, the method further comprises comparing the translational profile for the contacted biological sample with a control translational profile for a second biological sample that has been contacted with a known therapeutic agent. In some embodiments, the known therapeutic agent is a known inhibitor of an oncogenic signaling pathway. In some embodiments, the known therapeutic agent is a known inhibitor of the mammalian target of rapamycin (mTOR) pathway.

In still another aspect, the present invention relates to methods of identifying a subject as a candidate for treatment with an mTOR inhibitor. In some embodiments, the method comprises:
(a) determining a first translational profile in a sample from the subject, wherein the first translational profile comprises translational levels for one or more genes having a 5′ terminal oligopyrimidine tract (5′ TOP) and/or a pyrimidine-rich translational element (PRTE); and
(b) comparing the first translational profile to a second translational profile comprising translational levels for the one or more genes, wherein the second translational profile is from a control sample, wherein the control sample is from a known responder to the mTOR inhibitor prior to administration of the mTOR inhibitor to the known responder;
   wherein a translational level of the one or more genes in the first translational profile that is at least as high as the translational level of the one or more genes in the second translational profile identifies the subject as a candidate for treatment with the mTOR inhibitor.

In some embodiments, the method comprises:
(a) determining a first translational profile in a sample from the subject, wherein the first translational profile comprises translational levels for one or more genes selected from the group consisting of SEQ ID NOs:1-144; and
(b) comparing the first translational profile to a second translational profile comprising translational levels for the one or more genes, wherein the second translational profile is from a control sample, wherein the control sample is from a known responder to the mTOR inhibitor prior to administration of the mTOR inhibitor to the known responder; wherein a translational level of the one or more genes in the first translational profile that is at least as high as the translational level of the one or more genes in the second translational profile identifies the subject as a candidate for treatment with the mTOR inhibitor.

In some embodiments, the one or more genes are selected from the genes listed in Table 1, Table 2, and/or Table 3. In some embodiments, the one or more genes are cell invasion and/or metastasis genes. In some embodiments, the one or more genes are selected from Y-box binding protein 1 (YB1), vimentin, metastasis associated 1 (MTA1), and CD44.

In some embodiments, the method comprises:
(a) determining a first translational profile in a sample from the subject, wherein the first translational profile comprises translational levels for one or more genes of a biological pathway, wherein the biological pathway is selected from a protein synthesis pathway, a cell invasion/metastasis pathway, a cellular metabolism pathway, a cell division pathway, an apoptosis pathway, a signal transduction pathway, a cellular transport pathway, a post-translational protein modification pathway, a DNA repair pathway, and a DNA methylation pathway; and
(b) comparing the first translational profile to a second translational profile comprising translational levels for the one or more genes, wherein the second translational profile is from a control sample, wherein the control sample is from a known responder to the mTOR inhibitor prior to administration of the mTOR inhibitor to the known responder;
   wherein a translational level of the one or more genes in the first translational profile that is at least as high as the translational level of the one or more genes in the second translational profile identifies the subject as a candidate for treatment with the mTOR inhibitor.

In some embodiments, the translational level of one or more genes from each of at least two of the biological pathways is at least as high in the first translational profile as in the second translational profile. In some embodiments, the translational level of one or more genes from each of at least three of the biological pathways is at least as high in the first translational profile as in the second translational profile.

In some embodiments, there is at least a two-fold difference in translational level for the one or more genes in the first translational profile as compared to the second translational profile.

In some embodiments, the first and/or second translation profile comprises measuring the translational levels of at least 500 genes in the sample (e.g., at least 500, 600, 700, 800, 900, 1000, 2000, 3000, 4000, 5000, 6000, 7000, 8000, 9000, 10,000, 11,000, 12,000, 13,000, 14,000, or 15,000 genes or more). In some embodiments, the first and/or second translational profile comprises a genome-wide measurement of gene translational levels. In some embodiments, the first and second translational profiles are differential profiles from before and after administration of the mTOR inhibitor.

In some embodiments, the subject has a cancer. In some embodiments, the cancer is prostate cancer, breast cancer, bladder cancer, lung cancer, renal cell carcinoma, endometrial cancer, melanoma, ovarian cancer, thyroid cancer, or brain cancer.

In some embodiments, the method further comprises administering an mTOR inhibitor to the subject.

In still another aspect, the present invention relates to methods of identifying a subject as a candidate for treatment with a therapeutic agent. In some embodiments, the method comprises:
(a) determining a first translational profile in a sample from the subject, wherein the translational profile comprises translational levels for one or more genes of a biological pathway, wherein the biological pathway is selected from a protein synthesis pathway, a cell invasion/metastasis pathway, a cellular metabolism pathway, a cell division pathway, an apoptosis pathway, a signal transduction pathway, a cellular transport pathway, a post-translational protein modification pathway, a DNA repair pathway, and a DNA methylation pathway; and
(b) comparing the first translational profile to a second translational profile comprising translational levels for the one or more genes, wherein the second translational profile is from a control sample, wherein the control sample is from a known responder to the therapeutic agent prior to administration of the therapeutic agent to the known responder;
   wherein a translational level of the one or more genes that is at least as high as the translational level of the one or more genes in the second translational profile identifies the subject as a candidate for treatment with the therapeutic agent.

In some embodiments, the translational level of one or more genes from each of at least two of the biological pathways is at least as high in the first translational profile as in the second translational profile. In some embodiments, the translational level of one or more genes from each of at least three of the biological pathways is at least as high in the first translational profile as in the second translational profile.

In some embodiments, the first and second translational profiles are differential profiles from before and after administration of the therapeutic agent.

In some embodiments, the subject has a disease. In some embodiments, the disease is cancer. In some embodiments, the cancer is prostate cancer, breast cancer, bladder cancer, lung cancer, renal cell carcinoma, endometrial cancer, melanoma, ovarian cancer, thyroid cancer, or brain cancer. In some embodiments, the biological sample comprises diseased cells.

In yet another aspect, the present invention relates to methods of treating a subject having a cancer. In some embodiments, the method comprises:
administering an mTOR inhibitor to a subject that has been selected as having a first translational profile comprising a translational level of one or more genes that is at least as high as the translational level of the one or more genes in a second translational profile from a control sample;
wherein the first and second translational profiles comprise translational levels for one or more genes having a 5′ terminal oligopyrimidine tract (5′ TOP) and/or a pyrimidine-rich translational element (PRTE); and wherein the control sample is from a known responder to the mTOR inhibitor prior to administration of the mTOR inhibitor to the known responder;
thereby treating the cancer in the subject.

In some embodiments, the method of treating a subject having a cancer comprises:
administering an mTOR inhibitor to a subject that has been selected as having a first translational profile comprising a translational level of one or more genes that is at least as high as the translational level of the one or more genes in a second translational profile from a control sample;
wherein the first and second translational profiles comprise translational levels for one or more genes selected from the group consisting of SEQ ID NOs:1-144; and wherein the control sample is from a known responder to the mTOR inhibitor prior to administration of the mTOR inhibitor to the known responder;
thereby treating the cancer in the subject.

In some embodiments, the one or more genes are selected from the genes listed in Table 1, Table 2, and/or Table 3. In some embodiments, the one or more genes are cell invasion and/or metastasis genes. In some embodiments, the one or more genes are selected from Y-box binding protein 1 (YB1), vimentin, metastasis associated 1 (MTA1), and CD44.

In some embodiments, the method of treating a subject having a cancer comprises:
administering an mTOR inhibitor to a subject that has been selected as having a first translational profile comprising a translational level of one or more genes that is at least as high as the translational level of the one or more genes in a second translational profile from a control sample;
wherein the first and second translational profiles comprise translational levels for one or more genes of a biological pathway selected from a protein synthesis pathway, a cell invasion/metastasis pathway, a cellular metabolism pathway, a cell division pathway, an apoptosis pathway, a signal transduction pathway, a cellular transport pathway, a post-translational protein modification pathway, a DNA repair pathway, and a DNA methylation pathway; and wherein the control sample is from a known responder to the mTOR inhibitor prior to administration of the mTOR inhibitor to the known responder;
thereby treating the cancer in the subject.

In some embodiments, the translational level of one or more genes from each of at least two of the biological pathways is at least as high in the first translational profile as in the second translational profile. In some embodiments, the translational level of one or more genes from each of at least three of the biological pathways is at least as high in the first translational profile as in the second translational profile.

In some embodiments, the first and/or second translation profile comprises measuring the translational levels of at least 500 genes in the sample (e.g., at least 500, 600, 700, 800, 900, 1000, 2000, 3000, 4000, 5000, 6000, 7000, 8000, 9000, 10,000, 11,000, 12,000, 13,000, 14,000, or 15,000 genes or more). In some embodiments, the first and/or second translational profile comprises a genome-wide measurement of gene translational levels. In some embodiments, the first and second translational profiles are differential profiles from before and after administration of the mTOR inhibitor.

In some embodiments, the subject has a cancer. In some embodiments, the cancer is prostate cancer, breast cancer, bladder cancer, lung cancer, renal cell carcinoma, endometrial cancer, melanoma, ovarian cancer, thyroid cancer, or brain cancer. In some embodiments, the cancer is an invasive cancer.

In some embodiments, the method further comprises monitoring the translational levels of the one or more genes in the subject subsequent to administering the mTOR inhibitor.

In still another aspect, the present invention relates to methods of treating a subject in need thereof. In some embodiments, the method comprises:
administering a therapeutic agent to a subject that has been selected as having a first translational profile comprising a translational level of one or more genes that is at least as high as the translational level of the one or more genes in a second translational profile;
wherein the first and second translational profiles comprise translational levels for one or more genes of a biological pathway selected from a protein synthesis pathway, a cell invasion/metastasis pathway, a cellular metabolism pathway, a cell division pathway, an apoptosis pathway, a signal transduction pathway, a cellular transport pathway, a post-translational protein modification pathway, a DNA repair pathway, and a DNA methylation pathway; and wherein the control sample is from a known responder to the therapeutic agent prior to administration of the therapeutic agent to the known responder;
thereby treating the subject.

In some embodiments, the translational level of one or more genes from each of at least two of the biological pathways is at least as high in the first translational profile as in the second translational profile. In some embodiments, the translational level of one or more genes from each of at least three of the biological pathways is at least as high in the first translational profile as in the second translational profile.

In some embodiments, the first and second translational profiles are differential profiles from before and after administration of the therapeutic agent.

In some embodiments, the subject in need of treatment has a disease. In some embodiments, the disease is cancer. In some embodiments, the cancer is prostate cancer, breast cancer, bladder cancer, lung cancer, renal cell carcinoma, endometrial cancer, melanoma, ovarian cancer, thyroid cancer, or brain cancer. In some embodiments, the cancer is an invasive cancer. In some embodiments, the biological sample comprises diseased cells.

In still another aspect, the present invention relates to methods of identifying an agent for normalizing a translational profile in a subject in need thereof. In some embodiments, the method comprises:
(a) determining a first translational profile for a first biological sample from the subject, wherein the first translational profile comprises translational levels for a plurality of genes;
(b) comparing the first translational profile to a second translational profile comprising translational levels for the plurality of genes, wherein the second translational profile is from a control sample, wherein the control sample is from a non-diseased subject;
(c) identifying one or more genes of a biological pathway as differentially translated in the first translational profile as compared to the second translational profile, wherein the biological pathway is selected from a protein synthesis pathway, a cell invasion/metastasis pathway, a cellular metabolism pathway, a cell division pathway, an apoptosis pathway, a signal transduction pathway, a cellular transport pathway, a post-translational protein modification pathway, a DNA repair pathway, and a DNA methylation pathway;
(d) contacting a second biological sample from the subject with the agent;
(e) determining a third translational profile for the second biological sample, wherein the third translational profile comprises translational levels for the one or more genes identified as differentially translated in the first translational profile as compared to the second translational profile; and
(f) comparing the translational levels for the one or more genes in the third translational profile to the translational levels for the one or more genes in the first and second translational profiles;
wherein a translational level for the one or more genes in the third translational profile that is closer to the translational level for the one or more genes in the second translational profile than to the translational level for the one or more genes in the first translational profile identifies the agent as an agent for normalizing the translational profile in the subject.

In yet another aspect, the present invention relates to methods of normalizing a translational profile in a subject in need thereof. In some embodiments, the method comprises:
administering to the subject an agent that has been selected as an agent that normalizes the translational profile in the subject, wherein the agent is selected by:
   (a) determining a first translational profile for a first biological sample from the subject, wherein the first translational profile comprises translational levels for a plurality of genes;
   (b) comparing the first translational profile to a second translational profile comprising translational levels for the plurality of genes, wherein the second translational profile is from a control sample, wherein the control sample is from a non-diseased subject;
   (c) identifying one or more genes of a biological pathway as differentially translated in the first translational profile as compared to the second translational profile, wherein the biological pathway is selected from a protein synthesis pathway, a cell invasion/metastasis pathway, a cellular metabolism pathway, a cell division pathway, an apoptosis pathway, a signal transduction pathway, a cellular transport pathway, a post-translational protein modification pathway, a DNA repair pathway, and a DNA methylation pathway;
   (d) contacting a second biological sample form the subject with the agent;
   (e) determining a third translational profile for the second biological sample, wherein the third translational profile comprises translational levels for the one or more genes identified as differentially translated in the first translational profile as compared to the second translational profile; and
   (f) comparing the translational levels for the one or more genes in the third translational profile to the translational levels for the one or more genes in the first and second translational profiles; wherein a translational level for the one or more genes in the third translational profile that is closer to the translational level for the one or more genes in the second translational profile than to the translational level for the one or more genes in the first translational profile identifies the agent as an agent for normalizing the translational profile in the subject;
thereby normalizing the translational profile in the subject.

In some embodiments, one or more genes from each of at least two of the biological pathways is differentially translated in the first translational profile as compared to the second translational profile. In some embodiments, one or more genes from each of at least three of the biological pathways is differentially translated in the first translational profile as compared to the second translational profile. In some embodiments, there is at least a two-fold difference in translational level for the one or more genes in the first translational profile as compared to the second translational profile.

In some embodiments, the first and/or second translation profile comprises measuring the translational levels of at least 500 genes in the sample (e.g., at least 500, 600, 700, 800, 900, 1000, 2000, 3000, 4000, 5000, 6000, 7000, 8000, 9000, 10,000, 11,000, 12,000, 13,000, 14,000, or 15,000 genes or more). In some embodiments, the first, second, and/or third translational profiles comprise a genome-wide measurement of gene translational levels.

In some embodiments, the agent is a peptide, protein, inhibitory RNA, or small organic molecule.

In still another aspect, the present invention relates to methods for identifying a candidate therapeutic for treating a disease. In some embodiments, the method comprises:
(a) determining a first translational profile for a plurality of genes for a disease sample that has been contacted with a candidate agent;
(b) determining a second translational profile for a plurality of genes for a disease sample that has not been contacted with a candidate agent; and
(c) identifying the agent as a candidate therapeutic for treating the disease when one or more genes are differentially translated in the first translation profile as compared to the second translation profile and when the differential translation results in a biological benefit.

In some embodiments, the method for identifying a candidate therapeutic for treating a disease comprises:
(a) determining a first translational profile for a plurality of genes for a disease sample that has been contacted with a candidate agent;
(b) determining a second translational profile for a plurality of genes for a disease sample that has been contacted with a known active compound for treating the disease; and
(c) identifying the agent as a candidate therapeutic for use in treating the disease when the first translational profile is comparable to the second translational profile.

In some embodiments, the method for identifying a candidate therapeutic for treating a disease comprises:
(a) determining three independent translational profiles, each for a plurality of genes from a disease sample, wherein (i) a first translational profile is from a sample not contacted with any compound; (ii) a second translational profile is from a sample that has been contacted with a known active compound for treating the disease; and (iii) a third translational profile is from a sample that has been contacted with a candidate agent;
(b) identifying one or more genes as differentially translated in the first translational profile as compared to the second translational profile; and
(c) identifying the agent as a candidate therapeutic for use in treating the disease when the one or more differentially translated genes from step (b) are in the third translational profile and when the translational profile of the one or more genes in the third translational profile is closer to the translational profile of the one or more genes in the second translational profile than to the translational profile of the one or more genes in the first translational profile.

In some embodiments, the method for identifying a candidate therapeutic for treating a disease comprises:
(a) determining three independent translational profiles, each for a plurality of genes from a disease sample, wherein (i) a first translational profile is from a sample not contacted with any compound; (ii) a second translational profile is from a sample that has been contacted with a known active compound for treating the disease; and (iii) a third translational profile is from a sample that has been contacted with a candidate agent;
(b) determining a first differential translational profile comprising one or more genes differentially translated in the first translational profile as compared to the second translational profile, and determining a second differential translational profile comprising one or more genes differentially translated in the first translational profile as compared to the third translational profile; and
(c) identifying the agent as a candidate therapeutic for use in treating the disease when the first differential translational profile is comparable to the second differential translational profile.

In yet another aspect, the present invention relates to methods for identifying a candidate molecule for normalizing a translational profile associated with a disease. In some embodiments, the method comprises:
(a) determining a first translational profile for a plurality of genes from a disease sample that has been contacted with a candidate agent;
(b) determining a second translational profile for a plurality of genes from (i) a control non-diseased sample or (2) a control non-diseased sample that has been contacted with the candidate agent; and
(c) identifying the agent as a candidate therapeutic for normalizing a translational profile associated with the disease when the first translational profile is comparable to the second translational profile.

In some embodiments, the method for identifying a candidate molecule for normalizing a translational profile associated with a disease comprises:
(a) determining three independent translational profiles, each for a plurality of genes, wherein (i) a first translational profile is from a disease sample, (ii) a second translational profile is from (1) a control non-diseased sample or (2) a control non-diseased sample that has been contacted with a candidate agent, and (iii) a third translational profile is from a disease sample that has been contacted with the candidate agent;
(b) identifying one or more genes as differentially translated in the first translational profile as compared to the second profile; and
(c) identifying the agent as a candidate therapeutic for normalizing a translational profile associated with the disease when the one or more differentially translated genes from step (b) are in the third translational profile and when the translational profile of the one or more genes in the third translational profile is closer to the translational profile of the one or more genes in the second translational profile than to the translational profile of the one or more genes in the first translational profile.

In some embodiments, the method for identifying a candidate molecule for normalizing a translational profile associated with a disease comprises:
(a) determining three independent translational profiles, each for a plurality of genes, wherein (i) a first translational profile is from a disease sample, (ii) a second translational profile is from (1) a control non-diseased sample or (2) a control non-diseased sample that has been contacted with a candidate agent, and (iii) a third translational profile is from a disease sample that has been contacted with the candidate agent;
(b) determining a first differential translational profile comprising one or more genes differentially translated in the first translational profile as compared to the second translational profile, and determining a second differential translational profile comprising one or more genes differentially translated in the first translational profile as compared to the third translational profile; and
(c) identifying the agent as a candidate therapeutic for normalizing a translational profile associated with the disease when the first differential translational profile is comparable to the second differential translational profile.

In still another aspect, the present invention provides methods of validating a target for therapeutic intervention in disease. In some embodiments, the method comprises:
(a) determining a first translational profile for a plurality of genes from a disease sample that has been contacted with an agent that modulates a disease-associated target;
(b) determining a second translational profile for a plurality of genes from a control disease sample that has not been contacted with the agent; and
(c) validating the target for therapeutic intervention in the disease when one or more genes are differentially translated in the first translational profile as compared to the second translational profile and when the differential translation results in a biological benefit.

In some embodiments, the method of validating a target for therapeutic intervention in disease comprises:
(a) determining a first translational profile for a plurality of genes from a disease sample that has been contacted with an agent that modulates a disease-associated target;
(b) determining a second translational profile for a plurality of genes from a control disease sample that has been contacted with a known active compound for treating the disease; and
(c) validating the target as a target for therapeutic intervention in the disease when the first translational profile is comparable to the second translational profile.

In some embodiments, the method of validating a target for therapeutic intervention in disease comprises:
(a) determining three independent translational profiles, each for a plurality of genes from a disease sample, wherein (i) a first translational profile is from a sample not contacted with any compound, (ii) a second translational profile is from a sample contacted with an agent that modulates a disease-associated target, and (iii) a third translational profile is from a sample contacted with a known active compound for treating the disease;
(b) identifying one or more genes as differentially translated in the first translational profile as compared to the second translational profile; and
(c) validating the target as a target for therapeutic intervention in the disease when the one or more differentially translated genes from step (b) are in the third translational profile and when the translational profile of the one or more genes in the third translational profile is closer to the translational profile of the one or more genes in the second translational profile than to the translational profile of the one or more genes in the first translational profile.

In some embodiments, the method of validating a target for therapeutic intervention in disease comprises:
(a) determining three independent translational profiles, each for a plurality of genes from a disease sample, wherein (i) a first translational profile is from a sample not contacted with any compound, (ii) a second translational profile is from a sample contacted with an agent that modulates a disease-associated target, and (iii) a third translational profile is from a sample contacted with a known active compound for treating the disease;
(b) determining a first differential translational profile comprising one or more genes differentially translated in the first translational profile as compared to the second translational profile, and determining a second differential translational profile comprising one or more genes differentially translated in the first translational profile as compared to the third translational profile; and
(c) validating the target as a target for therapeutic intervention in the disease when the first differential translational profile is comparable to the second differential translational profile.

In still another aspect, the present invention provides methods for validating a target for normalizing a translational profile associated with a disease. In some embodiments, the method comprises:
(a) determining a first translational profile for a plurality of genes from a disease sample that has been contacted with an agent that modulates a disease-associated target;
(b) determining a second translational profile for a plurality of genes from (i) a control non-diseased sample or (ii) a control non-diseased sample that has been contacted with the agent that modulates a disease-associated target; and
(c) validating the target as a target for normalizing a translational profile associated with the disease when the first translational profile is comparable to the second translational profile.

In some embodiments, the method for validating a target for normalizing a translational profile associated with a disease comprises:
(a) determining three independent translational profiles, each for a plurality of genes, wherein (i) a first translational profile is from a disease sample, (ii) a second translational profile is from (1) a control non-diseased sample or (2) a control non-diseased sample that has been contacted with an agent that modulates a disease-associated target, and (iii) a third translational profile is from a disease sample that has been contacted with the agent that modulates the disease-associated target;
(b) identifying one or more genes as differentially translated in the first translational profile as compared to the second translational profile; and
(c) validating the target as a target for normalizing a translational profile associated with the disease when the one or more differentially translated genes from step (b) are in the third translational profile and when the translational profile of the one or more genes in the third translational profile is closer to the translational profile of the one or more genes in the second translational profile than to the translational profile of the one or more genes in the first translational profile.

In some embodiments, the method for validating a target for normalizing a translational profile associated with a disease comprises:
(a) determining three independent translational profiles, each for a plurality of genes, wherein (i) a first translational profile is from a disease sample, (ii) a second translational profile is from (1) a control non-diseased sample or (2) a control non-diseased sample that has been contacted with an agent that modulates a disease-associated target, and (iii) a third translational profile is from a disease sample that has been contacted with the agent that modulates the disease-associated target;
(b) determining a first differential translational profile comprising one or more genes differentially translated in the first translational profile as compared to the second translational profile, and determining a second differential translational profile comprising one or more genes differentially translated in the first translational profile as compared to the third translational profile; and
(c) validating the target as a target for normalizing a translational profile associated with the disease when the first differential translational profile is comparable to the second differential translational profile.

In yet another aspect, the present invention provides methods of identifying a subject as a candidate for treating a disease with a therapeutic agent. In some embodiments, the method comprises:
(a) determining a first translational profile for a plurality of genes in a sample from a subject having or suspected of having a disease selected from a cancer, an inflammatory disease, an autoimmune disease, a neurodegenerative disease, a neurodevelopmental disease, a metabolic disease, and a viral infection;
(b) determining a second translational profile for a plurality of genes in a control sample, wherein the control sample is from a subject known to respond to the therapeutic agent and wherein the sample has not been contacted with the therapeutic agent; and
(c) identifying the subject as a candidate for treating the disease with the therapeutic agent when the first translational profile is comparable to the second translational profile.

In another aspect, the present invention provides methods for treating a disease (e.g., a cancer, an inflammatory disease, an autoimmune disease, a neurodegenerative disease, a neurodevelopmental disease, a metabolic disease, or a viral infection) comprising administering a therapeutic agent to a subject identified according to any of the methods described herein.

In still another aspect, the present invention provides methods for treating a disease (e.g., a cancer, an inflammatory disease, an autoimmune disease, a neurodegenerative disease, a neurodevelopmental disease, a metabolic disease, or a viral infection) comprising administering to a subject having the disease a therapeutic agent identified according to any of the methods described herein.

In still yet another aspect, the present invention provides methods for treating a disease (e.g., a cancer, an inflammatory disease, an autoimmune disease, a neurodegenerative disease, a neurodevelopmental disease, a metabolic disease, or a viral infection) comprising administering to a subject having the disease an agent that modulates a disease-associated target, wherein the target was validated according to any of the methods described herein.

In yet another aspect, the present invention provides methods for treating a disease (e.g., a cancer, an inflammatory disease, an autoimmune disease, a neurodegenerative disease, a neurodevelopmental disease, a metabolic disease, or a viral infection) by normalizing the disease translational profile, comprising administering to a subject having the disease a therapeutic agent identified according to any of the methods described herein.

### BRIEF DESCRIPTION OF THE DRAWINGS

FIG. 1. Ribosome profiling reveals mTOR-dependent specialized translational control of the prostate cancer genome. (a) Representative comparison of mRNA abundance and translational efficiency after a 3 hr treatment with an ATP site inhibitor (PP242) versus an allosteric inhibitor (rapamycin). (b-d) Free energy, length and percentage G+C content of the 5′ UTRs of mTOR target versus non-target mRNAs (error bars indicate range, non-target n=5,022, target n=144, two-sided Wilcoxon). (e) Functional classification of translationally regulated mTOR-responsive mRNAs. (f) Chemical structure of INK128. (g) Representative Western blot from three independent experiments of mTOR-sensitive invasion genes in PC3 cells after a 48-hr drug treatment. Rapa: rapamycin.

FIG. 2. mTOR promotes prostate cancer cell migration and invasion through a translationally regulated gene signature. (a) Matrigel invasion assay in PC3 cells: 6-hr pre-treatment followed by 6 hr of cell invasion (n=6, ANOVA). (b, c) Migration patterns and average distance traveled by GFP-labeled PC3 cells during hours 3-4 and 6-7 of drug treatment (n=34 cells per condition, ANOVA). (d) Matrigel invasion assay in PC3 cells after 48 hr of knockdown of YB1, MTA1, CD44, or vimentin followed by 24 hr of cell invasion (n=7, t-test). (e) Matrigel invasion assay in BPH-1 cells after 48 hr of overexpression of YB1 and/or MTA1, followed by cell invasion for 24 hr (n=7, t-test). Rapa: rapamycin. All data represent mean±s.e.m. NS: not statistically significant.

FIG. 3. The 4EBP1-eIF4E axis controls the post-transcriptional expression of mTOR-sensitive invasion genes. (a) Schematic of the pharmacogenetic strategy to inhibit p70S6K1/2 or eIF4E hyperactivation. (b) Representative Western blot from three independent experiments of PC3 4EBP1M cells after 48-hr doxycycline induction of 4EBP1^{M}. (c) Representative Western blot from three independent experiments of PC3 cells after 48-hr DG-2 treatment. (d) Representative Western blot from three independent experiments of PC3 cells after 48 h of 4EBP1/4EBP2 knockdown followed by 24-hr treatment with an ATP site inhibitor of mTOR (see quantification of independent experiments in FIG. 21a). (e) Representative Western blot from three independent experiments of wild-type (WT) and 4EBP1/4EBP2 double knockout (DKO) MEFs treated with an ATP site inhibitor of mTOR for 24 hr. (f) Representative Western blot from two independent experiments of wild-type and mSin1^{-/-} (also called Mapkap1^{tm1Bisu}) MEFs after 24-hr treatment with an ATP site inhibitor of mTOR. (g) Matrigel invasion assay upon 48-hr doxycycline induction of 4EBP1^{M}, or treatment with DG-2 compared to control (n=6 per condition, t-test). All data represent mean±s.e.m.

FIG. 4. mTOR hyperactivation augments translation of YB1, MTA1, CD44, and vimentin mRNAs in a subset of pre-invasive prostate cancer cells in vivo. Left: immunofluorescent images of CK8/DAPI or CK5/DAPI with YB1 (a, b), MTA1 (c, d), or CD44 (e, f) co-staining in 14-month-old wild-type and Pten^{L/L} mouse prostate epithelial cells. White boxes outline the area magnified in the right panel. Right: magnified immunofluorescent images of YB1 (a, b), MTA1 (c, d) and CD44 (e, f) co-stained with DAPI in wild-type and Pten^{L/L} mouse prostate epithelial cells. Dotted lines encircle the cytoplasm (C) and/or the nucleus (N). (g) Representative immunofluorescent images of CK5 or CK8 co-staining with vimentin in 14-month-old wild-type and Pten^{L/L} mouse prostate epithelial cells. S: stroma; yellow arrows indicate perinuclear vimentin. (h) Box plot of YB1 (N=nuclear, C=cytoplasmic), MTA1, and CD44 mean fluorescence intensity (m.f.i.) per CK5⁺ or CK8⁺ prostate epithelial cell in wild-type and Pten^{L/L} mice (three mice per arm, n=43-303 cells quantified per target gene, error bars indicate range (see FIG. 23b); *P<0.0001, **P=0.0004, t-test).

FIG. 5. Complete mTOR inhibition by treatment with an ATP site inhibitor of mTOR prevents prostate cancer invasion and metastasis in vivo. (a) Diagram and images of normal prostate gland, pre-invasive PIN, and invasive prostate cancer. CK8/CK5, luminal/basal epithelial cells, respectively. Yellow arrowheads indicate invasive front. (b) Immunofluorescent images of 14-month-old Pten^{L/L} lymph node (LN) metastasis co-stained with CK8/androgen receptor (AR), CK8/YB1, and CK8/MTA1. (c) Left: human tissue microarray of YB1 protein levels in normal (n=59), PIN (n=5), cancer (n=99), and CRPC (n=3) (ANOVA). Right: immunohistochemistry of YB1 in human CRPC demarcated by the red line (inset shows nuclear and cytoplasmic YB1). (d) Quantification of invasive prostate glands in wild-type and Pten^{L/L} mice before (12-months old) and after (14-months old) 60 days of treatment with an ATP site inhibitor of mTOR (n=6 mice per arm, ANOVA). (e, f) Area and number of CK8/AR+ metastases in draining lymph nodes in 14-month-old Pten^{L/L} mice after 60 days of treatment with an ATP site inhibitor of mTOR (n=6 mice per arm, t-test). (g) Percentage decrease of YB1 (N=nuclear, C=cytoplasmic), MTA1, CD44, or vimentin protein levels (determined by quantitative immunofluorescence, see FIG. 23b) in CK8⁺ or CK5⁺ prostate cells (CK8′ only for vimentin) in ATP site inhibitor of mTOR-treated 14-month-old Pten^{L/L} mice normalized to vehicle-treated mice (n=3 mice per arm, t-test). All data represent mean±s.e.m.

FIG. 6. Validation of mTOR inhibitors in PC3 prostate cancer cell line. (a) Schematic of ribosome profiling of human prostate cancer cells. (b) Representative Western blot analysis from 3 independent experiments of PC3 prostate cancer cells treated with rapamycin (50 nM), PP242 (2.5 μM), or ATP site inhibitor of mTOR (200 nM) for 3 hours. (c) Representative [³⁵S]-methionine incorporation in PC3 cells after 6-hour treatment with rapamycin (50 nM) or an ATP site inhibitor of mTOR (200 nM) (left panel). Quantification of [³⁵S]-methionine incorporation (right panel, n=4, mean+SEM). (d) Representative [³⁵S]-methionine incorporation in PC3 cells after 14-hour treatment with rapamycin (50 nM) or an ATP site inhibitor of mTOR (200 nM) (left panel). Quantification of [³⁵S]-methionine incorporation (right panel, n=4, mean+SEM, * P<0.05 ANOVA). (e) Cell cycle analysis of PC3 cells after treatment with rapamycin (50 nM), PP242 (2.5 μM), or an ATP site inhibitor of mTOR (200 nM) for 48 hours (mean+SEM, n=3, * P<0.001 ANOVA). (f) Cell cycle analysis of PC3 cells after 0-, 6-, or 24-hour treatment with an ATP site inhibitor of mTOR (200 nM) (mean+SEM, n=3, * P<0.001 ANOVA). n.s.: not statistically significant. V: vehicle; R: rapamycin; I: ATP site inhibitor of mTOR.

FIG. 7. Inter-experimental correlation of ribosome profiling per treatment condition and tally of mTOR responsive genes. (a) Correlation plots from 2 independent ribosome profiling experiments after a 3-hour treatment with rapamycin (50 nM) or PP242 (2.5 μM). (b) Number of translationally and transcriptionally regulated mRNA targets of mTOR after 3-hour drug treatments. (c) The Pyrimidine Rich Translational Element (PRTE) (SEQ ID NO:145) is present within the 5′ UTRs of 63% of mTOR-responsive translationally regulated mRNAs. (d) Venn diagram of the number of mTOR sensitive genes that possess a PRTE (red), 5′ TOP (green), or both (yellow).

FIG. 8. Read count profiles for eEF2, vimentin, SLC38A2, and PAICS. (a) Ribosome footprint and RNA-Seq profiles for eEF2. Read count profiles are shown for each nucleotide position in the uc0021ze.2 transcript, with the eEF2 coding sequence marked. Ribosome footprints were assigned to specific A site nucleotide positions based on their length. (b) Ribosome footprint and RNA-Seq profiles for vimentin. (c) Ribosome footprint and RNA-Seq profiles for SLC38A2. (d) Ribosome footprint and RNA-Seq profiles for PAICS.

FIG. 9. False Discovery Rate computation. (a) The cumulative distribution of log₂ fold-change values is shown for three comparisons, considering only genes passing the minimum read count criterion in that comparison. The DMSO replicate represents a comparison of full biological replicates of the control DMSO-only treatment condition. The rapamycin and PP242 conditions show the ratio of drug-treated to DMSO-treated samples within a single experiment. The fold-change threshold chosen based on PP242 translational repression, described below, is shown. (b) The extremes of the log₂ fold-change cumulative distributions, showing the complementary cumulative distribution function for positive extreme values on the right. The cumulative distribution of fold-change values between the DMSO replicates was used as an estimate of the error distribution for measurements in drug treatment comparisons. That is, the fraction of genes above a given absolute value fold-change level in the comparison of biological replicates should reflect the fraction of genes above that level by chance in any measurement. At a cutoff of log₂ fold-change of +/−1.5, we detect 2.5% (95% CI, 2.1%-2.9% by Agresti-Coull) of genes in the PP242/DMSO comparison and only 0.044% (95% CI, 0.001%-0.172%) of genes in the DMSO replicate comparison. The estimated false discovery rate is therefore q=0.018 in the PP242/DMSO comparison at this fold-change threshold.

FIG. 10. Transcriptionally regulated mTOR targets. (a and b) qPCR validation of up-regulated or down-regulated transcripts identified by RNA-Seq upon 3-hour PP242 treatment (2.5 μM) in PC3 cells (mean+SEM, n=3). (c) qPCR validation of up-regulated transcript identified by RNA-Seq upon 3-hour rapamycin treatment (50 nM) in PC3 cells (mean+SEM, n=3).

FIG. 11. mTOR-sensitive translationally regulated gene invasion signature. Mutation of the PRTE abrogates sensitivity to eIF4E. (a) 4 known pro-invasion genes and 7 putative pro-invasion genes discovered through ribosome profiling. (b) Schematic of YB1 5′ UTR cloning (WT, transversion mutant, and deletion mutant of the PRTE (position +20-34, uc001chs.2)) into pGL3-Promoter (left panel). Firefly luciferase activity in PC3-4EBP1^{M} cells after a 24-hour pre-treatment with 1 μg/ml doxycycline followed by transfection of respective 5′ UTR constructs (mean+SEM, n=7, * P<0.0001, t-test) (right panel). n.s.: not statistically significant.

FIG. 12. ATP site inhibition of mTOR does not reduce transcript levels of the 4 invasion genes. ATP site inhibitor of mTOR time course. (a) mRNA expression of YB1, MTA1, vimentin, and CD44, relative to β-actin upon treatment with rapamycin (50 nM), PP242 (2.5 μM), or an ATP site inhibitor of mTOR (200 nM) for 48 hours in PC3 cells (mean+SEM, n=3). (b) Representative Western blot of 3 independent experiments showing a time course of invasion gene expression before and after treatment with ATP site inhibitor of mTOR (200 nM) in PC3 cells.

FIG. 13. Polysome analysis after 3-hour ATP site inhibitor of mTOR treatment. (a) Ethidium bromide staining of rRNA species in individual fractions. Fractions 7-13 were determined to be polysome-associated fractions. (b) Overlay of polysome profiles from PC3 cells treated with vehicle (solid line) or ATP site inhibitor of mTOR (100 nM) (dotted line). (c) qPCR analysis of YB1 and rpS19 mRNAs that show differential association in polysome fractions after ATP site inhibitor of mTOR (100 nM) treatment (mean+SEM, n=6). The bottom graph shows that there is no change in β-actin mRNA association in polysome fractions between treatments. P-values (t-test) for each polysome fraction are shown. (d) Representative Western blot of 3 independent experiments showing a time course of eEF2 and rpL28 expression before and after treatment with ATP site inhibitor of mTOR (200 nM) in PC3 cells.

FIG. 14. 4-gene invasion signature is responsive to ATP site inhibitor of mTOR but not rapamycin in metastatic cell lines. (a-b) Representative Western blot (a) and qPCR analysis (b) of MDA-MB-361 cells after 48-hour treatment with ATP site inhibitor of mTOR (200 nM). (c-d) Representative Western blot (c) and qPCR analysis (d) of SKOV3 cells after 48-hour treatment with ATP site inhibitor of mTOR (200 nM). (e-f) Representative Western blot (e) and qPCR analysis (f) of ACHN cells after 48-hour treatment with ATP site inhibitor of mTOR (200 nM). Westerns=representative Western blot of 2 independent experiments. qPCR−n=3. All data represent mean+SEM.

FIG. 15. PTEN gene silencing in the A498 PTEN positive renal carcinoma cell line induces the post-transcriptional expression of the 4-gene invasion signature. (a-b) Representative Western blot (a) and qPCR analysis (b) of A498 cells after stable silencing of PTEN and 24 hour treatment with an ATP site inhibitor of mTOR (200 nM). Western=representative Western blot of 2 independent experiments. qPCR−n=3. All data represent mean±SEM.

FIG. 16. ATP site inhibitor of mTOR inhibits cell migration in PC3 prostate cancer cells as early as 6 hours after drug treatment. (a) Representative wound healing assay of 3 independent experiments in PC3 cells treated with rapamycin (50 nM) or ATP site inhibitor of mTOR (200 nM) for 40 hours. Inset (red box) represents wound at 0 hours. (b) Migration patterns of individual GFP-labeled PC3 cells during hours 3-4 after treatment with rapamycin or ATP site inhibitor of mTOR (34 cells per condition). (c) Average velocity of GFP-labeled PC3 cells during hours 3-4 or 6-7 after treatment with rapamycin (50 nM) or ATP site inhibitor of mTOR (200 nM) (mean+SEM, n=34 cells per condition, * P<0.001, ANOVA).

FIG. 17. Knockdown of the 4 invasion genes in PC3 prostate cancer cells. YB1, CD44, MTA1, and Vimentin protein levels after 48 hours of gene silencing in PC3 cells.

FIG. 18. YB1 knockdown and ATP site inhibition of mTOR decreases the protein levels but not mRNA levels of YB1 target genes. (a) Snail1 immunofluorescence in PC3 cells after 48 hours of YB1 gene silencing. Representative Snail1 immunofluorescence (top panels), box plot of Snail1 mean fluorescence intensity per cell (MFI)(n=26 siCtrl cells, n=15 siYB1 cells, * P=0.001, t-test) (bottom panel). (b) Snail1 immunofluorescence in PC3 cells after treatment with rapamycin (50 nM), PP242 (2.5 μM), or ATP site inhibitor of mTOR (200 nM). Representative Snail1 immunofluorescence (left panel), box plot of Snail1 mean fluorescence intensity per cell (MFI) (n=16 vehicle treated cells, n=26 rapamycin treated cells, n=28 PP242 treated cells, n=27 ATP site inhibitor of mTOR treated cells, * P<0.05, ANOVA) (right panel). (c) Representative Western blot (left panel) and quantification of protein levels (right panel) for LEF1 and Twist1 after YB1 gene silencing (mean+SEM, n=6, * P<0.05, t-test). (d) Representative Western blot (left panel) and quantification of protein levels (right panel) for LEF1 and Twist1 after ATP site inhibitor of mTOR treatment (mean+SEM, n=6, * P<0.005, t-test). (e-g) Snail1 (e), LEF1 (f), or Twist1 (g) mRNA expression normalized to β-actin after YB1 gene knockdown or treatment with rapamycin (50 nM), PP242 (2.5 μM) or ATP site inhibitor of mTOR (200 nM) in PC3 cells (mean+SEM, n=3).

FIG. 19. Effects of invasion gene knockdown or overexpression in PC3 and BPH-1 cells, respectively, on the cell cycle. (a) HA-YB1 and Flag-MTA1 protein levels after 48 hours of overexpression in non-transformed BPH-1 prostate epithelial cells (Y=YB1, M=MTA1). (b) Cell cycle analysis in PC3 cells after knockdown of respective genes (mean+SEM, n=3). (c) Cell cycle analysis upon overexpression of YB1 and/or MTA1 in BPH-1 cells (mean+SEM, n=3).

FIG. 20. The 4EBP1^{M} does not augment mTORC1 function or global protein synthesis in PC3 cells. (a) Representative Western blot from 3 independent experiments of phospho-p70S6K^{T389} and phospho-rpS6^{S240/244} after a 48-hour treatment with and without 1 μg/ml doxycycline in PC3-4EBP1^{M} cells. (b) Representative [³⁵S]-methionine incorporation from 2 independent experiments in PC3-4EBP1^{M} cells (48 hours, doxycycline 1 μg/mL) (mean+SEM). (c) Representative cap-binding assay from 2 independent experiments after 48-hour treatment with 1 μg/ml doxycycline in PC3-4EBP1^{M} cells. (d) mRNA expression of YB1, MTA1, Vimentin, and CD44 relative to β-actin after 48-hour treatment with 1 μg/ml doxycycline in PC3-4EBP1^{M} cells (mean+SEM, n=3).

FIG. 21. The 4EBP/eIF4E axis imparts sensitivity to mTOR ATP site inhibition. (a) Quantification of Western blots from 3 independent experiments of PC3 cells after 48 hours of 4EBP1/4EBP2 knockdown followed by 24-hour treatment with an ATP site inhibitor of mTOR (n=3, * p<0.05, ** p<0.01, ANOVA). (b) mRNA expression of YB1, MTA1, vimentin, and CD44 relative to β-actin after 48 hours of gene silencing of 4EBP1 and 4EBP2 followed by a 24-hour treatment with an ATP site inhibitor of mTOR (200 nM) (mean+SEM, n=3). (c) mRNA expression of YB1, MTA1, and CD44 in WT and 4EBP1/4EBP2 DKO MEFs treated with 200 nM ATP site inhibitor of mTOR for 24 hours (mean+SEM, n=3).

FIG. 22. mTORC2 does not control the expression of the 4-gene invasion signature. (a) mRNA expression of YB1, MTA1, and CD44 relative to β-actin after a 24-hour treatment with ATP site inhibitor of mTOR (200 nM) in mSin1^{-/-} MEFs (mean+SEM, n=3). (b) Representative Western blot analysis from 2 independent experiments of PC3 prostate cancer cells after 48 hours of rictor gene silencing followed by a 24-hour treatment with ATP site inhibitor of mTOR (200 nM). (c) mRNA expression of YB1, MTA1, vimentin, and CD44 relative to β-actin in PC3 prostate cancer cells after 48 hours of rictor gene silencing followed by a 24-hour treatment with ATP site inhibitor of mTOR (200 nM) in PC3 (mean+SEM, n=3). (d) Cell cycle analysis of PC3-4EBP1^{M} cells after treatment with 1 μg/ml doxycycline for 48 hours (mean+SEM, n=3).

FIG. 23. Complete mTOR inhibition decreases the expression of the 4-gene invasion signature at the level of translational control in vivo in PTEN^{L/L} mice. (a) Validation of antibodies used for immunofluorescence after 48-hour gene silencing of respective genes in PC3 cells. (b) Number of individual CK5⁺ and/or CK8⁺ cells measured in 3 separate mice for mean fluorescence intensity of respective protein targets in WT and PTEN^{L/L} mouse prostates. (c) mRNA expression of YB1, MTA1, vimentin, and CD44 relative to β-actin in WT and PTEN^{L/L} mice after 28 days of treatment with ATP site inhibitor of mTOR (1 mg/kg daily) (mean+SEM, n=3 mice per arm). (d) Representative Western blot of MTA1 from whole prostate tissue in WT and PTEN^{L/L} mice after 28 days of treatment with ATP site inhibitor of mTOR (1 mg/kg daily) (left panel) and quantitation relative to β-actin protein levels (right panel) (mean+SEM, n=3 mice per arm, * P=0.02, ** P=0.04, t-test) (e) Representative Western blot of YB1 from whole prostate tissue in WT and PTEN^{L/L} mice after 28 days of treatment with ATP site inhibitor of mTOR (1 mg/kg daily) (left panel) and quantitation relative to β-actin protein levels (right panel) (mean+SEM, n=4 mice per arm, * P=0.002, ** P=0.04, t-test) (f) Semi-quantitative RT-PCR of vimentin and β-actin for WT and PTEN^{L/L} FACS sorted murine prostate luminal epithelial cells (top panel). RT-PCR of a serial dilution of WT prostate luminal epithelial cell (bottom panel) (g) Z-series of perinuclear vimentin in a PTEN^{L/L} CK8⁺ prostate epithelial cell (red: vimentin; blue: DAPI; 0.4 μm per section; yellow arrows point to perinuclear vimentin).

FIG. 24. Preclinical efficacy of complete mTOR blockade in vivo. (a) Mouse weights measured every 3 days over the course of the preclinical trial (mean+SEM, n=3 mice per arm). (b) Representative phospho-specific immunohistochemistry of downstream mTOR targets in the ventral prostate (VP) of 9-month-old WT or PTEN^{L/L} mice after 28 days of treatment with ATP site inhibitor of mTOR (1 mg/kg daily) or RAD001 (10 mg/kg daily) (n=6 mice per treatment arm). Scale bar=100 μm. (c) Representative histology of 9-month-old WT or PTEN^{L/L} mice VP after 28 days of treatment with vehicle, RAD001 (10 mg/kg daily), or ATP site inhibitor of mTOR (1 mg/kg daily). Yellow dotted lines encircle prostate glands. Black triangles refer to prostatic secretions. Scale bar=50 μm. (d) Quantification of PIN+ glands in treated mice (mean+SEM, n=6 mice/arm, * P<0.001, ANOVA). (e) Proliferation measured by phospho-histone H3 positive glands in the prostates of 9-month-old WT or PTEN^{L/L} mice treated with RAD001 (10 mg/kg daily) or ATP site inhibitor of mTOR (1 mg/kg daily) (mean+SEM, n=3 mice per arm, * P<0.01, ANOVA). (f) Apoptosis measured by cleaved caspase 3 (CC3) positive cells in the prostates of 9-month-old WT or PTEN^{L/L} mice treated with RAD001 (10 mg/kg daily) or ATP site inhibitor of mTOR (1 mg/kg daily) (mean+SEM, n=3 mice per arm, * P<0.01, ANOVA) (left panel). Representative CC3 images (right panel). Scale bar=25 μm.

FIG. 25. An ATP site inhibitor of mTOR induces apoptosis in specific cancer cell lines and decreases primary prostate cancer volume in vivo. (a) Apoptosis in LNCaP (n=3) and A498 (n=2) cancer cells after treatment with rapamycin (50 nM), or an ATP site inhibitor of mTOR (200 nM) for 48 hours (mean+SEM, * P<0.001, ** P<0.05, ANOVA, n.s.=not statistically significant). (b) Percentage decrease in ventral and lateral prostate volume in 9-month-old PTEN^{L/L} after a 28-day treatment with vehicle or the ATP site inhibitor of mTOR (1 mg/kg daily) measured by MRI (left panel) (mean+SEM, n=4 mice per arm, * P=0.0008, t-test). Representative MRI images of the PTEN^{L/L} ventral and lateral prostate on day 0 and day 28 of treatment with the ATP site inhibitor of mTOR (right panel) (red dotted lines encircle the ventral and lateral prostate). (c) Additional images of prostate cancer invasion in the PTEN^{L/L} prostate (14-month-old mouse).

FIG. 26. Two ATP site inhibitors of mTOR mimic effect on translational profiles. These correlation plots provide a representative comparison of change in translational efficiency versus DMSO control by (a) the allosteric mTOR inhibitor rapamycin and the ATP site inhibitor PP242 in PC3 cells, (b) the two ATP site inhibitors INK128 and PP242, and (c) the MEK inhibitor GSK212 and mTOR ATP site inhibitor PP242 in SW620 cells. Each data point represents a single gene. In panels (a) and (b), data points highlighted in red have statistically significant changes in translational efficiency for PP242 versus DMSO control as described herein. In panel (c), the data points highlighted in red correspond to the 144 genes listed in Table 4 below. The allosteric inhibitor, rapamycin, affects the translational efficiency of similar genes affected by PP242, the magnitude of the rapamycin effect is substantially less than with PP242. In contrast, treatment with the two ATP site inhibitors (PP242 and INK128) alters the same gene set and at the same magnitude of change on a gene by gene basis. Finally, the MEK inhibitor GSK212 has little impact on the set of genes with translational efficiencies modulated by PP242.

FIG. 27. Effect of mTOR and MEK inhibitors on phosphorylation of protein translation components. (a) SW620 cells were treated with DMSO or the MEK inhibitor GSK212 (250 nM) for 8 hrs; (b) PC3 and (c) SW620 cells were treated with DMSO or the mTOR inhibitor PP242 for 3 hrs. Actin was used as a loading control.

FIG. 28. Induction of procollagen release from fibroblasts by TGF-β. Procollagen Type 1 levels (Procollagen Type 1C-Peptide, “PIPC”) after 24 hrs of treatment of fibroblasts with various concentrations of a PI3K/AKT/mTOR inhibitor (“PAMi”) and 10 ng/mL TGF-β. The difference in absorbance at 450 and 540 nm (y-axis) is proportional to the procollagen concentration.

FIG. 29. Western blot of protein phosphorylation levels during fibroblast transformation. Western blot analysis of fibroblast transformation as monitored by α-SMA levels after 24 hrs of treatment with various concentrations of a PAMi and 10 ng/mL TGF-β.

FIG. 30. Translational and transcriptional profile of fibroblasts treated with TGF-β. Comparison of changes in mRNA levels (RNA) and translational rate (RPF) in fibroblasts treated with TGF-β. Data points in red have p≦0.05 for changes in translational efficiency.

FIG. 31. Hepatic Fibrosis/Hepatic Stellate cell activation from IPA pathway analysis. (a) Early signaling events in hepatic stellate cells. (b) Signaling events in activated hepatic stellate cells. Gene list used and gene signature identified in analysis is based on p-value from differential concentrations of protein-coding mRNAs from control and TGF-β treated fibroblasts. Color coding is based on log₂ fold change.

FIG. 32. Hepatic Fibrosis/Hepatic Stellate cell activation from IPA pathway analysis. (a) Early signaling events in hepatic stellate cells. (b) Signaling events in activated hepatic stellate cells. Gene list used and gene signature identified in analysis is based on p-value from differential translation rates from control and TGF-β treated fibroblasts. Color coding is based on log₂ fold change.

FIG. 33. Normalization of translational efficiencies of fibrotic disorder-associated gene signature. The bar graph shows the translational efficiencies of fibrotic disorder-associated gene signature in fibroblasts treated with TGF-β and fibroblasts treated with TGF-β and a PAMi. The normal translational efficiency is set at zero and the p-value upon TGF-β treatment was ≦0.05 for these genes having an altered translational efficiency.

FIG. 34. Translational profile of genes associated with fibrotic disorder. The bar graph shows the translational efficiencies of all 141 fibrotic disorder-associated genes showing (a) a differential translational profile in transforming fibroblasts (treated with TGF-β) as compared to untreated (normal) fibroblasts, and (b) how treatment of transforming fibroblasts with a PAMi normalizes most genes (when compared to normal fibroblasts, set at zero). The p-value for change in translational efficiency upon TGF-β treatment was ≦0.05 for this gene signature.

FIG. 35. Translation levels of proteins associated with a neurodevelopmental disease model. Western blot analysis of the protein levels of FMRP, TSC2 and β-actin after siRNA knockdown of the FMRP gene. SH-SY5Y cells were transfected with either siControl or siFMR1 at 100 nM for 3 days.

FIG. 36. Ribosomal profile of a neurodevelopmental disease model. Comparison of changes in mRNA levels (RNA) and translational rate (RPF) in SH-SY5Y neuronal cells transfected with either a control siRNA or test siFMR1. Data points in red have p≦0.05 for changes in translational efficiency.

FIG. 37. Top up and down translationally regulated genes in a neurodevelopmental disease model. siRNA knockdown of the FMRP gene in SH-SY5Y cells with siFMR1 versus siCONTROL. The top 20 up- or down-differentially translationally regulated genes show a 60 or 45%, respectively, enrichment for association with neurological disease and development (p-value≦0.05).

FIG. 38. Effect of PI3K/AKT/mTOR inhibitors on TNF-α production during presence or absence of an induced inflammatory response. RAW264.7 macrophages were pre-treated with PI3K/Atk/mTOR inhibitor PAMi (10 μM) or without PAMi for 2 hrs followed by challenge with or without LPS 1 ng/ml for an additional 1 hr. Culture media was collected and TNF-α levels were quantified.

FIG. 39. Effect of MEK/ERK and PI3K/AKT/mTOR inhibitors on TNF-α production during an induced inflammatory response. RAW264.7 macrophages were pre-treated with a MEK/ERK pathway inhibitor (“MEi”) (16 nM or 4 nM) or PAMi (2.5 μM) for 2 hrs followed by challenge with LPS 1 ng/ml for an additional 1 hr. Culture media was collected and TNF-α levels quantified.

FIG. 40. Dose-dependent effect of PI3K/AKT/mTOR inhibitor on protein translation components in the presence or absence of induced inflammatory response. RAW264.7 macrophages were pre-treated with PAMi (10, 2.5, or 0.6 μM) or without PAMi for 2 hrs followed by challenge with or without LPS 1 ng/ml for an additional 1 hr. Culture media was collected and TNF-α levels quantified. Actin was used as a loading control.

FIG. 41. Effect of MEK inhibitor on various protein translation components during induced inflammatory response. Western blot analysis of RAW264.7 macrophages pre-treated with MEi (250, 62.5, 16, or 4 nM), for 2 hrs followed by challenge with LPS (1 ng/ml) for an additional hour. Actin was used as a loading control.

FIG. 42. Translational and transcriptional profile of macrophages treated with LPS. Comparison of changes in mRNA levels (RNA) and translational rate (RPF) in macrophages treated with LPS. Data points in red have p≦0.05 for changes in translational efficiency.

FIG. 43. Translational profile of genes from primary tissue. These correlation plots show the translational efficiencies of genes in a healthy (normal) section of prostate tissue (lower panel) and a section of cancer prostate tissue (upper panel) from the same patient.

FIG. 44. Translational efficiency of genes from primary tissue. This bar graph shows the number of translationally regulated (up and down) mRNA targets in healthy versus cancer prostate tissue.

### DETAILED DESCRIPTION OF THE INVENTION

### I. Introduction

The present invention relates to methods of characterizing potential therapeutic agents and validating therapeutic targets using translational profiles from a biological sample. In some embodiments, the methods of the present invention provide a genome-wide characterization of translationally controlled mRNAs downstream of biological pathways (e.g., oncogenic signaling pathways such as the mTOR pathway). The translational profiles that are generated can be used in identifying agents that modulate the biological pathway or in identifying or validating targets for therapeutic intervention.

### II. Definitions

As used herein, the term “translational profile” refers to the amount of protein that is translated (i.e., translational level) for each gene in a given set of genes in a biological sample, collectively representing a set of individual translational rate values, translational efficiency values, or both translational rate and translational efficiency values for each of one or more genes in a given set of genes. In some embodiments, a translational profile comprises translational levels for a plurality of genes in a biological sample (e.g., in a cell), e.g., for at least about 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000 genes or more, or for at least about 1%, 2%, 3%, 4%, 5%, 6%, 7%, 8%, 9%, 10%, 15%, 20%, 25% of all genes in the sample or more. In some embodiments, a translational profile comprises a genome-wide measurement of translational levels in a biological sample. In certain embodiments, a translational profile refers to a quantitative measure of the amount of mRNA associated with one or more ribosomes for each gene (i.e., translational rate, efficiency or both) in a given set of genes in a biological sample, wherein the amount of ribosome-associated mRNA correlates to the amount of protein that is translated (i.e., translational level).

As used herein, “translation rate” or “rate of translation” or “translational rate” refers to the total count of ribosome engagement, association or occupancy of mRNA for a particular gene as compared to the total count of ribosome engagement, association or occupancy of mRNA for at least one other gene or set of genes, wherein the count of total ribosomal occupancy correlates to the level of protein synthesis. Examination of translation rate across individual genes may be quantitative or qualitative, which will reveal differences in translation. In certain embodiments, translational rate provides a measure of protein synthesis for one or more genes, a plurality of genes, or across an entire genome. In particular embodiments, a translation rate is the amount of mRNA fragments protected by ribosomes for a particular gene relative to the amount of mRNA fragments protected by ribosomes for one or more other genes or groups of genes. For example, the mRNA fragments protected by ribosomes may correspond to a portion of the 5′-untranslated region, a portion of the coding region, a portion of a splice variant coding region, or combinations thereof. In further embodiments, the translation rate is a measure of one, a plurality or all mRNA variants of a particular gene. Translation rates can be established for one or more selected genes or groups of genes within a single composition (e.g., biological sample), between different compositions, or between a composition that has been split into at least two portions and each portion exposed to different conditions.

As used herein, “mRNA level” refers to the amount, abundance, or concentration of mRNA or portions thereof for a particular gene in a composition (e.g., biological sample). In certain embodiments, mRNA level refers to a count of one form, a plurality of forms or all forms of mRNA for a particular gene, including pre-mRNA, mature mRNA, or splice variants thereof. In particular embodiments, an mRNA level for one or more genes or groups of genes corresponds to counts of unique mRNA sequences or portions thereof for a particular gene that map to a 5′-untranslated region, a coding region, a splice variant coding region, or any combination thereof.

As used herein, “translation efficiency” or “translational efficiency” refers to the ratio of the translation rate for a particular gene to the mRNA level for a particular gene in a given set of genes. For example, gene X may produce an equal abundance of mRNA (i.e., same or similar mRNA level) in normal and diseased tissue, but the amount of protein X produced may be greater in diseased tissue as compared to normal tissue. In this situation, the message for gene X is more efficiently translated in diseased tissue than in normal tissue (i.e., an increased translation rate without an increase in mRNA level). In another example, gene Y may produce half the mRNA level in normal tissue as compared to diseased tissue, and the amount of protein Y produced in normal tissue is half the amount of protein Y produced in diseased tissue. In this second situation, the message for gene Y is translated equally efficiently in normal and diseased tissue (i.e., a change in translation rate in diseased tissue that is proportional to the increase in mRNA level and, therefore, the translational efficiency is unchanged). In other words, the expression of gene X is altered at the translational level, while gene Y is altered at the transcriptional level. In certain situations, both the amount of mRNA and protein may change such that mRNA abundance (transcription), translation rate, translation efficiency, or a combination thereof is altered relative to a particular reference or standard.

In certain embodiments, translational efficiency may be standardized by measuring a ratio of ribosome-associated mRNA read density (i.e., translation level) to mRNA abundance read density (i.e., transcription level) for a particular gene (see, Example 6 in the Examples section below). As used herein, “read density” is a measure of mRNA abundance and protein synthesis (e.g., ribosome profiling reads) for a particular gene, wherein at least 5, 10, 15, 20, 25, 50, 100, 150, 175, 200, 225, 250, 300 reads or more per unique mRNA or portion thereof is performed in relevant samples to obtain single-gene quantification for one or more treatment conditions. In certain embodiments, translational efficiency is scaled to standardize or normalize the translational efficiency of a median gene to 1.0 after excluding regulated genes (e.g., log₂ fold-change ±1.5 after normalizing for the all-gene median), which corrects for differences in the absolute number of sequencing reads obtained for different libraries. In further embodiments, changes in protein synthesis, mRNA abundance and translational efficiency are similarly computed as the ratio of read densities between different samples and normalized to give a median gene a ratio of 1.0, normalized to the mean, normalized to the mean or median of log values, or the like.

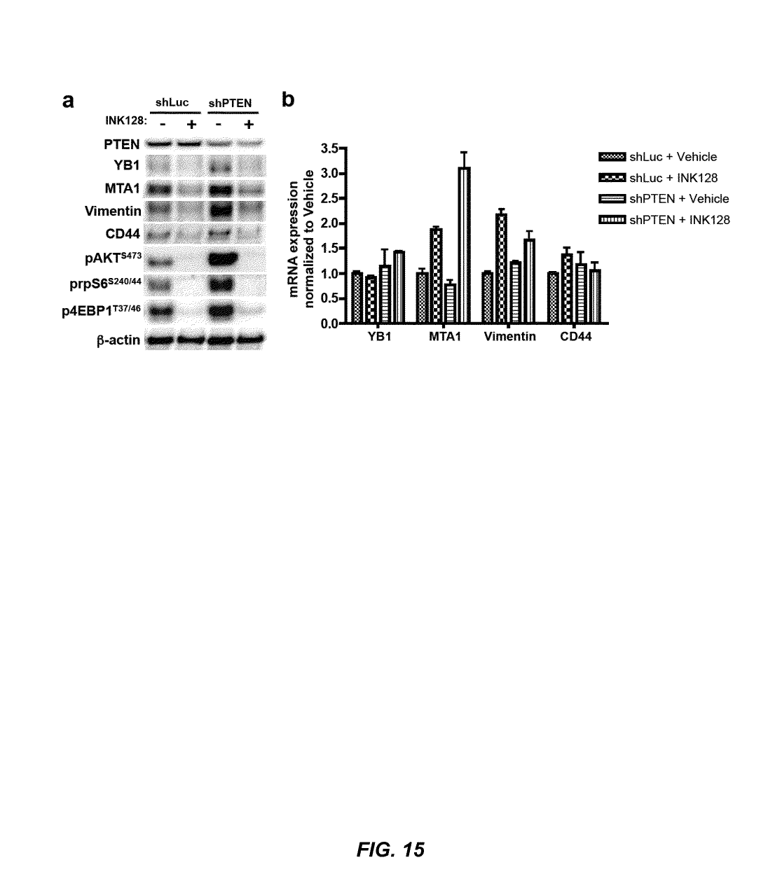

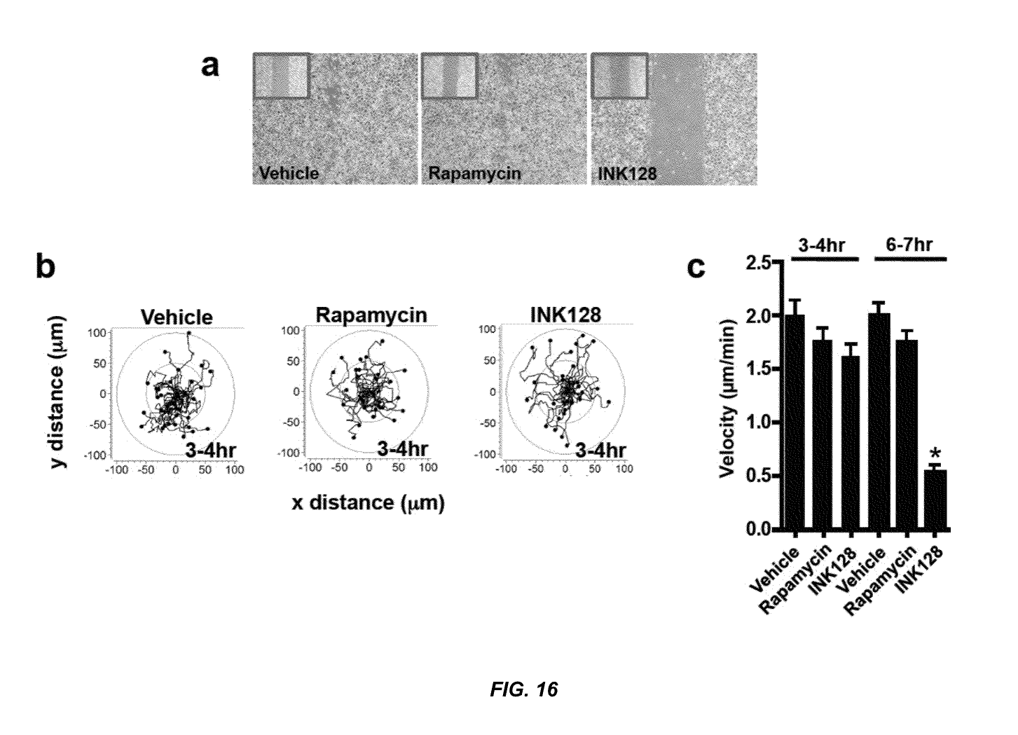

As used herein, “gene signature” or “gene cluster” refers to a plurality of genes that exhibit a generally coherent, systematic, coordinated, unified, collective, congruent, or signature expression pattern or translation efficiency. In certain embodiments, a gene signature is a plurality of genes that together comprise at least a detectable or identifiable portion of a biological pathway (e.g., 2, 3, 4, 5, or more genes; a cell invasion signature comprising 4 genes is illustrated in FIG. 15), comprise a complete set of genes associated with a biological pathway, or comprise a cluster or grouping of independent genes having a recognized pattern of expression (e.g., response to a known drug or active compound; related to a disease state such as a cancer, an inflammatory disease, an autoimmune disease, a fibrotic disorder, a neurodegenerative disease, a neurodevelopmental disease, a viral infection). One or more genes from a particular gene signature may be part of a different gene signature (e.g., a cell migration pathway may share a gene with a cell adhesion pathway)—that is, gene signatures may intersect or overlap but each signature can still be independently defined by its unique translation profile.

As used herein, the term “agent” refers to any molecule, either naturally occurring or synthetic, e.g., peptide, protein, oligopeptide (e.g., from about 5 to about 25 amino acids in length, preferably from about 10 to 20 or 12 to 18 amino acids in length, preferably 12, 15, or 18 amino acids in length), small organic molecule (e.g., an organic molecule having a molecular weight of less than about 2500 daltons, e.g., less than 2000, less than 1000, or less than 500 daltons), circular peptide, peptidomimetic, antibody, polysaccharide, lipid, fatty acid, inhibitory RNA (e.g., siRNA or shRNA), polynucleotide, oligonucleotide, aptamer, drug compound, or other compound.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2′-O-methyl ribonucleotides, and peptide-nucleic acids (PNAs).

Unless otherwise indicated, a particular nucleic acid sequence also implicitly encompasses conservatively modified variants thereof (e.g., degenerate codon substitutions), complementary sequences, splice variants, and nucleic acid sequences encoding truncated forms of proteins, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). The term nucleic acid is used interchangeably with gene, cDNA, mRNA, shRNA, siRNA, oligonucleotide, and polynucleotide.

The term “modulate” or “modulator,” as used with reference to modulating an activity of a target gene or signaling pathway, refers to increasing (e.g., activating, facilitating, enhancing, agonizing, sensitizing, potentiating, or upregulating) or decreasing (e.g., preventing, blocking, inactivating, delaying activation, desensitizing, antagonizing, attenuating, or downregulating) the activity of the target gene or signaling pathway. In certain embodiments, a modulator alters a translational profile at the translational level (i.e., increases or decreases translation rate or translation efficiency or both as described herein), at the transcriptional level, or both. In some embodiments, a modulator increases the activity of the target gene or signaling pathway, e.g., by at least about 1-fold, 1.5-fold, 2-fold, 2.5-fold, 3-fold, 3.5-fold, 4-fold, 4.5-fold, 5-fold, 6-fold, 7-fold, 8-fold, 9-fold, 10-fold, 15-fold, 20-fold or more. In some embodiments, a modulator decreases the activity of the target gene or signaling pathway, e.g., by at least about 1-fold, 1.5-fold, 2-fold, 2.5-fold, 3-fold, 3.5-fold, 4-fold, 4.5-fold, 5-fold, 6-fold, 7-fold, 8-fold, 9-fold, 10-fold, 15-fold, 20-fold or more.

A “biological sample” includes blood and blood fractions or products (e.g., serum, plasma, platelets, red blood cells, and the like); sputum or saliva; kidney, lung, liver, heart, brain, nervous tissue, thyroid, eye, skeletal muscle, cartilage, or bone tissue; cultured cells, e.g., primary cultures, explants, and transformed cells, stem cells, stool, urine, etc. Such biological samples (e.g., disease samples or normal samples) also include sections of tissues such as biopsy and autopsy samples, frozen sections taken for histologic purposes, and cells or other biological material used to model disease or to be representative of a pathogenic state (e.g., TGF-β treated fibroblasts as a model system for fibrosis; LPS treatment of cells as a model system for inflammation, etc.). A biological sample is typically obtained from a “subject,” i.e., a eukaryotic organism, most preferably a mammal such as a primate, e.g., chimpanzee or human; cow; dog; cat; a rodent, e.g., guinea pig, rat, or mouse; rabbit; or a bird; reptile; or fish.

As used herein, the terms “administer,” “administered,” or “administering” refer to methods of delivering agents or compositions to the desired site of biological action. These methods include, but are not limited to, topical delivery, parenteral delivery, intravenous delivery, transdermal delivery, intradermal delivery, transmucosal delivery, intramuscular delivery, oral delivery, nasal delivery, colonical delivery, rectal delivery, intrathecal delivery, ocular delivery, otic delivery, intestinal delivery, or intraperitoneal delivery. Administration techniques that are optionally employed with the agents and methods described herein, include e.g., as discussed in Goodman and Gilman, The Pharmacological Basis of Therapeutics, current ed.; Pergamon; and Remington's, Pharmaceutical Sciences (current edition), Mack Publishing Co., Easton, Pa.

As used herein, the term “normalize” or “normalizing” or “normalization” refers to adjusting the translational level (i.e., translational rate and/or translational efficiency) of one or more genes in a biological sample from a subject (e.g., a sample from a subject having a disease or condition) to a level that is more similar, closer to, or comparable to the translational level of those one or more genes in a control sample (e.g., a biological sample from a non-diseased tissue or subject). In certain embodiments, normalization refers to modulation of one or more translational regulators or translational system components to adjust or shift the translational efficiency of one or more genes in a biological sample (e.g., diseased, abnormal or other biologically altered condition) to a translational efficiency that is more similar, closer to or comparable to the translational efficiency of those one or more genes in a non-diseased or normal control sample. In some embodiments, normalization is evaluated by determining translational levels (i.e., translational rate and/or translational efficiency) of one or more genes in a biological sample from a subject (e.g., a sample from a subject having a disease or condition) before and after an agent (e.g., a therapeutic or known active agent) is administered to the subject and comparing the translational levels before and after administration to the translational levels from a control sample in the absence or presence of the agent. Exemplary methods of evaluating normalization of a translational profile associated with a disease or disorder includes identifying an agent, validating a target, or observing a shift in a gene signature. Further exemplary methods of normalization may be used for evaluating therapeutic intervention in a particular condition, disease or disorder.

As used herein, the term “undruggable target” refers to a gene, or a protein encoded by a gene, for which targeted therapy using a drug compound (e.g., a small molecule or antibody) does not successfully interfere with the biological function of the gene or protein encoded by the gene. Typically, an undruggable target is a protein that lacks a binding site for small molecules or for which binding of small molecules does not alter biological function (e.g., ribosomal proteins); a protein for which, despite having a small molecule binding site, successful targeting of said site has proven intractable in practice (e.g., GTP/GDP proteins); or a protein for which selectivity of small molecule binding has not been obtained due to close homology of the binding site with other proteins, and for which binding of the small molecule to these other proteins obviates the therapeutic benefit that is theoretically achievable with binding to the target protein (e.g., protein phosphatases). A target may be undruggable to antibody-based therapeutics for a variety of reasons, such as intracellular location of the target, masking of target antigenicity (e.g., due to modification with carbohydrate or other masking modifications) or to escape by competition (e.g., by shedding or release of decoy molecules).

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

Additional definitions are set forth throughout this disclosure.

### III. Translational Profiling

In one aspect, the present invention relates to the generation and analysis of translational profiles. A translational profile provides information about the identity of genes being translated in a biological sample (e.g., a cell) and/or the amount of protein that is translated (i.e., translational level in the form of translational rate, translational efficiency, or both) for each gene in a given set of genes in the biological sample, thereby providing information about the translational landscape in that biological sample. In certain embodiments, a translational profile is a biomarker, or comprises one or more biomarker genes, for a particular sample or condition.

In certain embodiments, a translational profile comprises one or more biologically meaningful groupings or clusters of genes, referred to as a “gene signature.” For example, a translational profile may comprise a plurality of gene signatures (e.g., 2, 3, 4, 5, 6, 7, 8, 9, 10, or more). In still further embodiments, a translational profile comprises one or more gene signatures in combination with one more additional gene not associated with or part of such gene signatures. In any of the aforementioned embodiments, particular genes, gene signatures, groups of genes, groups of gene signatures or any combination thereof comprise a biomarker. In certain embodiments, a translational profile comprises one or more gene signatures or gene clusters, wherein the one or more gene signatures or gene clusters individually or in a particular combination are a biomarker.

The expression pattern of one or more genes in one (e.g., a first) translational profile may be altered by an agent, compound, molecule, drug, or the like. In some cases, a test agent, compound, molecule, drug, or the like may mimic the action of an active compound known to have a particular function or induce a particular biological effect or phenotypic change in a cell or a subject. In certain embodiments, a test agent, compound, molecule, drug, or the like is identified as a mimic of a known active compound by causing a shift in the translational profile to be comparable or similar to the translational profile induced by the known active compound. In certain embodiments, a known active compound causes a translational profile to be more comparable or similar to normal. In other embodiments, a known active compound causes a translational profile to be more comparable or similar to a desired phenotype or effect, such as necrosis, apoptosis, or the like.

In some embodiments, a translational profile comprises translational levels for a plurality of genes in a biological sample, e.g., at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10,000 genes or more. In some embodiments, a translational profile comprises translational levels for one or more genes of one or more biological pathways in a biological sample (e.g., pathways such as protein synthesis, cell invasion/metastasis, cell division, apoptosis pathway, signal transduction, cellular transport, post-translational protein modification, DNA repair, and DNA methylation pathways). In some embodiments, a translational profile comprises translational levels for a subset of the genome, e.g., for about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50% of the genome or more. In some embodiments, a translational profile comprises a genome-wide measurement of translational levels.

A. Biological Samples

In some embodiments, a biological sample comprises a cell. In some embodiments, the cell is derived from a tissue or organ (e.g., prostate, breast, kidney, lung, liver, heart, brain, nervous tissue, thyroid, eye, skeletal muscle, cartilage, skin, or bone tissue). In some embodiments, the cell is derived from a biological fluid, e.g., blood (e.g., an erythrocyte), lymph (e.g., a monocyte, macrophage, neutrophil, eosinophil, basophil, mast cell, T cell, B cell, and/or NK cell), serum, urine, sweat, tears, or saliva. In some embodiments, the cell is derived from a biopsy (e.g., a skin biopsy, a muscle biopsy, a bone marrow biopsy, a liver biopsy, a gastrointestinal biopsy, a lung biopsy, a nervous system biopsy, or a lymph node biopsy). In some embodiments, the cell is derived from a cultured cell (e.g., a primary cell culture) or a cell line (e.g., PC3, HEK293T, NIH3T3, Jurkat, or Ramos). In some embodiments, the cell is a stem cell or is derived (e.g., differentiated) from a stem cell. In some embodiments, the cell is a cancer stem cell.

In some embodiments, the biological sample comprises a cancer cell (e.g., a cell obtained or derived from a tumor). In some embodiments, the cancer is prostate cancer, breast cancer, bladder cancer, urogenital cancer, lung cancer, renal cell carcinoma, endometrial cancer, melanoma, ovarian cancer, thyroid cancer, or brain cancer. In some embodiments, the cancer is a metastatic cancer.

In some embodiments, the biological sample is from a human subject. In some embodiments, the biological sample is from a non-human mammal (e.g., chimpanzee, dog, cat, pig, mouse, rat, sheep, goat, or horse), avian (e.g., pigeon, penguin, eagle, chicken, duck, or goose), reptile (e.g., snake, lizard, alligator, or turtle), amphibian (e.g., frog, toad, salamander, caecilian, or newt), or fish (e.g., shark, salmon, trout, or sturgeon).

B. Generating Translational Profiles

Various techniques for quantitating translational levels for a given set of genes and generating a translational profile are known in the art and can be used according to the methods of the present invention. These techniques include, but are not limited to, ribosomal profiling, polysome microarray, immunoassay, and mass spectrometry analysis, each of which is detailed below.

### Ribosomal Profiling

In some embodiments, one or more translational profiles are generated by ribosomal profiling. Ribosomal profiling provides a quantitative assessment of translational levels in a sample and can be used to measure translational levels on a genome-wide scale. Generally, ribosomal profiling identifies and/or measures the mRNA associated with ribosomes. Ribosome footprinting is used to measure the density of ribosome occupancy on a given mRNA and to identify the position of active ribosomes on mRNA. Using nuclease digestion, the ribosome position and translated message can be determined by analyzing the approximately 30-nucleotide region that is protected by the ribosome. In some embodiments, ribosome-protected mRNA fragments are analyzed and quantitated by a high-throughput sequencing method. For example, in some embodiments the protected fragments are analyzed by microarray. In some embodiments, the protected fragments are analyzed by deep sequencing; see, e.g., Bentley et al., Nature 456:53-59 (2008). Ribosomal profiling is described, for example, in US 2010/0120625; Ingolia et al., Science 324:218-223 (2009); and Ingolia et al., Nat Protoc 7:1534-1550 (2012); each of which is incorporated herein by reference in its entirety.

Ribosome profiling can comprise methods for detecting a plurality of RNA molecules that are bound by at least one ribosome, wherein the plurality of RNA molecules are associated with ribosomes. In some embodiments, the ribosome profile is of a group of ribosomes, for instance from a polysome. In some embodiments, the ribosome profile is from a group of ribosomes from the same cell or population of cells. For example, in some embodiments, a ribosome profile of a tumor sample can be determined.

In some embodiments, the ribosomal profiling comprises detecting a plurality of RNA molecules bound to at least one ribosome, by (a) contacting the plurality of RNA molecules with an enzymatic degradant or a chemical degradant, thereby forming a plurality of RNA fragments, wherein each RNA fragment comprises an RNA portion protected from the enzymatic degradant or the chemical degradant by a ribosome to which the RNA portion is bound; (b) amplifying the RNA fragments to form a detectable number of amplified nucleic acid fragments; and (c) detecting the detectable number of amplified nucleic acid fragments, thereby detecting the plurality of RNA molecules bound to at least one ribosome.

In some embodiments, nucleic acid fragments (e.g., mRNA fragments) are detected and/or analyzed by deep sequencing. Deep sequencing enables the simultaneous sequencing of multiple fragments, e.g., simultaneous sequencing of at least 500, 1000, 1500, 2000 fragments or more. In a typical deep sequencing protocol, nucleic acids (e.g., mRNA fragments) are attached to the surface of a reaction platform (e.g., flow cell, microarray, and the like). The attached DNA molecules may be amplified in situ and used as templates for synthetic sequencing (i.e., sequencing by synthesis) using a detectable label (e.g., a fluorescent reversible terminator deoxyribonucleotide). Representative reversible terminator deoxyribonucleotides may include 3′-O-azidomethyl-2′-deoxynucleoside triphosphates of adenine, cytosine, guanine and thymine, each labeled with a different recognizable and removable fluorophore, optionally attached via a linker. Where fluorescent tags are employed, after each cycle of incorporation, the identity of the inserted bases may be determined by excitation (e.g., laser-induced excitation) of the fluorophores and imaging of the resulting immobilized growing duplex nucleic acid. The fluorophore, and optionally linker, may be removed by methods known in the art, thereby regenerating a 3′ hydroxyl group ready for the next cycle of nucleotide addition. In some embodiments, the ribsome-protected mRNA fragments are detected and/or analyzed by a sequencing method described in US 2010/0120625, incorporated herein by reference in its entirety.

### Polysome Microarray

In some embodiments, one or more translational profiles are generated by polysome microarray. In a polysome microarray, mRNA is isolated and separated based on the number of associated ribosomes. Fractions of mRNA associated with several ribosomes are pooled to form a translationally active pool and are compared to cytosolic mRNA levels. Polysome microarray methods are described, for example, in Melamed and Arava, Methods in Enzymology, 431:177-201 (2007); and Larsson and Nadon, Biotech and Genet Eng Rev, 25:77-92 (2008); each of which is incorporated herein by reference in its entirety.

In some embodiments, polysome fractions having mRNA associated with multiple ribosomes (e.g., 3, 4, 5, 10 or more ribosomes) are pooled from a biological sample and RNA is isolated and labeled. The RNA samples from the translationally active pool are hybridized to a microarray with a control RNA sample (e.g., an unfractionated RNA sample). Ratios of polysome-to-free RNA are generated for each gene in the microarray to determine the relative levels of ribosomal association for each of the genes.

### Immunoassay

In some embodiments, one or more translational profiles are generated by immunoassay. Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. A variety of immunoassay techniques, including competitive and non-competitive immunoassays, can be used. See, e.g., Self et al., Curr. Opin. Biotechnol., 7:60-65 (1996). The term immunoassay encompasses techniques including, without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence. See, e.g., Schmalzing et al., Electrophoresis, 18:2184-93 (1997); Bao, J. Chromatogr. B. Biomed. Sci., 699:463-80 (1997).

A detectable moiety can be used in the assays described herein. A wide variety of detectable moieties can be used, with the choice of label depending on the sensitivity required, ease of conjugation with the antibody, stability requirements, and available instrumentation and disposal provisions. Suitable detectable moieties include, but are not limited to, radionuclides, fluorescent dyes (e.g., fluorescein, fluorescein isothiocyanate (FITC), Oregon Green™, rhodamine, Texas red, tetrarhodimine isothiocynate (TRITC), Cy3, Cy5, etc.), fluorescent markers (e.g., green fluorescent protein (GFP), phycoerythrin, etc.), autoquenched fluorescent compounds that are activated by tumor-associated proteases, enzymes (e.g., luciferase, horseradish peroxidase, alkaline phosphatase, etc.), nanoparticles, biotin, digoxigenin, and the like.

Useful physical formats comprise surfaces having a plurality of discrete, addressable locations for the detection of a plurality of different sequences. Such formats include microarrays and certain capillary devices. See, e.g., Ng et al., J. Cell Mol. Med., 6:329-340 (2002); U.S. Pat. No. 6,019,944. In these embodiments, each discrete surface location may comprise antibodies to immobilize one or more sequences for detection at each location. Surfaces may alternatively comprise one or more discrete particles (e.g., microparticles or nanoparticles) immobilized at discrete locations of a surface, where the microparticles comprise antibodies to immobilize one or more sequences for detection. Other useful physical formats include sticks, wells, sponges, and the like.

Analysis can be carried out in a variety of physical formats. For example, the use of microtiter plates or automation could be used to facilitate the processing of large numbers of samples (e.g., for determining the translational levels of 100, 500, 1000, 5000, 10,000 genes or more).

### Mass Spectrometry Analysis

In some embodiments, one or more translational profiles are generated by mass spectrometry analysis. Mass spectrometry (“MS”) generally involves the ionization of the analyte (e.g., a translated protein or portion thereof) to generate a charged analyte and measuring the mass-to-charge ratios of said analyte. During the procedure the sample containing the analyte is loaded onto a MS instrument and undergoes vaporization. The components of the sample are then ionized by one of a variety of methods.

As a non-limiting example, during Electrospray-MS (ESI) the analyte is initially dissolved in liquid aerosol droplets. Under the influence of high electromagnetic fields and elevated temperature and/or application of a drying gas the droplets get charged and the liquid matrix evaporates. After all liquid matrix is evaporated the charges remain localized at the analyte molecules that are transferred into the Mass Spectrometer. In matrix assisted laser desorption ionization (MALDI) a mixture of analyte and matrix is irradiated by a laser beam. This results in localized ionization of the matrix material and desorption of analyte and matrix. The ionization of the analyte is believed to happen by charge transfer from the matrix material in the gas phase. For a detailed description of ESI and MALDI, see, e.g., Mano N et al. Anal. Sciences 19 (1) (2003) 3-14. For a description of desorption electrospray ionization (DESI), see Takats Z et al. Science 306 (5695) (2004) 471-473. See also Karas, M.; Hillencamp, F. Anal. Chem. 60:2301 1988); Beavis, R. C. Org. Mass Spec. 27:653 (1992); and Creel, H. S. Trends Poly. Sci. 1(11):336 (1993).

Ionized sample components are then separated according to their mass-to-charge ratio in a mass analyzer. Examples of different mass analyzers used in LC/MS include, but are not limited to, single quadrupole, triple quadrupole, ion trap, TOF (time of Flight) and quadrupole-time of flight (Q-TOF).

The use of MS for analyzing proteins is also described, for example, in Mann et al., Annu. Rev. Biochem. 70:437-73 (2001).

C. Differential Translational Profiling

The expression pattern of one or more genes, gene signatures or combinations thereof from a (e.g., first) translational profile may differ from the expression pattern observed in one or more genes, gene signatures or combinations thereof from one or more different (e.g., second, third, etc.) translational profiles. In such situations, the one or more genes, gene signatures or combinations thereof showing different expression patterns between profiles are considered to be differentially translated. As used herein, the phrase “differentially translated” refers to the change or difference (e.g., increase, decrease or a combination thereof) in translation rate, translation efficiency, or both of one gene, a plurality of genes, a set of genes of interest (referred to as “gene markers” or “gene marker set”), one or more gene clusters, or one or more gene signatures under a particular condition as compared to the translation rate, translation efficiency, or both of the same gene, plurality of genes, set of gene markers, gene clusters, or gene signatures under a different condition, which is observed as a difference in expression pattern. For example, a translational profile of a diseased cell may reveal that one or more genes have higher translation rates and/or efficiencies (e.g., higher ribosome engagement of mRNA or higher protein abundance) than observed in a normal cell. In some embodiments, one or more gene signatures, gene clusters or sets of gene markers are differentially translated in a first translational profile as compared to one or more other translational profiles. In further embodiments, one or more genes, gene signatures, gene clusters or sets of gene markers in a first translational profile show at least a two-fold translation differential or at least a 1.1 log₂ change (i.e., increase or decrease) as compared to the same one or more genes in at least one other different (e.g., second, third, etc.) translational profile.

In some embodiments, two or more translational profiles are generated and compared to each other to determine the differences (i.e., increases and/or decreases in translational levels, such as translational rate and/or translational efficiency) for each gene in a given set of genes between the two or more translational profiles. The comparison between the two or more translational profiles is referred to as the “differential translational profile.” In certain embodiments, a differential translational profile comprises one or more genes, gene signatures (e.g., a biological or disease-associated pathway), or combinations thereof. In certain other embodiments, a differential translational profile comprises one or more clusters or groupings of independent genes having a recognized pattern of expression, such as an oncogenic signaling pathway, inflammatory disease-associated pathway, autoimmune disease-associated pathway, neurodegenerative disease-associated pathway, neurocognitive function disorder-associated pathway, fibrotic disorder-associated pathway, metabolic disease-associated pattern, or the like.

In some embodiments, methods are provided for identifying a gene signature associated with a disease. In some embodiments, the method comprises:
(a) determining a first translational profile for a plurality of genes from a disease sample;
(b) determining a second translational profile for a plurality of genes from a control non-diseased (e.g., normal) sample;
(c) identifying a differential translational profile between the first and second translational profiles; and
(d) identifying one or more gene signatures associated with a disease when the disease sample contacted with a known therapeutic has a translational profile for certain genes of a gene cluster or one or more biological pathways found in the differential translational profile that are closer to the translational profile of the same genes in the second translational profile.

The translational profiles that are generated for identifying a gene signature associated with a disease can be generated according to any of the methods described herein. In some embodiments, translational profiles are generated by ribosomal profiling, polysome microarray, immunoassay, or combinations thereof. In certain embodiments, translational profiles are generated by ribosomal profiling. In some embodiments, the disease sample is from a subject having or suspected of having a cancer, an inflammatory disease, an autoimmune disease, a fibrotic disorder, a neurodegenerative disease, a neurodevelopmental disease, a metabolic disease, a viral infection, or cardiomyopathy.

In some embodiments, a differential translational profile compares a first translational profile comprising gene translational levels for an experimental biological sample or subject, wherein the experimental biological sample or subject has been contacted with an agent as described herein (e.g., a peptide, protein, RNA, drug molecule, or small organic molecule) with a second translational profile comprising gene translational levels for a control biological sample or subject, e.g., a corresponding biological sample or subject of the same type that has not been contacted with the agent.

In some embodiments, a differential translational profile compares a first translational profile comprising gene translational levels for an experimental biological sample, wherein the experimental biological sample is from a subject having an unknown disease state (e.g., a cancer) or an unknown responsiveness to a therapeutic agent, with a second translational profile comprising gene translational levels for a control biological sample, e.g., a biological sample from a subject known to be positive for a disease state (e.g., a cancer) or from a subject that is a known responder to the therapeutic agent or from a non-diseased subject or tissue.

In some embodiments, differential profiles are generated for each of the first and second translational profiles, e.g., to compare the differences in translational levels for one or more genes in the presence or absence of a condition, or before and after administration of an agent, for the first translational profile (e.g., a translational profile from an experimental subject or sample) as compared to the second translational profile (e.g., a translational profile from a control subject or sample). For example, in some embodiments, differential profiles are generated for an experimental subject or sample (e.g., a subject having a cancer) before and after administration of a therapeutic agent and for a control subject or sample (e.g., a subject that is a known responder to the therapeutic agent, or a non-diseased (normal) subject or sample) before and after administration of the therapeutic agent. The first differential profile for the first translational profile (from the experimental subject or sample) is compared to the second differential profile for the second translational profile (from the control subject or sample) to determine the similarities in translational levels of one or more genes for the first differential profile as compared to the second differential profile. Based on the similarities between the differential profiles (e.g., whether the differential profiles are highly similar or comparable, or whether the translational level for one or more genes in the first differential profile is about the same as the translational level for the one or more genes in the second differential profile), it can be determined whether or not the experimental subject or control is likely to respond to the therapeutic agent.

In certain embodiments, differential translation between genes or translational profiles may involve or result in a biological (e.g., phenotypic, physiological, clinical, therapeutic, prophylactic) benefit. For example, when identifying a therapeutic, validating a target, or treating a subject, a “biological benefit” means that the effect on translation rate and/or translation efficiency or on the translation rate and/or translation efficiency of one or more genes of a translational profile allows for intervention or management of a disease, disorder, or condition of a subject (e.g., a human or non-human mammal, such as a primate, horse, dog, mouse, rat). In general, one or more differential translations or differential translation profiles indicate that a “biological benefit” will be in the form, for example, of an improved clinical outcome; lessening or alleviation of symptoms associated with disease; decreased occurrence of symptoms; improved quality of life; longer disease-free status; diminishment of extent of disease; stabilization of a disease state; delay of disease progression; remission; survival; or prolonging survival. In certain embodiments, a biological benefit comprises normalization of a differential translation profile, or comprises a shift in translational profile to one closer to or comparable to a translational profile induced by a known active compound or therapeutic, or comprises inducing, stimulating or promoting a desired phenotype or outcome (e.g., apoptosis, necrosis, cytotoxicity), or reducing, inhibiting or preventing an undesired phenotype or outcome (e.g., proliferation, migration).

### IV. Methods of Identifying Agents that Modulate Translation

In one aspect, the present invention relates to methods of identifying an agent that modulates translation in a biological pathway (e.g., an oncogenic signaling pathway) in a biological sample. In some embodiments, the present invention relates to methods of identifying an agent that inhibits, antagonizes, or downregulates translation in a biological pathway (e.g., an oncogenic signaling pathway) or disease. In some embodiments, the present invention relates to methods of identifying an agent that modulates, i.e., potentiates, agonizes, inhibits, or upregulates, translation in a biological pathway (e.g., an oncogenic signaling pathway) or disease.

A. Translational Profiles for Identifying Agents that Modulate Translation

In some embodiments, a method for identifying an agent that modulates translation in a disease comprises:
(a) determining a first translational profile for a plurality of genes from a disease sample contacted with a candidate agent;
(b) determining a second translational profile for a plurality of genes from a control disease sample not contacted with the agent; and
(c) identifying the agent as a modulator of translation in a disease when one or more genes, one or more gene signatures or combinations thereof are differentially translated in the first translational profile as compared to the second translational profile and when the differential translation results in a biological benefit.

The translational profiles that are generated for identifying an agent that modulates translation in a disease can be generated according to any of the methods described herein. In some embodiments, translational profiles are generated by ribosomal profiling, polysome microarray, immunoassay, or combinations thereof. In certain embodiments, translational profiles are generated by ribosomal profiling.

In some embodiments, translational profiles comprise translational efficiencies, translational rates, or a combination thereof for at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10,000 genes or more in the biological sample. In some embodiments, a first or second translational profile or both comprise translational efficiencies, translational rates, or combinations thereof for at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50% or more of all genes in a biological sample. In some embodiments, translational profiles comprise genome-wide measurements of gene translational levels.

In some embodiments, an agent that modulates translation in a disease is identified as suitable for use when one or more genes of one or more biological pathways, gene signatures or combinations thereof are differentially translated by at least 1.5-fold or at least 2-fold (e.g., at least 1.5-fold, at least 2-fold, at least 2.5-fold, at least 3-fold, at least 3.5-fold, at least 4-fold, at least 4.5-fold, at least 5-fold, at least 6-fold, at least 7-fold, at least 8-fold, at least 9-fold, at least 10-fold or more) in the first translational profile (e.g., treated disease sample) as compared to the second translational profile (e.g., untreated disease sample). In some embodiments, an agent that modulates translation in a disease is identified as suitable for use when the translational rate, translational efficiency or both for one or more genes of one or more biological pathways, gene signatures or combinations thereof are decreased by at least 1.5-fold or at least 2-fold (e.g., at least 1.5-fold, at least 2-fold, at least 2.5-fold, at least 3-fold, at least 3.5-fold, at least 4-fold, at least 4.5-fold, at least 5-fold, at least 6-fold, at least 7-fold, at least 8-fold, at least 9-fold, at least 10-fold or more) in the first translational profile as compared to the second translational profile. In some embodiments, an agent that modulates translation in a disease is identified as suitable for use when the translational rate, translational efficiency or both for one or more genes of one or more biological pathways, gene signatures or combinations thereof are increased by at least 1.5-fold or at least 2-fold (e.g., at least 1.5-fold, at least 2-fold, at least 2.5-fold, at least 3-fold, at least 3.5-fold, at least 4-fold, at least 4.5-fold, at least 5-fold, at least 6-fold, at least 7-fold, at least 8-fold, at least 9-fold, at least 10-fold or more) in the first translational profile as to the second translational profile.

In some embodiments, less than about 20% of the genes in the genome are differentially translated by at least 1.5-fold or at least 2-fold in the first translational profile as compared to the second translational profile. In some embodiments, less than about 5% of the genes in the genome are differentially translated by at least 1.5-fold or at least 2-fold in the first translational profile as compared to the second translational profile. In some embodiments, less than about 1% of the genes in the genome are differentially translated by at least 1.5-fold, at least 2-fold, at least 3-fold, at least 4-fold, or at least 5-fold in the first translational profile as compared to the second translational profile. In some embodiments, less than 1% of the genes in the genome are differentially translated by at least 3-fold in the first translational profile as compared to the second translational profile. In some embodiments, less than 1% of the genes in the genome are differentially translated by at least 5-fold in the first translational profile as compared to the second translational profile.

In some embodiments, the differentially translated genes comprise one or more biological pathways, such as at least two or at least three biological pathways. In certain embodiments, the one or more differentially translated genes comprise a plurality of genes and optionally the plurality of differentially translated genes may comprise one or more gene signatures. In further embodiments, the one or more genes are differentially translated at least a two-fold or more. In still further embodiments, each translational profile comprises at least 100 genes, at least 200 genes, at least 300 genes, at least 400 genes, at least 500 genes, or each translational profile comprises a genome-wide translational profile. For example, less than about 25%, about 20%, about 15%, about 10%, about 5%, about 4%, about 3%, about 2% or about 1% of the genes in the genome are differentially translated in a translational profile from a disease sample treated with a candidate agent as compared to a translational profile of an untreated disease sample.

A disease sample may be obtained from any subject having a disease of interest to identify agents that affect translational profiles in such samples. In certain embodiments, the subject has or is suspected of having a disease, such as a cancer, an inflammatory disease, an autoimmune disease, a fibrotic disorder, a neurodegenerative disease, a neurodevelopmental disease, a metabolic disease, a viral infection, or cardiomyopathy.

B. Translational Profiles for Identifying Agents that Modulate an Oncogenic Signaling Pathway

In some embodiments, the method of identifying an agent that modulates an oncogenic signaling pathway comprises:
(a) contacting the biological sample with an agent;
(b) determining a first translational profile for the contacted biological sample, wherein the translational profile comprises translational levels for one or more genes having a 5′ terminal oligopyrimidine tract (5′ TOP) and/or a pyrimidine-rich translational element (PRTE); and
(c) comparing the first translational profile to a second translational profile comprising translational levels for the one or more genes in a control sample that has not been contacted with the agent;
wherein a difference in the translational levels of the one or more genes in the first translation profile as compared to the second translation profile identifies the agent as a modulator of the oncogenic signaling pathway.

In some embodiments, a gene that has a different translational level in the first translational profile as compared to the second translational profile is a gene having a 5′ terminal oligopyrimidine tract (5′ TOP) sequence. A 5′ TOP sequence is a sequence that occurs in the 5′ untranslated region (5′ UTR) of mRNA. This element is comprised of a cytidine residue at the cap site followed by an uninterrupted stretch of up to 13 pyrimidines. Non-limiting examples of genes having a 5′ TOP sequence are shown in Table 1 below. In some embodiments, translational levels are compared for the first translational profile and the second translational profile for 1, 2, 3, 4, 5, 6, 7, 8, 9, 10, 11, 12, 13, 14, 15, 16, 17, 18, 19, 20 or more genes selected from the genes listed in Table 1.

TABLE 1
Translationally regulated mTOR-responsive genes having a 5′ TOP sequence

| Gene | Description | SEQ ID NO |
|---|---|---|
| AP2A1 | adaptor-related protein complex 2, alpha 1 subunit | 92 |
| CCNI | cyclin I | 96 |
| CD44 | CD44 antigen | 123 |
| CHP | calcineurin-like EF hand protein 1 | 116 |
| CRTAP | cartilage associated protein | 31 |
| EEF1A2 | eukaryotic translation elongation factor 1, alpha 2 | 45 |
| EEF1B2 | eukaryotic translation elongation factor 1, beta 2 | 129 |
| EEF1G | eukaryotic translation elongation factor 1, gamma | 34 |
| EEF2 | eukaryotic translation elongation factor 2 | 1 |
| EIF4B | eukaryotic translation initiation factor 4B | 37 |
| GAPDH | glyceraldehyde-3-phosphate dehydrogenase | 58 |
| GNB2L1 | guanine nucleotide binding protein (G protein), beta | 22 |
| | polypeptide 2-like 1 | |
| HNRNPA1 | heterogeneous nuclear ribonucleoprotein A1 | 56 |
| HSPA8 | heat shock 70 kDa protein 8 | 42 |
| IPO7 | importin 7 | 109 |
| LCMT1 | leucine carboxyl methyltransferase 1 | 107 |
| NAP1L1 | nucleosome assembly protein 1-like 1 | 93 |
| PABPC1 | poly(A) binding protein, cytoplasmic 1 | 17 |
| PACS1 | phosphofurin acidic cluster sorting protein 1 | 117 |
| PGM1 | phosphoglucomutase 1 | 121 |
| RABGGTB | Rab geranylgeranyltransferase, beta subunit | 139 |
| RPL10 | ribosomal protein L10 | 13 |
| RPL12 | ribosomal protein L12 | 3 |
| RPL13 | ribosomal protein L13 | 70 |
| RPL14 | ribosomal protein L14 | 53 |
| RPL15 | ribosomal protein L15 | 126 |
| RPL17 | ribosomal protein L17 | 79 |
| RPL22 | ribosomal protein L22 | 91 |
| RPL22L1 | ribosomal protein L22 L1 | 35 |
| RPL23 | ribosomal protein L23 | 74 |
| RPL29 | ribosomal protein L29 | 60 |
| RPL31 | ribosomal protein L31 isoform 2 | 49 |
| RPL32 | ribosomal protein L32 | 33 |
| RPL34 | ribosomal protein L34 | 11 |
| RPL36 | ribosomal protein L36 | 63 |
| RPL36A | ribosomal protein L36A | 66 |
| RPL37 | ribosomal protein L37 | 54 |
| RPL37A | ribosomal protein L37A | 18 |
| RPL39 | ribosomal protein L39 | 43 |
| RPL4 | ribosomal protein L4 | 104 |
| RPL41 | ribosomal protein L41 | 113 |
| RPL5 | ribosomal protein L5 | 86 |
| RPL6 | ribosomal protein L6 | 89 |
| RPL8 | ribosomal protein L8 | 59 |
| RPLP0 | ribosomal protein, large, P0 | 28 |
| RPLP2 | ribosomal protein, large, P2 | 38 |
| RPS10 | ribosomal protein S10 | 77 |
| RPS11 | ribosomal protein S11 | 51 |
| RPS14 | ribosomal protein S14 | 94 |
| RPS15A | ribosomal protein S15A | 21 |
| RPS2 | ribosomal protein S2 | 4 |
| RPS20 | ribosomal protein S20 | 24 |
| RPS3A | ribosomal protein S3A | 61 |
| RPS5 | ribosomal protein S5 | 19 |
| RPS6 | ribosomal protein S6 | 101 |
| RPS9 | ribosomal protein S9 | 29 |
| SECTM1 | secreted and transmembrane 1 | 112 |
| TPT1 | tumor protein, translationally-controlled 1 | 65 |
| UBA52 | ubiquitin A-52 residue ribosomal protein fusion product 1 | 84 |
| VIM | vimentin | 40 |
| ABCB7 | ATP-binding cassette, sub-family B (MDR/TAP), member 7 | 134 |
| ALKBH7 | alkB, alkylation repair homolog 7 | 85 |
| ATP5G2 | ATP synthase, H+ transporting, mitochondrial Fo | 144 |
| | complex, subunit C2 (subunit 9) | |
| EEF1A1 | eukaryotic translation elongation factor 1 alpha 1 | 7 |
| EIF2S3 | eukaryotic translation initiation factor 2, subunit 3 gamma, | 80 |
| | 52 kDa | |
| EIF3H | eukaryotic translation initiation factor 3, subunit H | 98 |
| EIF3L | eukaryotic translation initiation factor 3, subunit L | 108 |
| GLTSCR2 | glioma tumor suppressor candidate region gene 2 | 15 |
| IMPDH2 | IMP (inosine 5′-monophosphate) dehydrogenase 2 | 142 |
| PFDN5 | prefoldin subunit 5 | 130 |
| RPL10A | ribosomal protein L10a | 46 |
| RPL11 | ribosomal protein L11 | 23 |
| RPL13A | ribosomal protein L13a | 5 |
| RPL18 | ribosomal protein L18 | 62 |
| RPL19 | ribosomal protein L19 | 103 |
| RPL21 | ribosomal protein L21 | 20 |
| RPL24 | ribosomal protein L24 | 124 |
| RPL26 | ribosomal protein L26 | 52 |
| RPL27A | ribosomal protein L27A | 12 |
| RPL28 | ribosomal protein L28 | 8 |
| RPL3 | ribosomal protein L3 | 16 |
| RPL30 | ribosomal protein L30 | 81 |
| RPL7A | ribosomal protein L7a | 25 |
| RPLP1 | ribosomal protein, large, P1 | 50 |
| RPS12 | ribosomal protein S12 | 2 |
| RPS13 | ribosomal protein S13 | 105 |
| RPS16 | ribosomal protein S16 | 39 |
| RPS19 | ribosomal protein S19 | 26 |
| RPS21 | ribosomal protein S21 | 27 |
| RPS23 | ribosomal protein S23 | 100 |
| RPS24 | ribosomal protein S24 | 90 |
| RPS25 | ribosomal protein S25 | 75 |
| RPS27 | ribosomal protein S27 | 10 |
| RPS28 | ribosomal protein S28 | 9 |
| RPS29 | ribosomal protein S29 | 73 |
| RPS3 | ribosomal protein S3A | 61 |
| RPS7 | ribosomal protein S7 | 102 |

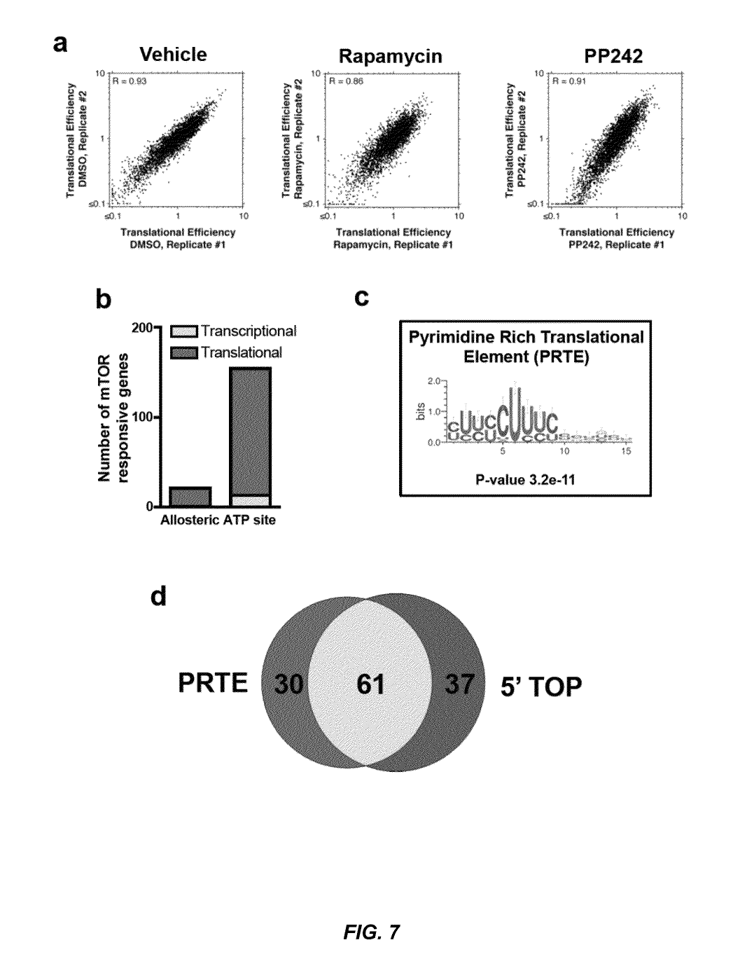

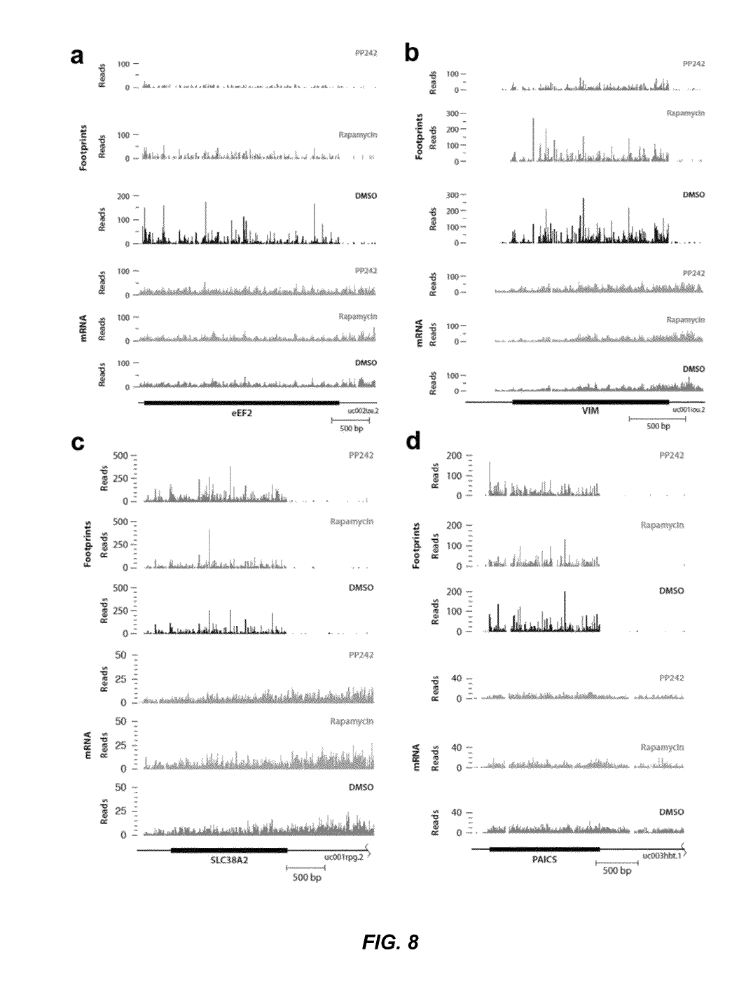

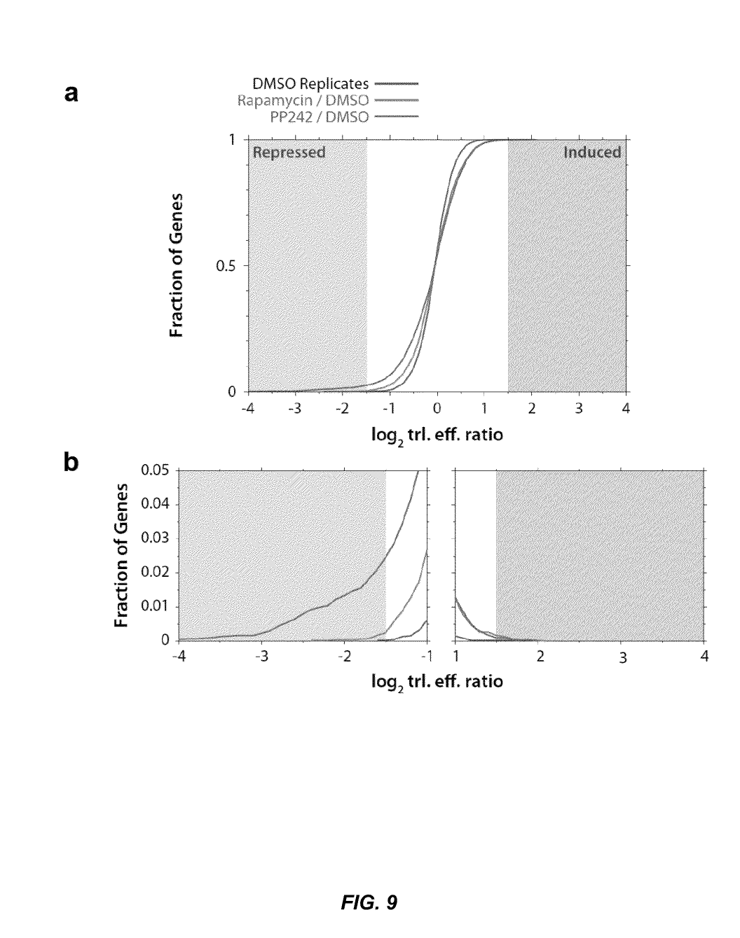

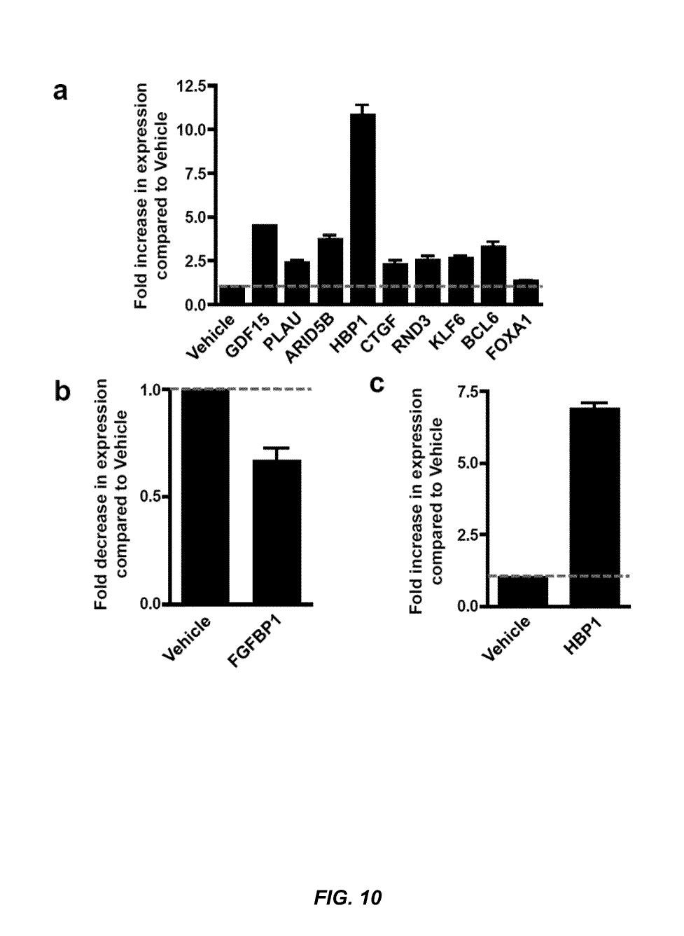

In some embodiments, a gene that has a different translational level in the first translational profile as compared to the second translational profile is a gene having a pyrimidine-rich translational element (PRTE). This element consists of an invariant uridine at its position 6 and does not reside at position +1 of the 5′ UTR. See, e.g., FIG. 7(c). Non-limiting examples of genes having a PRTE sequence are shown in Table 2 below. In some embodiments, translational levels are compared for the first translational profile and the second translational profile for 1, 2, 3, 4, 5, 6, 7, 8, 9, 10, 11, 12, 13, 14, 15, 16, 17, 18, 19, 20 or more genes selected from the genes listed in Table 2.

TABLE 2
Translationally regulated mTOR-responsive
genes having a PRTE sequence

| | | SEQ |
|---|---|---|
| Gene | Description | ID NO |
| EEF2 | eukaryotic translation elongation factor 2 | 1 |
| RPL12 | ribosomal protein L12 | 3 |
| RPS2 | ribosomal protein S2 | 4 |
| RPL18A | ribosomal protein L18a | 6 |
| RPL34 | ribosomal protein L34 | 11 |
| RPL10 | ribosomal protein L10 | 13 |
| EEF1D | eukaryotic translation elongation factor 1 delta | 14 |
| PABPC1 | poly(A) binding protein, cytoplasmic 1 | 17 |
| RPL37A | ribosomal protein L37a | 18 |
| RPS5 | ribosomal protein S5 | 19 |
| RPS15A | ribosomal protein S15a | 21 |
| GNB2L1 | guanine nucleotide binding protein (G protein) | 22 |
| RPS20 | ribosomal protein S20 isoform 1 | 24 |
| RPLP0 | ribosomal protein P0 | 28 |
| RPS9 | ribosomal protein S9 | 29 |
| CRTAP | cartilage associated protein | 31 |
| RPL32 | ribosomal protein L32 | 33 |
| EEF1G | eukaryotic translation elongation factor 1, gamma | 34 |
| RPL22L1 | ribosomal protein L22-like 1 | 35 |
| YB1 | Y-box binding protein 1 | 36 |
| EIF4B | eukaryotic translation initiation factor 4B | 37 |
| RPLP2 | ribosomal protein P2 | 38 |
| VIM | vimentin | 40 |
| HSPA8 | heat shock 70 kDa protein 8 isoform 1 | 42 |
| RPL39 | ribosomal protein L39 | 43 |
| AHCY | adenosylhomocysteinase isoform 1 | 44 |
| EEF1A2 | eukaryotic translation elongation factor 1 alpha 2 | 45 |
| PABPC4 | poly A binding protein, cytoplasmic 4 isoform 1 | 47 |
| RPS4X | ribosomal protein S4, X-linked X isoform | 48 |
| RPL31 | ribosomal protein L31 isoform 2 | 49 |
| RPS11 | ribosomal protein S11 | 51 |
| RPL14 | ribosomal protein L14 | 53 |
| RPL37 | ribosomal protein L37 | 54 |
| RPL7 | ribosomal protein L7 | 55 |
| HNRNPA1 | heterogeneous nuclear ribonucleoprotein A1 | 56 |
| RPS8 | ribosomal protein S8 | 57 |
| GAPDH | glyceraldehyde-3-phosphate dehydrogenase | 58 |
| RPL8 | ribosomal protein L8 | 59 |
| RPL29 | ribosomal protein L29 | 60 |
| RPS3A | ribosomal protein S3a | 61 |
| RPL36 | ribosomal protein L36 | 63 |
| TPT1 | tumor protein, translationally-controlled 1 | 65 |
| RPL36A | ribosomal protein L36a | 66 |
| TKT | transketolase isoform 1 | 68 |
| LMF2 | lipase maturation factor 2 | 69 |
| RPL13 | ribosomal protein L13 | 70 |
| RPL23 | ribosomal protein L23 | 74 |
| TUBB3 | tubulin, beta, 4 | 76 |
| RPS10 | ribosomal protein S10 | 77 |
| FASN | fatty acid synthase | 78 |
| RPL17 | ribosomal protein L17 | 79 |
| ACTG1 | actin, gamma 1 propeptide | 82 |
| COL6A2 | alpha 2 type VI collagen isoform 2C2 | 83 |
| UBA52 | ubiquitin and ribosomal protein L40 precursor | 84 |
| RPL5 | ribosomal protein L5 | 86 |
| PGLS | 6-phosphogluconolactonase | 87 |
| RPL6 | ribosomal protein L6 | 89 |
| RPL22 | ribosomal protein L22 | 91 |
| AP2A1 | adaptor-related protein complex 2, alpha 1 | 92 |
| NAP1L1 | nucleosome assembly protein 1-like 1 | 93 |
| RPS14 | ribosomal protein S14 | 94 |
| CCNI | cyclin I | 96 |
| MTA1 | metastasis associated 1 | 97 |
| RPL9 | ribosomal protein L9 | 99 |
| RPL4 | ribosomal protein L4 | 104 |
| LCMT1 | leucine carboxyl methyltransferase 1 isoform a | 107 |
| IPO7 | importin 7 | 109 |
| PC | pyruvate carboxylase | 110 |
| RPS27A | ubiquitin and ribosomal protein S27a | 111 |
| SECTM1 | secreted and transmembrane 1 precursor | 112 |
| RPL41 | ribosomal protein L41 | 113 |
| TSC2 | tuberous sclerosis 2 isoform 1 | 114 |
| COL18A1 | alpha 1 type XVIII collagen isoform 3 | 115 |
| CHP | calcium binding protein P22 | 116 |
| PACS1 | phosphofurin acidic cluster sorting protein 1 | 117 |
| BRF1 | transcription initiation factor IIIB | 118 |
| PTGES2 | prostaglandin E synthase 2 | 119 |
| PGM1 | phosphoglucomutase 1 | 121 |
| SLC19A1 | solute carrier family 19 member 1 | 122 |
| CD44 | CD44 antigen isoform 1 | 123 |
| RPL15 | ribosomal protein L15 | 126 |
| EEF1B2 | eukaryotic translation elongation factor 1 beta 2 | 129 |
| PNKP | polynucleotide kinase 3′ phosphatase | 131 |
| SEPT8 | septin 8 isoform a | 132 |
| EVPL | envoplakin | 136 |
| MYH14 | myosin, heavy chain 14 isoform 3 | 138 |
| RABGGTB | RAB geranylgeranyltransferase, beta subunit | 139 |
| RPL27 | ribosomal protein L27 | 140 |
| SIGMAR1 | sigma non-opioid intracellular receptor 1 | 143 |

In some embodiments, a gene that has a different translational level in the first translational profile as compared to the second translational profile is a gene having both a 5′ TOP sequence and a PRTE sequence. Non-limiting examples of genes having both a 5′ TOP sequence and a PRTE sequence are shown in Table 3 below. In some embodiments, translational levels are compared for the first translational profile and the second translational profile for 1, 2, 3, 4, 5, 6, 7, 8, 9, 10, 11, 12, 13, 14, 15, 16, 17, 18, 19, 20 or more genes selected from the genes listed in Table 3.

TABLE 3
5′ TOP and PRTE genomic positions in translationally regulated mTOR-
responsive genes having both 5′ TOP and PRTE

| | | | Strand | | PRTE |
|---|---|---|---|---|---|
| Gene | RefSeq ID | Chromosome | (+/−) | 5′ TOP Position | Position |
| AP2A1 | NM_014203 | 19 | + | 50270268 | 50270306 |
| CCNI | NM_006835 | 4 | − | 77997142 | 77997076 |
| CD44 | NM_000610 | 11 | + | 35160717 | 35160813 |
| CHP | NM_007236 | 15 | + | 41523519 | 41523536 |
| CRTAP | NM_006371 | 3 | + | 33155506/ | 33155540 |
| | | | | 33155554 | |
| eEF1A2 | NM_001958 | 20 | − | 62130436 | 62129175 |
| eEF1B2 | NM_021121 | 2 | + | 207024619 | 207024665 |
| eEF1G | NM_001404 | 11 | − | 62341490/ | 62341383 |
| | | | | 62341335 | |
| eEF2 | NM_001961 | 19 | − | 3985461 | 3985423 |
| eIF4B | NM_001417 | 12 | + | 53400240 | 53400250 |
| GAPDH | NM_002046 | 12 | + | 6643684 | 6643717 |
| GNB2L1 | NM_006098 | 5 | − | 180670906 | 180670818 |
| HNRNPA1 | NM_031157 | 12 | + | 54674529 | 54674571 |
| HSPA8 | NM_006597 | 11 | − | 122932844 | 122932806 |
| IPO7 | NM_006391 | 11 | + | 9406199 | 9406255 |
| LCMT1 | NM_016309 | 16 | + | 25123101 | 25123114 |
| NAP1L1 | NM_004537 | 12 | − | 76478465 | 76478429 |
| PABPC1 | NM_002568 | 8 | − | 101734315 | 101734151 |
| PACS1 | NM_018026 | 11 | + | 65837839 | 65837922 |
| PGM1 | NM_002633 | 1 | + | 64059078 | 64059107 |
| RABGGTB | NM_004582 | 1 | + | 76251941 | 76251928 |
| RPL10 | NM_006013 | X | + | 153626718 | 153626846 |
| RPL12 | NM_000976 | 9 | − | 130213677 | 130213648 |
| RPL13 | NM_000977/ | 16 | + | 89627090 | 89627102/ |
| | NM_033251 | | | | 89627202 |
| RPL14 | NM_001034996 | 3 | + | 40498830 | 40498906 |
| RPL15 | NM_002948 | 3 | + | 23958639 | 23958711 |
| RPL17 | NM_000985 | 18 | − | 47018849 | 47017964 |
| RPL22 | NM_000983 | 1 | − | 6259654 | 6259645 |
| RPL22L1 | NM_001099645 | 3 | − | 170587984 | 170587976 |
| RPL23 | NM_000978 | 17 | − | 37009989 | 37010013 |
| RPL29 | NM_000992 | 3 | − | 52029911 | 52029904 |
| RPL31 | NM_001098577 | 2 | + | 101618755 | 101618739 |
| RPL32 | NM_001007074 | 3 | − | 12883040 | 12883002 |
| RPL34 | NM_000995/ | 4 | + | 109541733 | 109541743/ |
| | NM_033625 | | | | 109541769 |
| RPL36 | NM_033643/ | 19 | + | 5690307 | 5690319/ |
| | NM_015414 | | | | 5690493 |
| RPL36A | NM_021029 | X | + | 100645999 | 100645981 |
| RPL37 | NM_000997 | 5 | − | 40835324 | 40835314 |
| RPL37A | NM_000998 | 2 | + | 217363567 | 217363526 |
| RPL39 | NM_001000 | X | − | 118925591 | 118925564 |
| RPL4 | NM_000968 | 15 | − | 66797185 | 66797143 |
| RPL41 | NM_001035267 | 12 | + | 56510417 | 56510539 |
| RPL5 | NM_000969 | 1 | + | 93297597 | 93297656 |
| RPL6 | NM_000970 | 12 | − | 112847409 | 112847256 |
| RPL8 | NM_000973/ | 8 | − | 146017775 | 146017709 |
| | NM_033301 | | | | |
| RPLP0 | NM_053275 | 12 | − | 120638910 | 120638652 |
| RPLP2 | NM_001004 | 11 | + | 809968 | 810006 |
| RPS10 | NM_001014 | 6 | − | 34393846 | 34393715 |
| RPS11 | NM_001015 | 19 | + | 49999690 | 49999677 |
| RPS14 | NM_001025070 | 5 | − | 149829300/ | 149829107 |
| | | | | 149829186 | |
| RPS15A | NM_001030009 | 16 | − | 18801656 | 18801604 |
| RPS2 | NM_002952 | 16 | − | 2014827 | 2014653 |
| RPS20 | NM_001146227 | 8 | − | 56987065 | 56986992 |
| RPS27A | NM_001177413 | 2 | + | 55459824 | 55459920 |
| RPS3A | NM_001006 | 4 | + | 152020780 | 152020789 |
| RPS5 | NM_001009 | 19 | + | 58898636 | 58898691 |
| RPS6 | NM_001010 | 9 | − | 19380234 | 19380207 |
| RPS9 | NM_001013 | 19 | + | 54704726 | 54704775 |
| SECTM1 | NM_003004 | 17 | − | 80291646 | 80291674/ |
| | | | | | 80291639 |
| TPT1 | NM_003295 | 13 | − | 45915318 | 45915222 |
| UBA52 | NM_003333 | 19 | + | 18682670 | 18683218 |
| VIM | NM_003380 | 10 | + | 17271277 | 17271358 |

In some embodiments, the method comprises:
(a) contacting the biological sample with an agent;
(b) determining a first translational profile for the contacted biological sample, wherein the translational profile comprises translational levels for one or more genes selected from the group consisting of SEQ ID NOs:1-144; and
(c) comparing the first translational profile to a second translational profile comprising translational levels for the one or more genes in a control sample that has not been contacted with the agent;
wherein a difference in the translational levels of the one or more genes in the first translation profile as compared to the second translation profile identifies the agent as a modulator of the oncogenic signaling pathway.

In some embodiments, translational levels are compared for the first translational profile and the second translational profile for 1, 2, 3, 4, 5, 6, 7, 8, 9, 10, 11, 12, 13, 14, 15, 16, 17, 18, 19, 20 or more genes selected from the group consisting of SEQ ID NOs:1-144. SEQ ID NOs:1-144 are listed in Table 4 below:

TABLE 4
Translationally regulated mTOR-responsive genes

| | | SEQ |
|---|---|---|
| Gene | Description | ID NO |
| EEF2 | eukaryotic translation elongation factor 2 | 1 |
| RPS12 | ribosomal protein S12 | 2 |
| RPL12 | ribosomal protein L12 | 3 |
| RPS2 | ribosomal protein S2 | 4 |
| RPL13A | ribosomal protein L13a | 5 |
| RPL18A | ribosomal protein L18a | 6 |
| EEF1A1 | eukaryotic translation elongation factor 1 alpha 1 | 7 |
| RPL28 | ribosomal protein L28 isoform 1 | 8 |
| RPS28 | ribosomal protein S28 | 9 |
| RPS27 | ribosomal protein S27 | 10 |
| RPL34 | ribosomal protein L34 | 11 |
| RPL27A | ribosomal protein L27a | 12 |
| RPL10 | ribosomal protein L10 | 13 |
| EEF1D | eukaryotic translation elongation factor 1 delta | 14 |
| GLTSCR2 | glioma tumor suppressor candidate region gene 2 | 15 |
| RPL3 | ribosomal protein L3 isoform a | 16 |
| PABPC1 | poly(A) binding protein, cytoplasmic 1 | 17 |
| RPL37A | ribosomal protein L37a | 18 |
| RPS5 | ribosomal protein S5 | 19 |
| RPL21 | ribosomal protein L21 | 20 |
| RPS15A | ribosomal protein S15a | 21 |
| GNB2L1 | guanine nucleotide binding protein (G protein) | 22 |
| RPL11 | ribosomal protein L11 | 23 |
| RPS20 | ribosomal protein S20 isoform 1 | 24 |
| RPL7A | ribosomal protein L7a | 25 |
| RPS19 | ribosomal protein S19 | 26 |
| RPS21 | ribosomal protein S21 | 27 |
| RPLP0 | ribosomal protein P0 | 28 |
| RPS9 | ribosomal protein S9 | 29 |
| RPS3 | ribosomal protein S3 | 30 |
| CRTAP | cartilage associated protein | 31 |
| FAM128B | hypothetical protein LOC80097 | 32 |
| RPL32 | ribosomal protein L32 | 33 |
| EEF1G | eukaryotic translation elongation factor 1, gamma | 34 |
| RPL22L1 | ribosomal protein L22-like 1 | 35 |
| YB1 | Y-box binding protein 1 | 36 |
| EIF4B | eukaryotic translation initiation factor 4B | 37 |
| RPLP2 | ribosomal protein P2 | 38 |
| RPS16 | ribosomal protein S16 | 39 |
| VIM | vimentin | 40 |
| GAMT | guanidinoacetate N-methyltransferase isoform b | 41 |
| HSPA8 | heat shock 70 kDa protein 8 isoform 1 | 42 |
| RPL39 | ribosomal protein L39 | 43 |
| AHCY | adenosylhomocysteinase isoform 1 | 44 |
| EEF1A2 | eukaryotic translation elongation factor 1 alpha 2 | 45 |
| RPL10A | ribosomal protein L10a | 46 |
| PABPC4 | poly A binding protein, cytoplasmic 4 isoform 1 | 47 |
| RPS4X | ribosomal protein S4, X-linked X isoform | 48 |
| RPL31 | ribosomal protein L31 isoform 2 | 49 |
| RPLP1 | ribosomal protein P1 isoform 1 | 50 |
| RPS11 | ribosomal protein S11 | 51 |
| RPL26 | ribosomal protein L26 | 52 |
| RPL14 | ribosomal protein L14 | 53 |
| RPL37 | ribosomal protein L37 | 54 |
| RPL7 | ribosomal protein L7 | 55 |
| HNRNPA1 | heterogeneous nuclear ribonucleoprotein A1 | 56 |
| RPS8 | ribosomal protein S8 | 57 |
| GAPDH | glyceraldehyde-3-phosphate dehydrogenase | 58 |
| RPL8 | ribosomal protein L8 | 59 |
| RPL29 | ribosomal protein L29 | 60 |
| RPS3A | ribosomal protein S3a | 61 |
| RPL18 | ribosomal protein L18 | 62 |
| RPL36 | ribosomal protein L36 | 63 |
| AGRN | agrin precursor | 64 |
| TPT1 | tumor protein, translationally-controlled 1 | 65 |
| RPL36A | ribosomal protein L36a | 66 |
| SLC25A5 | adenine nucleotide translocator 2 | 67 |
| TKT | transketolase isoform 1 | 68 |
| LMF2 | lipase maturation factor 2 | 69 |
| RPL13 | ribosomal protein L13 | 70 |
| CTSH | cathepsin H isoform b | 71 |
| FAM83H | FAM83H | 72 |
| RPS29 | ribosomal protein S29 isoform 2 | 73 |
| RPL23 | ribosomal protein L23 | 74 |
| RPS25 | ribosomal protein S25 | 75 |
| TUBB3 | tubulin, beta, 4 | 76 |
| RPS10 | ribosomal protein S10 | 77 |
| FASN | fatty acid synthase | 78 |
| RPL17 | ribosomal protein L17 | 79 |
| EIF2S3 | eukaryotic translation initiation factor 2, S3 | 80 |
| RPL30 | ribosomal protein L30 | 81 |
| ACTG1 | actin, gamma 1 propeptide | 82 |
| COL6A2 | alpha 2 type VI collagen isoform 2C2 | 83 |
| UBA52 | ubiquitin and ribosomal protein L40 precursor | 84 |
| ALKBH7 | spermatogenesis associated 11 precursor | 85 |
| RPL5 | ribosomal protein L5 | 86 |
| PGLS | 6-phosphogluconolactonase | 87 |
| CSDA | cold shock domain protein A | 88 |
| RPL6 | ribosomal protein L6 | 89 |
| RPS24 | ribosomal protein S24 isoform d | 90 |
| RPL22 | ribosomal protein L22 | 91 |
| AP2A1 | adaptor-related protein complex 2, alpha 1 | 92 |
| NAP1L1 | nucleosome assembly protein 1-like 1 | 93 |
| RPS14 | ribosomal protein S14 | 94 |
| ETHE1 | ETHE1 protein | 95 |
| CCNI | cyclin I | 96 |
| MTA1 | metastasis associated 1 | 97 |
| EIF3H | eukaryotic translation initiation factor 3, H | 98 |
| RPL9 | ribosomal protein L9 | 99 |
| RPS23 | ribosomal protein S23 | 100 |
| RPS6 | ribosomal protein S6 | 101 |
| RPS7 | ribosomal protein S7 | 102 |
| RPL19 | ribosomal protein L19 | 103 |
| RPL4 | ribosomal protein L4 | 104 |
| RPS13 | ribosomal protein S13 | 105 |
| C21orf66 | GC-rich sequence DNA-binding factor candidate | 106 |
| LCMT1 | leucine carboxyl methyltransferase 1 isoform a | 107 |
| EIF3L | eukaryotic translation initiation factor 3, L | 108 |
| IPO7 | importin 7 | 109 |
| PC | pyruvate carboxylase | 110 |
| RPS27A | ubiquitin and ribosomal protein S27a | 111 |
| SECTM1 | secreted and transmembrane 1 precursor | 112 |
| RPL41 | ribosomal protein L41 | 113 |
| TSC2 | tuberous sclerosis 2 isoform 1 | 114 |
| COL18A1 | alpha 1 type XVIII collagen isoform 3 | 115 |
| CHP | calcium binding protein P22 | 116 |
| PACS1 | phosphofurin acidic cluster sorting protein 1 | 117 |
| BRF1 | transcription initiation factor IIIB | 118 |
| PTGES2 | prostaglandin E synthase 2 | 119 |
| C2orf79 | hypothetical protein LOC391356 | 120 |
| PGM1 | phosphoglucomutase 1 | 121 |
| SLC19A1 | solute carrier family 19 member 1 | 122 |
| CD44 | CD44 antigen isoform 1 | 123 |
| RPL24 | ribosomal protein L24 | 124 |
| NCLN | nicalin | 125 |
| RPL15 | ribosomal protein L15 | 126 |
| CLPTM1 | cleft lip and palate associated transmembrane | 127 |
| ECSIT | evolutionarily conserved signaling intermediate | 128 |
| EEF1B2 | eukaryotic translation elongation factor 1 beta 2 | 129 |
| PFDN5 | prefoldin subunit 5 isoform alpha | 130 |
| PNKP | polynucleotide kinase 3′ phosphatase | 131 |
| SEPT8 | septin 8 isoform a | 132 |
| CIRBP | cold inducible RNA binding protein | 133 |
| ABCB7 | ATP-binding cassette, sub-family B, member 7 | 134 |
| ARD1A | alpha-N-acetyltransferase 1A | 135 |
| EVPL | envoplakin | 136 |
| LAMA5 | laminin alpha 5 | 137 |
| MYH14 | myosin, heavy chain 14 isoform 3 | 138 |
| RABGGTB | RAB geranylgeranyltransferase, beta subunit | 139 |
| RPL27 | ribosomal protein L27 | 140 |
| RPS15 | ribosomal protein S15 | 141 |
| IMPDH2 | inosine monophosphate dehydrogenase 2 | 142 |
| SIGMAR1 | sigma non-opioid intracellular receptor 1 | 143 |
| ATP5G2 | ATP synthase, H+ transporting, mitochondrial F0 | 144 |

In some embodiments, the first and/or second translational profile comprises 1, 2, 3, 4, 5, 6, 7, 8, 9, 10 or more genes that are functionally classified as a protein synthesis gene, a cell invasion/metastasis gene, a metabolism gene, a signal transduction gene, a cellular transport gene, a post-translational modification gene, an RNA synthesis and processing gene, a regulation of cell proliferation gene, a development gene, an apoptosis gene, a DNA repair gene, a DNA methylation gene, or an amino acid biosynthesis gene. In some embodiments, the first and/or second translational profile comprises 1, 2, 3, 4, 5, 6, 7, 8, 9, 10 or more genes from each of two, three, four, five, or more of these functional categories of genes. In some embodiments, first and/or second translational profile comprises 1, 2, 3, 4, 5, 6, 7, 8, 9, 10 more genes that are functionally classified as a cell invasion or metastasis gene. In some embodiments, the first and/or second translational profile comprises one or more of the cell invasion/metastasis genes YB1, vimentin, MTA1, and CD44. In some embodiments, the first and/or second translational profile comprises YB1, vimentin, MTA1, and CD44.

In some embodiments, the method comprises:
(a) contacting the biological sample with an agent;
(b) determining a first translational profile for the contacted biological sample, wherein the translational profile comprises a measurement of gene translational levels for a substantial portion of the genome;
(c) comparing the first translational profile to a second translational profile comprising a measurement of gene translational levels for the substantial portion of the genome translational levels for the one or more genes in a control sample that has not been contacted with the agent;
(d) identifying in the first translational profile a plurality of genes having decreased translational levels as compared to the translational levels of the plurality of genes in the second translational profile; and
(e) determining whether, for the plurality of genes identified in step (d), there is a common consensus sequence and/or regulatory element in the untranslated regions (UTRs) of the genes that is shared by at least 10% of the plurality of genes identified in step (d);
wherein a decrease in the translational levels of at least 10% of the genes sharing the common consensus sequence and/or UTR regulatory element in the first translational profile as compared to the second translational profile identifies the agent as an inhibitor of an oncogenic signaling pathway.

As used herein, the term “substantial portion of the genome,” with reference to a biological sample, can refer to an empirical number of genes being measured in the biological sample or to a percentage of the genes in the genome being measured in the biological sample. In some embodiments, a substantial portion of the genome comprises at least 500 genes, e.g., at least 500, 600, 700, 800, 900, 1000, 2000, 3000, 4000, 5000, 6000, 7000, 8000, 9000, 10,000, 11,000, 12,000, 13,000, 14,000, or 15,000 genes or more. In some embodiments, a substantial portion of the genome comprises at least about 0.01%, at least about 0.05%, at least about 0.1%, at least about 0.5%, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 25%, at least about 30%, at least about 35%, at least about 40%, at least about 45%, or at least about 50% of all genes in the genome for the biological sample.

In some embodiments, the oncogenic signaling pathway that is modulated is the mammalian target of rapamycin (mTOR) pathway, the PI3K pathway, the AKT pathway, the Ras pathway, the Myc pathway, the Wnt pathway, or the BRAF pathway. In some embodiments, the oncogenic signaling pathway that is modulated is the mTOR pathway.

In some embodiments, there is at least a 1.5-fold or at least 2-fold (e.g., at least 1.5-fold, at least 2-fold, at least 2.5-fold, at least 3-fold, at least 3.5-fold, at least 4-fold, at least 4.5-fold, at least 5-fold, at least 6-fold, at least 7-fold, at least 8-fold, at least 9-fold, at least 10-fold difference or more) in translational level for the one or more genes in the first translational profile as compared to the second translational profile. In some embodiments, there is at least a 1.5-fold or at least a 2-fold difference in translational level for 1, 2, 3, 4, 5, 6, 7, 8, 9, 10, 11, 12, 13, 14, 15, 16, 17, 18, 19, 20, 30, 40, 50, 60, 70, 80, 90, 100 or more genes in the first translational profile as compared to the second translational profile. In some embodiments, the translational level of one or more genes is decreased in the first translational profile as compared to the second translational profile. In some embodiments, the translational level of one or more genes in the first translational profile is decreased by at least 1.5-fold, at least two-fold, at least three-fold, at least four-fold, at least five-fold, at least six-fold, at least seven-fold, at least eight-fold, at least nine-fold, at least ten-fold or more as compared to the second translational profile. In some embodiments, the translational level of one or more genes is increased in the first translational profile as compared to the second translational profile. In some embodiments, the translational level of one or more genes in the first translational profile is increased by at least 1.5-fold, at least two-fold, at least three-fold, at least four-fold, at least five-fold, at least six-fold, at least seven-fold, at least eight-fold, at least nine-fold, at least ten-fold or more as compared to the second translational profile. In some embodiments, the translational level of one or more genes is decreased (e.g., by at least 1.5-fold, at least two-fold, at least three-fold, at least four-fold, at least five-fold, at least six-fold, at least seven-fold, at least eight-fold, at least nine-fold, at least ten-fold or more) in the first translational profile, while the translational level of another one or more genes is increased (e.g., by at least 1.5-fold, at least two-fold, at least three-fold, at least four-fold, at least five-fold, at least six-fold, at least seven-fold, at least eight-fold, at least nine-fold, at least ten-fold or more) in the first translational profile, as compared to the second translational profile.

C. Agents

In some embodiments, an agent that can be used according to the methods of the present invention is a peptide, protein, oligopeptide, circular peptide, peptidomimetic, antibody, polysaccharide, lipid, fatty acid, inhibitory RNA (e.g., siRNA, miRNA, or shRNA), polynucleotide, oligonucleotide, aptamer, small organic molecule, or drug compound. The agent can be either synthetic or naturally-occurring.

In some embodiments, the agent acts as a specific regulator of translational machinery or a component of translational machinery that alters the program of protein translation in cells (e.g., a small molecule inhibitor or inhibitory RNA). In some embodiments, the agent binds at the active site of a protein (e.g., an ATP site inhibitor of mTOR).

In some embodiments, multiple agents (e.g., 2, 3, 4, 5, or more agents) are used. In some embodiments, multiple agents are administered to a subject or contacted to a biological sample sequentially. In some embodiments, multiple agents are administered to a subject or contacted to a biological sample concurrently.

The agents described herein can be used at varying concentrations. In some embodiments, an agent is administered to a subject or contacted to a biological sample at a concentration that is known or expected to be a therapeutic dose. In some embodiments, an agent is administered to a subject or contacted to a biological sample at a concentration that is known or expected to be a sub-therapeutic dose. In some embodiments, an agent is administered to a subject or contacted to a biological sample at a concentration that is lower than a concentration that would typically be administered to an organism or applied to a sample, e.g., at a concentration that is 2, 3, 4, 5, 6, 7, 8, 9, 10, 15, 20, 25, 30, 35, 40, 45, 50, 60, 70, 80, 90, or 100 times less than the concentration that would typically be administered to an organism or applied to a sample.

In some embodiments, an agent can be identified from a library of agents. In some embodiments, the library of agents comprises at least about 5, 10, 15, 20, 25, 30, 35, 40, 45, 50, 60, 70, 80, 90, 100, 200, 300, 400, 500, 600, 700, 800, 900, 1000, 5000, 10,000, 20,000, 30,000, 40,000, 50,000 agents or more. It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland), as well as providers of small organic molecule and peptide libraries ready for screening, including Chembridge Corp. (San Diego, Calif.), Discovery Partners International (San Diego, Calif.), Triad Therapeutics (San Diego, Calif.), Nanosyn (Menlo Park, Calif.), Affymax (Palo Alto, Calif.), ComGenex (South San Francisco, Calif.), and Tripos, Inc. (St. Louis, Mo.). In some embodiments, the library is a combinatorial chemical or peptide library. A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks. The preparation and screening of chemical libraries is well known to those of skill in the art (see, e.g., Beeler et al., Curr Opin Chem. Biol., 9:277 (2005); and Shang et al., Curr Opin Chem. Biol., 9:248 (2005)).

In some embodiments, an agent for use in the methods of the present invention (e.g., an agent that modulates an oncogenic signaling pathway) can be identified by screening a library containing a large number of potential therapeutic compounds. The library can be screened in one or more assays, as described herein, to identify those library members that display a desired characteristic activity. The compounds thus identified can serve as conventional “lead compounds” (e.g., for identifying other potential therapeutic compounds) or can themselves be used as potential or actual therapeutics. Libraries of use in the present invention can be composed of amino acid compounds, nucleic acid compounds, carbohydrates, or small organic compounds. Carbohydrate libraries have been described in, for example, Liang et al., Science, 274:1520-1522 (1996); and U.S. Pat. No. 5,593,853.

Representative amino acid compound libraries include, but are not limited to, peptide libraries (see, e.g., U.S. Pat. Nos. 5,010,175; 6,828,422; and 6,844,161; Furka, Int. J. Pept. Prot. Res., 37:487-493 (1991); Houghton et al., Nature, 354:84-88 (1991); and Eichler, Comb Chem High Throughput Screen., 8:135 (2005)), peptoids (PCT Publication No. WO 91/19735), encoded peptides (PCT Publication No. WO 93/20242), random bio-oligomers (PCT Publication No. WO 92/00091), vinylogous polypeptides (Hagihara et al., J. Amer. Chem. Soc., 114:6568 (1992)), nonpeptidal peptidomimetics with β-D-glucose scaffolding (Hirschmann et al., J. Amer. Chem. Soc., 114:9217-9218 (1992)), peptide nucleic acid libraries (see, e.g., U.S. Pat. No. 5,539,083), antibody libraries (see, e.g., U.S. Pat. Nos. 6,635,424 and 6,555,310; PCT Application No. PCT/US96/10287; and Vaughn et al., Nature Biotechnology, 14:309-314 (1996)), and peptidyl phosphonates (Campbell et al., J. Org. Chem., 59:658 (1994)).

Representative nucleic acid compound libraries include, but are not limited to, genomic DNA, cDNA, mRNA, inhibitory RNA (e.g., RNAi, siRNA), and antisense RNA libraries. See, e.g., Ausubel, Current Protocols in Molecular Biology, eds. 1987-2005, Wiley Interscience; and Sambrook and Russell, Molecular Cloning: A Laboratory Manual, 2000, Cold Spring Harbor Laboratory Press. Nucleic acid libraries are described in, for example, U.S. Pat. Nos. 6,706,477; 6,582,914; and 6,573,098. cDNA libraries are described in, for example, U.S. Pat. Nos. 6,846,655; 6,841,347; 6,828,098; 6,808,906; 6,623,965; and 6,509,175. RNA libraries, for example, ribozyme, RNA interference, or siRNA libraries, are described in, for example, Downward, Cell, 121:813 (2005) and Akashi et al., Nat. Rev. Mol. Cell. Biol., 6:413 (2005). Antisense RNA libraries are described in, for example, U.S. Pat. Nos. 6,586,180 and 6,518,017.

Representative small organic molecule libraries include, but are not limited to, diversomers such as hydantoins, benzodiazepines, and dipeptides (Hobbs et al., Proc. Nat. Acad. Sci. USA, 90:6909-6913 (1993)); analogous organic syntheses of small compound libraries (Chen et al., J. Amer. Chem. Soc., 116:2661 (1994)); oligocarbamates (Cho et al., Science, 261:1303 (1993)); benzodiazepines (e.g., U.S. Pat. No. 5,288,514; and Baum, C& EN, January 18, page 33 (1993)); isoprenoids (e.g., U.S. Pat. No. 5,569,588); thiazolidinones and metathiazanones (e.g., U.S. Pat. No. 5,549,974); pyrrolidines (e.g., U.S. Pat. Nos. 5,525,735 and 5,519,134); morpholino compounds (e.g., U.S. Pat. No. 5,506,337); tetracyclic benzimidazoles (e.g., U.S. Pat. No. 6,515,122); dihydrobenzpyrans (e.g., U.S. Pat. No. 6,790,965); amines (e.g., U.S. Pat. No. 6,750,344); phenyl compounds (e.g., U.S. Pat. No. 6,740,712); azoles (e.g., U.S. Pat. No. 6,683,191); pyridine carboxamides or sulfonamides (e.g., U.S. Pat. No. 6,677,452); 2-aminobenzoxazoles (e.g., U.S. Pat. No. 6,660,858); isoindoles, isooxyindoles, or isooxyquinolines (e.g., U.S. Pat. No. 6,667,406); oxazolidinones (e.g., U.S. Pat. No. 6,562,844); and hydroxylamines (e.g., U.S. Pat. No. 6,541,276).

Devices for the preparation of libraries are commercially available. See, e.g., 357 MPS and 390 MPS from Advanced Chem. Tech (Louisville, Ky.), Symphony from Rainin Instruments (Woburn, Mass.), 433A from Applied Biosystems (Foster City, Calif.), and 9050 Plus from Millipore (Bedford, Mass.).

D. Undruggable Targets

In some embodiments, the methods of the present invention relate to identifying an agent that modulates an undruggable target. It is estimated that only about 10-15% of human proteins are disease modifying, and of these proteins, as many as 85-90% are “undruggable,” meaning that even though theoretical therapeutic benefits may be experimentally observed for these target proteins (e.g., in vitro or in a model system in vivo using techniques such as shRNA), targeted therapy using a drug compound (e.g., a small molecule or antibody) does not successfully interfere with the biological function of the protein (or of the gene encoding the protein). Typically, an undruggable target is a protein that lacks a binding site for small molecules or for which binding of small molecules does not alter biological function (e.g., ribosomal proteins); a protein for which, despite having a small molecule binding site, successful targeting of said site has proven intractable in practice (e.g., GTP/GDP proteins); or a protein for which selectivity of small molecule binding has not been obtained due to close homology of the binding site with other proteins, and for which binding of the small molecule to these other proteins obviates the therapeutic benefit that is theoretically achievable with binding to the target protein (e.g., protein phosphatases). A target may be undruggable to antibody-based therapeutics for a variety of reasons, such as intracellular location of the target, masking of target antigenicity (e.g., due to modification with carbohydrate or other masking modifications), escape by competition (e.g., by shedding or release of decoy molecules), or the like. By preferentially inhibiting the synthesis of such a target protein by selectively inhibiting programmed translation of a small set of proteins (e.g., about 1, 2, 3, 4, 5, 6, 7, 8, 9, 10, 15, or 20 proteins), it is possible to modulate (e.g., inhibit) the activity of the “undruggable” target protein.

In some embodiments, a method of identifying an agent that modulates an undruggable target comprises:
(a) contacting a biological sample with an agent;
(b) determining a first translational profile for the contacted biological sample, wherein the translational profile comprises translational levels for a plurality of genes; and
(c) comparing the first translational profile to a second translational profile comprising translational levels for the plurality of genes in a control sample that has not been contacted with the agent;
wherein identifying one or more genes as differentially translated in the first translational profile as compared to the second translational profile identifies the agent as modulating the activity of the undruggable target. In some embodiments, one or more genes of a biological pathway are differentially translated in the first translational profile as compared to the second translational profile, wherein the biological pathway is selected from a protein synthesis pathway, a cell invasion/metastasis pathway, a cell division pathway, an apoptosis pathway, a signal transduction pathway, a cellular transport pathway, a post-translational protein modification pathway, a DNA repair pathway, and DNA methylation pathway.

In some embodiments, one or more genes from each of at least two, at least three, at least four, at least five, or more of the biological pathways is differentially translated in the first translational profile as compared to the second translational profile. In some embodiments, two, three, four, five or more genes (e.g., 1, 2, 3, 4, 5, 6, 7, 8, 9, 10, 11, 12, 13, 14, 15, 16, 17, 18, 19, 20, 30, 40, 50, 60, 70, 80, 90, 100 or more genes) from one or more of the biological pathways are differentially translated in the first translational profile as compared to the second translational profile. Non-limiting examples of protein synthesis, cell invasion/metastasis, cell division, apoptosis pathway, signal transduction, cellular transport, post-translational protein modification, DNA repair, and DNA methylation pathways are described herein.

In some embodiments, the first and/or second translational profile comprises translational levels for a plurality of genes in the biological sample. In some embodiments, the first and/or second translational profile comprises translational levels for at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10,000 genes or more in the biological sample. In some embodiments, the first and/or second translational profile comprises translational levels for at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50% of all genes in the biological sample or more. In some embodiments, the first and/or second translational profile comprises a genome-wide measurement of gene translational levels in the biological sample.

In some embodiments, there is at least a 1.5-fold or at least a two-fold difference in translational level for the one or more genes (e.g., for 1, 2, 3, 4, 5, 6, 7, 8, 9, 10, 11, 12, 13, 14, 15, 16, 17, 18, 19, 20, 30, 40, 50, 60, 70, 80, 90, 100 or more genes) in the first translational profile as compared to the second translational profile. In some embodiments, there is at least a three-fold difference, at least a four-fold difference, at least a five-fold difference, at least a six-fold difference, at least a seven-fold difference, at least an eight-fold difference, at least a nine-fold difference, at least a ten-fold difference or more in the translational level for the one or more genes in the first translational profile as compared to the second translational profile. In some embodiments, the translational level of the one or more genes is decreased in the first translational profile as compared to the second translational profile. In some embodiments, the translational level of the one or more genes is increased in the first translational profile as compared to the second translational profile. In some embodiments, the translational level of one or more genes is decreased in the first translational profile, while the translational level of another one or more genes is increased in the first translational profile, as compared to the second translational profile.

In some embodiments, the agent is an RNA molecule. In some embodiments, the agent is an shRNA, siRNA, or miRNA molecule.

E. Synthesizing and Validating Agents Based on Identified Agents

In some embodiments, an agent that is identified as modulating an oncogenic signaling pathway is optimized in order to improve the agent's biological and/or pharmacological properties. To optimize the agent, structurally related analogs of the agent can be chemically synthesized to systematically modify the structure of the initially-identified agent.

For chemical synthesis, solid phase synthesis can be used for compounds such as peptides, nucleic acids, organic molecules, etc., since in general solid phase synthesis is a straightforward approach with excellent scalability to commercial scale. Techniques for solid phase synthesis are described in the art. See, e.g., Seneci, Solid Phase Synthesis and Combinatorial Technologies (John Wiley & Sons 2002); Barany & Merrifield, Solid-Phase Peptide Synthesis, pp. 3-284 in The Peptides: Analysis, Synthesis, Biology, Vol. 2 (E. Gross and J. Meienhofer, eds., Academic Press 1979).

The synthesized structurally related analogs can be screened to determine whether the analogs induce a similar translational profile when contacted to a biological sample as compared to the initial agent from which the analog was derived. In some embodiments, a selected-for structurally related analog is one that induces an identical or substantially identical translational profile in a biological sample as the initial agent from which the structurally related analog was derived.

A structurally related analog that is determined to induce a sufficiently similar translational profile in a biological sample as the initial agent from which the structurally related analog was derived can be further screened for biological and pharmacological properties, including but not limited to oral bioavailability, half-life, metabolism, toxicity, and pharmacodynamic activity (e.g., duration of the therapeutic effect) according to methods known in the art. Typically, the screening of the structurally related analogs is performed in vivo in an appropriate animal model (e.g., a mammal such as a mouse or rat). Animal models for analyzing pharmacological and pharmacokinetic properties, including animal models for various disease states, are well known in the art and are commercially available, e.g., from Charles River Laboratories Intl, Inc. (Wilmington, Mass.).

In some embodiments, an agent that is identified as having a suitable biological profile, or a structurally related analog thereof, is used for the preparation of a medicament for the treatment of a disease or condition associated with the modulation of the biological pathway (e.g., a cancer associated with the modulation of the mTOR pathway).

### V. Methods of Validating a Target for Therapeutic Intervention

In another aspect, the present invention provides methods of validating a target for therapeutic intervention. In some embodiments, the present invention provides a method of validating a target for therapeutic intervention when treatment mimics the translational effect of a known active compound. In some embodiments, the method comprises:
(a) contacting a biological sample with an agent that modulates the target;
(b) determining a first translational profile for the contacted biological sample, wherein the first translational profile comprises translational levels for a plurality of genes; and
(c) comparing the first translational profile to a second translational profile comprising translational levels for the plurality of genes in a control sample that has not been contacted with the agent;
wherein identifying one or more genes of a biological pathway as differentially translated in the first translational profile as compared to the second translational profile validates the target for therapeutic intervention, wherein said biological pathway is selected from a protein synthesis pathway, a cell invasion/metastasis pathway, a cell division pathway, an apoptosis pathway, a signal transduction pathway, a cellular transport pathway, a post-translational protein modification pathway, a DNA repair pathway, and a DNA methylation pathway.

In some embodiments, translational levels are compared for the first translational profile and the second translational profile for 1, 2, 3, 4, 5, 6, 7, 8, 9, 10, 11, 12, 13, 14, 15, 16, 17, 18, 19, 20 or more genes in one or more biological pathways selected from a protein synthesis pathway, a cell invasion/metastasis pathway, a cell division pathway, an apoptosis pathway, a signal transduction pathway, a cellular transport pathway, a post-translational protein modification pathway, a DNA repair pathway, and a DNA methylation pathway. In some embodiments, one or more genes from each of at least two of the biological pathways are differentially translated in the first translational profile as compared to the second translational profile. In some embodiments, one or more genes from each of at least three of the biological pathways are differentially translated in the first translational profile as compared to the second translational profile. In some embodiments, the biological pathway, or one of the biological pathways, is the mTOR pathway.

In some embodiments, translational levels are compared for the first and second translational profiles for one or more genes in a protein synthesis pathway. Examples of protein synthesis pathway genes include, but are not limited to, EEF2, RPS12, RPL12, RPS2, RPL13A, RPL18A, EEF1A1, RPL28, RPS28, and RPS27. In some embodiments, translational levels are compared for the first and second translational profiles for one or more genes in a cell invasion/metastasis pathway. Examples of cell invasion/metastasis pathway genes include, but are not limited to, YB1, MTA1, Vimentin, and CD44. In some embodiments, translational levels are compared for the first and second translational profiles for one or more genes in a cell division pathway. Examples of cell division pathway genes include, but are not limited to, CCNI. In some embodiments, translational levels are compared for the first and second translational profiles for one or more genes in an apoptosis pathway. Examples of apoptosis pathway genes include, but are not limited to, ARF, FADD, TNFRSF21, BAX, DAPK, TMS-1, BCL2, RASSF1A, and TERT. In some embodiments, translational levels are compared for the first and second translational profiles for one or more genes in a signal transduction pathway. Examples of signal transduction pathway genes include, but are not limited to, MAPK, MYC, RAS, and RAF. In some embodiments, translational levels are compared for the first and second translational profiles for one or more genes in a cellular transport pathway. Examples of cellular transport pathway genes include, but are not limited to, SLC25A5. In some embodiments, translational levels are compared for the first and second translational profiles for one or more genes in a post-translational protein modification pathway. Examples of post-translational protein modification pathway genes include, but are not limited to, LCMT1 and RABGGTB. In some embodiments, translational levels are compared for the first and second translational profiles for one or more genes in a DNA repair pathway. Examples of DNA repair pathway genes include, but are not limited to, PNKP. In some embodiments, translational levels are compared for the first and second translational profiles for one or more genes in a DNA methylation pathway. Examples of DNA methylation pathway genes include, but are not limited to, AHCY.

In some embodiments, the one or more genes has a 5′ TOP sequence, a PRTE sequence, or both a 5′ TOP sequence and a PRTE sequence. In some embodiments, the one or more genes is selected from the genes listed in Table 1, Table 2, and/or Table 3. In some embodiments, the one or more genes is selected from the group consisting of SEQ ID NOs:1-144.

In some embodiments, the target for therapeutic intervention is a part of an oncogenic signaling pathway. In some embodiments, the oncogenic signaling pathway is the mammalian target of rapamycin (mTOR) pathway, the PI3K pathway, the AKT pathway, the Ras pathway, the Myc pathway, the Wnt pathway, or the BRAF pathway. In some embodiments, the oncogenic signaling pathway that is modulated is the mTOR pathway.

In some embodiments, a method for validating a target for therapeutic intervention in a disease (e.g., an inflammatory disease, an autoimmune disease, a fibrotic disorder, a neurodegenerative disease, a neurodevelopmental disease, a metabolic disease, a viral infection, a cardiomyopathy or a cancer), comprises:
(a) determining a first translational profile for a plurality of genes from a disease sample contacted with an agent that modulates a target;
(b) determining a second translational profile for a plurality of genes from a control disease sample not contacted with the agent; and
(c) validating the target for therapeutic intervention in the disease (e.g., an inflammatory disease, an autoimmune disease, a fibrotic disorder, a neurodegenerative disease, a neurodevelopmental disease, a metabolic disease, a viral infection, a cardiomyopathy or a cancer, respectively) when one or more genes are differentially translated in the first translational profile as compared to the second translational profile and when the differential translation results in a biological benefit.

In some embodiments, a method for validating a target for therapeutic intervention in a disease (e.g., an inflammatory disease, an autoimmune disease, a fibrotic disorder, a neurodegenerative disease, a neurodevelopmental disease, a metabolic disease, a viral infection, a cardiomyopathy or a cancer), comprises:
(a) determining a first translational profile for a plurality of genes from a disease sample contacted with an agent that modulates a target;
(b) determining a second translational profile for a plurality of genes from a disease sample contacted with a known active compound for treating the disease; and
(c) validating the target for therapeutic intervention in a disease (e.g., an inflammatory disease, an autoimmune disease, a fibrotic disorder, a neurodegenerative disease, a neurodevelopmental disease, a metabolic disease, a viral infection, a cardiomyopathy or a cancer, respectively) when the first translational profile is comparable to the second translational profile.

In certain embodiments, a method for validating a target for therapeutic intervention in a disease (e.g., an inflammatory disease, an autoimmune disease, a fibrotic disorder, a neurodegenerative disease, a neurodevelopmental disease, a metabolic disease, a viral infection, a cardiomyopathy or a cancer), comprises:
(a) determining three independent translational profiles, each for a plurality of genes from a disease sample, wherein (i) a first translational profile is from the sample not contacted with any compound, (ii) a second translational profile is from the sample contacted with an agent that modulates a target, and (iii) a third translational profile is from the sample contacted with a known active compound for treating the disease;
(b) identifying one or more genes as differentially translated in the first translational profile as compared to the second translational profile; and
(c) validating the target as a target for therapeutic intervention in the disease (e.g., an inflammatory disease, an autoimmune disease, a fibrotic disorder, a neurodegenerative disease, a neurodevelopmental disease, a metabolic disease, a viral infection, a cardiomyopathy or a cancer, respectively) when the one or more differentially translated genes from step (b) are in the third translational profile and have a translational profile closer to the translational profile of the one or more genes in the second translational profile than to the translational profile of the one or more genes in the first translational profile.

In certain embodiments, a method for validating a target for therapeutic intervention in a disease (e.g., an inflammatory disease, an autoimmune disease, a fibrotic disorder, a neurodegenerative disease, a neurodevelopmental disease, a metabolic disease, a viral infection, a cardiomyopathy or a cancer), comprises:
(a) determining three independent translational profiles, each for a plurality of genes from a disease sample, wherein (i) a first translational profile is from the sample not contacted with any compound, (ii) a second translational profile is from the sample contacted with an agent that modulates a target, and (iii) a third translational profile is from the sample contacted with a known active compound for treating the disease;
(b) determining a first differential translational profile comprising one or more genes differentially translated in the first translational profile as compared to the second translational profile, and determining a second differential translational profile comprising one or more genes differentially translated in the first translational profile as compared to the third translational profile; and
(c) validating the target as a target for therapeutic intervention in the disease (e.g., an inflammatory disease, an autoimmune disease, a fibrotic disorder, a neurodegenerative disease, a neurodevelopmental disease, a metabolic disease, a viral infection, a cardiomyopathy or a cancer, respectively) when the first differential translational profile is comparable to the second differential translational profile.

In any of the aforementioned embodiments for validating a target, the target is suspected of being associated with a disease, is indirectly associated with a disease, or is associated with a disease (e.g., an inflammatory disease, an autoimmune disease, a fibrotic disorder, a neurodegenerative disease, a neurodevelopmental disease, a metabolic disease, a viral infection, a cardiomyopathy or a cancer).

Agents that can be used to validate a target for therapeutic intervention include any agent described herein (e.g., in Section IV(C) above), and include but are not limited to, peptides, proteins, oligopeptides, circular peptides, peptidomimetics, antibodies, polysaccharides, lipids, fatty acids, inhibitory RNAs (e.g., siRNA, miRNA, or shRNA), polynucleotides, oligonucleotides, aptamers, small organic molecules, or drug compounds. In some embodiments, the agent is a small organic molecule. In some embodiments, the agent is a peptide or protein. In some embodiments, the agent is an RNA or inhibitory RNA.

The translational profiles that are generated for validating a target for therapeutic intervention can be generated according to any of the methods described herein. In some embodiments, the translational profiles are generated by ribosomal profiling. In some embodiments, the translational profiles are generated by polysome microarray. In some embodiments, the translational profiles are generated by immunoassay. In some embodiments, the translational profiles comprise translational levels for 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10,000 genes or more in the biological sample. In some embodiments, the first and/or second translational profile comprises translational levels for at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50% or more of all genes in the biological sample. In some embodiments, the translational profiles comprise genome-wide measurements of gene translational levels.

In some embodiments, a target is validated when one or more genes of one or more biological pathways is differentially translated by at least 1.5-fold or at least two-fold (e.g., at least 1.5-fold, at least two-fold, at least three-fold, at least four-fold, at least five-fold, at least six-fold, at least seven-fold, at least eight-fold, at least nine-fold, at least ten-fold or more) in the first translational profile as to the second translational profile. In some embodiments, a target is validated when the translational level for one or more genes of one or more biological pathways is decreased by at least 1.5-fold or at least two-fold (e.g., at least 1.5-fold, at least two-fold, at least three-fold, at least four-fold, at least five-fold, at least six-fold, at least seven-fold, at least eight-fold, at least nine-fold, at least ten-fold or more) in the first translational profile as to the second translational profile. In some embodiments, a target is validated when the translational level for one or more genes of one or more biological pathways is increased by at least 1.5-fold or at least two-fold (e.g., at least 1.5-fold, at least two-fold, at least three-fold, at least four-fold, at least five-fold, at least six-fold, at least seven-fold, at least eight-fold, at least nine-fold, at least ten-fold or more) in the first translational profile as to the second translational profile. In some embodiments, less than 20% of the genes in the genome are differentially translated by at least 1.5-fold or at least two-fold in the first translational profile as compared to the second translational profile. In some embodiments, less than 5% of the genes in the genome are differentially translated by at least 1.5-fold, at least 2-fold, at least 3-fold, or at least 4-fold in the first translational profile as compared to the second translational profile. In some embodiments, less than 1% of the genes in the genome are differentially translated by at least 1.5-fold, at least 2-fold, at least 3-fold, at least 4-fold, or at least 5-fold in the first translational profile as compared to the second translational profile.

In some embodiments, a target is validated when a first differential translational profile is comparable to a second differential translational profile, e.g., when at least of 99%, 95%, 90%, 85%, 80%, 75%, 70%, 65%, 60%, 55%, or 50% of a selected portion of differentially translated genes, a majority of differentially translated genes, or all differentially translated genes show a translational profile within 75%, 70%, 65%, 60%, 55%, 50%, 45%, 40%, 35%, 30%, or 25%, respectively, of their corresponding genes in the reference translational profile. In further embodiments, a first differential translational profile comprising a selected portion of the differentially translated genes or all the differentially translated genes has a differential translational profile comparable to the differential translational profile of the same genes in a second differential translational profile when the amount of protein translated in the first and second differential translational profiles are within about 3.0 log₂, 2.5 log₂, 2.0 log₂, 1.5 log₂, 1.1 log₂, 0.5 log₂, 0.2 log₂ or closer. In still further embodiments, a first differential translational profile comprising a selected portion of the differentially translated genes or all the differentially translated genes has a differential translational profile comparable to the differential translational profile of the same genes in a second differential translational profile when the amount of protein translated in the first and second differential translational profiles differs by no more than about 50%, 40%, 35%, 30%, 25%, 20%, 15%, 10%, 5%, 1% or less.

In some embodiments, a target is validated when the translational profiles comprise one or more gene signatures, wherein one or more gene signatures are comparable in the first and second translational profiles. In certain embodiments, the first and second translational profiles are comparable when an amount of protein translated from one or more differentially translated genes in the first and second translational profiles differs by no more than about 50%, 40%, 35%, 30%, 25%, 20%, 15%, 10%, 5%, 1% or less. In further embodiments, the one or more differentially translated genes from the third translational profile have a translational profile closer to the translational profile of the one or more genes in the second translational profile when the amount of protein translated from the one or more differentially translated genes in the third and second translational profiles differs by no more than about 50%, 40%, 35%, 30%, 25%, 20%, 15%, 10%, 5%, 1% or less.

### VI. Methods of Identifying Drug Candidate Molecules or Agents

In another aspect, the present invention comprises a method of identifying a drug candidate molecule. In some embodiments, the method comprises:
(a) contacting a biological sample with the drug candidate molecule;
(b) determining a translational profile for the contacted biological sample, wherein the translational profile comprises translational levels for a plurality of genes; and
(c) comparing the first translational profile to a second translational profile comprising translational levels for the plurality of genes in a control sample that has not been contacted with the drug candidate molecule,
wherein the drug candidate molecule is identified as suitable for use in a therapeutic intervention when one or more genes of a biological pathway is differentially translated in the first translational profile as compared to the second translational profile, wherein the biological pathway is selected from a protein synthesis pathway, a cell invasion/metastasis pathway, a cell division pathway, an apoptosis pathway, a signal transduction pathway, a cellular transport pathway, a post-translational protein modification pathway, a DNA repair pathway, and DNA methylation pathway.

In some embodiments, the one or more genes (e.g., 1, 2, 3, 4, 5, 6, 7, 8, 9, 10 or more genes) have a 5′ TOP sequence, a PRTE sequence, or both a 5′ TOP sequence and a PRTE sequence. In some embodiments, the one or more genes is selected from the genes listed in Table 1, Table 2, and/or Table 3. In some embodiments, the one or more genes (e.g., 1, 2, 3, 4, 5, 6, 7, 8, 9, 10 or more genes) are selected from the group consisting of SEQ ID NOs:1-144. In some embodiments, one or more genes from each of at least two of the biological pathways are differentially translated in the first translational profile as compared to the second translational profile. In some embodiments, one or more genes from each of at least three of the biological pathways are differentially translated in the first translational profile as compared to the second translational profile. In certain embodiments, the one or more differentially translated genes comprise a plurality of genes and optionally the plurality of differentially translated genes may comprise one or more gene signatures.

In some embodiments, translational levels are compared for the first and second translational profiles for one or more genes in a protein synthesis pathway. Examples of protein synthesis pathway genes include, but are not limited to, EEF2, RPS12, RPL12, RPS2, RPL13A, RPL18A, EEF1A1, RPL28, RPS28, and RPS27. In some embodiments, translational levels are compared for the first and second translational profiles for one or more genes in a cell invasion/metastasis pathway. Examples of cell invasion/metastasis pathway genes include, but are not limited to, YB1, MTA1, Vimentin, and CD44. In some embodiments, translational levels are compared for the first and second translational profiles for one or more genes in a cell division pathway. Examples of cell division pathway genes include, but are not limited to, CCNI. In some embodiments, translational levels are compared for the first and second translational profiles for one or more genes in an apoptosis pathway. Examples of apoptosis pathway genes include, but are not limited to, ARF, FADD, TNFRSF21, BAX, DAPK, TMS-1, BCL2, RASSF1A, and TERT. In some embodiments, translational levels are compared for the first and second translational profiles for one or more genes in a signal transduction pathway. Examples of signal transduction pathway genes include, but are not limited to, MAPK, MYC, RAS, and RAF. In some embodiments, translational levels are compared for the first and second translational profiles for one or more genes in a cellular transport pathway. Examples of cellular transport pathway genes include, but are not limited to, SLC25A5. In some embodiments, translational levels are compared for the first and second translational profiles for one or more genes in a post-translational protein modification pathway. Examples of post-translational protein modification pathway genes include, but are not limited to, LCMT1 and RABGGTB. In some embodiments, translational levels are compared for the first and second translational profiles for one or more genes in a DNA repair pathway. Examples of DNA repair pathway genes include, but are not limited to, PNKP. In some embodiments, translational levels are compared for the first and second translational profiles for one or more genes in a DNA methylation pathway. Examples of DNA methylation pathway genes include, but are not limited to, AHCY.

In some embodiments, a method for identifying a drug candidate molecule or agent for treating a disease (e.g., an inflammatory disease, an autoimmune disease, a fibrotic disorder, a neurodegenerative disease, a neurodevelopmental disease, a metabolic disease, a viral infection, a cardiomyopathy or a cancer), comprises:
(a) determining a first translational profile for a plurality of genes from a disease sample contacted with a candidate agent;
(b) determining a second translational profile for a plurality of genes from a control disease sample not contacted with the agent; and
(c) identifying the agent as a candidate therapeutic for use in treating a disease (e.g., an inflammatory disease, an autoimmune disease, a fibrotic disorder, a neurodegenerative disease, a neurodevelopmental disease, a metabolic disease, a viral infection, a cardiomyopathy or a cancer, respectively) when one or more genes are differentially translated in the first translational profile as compared to the second translational profile and when the differential translation results in a biological benefit.

In certain embodiments, the plurality of differentially translated genes may comprise a plurality of genes, one or more biological pathways, one or more gene signatures, or any combination thereof. A disease sample may be obtained from any subject having a disease of interest to identify drug candidate molecules or agents that affect translational profiles in such samples. In certain embodiments, a biological sample is obtained from a subject who has or is suspected of having a disease, such as an inflammatory disease, an autoimmune disease, a fibrotic disorder, a neurodegenerative disease, a neurodevelopmental disease, a metabolic disease, a viral infection, a cardiomyopathy, or a cancer.

The translational profiles that are generated for identifying a drug candidate molecule or agent can be generated according to any of the methods described herein. In some embodiments, the translational profiles are generated by ribosomal profiling. In some embodiments, the translational profiles are generated by polysome microarray. In some embodiments, the translational profiles are generated by immunoassay. In some embodiments, the translational profiles comprise translational levels for at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10,000 genes or more in the biological sample. In some embodiments, the first and/or second translational profile comprises translational levels for at least about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50% or more of all genes in the biological sample. In some embodiments, the translational profiles comprise genome-wide measurements of gene translational levels.

In some embodiments, a drug candidate molecule or agent is identified as suitable for use in a therapeutic intervention when one or more genes of one or more biological pathways is differentially translated by at least 1.5-fold or at least two-fold (e.g., at least 1.5-fold, at least two-fold, at least three-fold, at least four-fold, at least five-fold, at least six-fold, at least seven-fold, at least eight-fold, at least nine-fold, at least ten-fold or more) in the first translational profile as to the second translational profile. In some embodiments, a drug candidate molecule is identified as suitable for use in a therapeutic intervention when the translational level for one or more genes of one or more biological pathways is decreased by at least 1.5-fold or at least two-fold (e.g., at least 1.5-fold, at least two-fold, at least three-fold, at least four-fold, at least five-fold, at least six-fold, at least seven-fold, at least eight-fold, at least nine-fold, at least ten-fold or more) in the first translational profile as to the second translational profile. In some embodiments, a drug candidate molecule is identified as suitable for use in a therapeutic intervention when the translational level for one or more genes of one or more biological pathways is increased by at least 1.5-fold or at least two-fold (e.g., at least 1.5-fold, at least two-fold, at least three-fold, at least four-fold, at least five-fold, at least six-fold, at least seven-fold, at least eight-fold, at least nine-fold, at least ten-fold or more) in the first translational profile as to the second translational profile. In some embodiments, less than 20% of the genes in the genome are differentially translated by at least 1.5-fold or at least two-fold in the first translational profile as compared to the second translational profile. In some embodiments, less than 5% of the genes in the genome are differentially translated by at least 1.5-fold, at least 2-fold, at least 3-fold, or at least 4-fold in the first translational profile as compared to the second translational profile. In some embodiments, less than 1% of the genes in the genome are differentially translated by at least 1.5-fold, at least 2-fold, at least 3-fold, at least 4-fold, or at least 5-fold in the first translational profile as compared to the second translational profile.

Drug candidate molecules or agents are not limited by therapeutic category, and can include, for example, analgesics, anti-inflammatory agents, antihelminthics, anti-arrhythmic agents, anti-bacterial agents, anti-viral agents, anti-coagulants, anti-depressants, anti-diabetics, anti-epileptics, anti-fungal agent, anti-gout agents, anti-hypertensive agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-blockers, cardiac inotropic agents, corticosteroids, diuretics, anti-parkinsonian agents, gastro-intestinal agents, histamine receptor antagonists, keratolytics, lipid regulating agents, anti-anginal agents, Cox-2 inhibitors, leukotriene inhibitors, macrolides, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, and the like, as well as mixtures thereof.

In some embodiments, the method further comprises comparing the translational profile for the contacted biological sample with a control translational profile for a second biological sample that has been contacted with a known active compound or therapeutic agent. For example, an active compound or therapeutic agent may be known as useful for treating a cancer, a fibrotic disorder, a neurodegenerative disease or disorder, a neurocognitive or neurodevelopmental disorder, an inflammatory disease or disorder, an autoimmune disease or disorder, a viral infection, or the like. In some cases, a candidate agent may mimic the action of an active compound or therapeutic agent known to have a particular function or induce a particular biological effect or phenotypic change in a cell or a subject. In certain embodiments, a candidate agent is identified as a mimic of a known active compound or therapeutic agent by causing a shift in the translational profile to be comparable or similar to the translational profile induced by the known active compound or therapeutic agent. In some embodiments, the known therapeutic agent is a known inhibitor of an oncogenic pathway. In some embodiments, the known therapeutic agent is a known inhibitor of the mammalian target of rapamycin (mTOR) pathway, the PI3K pathway, the AKT pathway, the Ras pathway, the Myc pathway, the Wnt pathway, or the BRAF pathway. In some embodiments, the known therapeutic agent is a known inhibitor of the mTOR pathway.

In some embodiments, a method for identifying a drug candidate molecule or agent useful for treating a disease (e.g., an inflammatory disease, an autoimmune disease, a fibrotic disorder, a neurodegenerative disease, a neurodevelopmental disease, a metabolic disease, a viral infection, a cardiomyopathy or a cancer), comprises:
(a) determining a first translational profile for a plurality of genes from a disease sample contacted with a candidate agent;
(b) determining a second translational profile for a plurality of genes from a control disease sample contacted with a known active compound; and
(c) identifying the agent as a candidate therapeutic for use in treating a disease (e.g., an inflammatory disease, an autoimmune disease, a fibrotic disorder, a neurodegenerative disease, a neurodevelopmental disease, a metabolic disease, a viral infection, a cardiomyopathy or a cancer, respectively) when one or more genes are differentially translated in the first translational profile as compared to the second translational profile and when the differential translation results in a biological benefit.

In some embodiments, a method for identifying a drug candidate molecule or agent useful for treating a disease (e.g., an inflammatory disease, an autoimmune disease, a fibrotic disorder, a neurodegenerative disease, a neurodevelopmental disease, a metabolic disease, a viral infection, a cardiomyopathy or a cancer), comprises:
(a) determining a first translational profile for a plurality of genes from a disease sample contacted with a candidate agent;
(b) determining a second translational profile for a plurality of genes from a control disease sample, respectively, contacted with a known active compound; and
(c) identifying the agent as a candidate therapeutic for use in treating a disease (e.g., an inflammatory disease, an autoimmune disease, a fibrotic disorder, a neurodegenerative disease, a neurodevelopmental disease, a metabolic disease, a viral infection, a cardiomyopathy or a cancer, respectively), when the first translational profile is comparable to the second translational profile.

In still more embodiments, a method for identifying a drug candidate molecule or agent useful for treating a disease (e.g., an inflammatory disease, an autoimmune disease, a fibrotic disorder, a neurodegenerative disease, a neurodevelopmental disease, a metabolic disease, a viral infection, a cardiomyopathy or a cancer), comprises:
(a) determining three independent translational profiles, each for a plurality of genes from a disease sample, wherein (i) a first translational profile is from the sample not contacted with any compound, (ii) a second translational profile is from the sample contacted with a known active compound, and (iii) a third translational profile is from the sample contacted with a candidate agent;
(b) identifying one or more genes as differentially translated in the first translational profile as compared to the second translational profile; and
(c) identifying the agent as a candidate therapeutic for use in treating a disease (e.g., an inflammatory disease, an autoimmune disease, a fibrotic disorder, a neurodegenerative disease, a neurodevelopmental disease, a metabolic disease, a viral infection, a cardiomyopathy or a cancer, respectively) when the one or more differentially translated genes from step (b) are in the third translational profile and have a translational profile closer to the translational profile of the one or more genes in the second translational profile than to the translational profile of the one or more genes in the first translational profile.

In further embodiments, a method for identifying a drug candidate molecule or agent useful for treating a disease (e.g., a cancer, an inflammatory disease, an autoimmune disease, a fibrotic disorder, a neurodegenerative disease, a neurodevelopmental disease, a metabolic disease, a viral infection, or a cardiomyopathy), comprises:
(a) determining three independent translational profiles, each for a plurality of genes from a disease sample, wherein (i) a first translational profile is from the sample not contacted with any compound, (ii) a second translational profile is from the sample contacted with a known active compound, and (iii) a third translational profile is from the sample contacted with a candidate agent;
(b) determining a first differential translational profile comprising one or more genes differentially translated in the first translational profile as compared to the second translational profile, and determining a second differential translational profile comprising one or more genes differentially translated in the first translational profile as compared to the third translational profile; and
(c) identifying the agent as a candidate therapeutic for use in treating a disease (e.g., a cancer, an inflammatory disease, an autoimmune disease, a fibrotic disorder, a neurodegenerative disease, a neurodevelopmental disease, a metabolic disease, a viral infection, or a cardiomyopathy, respectively) when the first differential translational profile is comparable to the second differential translational profile.

In some embodiments, the differentially translated genes comprise one or more biological pathways, such as at least two or at least three biological pathways. In certain embodiments, the one or more differentially translated genes comprise a plurality of genes and optionally the plurality of differentially translated genes may comprise one or more gene signatures. In further embodiments, the one or more genes are differentially translated at least a two-fold or more. In still further embodiments, each translational profile comprises at least 100 genes, at least 200 genes, at least 300 genes, at least 400 genes, at least 500 genes, or each translational profile comprises a genome-wide translational profile. For example, less than about 25%, about 20%, about 15%, about 10%, about 5%, about 4%, about 3%, about 2% or about 1% of the genes in the genome are differentially translated in a translational profile from a disease sample treated with a drug candidate molecule or agent as compared to a translational profile of an untreated disease sample.

In some embodiments, the known active compound is for use in treating an inflammatory disease, autoimmune disease, fibrotic disorder, neurodegenerative disease, neurodevelopmental disease, metabolic disease, viral infection, cardiomyopathy or cancer. In some embodiments, the known active compound is a therapeutic for use in treating a cancer selected from prostate cancer, breast cancer, bladder cancer, lung cancer, renal cell carcinoma, endometrial cancer, melanoma, ovarian cancer, thyroid cancer, or brain cancer. In some embodiments, the known active compound is a therapeutic for use in treating an inflammatory disease selected from ankylosing spondylitis, atherosclerosis, multiple sclerosis, systemic lupus erythematosus (SLE), psoriasis, psoriatic arthritis, rheumatoid arthritis, ulcerative colitis, inflammatory bowel disease, or Crohn's disease. In some embodiments, the known active compound is a therapeutic for use in treating a fibrotic disorder selected from pulmonary fibrosis, idiopathic pulmonary fibrosis, cystic fibrosis, liver fibrosis, cardiac fibrosis, endomyocardial fibrosis, atrial fibrosis, mediastinal fibrosis, myelofibrosis, retroperitoneal fibrosis, chronic kidney disease, nephrogenic systemic fibrosis, Crohn's disease, hypertrophic scarring, keloid, scleroderma, organ transplant associated fibrosis, or ischemia associated fibrosis. In some embodiments, the known active compound is a therapeutic for use in treating a neurodegenerative disease selected from Parkinson's disease, Alzheimer's disease, Amyotrophic Lateral Sclerosis, Creutzfeldt-Jakob disease, Huntington's disease, Lewy body dementia, frontotemporal dementia, corticobasal degeneration, primary progressive aphasia or progressive supranuclear palsy. In some embodiments, the known active compound is a therapeutic for use in treating a neurodevelopmental disease selected from autism, autism spectrum disorders, Fragile X Syndrome, attention deficit disorder, or a pervasive development disorder. In some embodiments, the known active compound is a therapeutic for use in treating a viral infection selected from adenovirus, bunyavirus, herpesvirus, papovavirus, paramyxovirus, picornavirus, rhabdovirus, orthomyxovirus, poxvirus, reovirus, retrovirus, lentivirus, or flavivirus.

In some embodiments, the translational profiles comprise one or more gene signatures, wherein one or more gene signatures are comparable in the first and second translational profiles. In certain embodiments, the first and second translational profiles are comparable when an amount of protein translated from one or more differentially translated genes in the first and second translational profiles differs by no more than about 25%, 20%, 15%, 10%, 5%, 1% or less. In further embodiments, the one or more differentially translated genes from the third translational profile have a translational profile closer to the translational profile of the one or more genes in the second translational profile when the amount of protein translated from the one or more differentially translated genes in the third and second translational profiles differs by no more than about 25%, 20%, 15%, 10%, 5%, 1% or less.

In some embodiments, the methods of identifying a drug candidate molecule as described herein are used to compare a group of drug candidate molecules and select one drug candidate molecule or a smaller subgroup of drug candidate molecules from this group. In some embodiments, the methods described herein are used to compare drug candidate molecules and select one candidate molecule or a subgroup of drug candidate molecules which alter the translation of a relatively smaller number of proteins, as compared to the number of proteins for which translational is altered for the larger group of drug candidate molecules. In some embodiments, the methods described herein are used to compare drug candidate molecules and select one candidate molecule or a subgroup of drug candidate molecules for which altered translation resides in a relatively smaller number of pathways, as compared to the number of pathways for which translation is altered for the larger group of drug candidate molecules. In some embodiments, the methods described herein are used to compare drug candidate molecules and select one candidate molecule or a subgroup of drug candidate molecules which alter the translation of several proteins within one specific pathway, as compared to the larger group of drug candidate molecules for which a smaller number of proteins within that one specific pathway have altered translation.

### VII. Therapeutic Methods

In yet another aspect, the present invention provides therapeutic methods for identifying subjects for treatment and treating subjects in need thereof. In some embodiments, the present invention relates to methods of identifying a subject as a candidate for treatment, e.g., for treatment with an mTOR inhibitor. In some embodiments, the present invention relates to methods of treating a subject, e.g., a subject having a cancer, an inflammatory disease, an autoimmune disease, a fibrotic disorder, a neurodegenerative disease, a neurodevelopmental disease, a metabolic disease, or a viral infection.

A. Identifying Subjects for Treatment

In some embodiments, the present invention relates to a method of identifying a subject as a candidate for treatment with an mTOR inhibitor. In some embodiments, the method comprises:
(a) determining a first translational profile in a sample from the subject, wherein the first translational profile comprises translational levels for one or more genes having a 5′ terminal oligopyrimidine tract (5′ TOP) and/or a pyrimidine-rich translational element (PRTE); and
(b) comparing the first translational profile to a second translational profile comprising translational levels for the one or more genes, wherein the second translational profile is from a control sample, wherein the control sample is from a known responder to the mTOR inhibitor prior to administration of the mTOR inhibitor to the known responder;
wherein a translational level of the one or more genes in the first translational profile that is at least as high as the translational level of the one or more genes in the second translational profile identifies the subject as a candidate for treatment with the mTOR inhibitor.

In some embodiments, the one or more genes (e.g., the 1, 2, 3, 4, 5, 6, 7, 8, 9, 10, 11, 12, 13, 14, 15, 16, 17, 18, 19, 20 or more genes) are selected from the genes listed in any of Table 1, Table 2, or Table 3.

In some embodiments, a method of identifying a subject as a candidate for treatment with an mTOR inhibitor comprises:
(a) determining a first translational profile in a sample from the subject, wherein the first translational profile comprises translational levels for one or more genes selected from the group consisting of SEQ ID NOs:1-144; and
(b) comparing the first translational profile to a second translational profile comprising translational levels for the one or more genes, wherein the second translational profile is from a control sample, wherein the control sample is from a known responder to the mTOR inhibitor prior to administration of the mTOR inhibitor to the known responder;
wherein a translational level of the one or more genes in the first translational profile that is at least as high as the translational level of the one or more genes in the second translational profile identifies the subject as a candidate for treatment with the mTOR inhibitor.

In some embodiments, the one or more genes (e.g., the 1, 2, 3, 4, 5, 6, 7, 8, 9, 10, 11, 12, 13, 14, 15, 16, 17, 18, 19, 20 or more genes) are cell invasion/metastasis genes. In some embodiments, the one or more genes are selected from YB1, vimentin, MTA1, and CD44.

In some embodiments, a method of identifying a subject as a candidate for treatment with an mTOR inhibitor comprises:
(a) determining a first translational profile in a sample from the subject, wherein the first translational profile comprises translational levels for one or more genes of a biological pathway, wherein the biological pathway is selected from a protein synthesis pathway, a cell invasion/metastasis pathway, a cell division pathway, an apoptosis pathway, a signal transduction pathway, a cellular transport pathway, a post-translational protein modification pathway, a DNA repair pathway, and a DNA methylation pathway; and
(b) comparing the first translational profile to a second translational profile comprising translational levels for the one or more genes, wherein the second translational profile is from a control sample, wherein the control sample is from a known responder to the mTOR inhibitor prior to administration of the mTOR inhibitor to the known responder;
wherein a translational level of the one or more genes in the first translational profile that is at least as high as the translational level of the one or more genes in the second translational profile identifies the subject as a candidate for treatment with the mTOR inhibitor.

In some embodiments, the methods of the present invention relate to a method of identifying a subject as a candidate for treatment with a therapeutic agent. In some embodiments, the method comprises:
(a) determining a first translational profile in a sample from the subject, wherein the translational profile comprises translational levels for one or more genes of a biological pathway, wherein the biological pathway is selected from a protein synthesis pathway, a cell invasion/metastasis pathway, a cell division pathway, an apoptosis pathway, a signal transduction pathway, a cellular transport pathway, a post-translational protein modification pathway, a DNA repair pathway, and a DNA methylation pathway; and
(b) comparing the first translational profile to a second translational profile comprising translational levels for the one or more genes, wherein the second translational profile is from a control sample, wherein the control sample is from a known responder to the therapeutic agent prior to administration of the therapeutic agent to the known responder;
wherein a translational level of the one or more genes that is at least as high as the translational level of the one or more genes in the second translational profile identifies the subject as a candidate for treatment with the therapeutic agent.

In further embodiments, a method for identifying a subject as a candidate for treating a disease (e.g., a cancer, an inflammatory disease, an autoimmune disease, a fibrotic disorder, a neurodegenerative disease, a neurodevelopmental disease, a metabolic disease, a viral infection, or a cardiomyopathy), comprises:
(a) determining a first translational profile for a plurality of genes in a sample from a subject having or suspected of having a disease;
(b) determining a second translational profile for a plurality of genes in a control disease sample, wherein the control sample is from a subject known to respond to the therapeutic agent and the sample has not been contacted with a therapeutic agent for a disease; and
(c) identifying the subject as a candidate for treating a disease (e.g., a cancer, an inflammatory disease, an autoimmune disease, a fibrotic disorder, a neurodegenerative disease, a neurodevelopmental disease, a metabolic disease, a viral infection, or a cardiomyopathy) (e.g., a cancer, an inflammatory disease, an autoimmune disease, a fibrotic disorder, a neurodegenerative disease, a neurodevelopmental disease, a metabolic disease, a viral infection, or a cardiomyopathy, respectively) with a therapeutic agent when the first translational profile is comparable to the second translational profile.

In some embodiments, the translational profiles comprise one or more gene signatures, wherein one or more gene signatures are comparable in the first and second translational profiles. In certain embodiments, the first and second translational profiles are comparable when an amount of protein translated from one or more differentially translated genes in the first and second translational profiles differs by no more than about 25%, 20%, 15%, 10%, 5%, 1% or less.

In some embodiments, translational levels are compared for the first translational profile and the second translational profile for 1, 2, 3, 4, 5, 6, 7, 8, 9, 10, 11, 12, 13, 14, 15, 16, 17, 18, 19, 20 or more genes in one or more biological pathways. In some embodiments, the translational level of one or more genes from each of at least two of the biological pathways is at least as high in the first translational profile as compared to the second translational profile. In some embodiments, the translational level of one or more genes from each of at least three of the biological pathways is at least as high in the first translational profile as compared to the second translational profile.

In some embodiments, the first and/or second translational profiles comprise translational levels for at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1500, 2000, 2500, 3000, 3500, 4000, 4500, 5000, 5500, 6000, 6500, 7000, 7500, 8000, 8500, 9000, 9500, 10,000 genes or more in the biological sample. In some embodiments, the first and/or second translational profile comprises translational levels for at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50% or more of all genes in the biological sample. In some embodiments, the translational profiles comprise genome-wide measurements of gene translational levels. In some embodiments, the translational level of the one or more genes is increased by at least 1.5-fold or at least two-fold (e.g., at least 1.5-fold, at least two-fold, at least three-fold, at least four-fold, at least five-fold, at least six-fold, at least seven-fold, at least eight-fold, at least nine-fold, at least ten-fold or more) in the first translational profile as to the second translational profile. In some embodiments, the translational level of the one or more genes is decreased by at least 1.5-fold or at least two-fold (e.g., at least 1.5-fold, at least two-fold, at least three-fold, at least four-fold, at least five-fold, at least six-fold, at least seven-fold, at least eight-fold, at least nine-fold, at least ten-fold or more) in the first translational profile as to the second translational profile.

In some embodiments, the disease is a cancer. Non-limiting examples of cancers that can be treated according to the methods of the present invention include, but are not limited to, anal carcinoma, bladder carcinoma, breast carcinoma, cervix carcinoma, chronic lymphocytic leukemia, chronic myelogenous leukemia, endometrial carcinoma, hairy cell leukemia, head and neck carcinoma, lung (small cell) carcinoma, multiple myeloma, non-Hodgkin's lymphoma, follicular lymphoma, ovarian carcinoma, brain tumors, colorectal carcinoma, hepatocellular carcinoma, Kaposi's sarcoma, lung (non-small cell carcinoma), melanoma, pancreatic carcinoma, prostate carcinoma, renal cell carcinoma, and soft tissue sarcoma.

In some embodiments, the disease is an inflammatory disease (e.g., an autoimmune disease, arthritis, or MS). In some embodiments, the disease is a fibrotic disorder (e.g., pulmonary fibrosis, idiopathic pulmonary fibrosis, cystic fibrosis, liver fibrosis, cardiac fibrosis, mediastinal fibrosis, myelofibrosis, keloids, scleroderma, organ transplant associated fibrosis, or ischemia associated fibrosis). In some embodiments, the disease is a neurodegenerative disease (e.g., Parkinson's disease, Amyotrophic Lateral Sclerosis (ALS), Creutzfeldt-Jakob disease, Huntington's disease, Lewy body dementia, frontotemporal dementia, corticobasal degeneration, primary progressive aphasia, progressive supranuclear palsy or Alzheimer's disease). In some embodiments, the disease is a neurodevelopmental disease (e.g., autism, autism spectrum disorders, Fragile X Syndrome, attention deficit disorder, pervasive development disorders). In some embodiments, the disease is a metabolic disease (e.g., diabetes, metabolic syndrome, or a cardiovascular disease). In some embodiments, the disease is a viral infection (e.g., adenovirus, herpesvirus, papovavirus, poxvirus, retrovirus, lentivirus, or flavivirus). In some embodiments, the disease is a cardiomyopathy.

In some embodiments, a disease is associated with one or more altered biological pathways. In some embodiments, wherein a cell communication pathway is altered, the disease is an immune or inflammatory disease, a fibrotic disorder, a neurodegenerative disease, a neurodevelopment disease, a cancer, a metabolic disorder, or a viral disease. In some embodiments, wherein a cell communication pathway is altered, the disease is an immune or inflammatory disease (e.g., an autoimmune disease, arthritis, or MS).

In some embodiments, wherein a cellular process pathway is altered, the disease is an immune or inflammatory disease (e.g., an autoimmune disease, arthritis, or MS), a fibrotic disorder, a neurodegenerative disease (e.g., Parkinson's disease, Amyotrophic Lateral Sclerosis (ALS), Creutzfeldt-Jakob disease, Huntington's disease, Lewy body dementia, frontotemporal dementia, corticobasal degeneration, primary progressive aphasia, progressive supranuclear palsy, or Alzheimer's disease), a neurodevelopmental disease (e.g., autism, autism spectrum disorders, Fragile X Syndrome, attention deficit disorder, pervasive development disorders), a cancer, a metabolic disorder, or a viral disease.

In some embodiments, wherein an immune system process pathway is altered, the disease is an immune or inflammatory disease, a fibrotic disorder, a neurodegenerative disease, a neurodevelopmental disease, a cancer, a metabolic disorder, or a viral disease. In some embodiments, wherein an immune system process pathway is altered, the disease is an immune or inflammatory disease (e.g., an autoimmune disease, arthritis, or MS).

In some embodiments, wherein a response to stimulus pathway is altered, the disease is an immune or inflammatory disease, a fibrotic disorder, a neurodegenerative disease, a neurodevelopmental disease, a metabolic disorder, or a viral disease. In some embodiments, wherein a response to stimulus pathway is altered, the disease is an immune or inflammatory disease (e.g., an autoimmune disease, arthritis, or MS) or a viral disease.

In some embodiments, wherein a transport pathway is altered, the disease is an immune or inflammatory disease, a fibrotic disorder, a neurodegenerative disease, or a metabolic disorder. In some embodiments, wherein a transport pathway is altered, the disease is an immune or inflammatory disease (e.g., an autoimmune disease, arthritis, or MS) or a metabolic disorder (e.g., diabetes, metabolic syndrome, or a cardiovascular disease).

In some embodiments, wherein a metabolic process pathway is altered, the disease is a fibrotic disorder, a neurodegenerative disease, a neurodevelopmental disease, a cancer, or a metabolic disorder. In some embodiments, wherein a metabolic process pathway is altered, the disease is a metabolic disorder (e.g., diabetes, metabolic syndrome, or a cardiovascular disease).

In some embodiments, a metabolic process pathway is a carbohydrate metabolic process pathway, a lipid metabolic process pathway, a nucleobase, nucleoside, or nucleotide pathway, or a protein metabolic process pathway (e.g., a proteolysis pathway, a protein complex assembly pathway, a protein folding pathway, a protein modification process pathway, or a translation pathway). In some embodiments, wherein a carbohydrate metabolic process pathway is altered, the disease is a fibrotic disorder, a neurodegenerative disease, or a metabolic disorder. In some embodiments, wherein a lipid metabolic process pathway is altered, the disease is an immune or inflammatory disease, a fibrotic disorder, a neurodegenerative disease, or a metabolic disorder. In some embodiments, wherein a nucleobase, nucleoside, or nucleotide pathway is altered, the disease is a cancer or a viral disease. In some embodiments, wherein a protein metabolic process pathway is altered, the disease is an immune or inflammatory disease, a fibrotic disorder, a neurodegenerative disease, a neurodevelopmental disease, a cancer, a metabolic disorder, or a viral disease. In some embodiments, wherein a proteolysis process pathway is altered, the disease is an immune or inflammatory disease, a fibrotic disorder, a neurodegenerative disease, a neurodevelopmental disease, a cancer, or a metabolic disorder. In some embodiments, wherein a protein complex assembly pathway is altered, the disease is a metabolic disorder. In some embodiments, wherein a protein folding pathway is altered, the disease is a neurodegenerative disease. In some embodiments, wherein a protein modification process pathway is altered, the disease is an immune or inflammatory disease, a fibrotic disorder, a neurodevelopmental disease, a neurodegenerative disease, a cancer, a metabolic disorder, or a viral disease. In some embodiments, wherein a protein translation pathway is altered, the disease is an immune or inflammatory disease, a fibrotic disorder, a neurodegenerative disease, a neurodevelopmental disease, or a cancer.

In some embodiments, the method further comprises administering a therapeutic agent to the identified subject. In some embodiments, the method further comprises administering an mTOR inhibitor to the identified subject.

B. Administration of Therapeutic Agents

In some embodiments, the present invention relates to a method of treating a subject having a cancer. In some embodiments, the method comprises:
administering an mTOR inhibitor to a subject that has been selected as having a first translational profile comprising a translational level of one or more genes that is at least as high as the translational level of the one or more genes in a second translational profile from a control sample;
wherein the first and second translational profiles comprise translational levels for one or more genes having a 5′ terminal oligopyrimidine tract (5′ TOP) and/or a pyrimidine-rich translational element (PRTE); and wherein the control sample is from a known responder to the mTOR inhibitor prior to administration of the mTOR inhibitor to the known responder;
thereby treating the cancer in the subject.

In some embodiments, the method of treating a subject having a cancer comprises:
administering an mTOR inhibitor to a subject that has been selected as having a first translational profile comprising a translational level of one or more genes that is at least as high as the translational level of the one or more genes in a second translational profile from a control sample;
wherein the first and second translational profiles comprise translational levels for one or more genes selected from the group consisting of SEQ ID NOs:1-144; and wherein the control sample is from a known responder to the mTOR inhibitor prior to administration of the mTOR inhibitor to the known responder;
thereby treating the cancer in the subject.

In some embodiments, the method of treating a subject having a cancer comprises:
administering an mTOR inhibitor to a subject that has been selected as having a first translational profile comprising a translational level of one or more genes that is at least as high as the translational level of the one or more genes in a second translational profile from a control sample;
wherein the first and second translational profiles comprise translational levels for one or more genes of a biological pathway selected from a protein synthesis pathway, a cell invasion/metastasis pathway, a cell division pathway, an apoptosis pathway, a signal transduction pathway, a cellular transport pathway, a post-translational protein modification pathway, a DNA repair pathway, and a DNA methylation pathway; and wherein the control sample is from a known responder to the mTOR inhibitor prior to administration of the mTOR inhibitor to the known responder;
thereby treating the cancer in the subject.

In some embodiments, the present invention relates to a method of treating a subject in need thereof. In some embodiments, the method comprises:
administering a therapeutic agent to a subject that has been selected as having a first translational profile comprising a translational level of one or more genes that is at least as high as the translational level of the one or more genes in a second translational profile;
wherein the first and second translational profiles comprise translational levels for one or more genes of a biological pathway selected from a protein synthesis pathway, a cell invasion/metastasis pathway, a cell division pathway, an apoptosis pathway, a signal transduction pathway, a cellular transport pathway, a post-translational protein modification pathway, a DNA repair pathway, and a DNA methylation pathway; and wherein the control sample is from a known responder to the therapeutic agent prior to administration of the therapeutic agent to the known responder;
thereby treating the subject.

In certain embodiments, a method for treating a disease (e.g., a cancer, an inflammatory disease, an autoimmune disease, a fibrotic disorder, a neurodegenerative disease, a neurodevelopmental disease, a metabolic disease, a viral infection, or a cardiomyopathy), comprises administering to a subject identified by:
(a) determining a first translational profile for a plurality of genes in a sample from a subject having or suspected of having a disease;
(b) determining a second translational profile for a plurality of genes in a control disease sample, wherein the control sample is from a subject known to respond to the therapeutic agent and the sample has not been contacted with a therapeutic agent for a disease; and
(c) identifying the subject as a candidate for treating a disease (e.g., a cancer, an inflammatory disease, an autoimmune disease, a fibrotic disorder, a neurodegenerative disease, a neurodevelopmental disease, a metabolic disease, a viral infection, or a cardiomyopathy, respectively) with a therapeutic agent when the first translational profile is comparable to the second translational profile;
thereby treating the subject.

In further embodiments, a method for treating a cancer, an inflammatory disease, an autoimmune disease, a fibrotic disorder, a neurodegenerative disease, a neurodevelopmental disease, a metabolic disease, a viral infection, or cardiomyopathy, comprises administering to a subject having a disease an agent or drug candidate molecule identified according to any one of the methods provided herein, thereby treating the subject.

In still further embodiments, a method for treating a cancer, an inflammatory disease, an autoimmune disease, a fibrotic disorder, a neurodegenerative disease, a neurodevelopmental disease, a metabolic disease, a viral infection, or a cardiomyopathy, comprises administering to a subject having a disease an agent that modulates a target, wherein the target was validated according to any one of the methods provided herein, thereby treating the subject.

In yet further embodiments, a method for treating a cancer, an inflammatory disease, an autoimmune disease, a fibrotic disorder, a neurodegenerative disease, a neurodevelopmental disease, a metabolic disease, a viral infection, or cardiomyopathy, by normalizing the disease translational profile, comprises administering to a subject having a disease an agent that modulates a target, wherein the target was validated according to any one of the methods provided herein, thereby treating the subject.

In any of the aforementioned embodiments for treating a cancer, an inflammatory disease, an autoimmune disease, a fibrotic disorder, a neurodegenerative disease, a neurodevelopmental disease, a metabolic disease, a viral infection, or cardiomyopathy according to a validated target, the target that was validated was suspected of being associated with a disease, was indirectly associated with a disease, or was associated with a disease (e.g., an inflammatory disease, an autoimmune disease, a fibrotic disorder, a neurodegenerative disease, a neurodevelopmental disease, a metabolic disease, a viral infection, a cardiomyopathy or a cancer, respectively).

In some embodiments, the translational profiles comprise one or more gene signatures, wherein one or more gene signatures are comparable in the first and second translational profiles. In certain embodiments, the first and second translational profiles are comparable when an amount of protein translated from one or more differentially translated genes in the first and second translational profiles differs by no more than about 50%, 45%, 40%, 35%, 30%, 25%, 20%, 15%, 10%, 5%, 1% or less. In further embodiments, the one or more differentially translated genes from the third translational profile have a translational profile closer to the translational profile of the one or more genes in the second translational profile when the amount of protein translated from the one or more differentially translated genes in the third and second translational profiles differs by no more than about 50%, 45%, 40%, 35%, 30%, 25%, 20%, 15%, 10%, 5%, 1% or less.

A subject is selected for therapeutic treatment based on any of the translational profiling methods as described herein. In some embodiments, the subject has a disease. In some embodiments, the disease is an inflammatory disease. In some embodiments, the disease is a fibrotic disorder. In some embodiments, the disease is a neurodegenerative disease. In some embodiments, the disease is a neurodevelopmental disease. In some embodiments, the disease is a metabolic disease. In some embodiments, the disease is viral infection. In some embodiments, the disease is a cardiomyopathy. In some embodiments, the disease is cancer.

Non-limiting examples of cancers that can be treated according to the methods of the present invention include, but are not limited to, anal carcinoma, bladder carcinoma, breast carcinoma, cervix carcinoma, chronic lymphocytic leukemia, chronic myelogenous leukemia, endometrial carcinoma, hairy cell leukemia, head and neck carcinoma, lung (small cell) carcinoma, multiple myeloma, non-Hodgkin's lymphoma, follicular lymphoma, ovarian carcinoma, brain tumors, colorectal carcinoma, hepatocellular carcinoma, Kaposi's sarcoma, lung (non-small cell carcinoma), melanoma, pancreatic carcinoma, prostate carcinoma, renal cell carcinoma, and soft tissue sarcoma. In some embodiments, the cancer is prostate cancer, breast cancer, bladder cancer, lung cancer, renal cell carcinoma, endometrial cancer, melanoma, ovarian cancer, thyroid cancer, or brain cancer. In some embodiments, the cancer is an invasive cancer.

Non-limiting examples of inflammatory and autoimmune diseases that can be treated according to the methods of the present disclosure include, but are not limited to, arthritis, rheumatoid arthritis, juvenile rheumatoid arthritis, osteoarthritis, polychondritis, psoriatic arthritis, psoriasis, dermatitis, polymyositis/dermatomyositis, inclusion body myositis, inflammatory myositis, toxic epidermal necrolysis, systemic scleroderma and sclerosis, CREST syndrome, inflammatory bowel disease, Crohn's disease, ulcerative colitis, respiratory distress syndrome, adult respiratory distress syndrome (ARDS), chronic obstructive pulmonary disease, meningitis, encephalitis, uveitis, colitis, glomerulonephritis, allergic conditions, eczema, asthma, conditions involving infiltration of T cells and chronic inflammatory responses, atherosclerosis, autoimmune myocarditis, leukocyte adhesion deficiency, systemic lupus erythematosus (SLE), subacute cutaneous lupus erythematosus, discoid lupus, lupus myelitis, lupus cerebritis, juvenile onset diabetes, multiple sclerosis (MS), allergic encephalomyelitis, neuromyelitis optica, rheumatic fever, Sydenham's chorea, immune responses associated with acute and delayed hypersensitivity mediated by cytokines and T-lymphocytes, tuberculosis, sarcoidosis, granulomatosis including Wegener's granulomatosis and Churg-Strauss disease, agranulocytosis, vasculitis (including hypersensitivity vasculitis/angiitis, ANCA and rheumatoid vasculitis), aplastic anemia, Diamond Blackfan anemia, immune hemolytic anemia including autoimmune hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, central nervous system (CNS) inflammatory disorders, multiple organ injury syndrome, myasthenia gravis, antigen-antibody complex mediated diseases, anti-glomerular basement membrane disease, anti-phospholipid antibody syndrome, allergic neuritis, Behcet disease, Castleman's syndrome, Goodpasture's syndrome, Lambert-Eaton Myasthenic Syndrome, Reynaud's syndrome, Sjorgen's syndrome, Stevens-Johnson syndrome, solid organ transplant rejection, graft-versus-host disease (GVHD), bullous pemphigoid, pemphigus, autoimmune polyendocrinopathies, seronegative spondyloarthropathies, Reiter's disease, stiff-man syndrome, giant cell arteritis, immune complex nephritis, IgA nephropathy, IgM polyneuropathies or IgM mediated neuropathy, idiopathic thrombocytopenic purpura (ITP), thrombotic throbocytopenic purpura (TTP), Henoch-Schonlein purpura, autoimmune thrombocytopenia, autoimmune disease of the testis and ovary including autoimmune orchitis and oophoritis, primary hypothyroidism; autoimmune endocrine diseases including autoimmune thyroiditis, chronic thyroiditis (Hashimoto's Thyroiditis), subacute thyroiditis, idiopathic hypothyroidism, Addison's disease, Grave's disease, autoimmune polyglandular syndromes (or polyglandular endocrinopathy syndromes), Type I diabetes (also referred to as insulin-dependent diabetes mellitus or IDDM); autoimmune hepatitis, lymphoid interstitial pneumonitis (HIV), bronchiolitis obliterans (non-transplant), non-specific interstitial pneumonia (NSIP), Guillain-BarréSyndrome, large vessel vasculitis (including polymyalgia rheumatica and giant cell (Takayasu's) arteritis), medium vessel vasculitis (including Kawasaki's disease and polyarteritis nodosa), polyarteritis nodosa (PAN), ankylosing spondylitis, Berger's disease (IgA nephropathy), rapidly progressive glomerulonephritis, primary biliary cirrhosis, Celiac sprue (gluten enteropathy), cryoglobulinemia, cryoglobulinemia associated with hepatitis, amyotrophic lateral sclerosis (ALS), coronary artery disease, familial Mediterranean fever, microscopic polyangiitis, Cogan's syndrome, Whiskott-Aldrich syndrome and thromboangiitis obliterans. In some embodiments, the inflammatory disease is ankylosing spondylitis, multiple sclerosis, systemic lupus erythematosus (SLE), rheumatoid arthritis, atherosclerosis, inflammatory bowel disease, or Crohn's disease.

Non-limiting examples of infectious viruses include adenovirus, bunyavirus (e.g., Hantavirus), herpesvirus, papovavirus, paramyxovirus, picornavirus, rhabdovirus (e.g., Rabies), orthomyxovirus (e.g., influenza), poxvirus (e.g., Vaccinia), reovirus, retrovirus, lentivirus (e.g., HIV), flavivirus (e.g., HCV), or the like).

The term “fibrotic disorder” or “fibrotic disease” refers to a medical condition featuring progressive and/or irreversible fibrosis, wherein excessive deposition of extracellular matrix occurs in and around inflamed or damaged tissue. Excessive and persistent fibrosis can progressively remodel and destroy normal tissue, which may lead to dysfunction and failure of affected organs, and ultimately death. A fibrotic disorder may affect any tissue in the body and is generally initiated by an injury. It is to be understood that fibrosis alone triggered by normal wound healing processes that has not progressed to a pathogenic state is not considered a fibrotic disorder or disease of this disclosure. A “fibrotic lesion” or “fibrotic plaque” refers to a focal area of fibrosis. As used herein, “injury” refers to an event that damages tissue and initiates fibrosis. An injury may be caused by an external factor, such as mechanical insult (e.g., cut, surgery), exposure to radiation, chemicals (e.g., chemotherapy, toxins, irritants, smoke), or infectious agent (e.g., bacteria, virus, or parasite). An injury may be caused by, for example, chronic autoimmune inflammation, allergic response, HLA mismatching (e.g., transplant recipients), or ischemia (i.e., an “ischemic event” or “ischemia” refers to an injury that restricts in blood supply to a tissue, resulting in damage to or dysfunction of tissue, which may be caused by problems with blood vessels, atherosclerosis, thrombosis or embolism, and may affect a variety of tissues and organs; an ischemic event may include, for example, a myocardial infarction, stroke, organ or tissue transplant, or renal artery stenosis). In certain embodiments, an injury leading to a fibrotic disorder may be of unknown etiology (i.e., idiopathic).

Non-limiting examples of fibrotic disorders or fibrotic diseases include pulmonary fibrosis, idiopathic pulmonary fibrosis, cystic fibrosis, liver fibrosis (e.g., cirrhosis), cardiac fibrosis, endomyocardial fibrosis, atrial fibrosis, mediastinal fibrosis, myelofibrosis, retroperitoneal fibrosis, progressive massive fibrosis (e.g., lungs), chronic kidney disease, nephrogenic systemic fibrosis, Crohn's disease, hypertrophic scarring, keloid, scleroderma, systemic sclerosis (e.g., skin, lungs), athrofibrosis (e.g., knee, shoulder, other joints), Peyronie's disease, Dupuytren's contracture, adhesive capsulitis, organ transplant associated fibrosis, ischemia associated fibrosis, or the like.

A therapeutic agent for use according to any of the methods of the present invention can be any composition that has or may have a pharmacological activity. Agents include compounds that are known drugs, compounds for which pharmacological activity has been identified but which are undergoing further therapeutic evaluation, and compounds that are members of collections and libraries that are screened for a pharmacological activity. In some embodiments, the therapeutic agent is an anti-cancer, e.g., an anti-signaling agent (e.g., a cytostatic drug) such as a monoclonal antibody or a tyrosine kinase inhibitor; an anti-proliferative agent; a chemotherapeutic agent (i.e., a cytotoxic drug); a hormonal therapeutic agent; and/or a radiotherapeutic agent.

Generally, the therapeutic agent is administered at a therapeutically effective amount or dose. A therapeutically effective amount or dose will vary according to several factors, including the chosen route of administration, the formulation of the composition, patient response, the severity of the condition, the subject's weight, and the judgment of the prescribing physician. The dosage can be increased or decreased over time, as required by an individual patient. In certain instances, a patient initially is given a low dose, which is then increased to an efficacious dosage tolerable to the patient. Determination of an effective amount is well within the capability of those skilled in the art.

The route of administration of a therapeutic agent can be oral, intraperitoneal, transdermal, subcutaneous, by intravenous or intramuscular injection, by inhalation, topical, intralesional, infusion; liposome-mediated delivery; topical, intrathecal, gingival pocket, rectal, intrabronchial, nasal, transmucosal, intestinal, ocular or otic delivery, or any other methods known in the art.

In some embodiments, a therapeutic agent is formulated as a pharmaceutical composition. In some embodiments, a pharmaceutical composition incorporates particulate forms, protective coatings, protease inhibitors, or permeation enhancers for various routes of administration, including parenteral, pulmonary, nasal and oral. The pharmaceutical compositions can be administered in a variety of unit dosage forms depending upon the method/mode of administration. Suitable unit dosage forms include, but are not limited to, powders, tablets, pills, capsules, lozenges, suppositories, patches, nasal sprays, injectibles, implantable sustained-release formulations, etc.

In some embodiments, a pharmaceutical composition comprises an acceptable carrier and/or excipients. A pharmaceutically acceptable carrier includes any solvents, dispersion media, or coatings that are physiologically compatible and that preferably does not interfere with or otherwise inhibit the activity of the therapeutic agent. Preferably, the carrier is suitable for intravenous, intramuscular, oral, intraperitoneal, transdermal, topical, or subcutaneous administration. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers. Other pharmaceutically acceptable carriers and their formulations are well-known and generally described in, for example, Remington: The Science and Practice of Pharmacy, 21st Edition, Philadelphia, Pa. Lippincott Williams & Wilkins, 2005. Various pharmaceutically acceptable excipients are well-known in the art and can be found in, for example, Handbook of Pharmaceutical Excipients (5^{th} ed., Ed. Rowe et al., Pharmaceutical Press, Washington, D.C.).

C. Normalizing Translational Profiles

In another aspect, the methods of the present invention relate to normalizing a translational profile in a subject. In some embodiments, the present invention provides a method of identifying an agent or therapeutic for normalizing a translational profile in a subject. In some embodiments, the present invention provides a method of validating a target for normalizing a translational profile associated with a disease. In some embodiments, the method comprises:
(a) determining a first translational profile for a first biological sample from the subject, wherein the first translational profile comprises translational levels for a plurality of genes;
(b) comparing the first translational profile to a second translational profile comprising translational levels for the plurality of genes, wherein the second translational profile is from a control sample, wherein the control sample is from a non-diseased subject;
(c) identifying one or more genes of a biological pathway as differentially translated in the first translational profile as compared to the second translational profile, wherein the biological pathway is selected from a protein synthesis pathway, a cell invasion/metastasis pathway, a cell division pathway, an apoptosis pathway, a signal transduction pathway, a cellular transport pathway, a post-translational protein modification pathway, a DNA repair pathway, and a DNA methylation pathway;
(d) contacting a second biological sample from the subject with the agent;
(e) determining a third translational profile for the second biological sample, wherein the third translational profile comprises translational levels for the one or more genes identified as differentially translated in the first translational profile as compared to the second translational profile; and
(f) comparing the translational levels for the one or more genes in the third translational profile to the translational levels for the one or more genes in the first and second translational profiles;
wherein a translational level for the one or more genes in the third translational profile that is closer to the translational level for the one or more genes in the second translational profile than to the translational level for the one or more genes in the first translational profile identifies the agent as an agent for normalizing the translational profile in the subject.

In some embodiments, the present invention provides a method of normalizing a translational profile in a subject. In some embodiments, the method comprises:
administering to the subject an agent that has been selected as an agent that normalizes the translational profile in the subject, wherein the agent is selected by:
   (a) determining a first translational profile for a first biological sample from the subject, wherein the first translational profile comprises translational levels for a plurality of genes;
   (b) comparing the first translational profile to a second translational profile comprising translational levels for the plurality of genes, wherein the second translational profile is from a control sample, wherein the control sample is from a non-diseased subject;
   (c) identifying one or more genes of a biological pathway as differentially translated in the first translational profile as compared to the second translational profile, wherein the biological pathway is selected from a protein synthesis pathway, a cell invasion/metastasis pathway, a cell division pathway, an apoptosis pathway, a signal transduction pathway, a cellular transport pathway, a post-translational protein modification pathway, a DNA repair pathway, and a DNA methylation pathway;
   (d) contacting a second biological sample form the subject with the agent;
   (e) determining a third translational profile for the second biological sample, wherein the third translational profile comprises translational levels for the one or more genes identified as differentially translated in the first translational profile as compared to the second translational profile; and
   (f) comparing the translational levels for the one or more genes in the third translational profile to the translational levels for the one or more genes in the first and second translational profiles; wherein a translational level for the one or more genes in the third translational profile that is closer to the translational level for the one or more genes in the second translational profile than to the translational level for the one or more genes in the first translational profile identifies the agent as an agent for normalizing the translational profile in the subject;
thereby normalizing the translational profile in the subject.

In certain embodiments, the present invention provides a method for identifying an agent or drug candidate molecule (i.e., a candidate therapeutic) for normalizing a translational profile associated with a disease (e.g., a cancer, an inflammatory disease, an autoimmune disease, a fibrotic disorder, a neurodegenerative disease, a neurodevelopmental disease, a metabolic disease, a viral infection, or a cardiomyopathy), comprising:
(a) determining a first translational profile for a plurality of genes from a disease sample contacted with an agent or drug candidate molecule;
(b) determining a second translational profile for a plurality of genes from (i) a control non-diseased sample or (ii) a control non-diseased sample contacted with the agent or drug candidate molecule; and
(c) identifying the agent or drug candidate molecule as useful for normalizing a translational profile associated with a disease (e.g., a cancer, an inflammatory disease, an autoimmune disease, a fibrotic disorder, a neurodegenerative disease, a neurodevelopmental disease, a metabolic disease, a viral infection, or a cardiomyopathy, respectively) when the first translational profile is comparable to the second translational profile.

In certain embodiments, the present invention provides a method for identifying an agent or drug candidate molecule (i.e., a candidate therapeutic) for normalizing a translational profile associated with a disease (e.g., a cancer, an inflammatory disease, an autoimmune disease, a fibrotic disorder, a neurodegenerative disease, a neurodevelopmental disease, a metabolic disease, a viral infection, or a cardiomyopathy), comprising:
(a) determining three independent translational profiles, each for a plurality of genes, wherein (i) a first translational profile is from a disease sample, (ii) a second translational profile is from (1) a control non-diseased sample or (2) a control non-diseased sample contacted with an agent or drug candidate molecule, and (iii) a third translational profile is from the disease sample contacted with the agent or drug candidate molecule;
(b) identifying one or more genes as differentially translated in the first translational profile as compared to the second translational profile; and
(c) identifying the agent as an agent or drug candidate molecule for normalizing a translational profile associated with a disease (e.g., a cancer, an inflammatory disease, an autoimmune disease, a fibrotic disorder, a neurodegenerative disease, a neurodevelopmental disease, a metabolic disease, a viral infection, or a cardiomyopathy, respectively) when the one or more differentially translated genes from step (b) are in the third translational profile and have a translational profile closer to the translational profile of the one or more genes in the second translational profile than to the translational profile of the one or more genes in the first translational profile.

In certain embodiments, the present invention provides a method for identifying an agent or drug candidate molecule (i.e., a candidate therapeutic agent) for normalizing a translational profile associated with a disease, comprising:
(a) determining three independent translational profiles, each for a plurality of genes, wherein (i) a first translational profile is from a disease sample, (ii) a second translational profile is from (1) a control non-diseased sample or (2) a control non-diseased sample contacted with an agent or drug candidate molecule, and (iii) a third translational profile is from the disease sample contacted with the agent or drug candidate molecule;
(b) determining a first differential translational profile comprising one or more genes differentially translated in the first translational profile as compared to the second translational profile, and determining a second differential translational profile comprising one or more genes differentially translated in the first translational profile as compared to the third translational profile; and
(c) identifying the agent as a candidate therapeutic for normalizing a translational profile associated with a disease when the first differential translational profile is comparable to the second differential translational profile.

In certain embodiments, the present invention provides a method for validating a target for normalizing a translational profile associated with a disease, comprising:
(a) determining a first translational profile for a plurality of genes from a disease sample contacted with an agent that modulates a target;
(b) determining a second translational profile for a plurality of genes from (i) a control non-diseased sample or (ii) a control non-diseased sample contacted with the agent that modulates a target; and
(c) validating the target as a target for normalizing a translational profile associated with a disease when the first translational profile is comparable to the second translational profile.

In certain embodiments, the present invention provides a method for validating a target for normalizing a translational profile associated with a disease, comprising:
(a) determining three independent translational profiles, each for a plurality of genes, wherein (i) a first translational profile is from a disease sample, (ii) a second translational profile is from (1) a control non-diseased sample or (2) a control non-diseased sample contacted with an agent that modulates a target, and (iii) a third translational profile is from the disease sample contacted with the agent that modulates a target;
(b) identifying one or more genes as differentially translated in the first translational profile as compared to the second translational profile; and
(c) validating the target as a target for normalizing a translational profile associated with a disease when the one or more differentially translated genes from step (b) are in the third translational profile and have a translational profile closer to the translational profile of the one or more genes in the second translational profile than to the translational profile of the one or more genes in the first translational profile.

In certain embodiments, the present invention provides a method for validating a target for normalizing a translational profile associated with a disease, comprising:
(a) determining three independent translational profiles, each for a plurality of genes, wherein (i) a first translational profile is from a disease sample, (ii) a second translational profile is from (1) a control non-diseased sample or (2) a control non-diseased sample contacted with an agent that modulates a target, and (iii) a third translational profile is from the disease sample contacted with the agent that modulates a target;
(b) determining a first differential translational profile comprising one or more genes differentially translated in the first translational profile as compared to the second translational profile, and determining a second differential translational profile comprising one or more genes differentially translated in the first translational profile as compared to the third translational profile; and
(c) validating the target as a target for normalizing a translational profile associated with a disease when the first differential translational profile is comparable to the second differential translational profile.

In any of the aforementioned embodiments for validating a target for normalizing a translational profile associated with a disease, the target is suspected of being associated with a disease, is indirectly associated with a disease, or is associated with a disease (e.g., an inflammatory disease, an autoimmune disease, a fibrotic disorder, a neurodegenerative disease, a neurodevelopmental disease, a metabolic disease, a viral infection, a cardiomyopathy or a cancer, respectively).

In some embodiments, one or more genes from each of at least two of the biological pathways are differentially translated in the first translational profile as compared to the second translational profile. In some embodiments, one or more genes from each of at least three of the biological pathways are differentially translated in the first translational profile as compared to the second translational profile. In some embodiments, there is at least a 1.5-fold or at least a two-fold difference (e.g., at least 1.5-fold, at least two-fold, at least three-fold, at least four-fold, at least five-fold, at least six-fold, at least seven-fold, at least eight-fold, at least nine-fold, at least ten-fold difference or more) in translational level for the one or more genes in the first translational profile as compared to the second translational profile. In some embodiments, the first, second, and/or third translational profiles comprise translational levels for a subset of the genome, e.g., for about 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50% of the genome or more. In some embodiments, the first, second, and/or third translational profiles comprise a genome-wide measurement of gene translational levels.

The agent can be any agent as described herein. In some embodiments, the agent is a peptide, protein, inhibitory RNA, or small organic molecule.

For comparing multiple translational profiles, for example, for determining to which translational profile a given experimentation translational profile is “closer” to, in some embodiments, an experimental translational profile has at least a 1.5 log₂ change or difference (e.g., at least 1.5, at least 2.5, at least 3, at least 4, at least 5, at least 6, at least 7, at least 8, at least 9, at least 10 or more log₂ change or difference, e.g., increase or decrease) in translational rate, translational efficiency, or both for one or more genes or for a set of selected marker genes as compared to the same genes or gene markers from one or more reference translational profiles of interest. In some embodiments, an experimental translational profile has at least a 2.5 log₂ change or difference in translational rate, translational efficiency, or both for one or more genes or for a set of selected marker genes as compared to the same genes or gene markers from one or more reference translational profiles of interest. In some embodiments, an experimental translational profile has at least a 3 log₂ change or difference in translational rate, translational efficiency, or both for one or more genes or for a set of selected marker genes as compared to the same genes or gene markers from one or more reference translational profiles of interest.

In some embodiments, an experimental profile as compared to one or more reference translational profiles of interest has at least a 1.1 log₂ change in translational rate, translational efficiency, or both for at least 0.05%, 0.1%, 0.25%, 0.5%, at least 1%, at least 2%, at least 3%, at least 4%, at least 5%, at least 6%, at least 7%, at least 8%, at least 9%, at least 10%, at least 15%, at least 20%, at least 30%, at least 40%, at least 50%, at least 60%, at least 70%, at least 80%, or at least 90% or more of a set of selected marker genes or for the entire set of selected marker genes. In some embodiments, an experimental profile as compared to one or more reference translational profiles of interest has at least a 2 log₂ change in translational rate, translational efficiency, or both for at least 0.05%, 0.1%, 0.25%, 0.5%, at least 1%, at least 2%, at least 3%, at least 4%, at least 5%, at least 6%, at least 7%, at least 8%, at least 9%, at least 10%, at least 15%, at least 20%, at least 30%, at least 40%, at least 50%, at least 60%, at least 70%, at least 80%, or at least 90% or more of a set of selected marker genes or for the entire set of selected marker genes. In some embodiments, an experimental profile as compared to one or more reference translational profiles of interest has at least a 2.5 log₂ change in translational rate, translational efficiency, or both for at least 0.05%, 0.1%, 0.25%, 0.5%, at least 1%, at least 2%, at least 3%, at least 4%, at least 5%, at least 6%, at least 7%, at least 8%, at least 9%, at least 10%, at least 15%, at least 20%, at least 30%, at least 40%, at least 50%, at least 60%, at least 70%, at least 80%, or at least 90% or more of a set of selected marker genes or for the entire set of selected marker genes. In some embodiments, an experimental profile as compared to one or more reference translational profiles of interest has at least a 4 log₂ change in translational rate, translational efficiency, or both for at least 0.5%, at least 1%, at least 2%, at least 3%, at least 4%, at least 5%, at least 6%, at least 7%, at least 8%, at least 9%, at least 10%, at least 15%, at least 20%, at least 30%, at least 40%, at least 50%, at least 60%, at least 70%, at least 80%, or at least 90% or more of a set of selected marker genes or for the entire set of selected marker genes.

As described herein, differentially translated genes between first and second translational profiles under a first condition may exhibit translational profiles “closer to” each other (i.e., identified through a series of pair-wise comparisons to confirm a similarity of pattern) under one or more different conditions (e.g., differentially translated genes between a normal sample and a disease sample may have a more similar translational profile when the normal sample is compared to a disease sample contacted with a candidate agent; differentially translated genes between a disease sample and a disease sample treated with a known active agent may have a more similar translational profile when the disease sample treated with a known active agent is compared to the disease sample contacted with a candidate agent). In certain embodiments, a test translational profile is “closer to” a reference translational profile when at least of 99%, 95%, 90%, 85%, 80%, 75%, 70%, 65%, 60%, 55%, or 50% of a selected portion of differentially translated genes, a majority of differentially translated genes, or all differentially translated genes show a translational profile within 75%, 70%, 65%, 60%, 55%, 50%, 45%, 40%, 35%, 30%, or 25%, respectively, of their corresponding genes in the reference translational profile. In further embodiments, a selected portion of differentially translated genes, a majority of differentially translated genes, or all differentially translated genes from an experimental translational profile have a translational profile “closer to” the translational profile of the same genes in a reference translational profile when the amount of protein translated in the experimental and reference translational profiles are within about 3.0 log₂, 2.5 log₂, 2.0 log₂, 1.5 log₂, 1.1 log₂, 0.5 log₂, 0.2 log₂ or closer. In still further embodiments, a selected portion of differentially translated genes, a majority of differentially translated genes, or all differentially translated genes from an experimental translational profile have a translational profile “closer to” the translational profile of the same genes in a reference translational profile when the amount of protein translated in the experimental and reference translational profiles differs by no more than about 30%, 25%, 20%, 15%, 10%, 5%, 1% or less.

In some embodiments, an experimental differential profile as compared to a reference differential translational profile of interest has at least a 1.0 log₂ change in translational rate, translational efficiency, or both for at least 0.05%, at least 0.1%, at least 0.25%, at least 0.5%, at least 1%, at least 2%, at least 3%, at least 4%, at least 5%, at least 6%, at least 7%, at least 8%, at least 9%, at least 10%, at least 15%, at least 20%, at least 30%, at least 40%, at least 50%, at least 60%, at least 70%, at least 80%, or at least 90% or more of a set of selected differentially translated genes or for the entire set of selected differentially translated genes. In some embodiments, an experimental differential profile as compared to a reference differential translational profile of interest has at least a 2 log₂ change in translational rate, translational efficiency, or both for at least 0.05%, at least 0.1%, at least 0.25%, at least 0.5%, at least 1%, at least 5%, at least 10%, at least 15%, at least 20%, at least 30%, at least 40%, at least 50%, at least 60%, at least 70%, at least 80%, or at least 90% or more of a set of selected differentially translated genes or for the entire set of differentially translated genes. In some embodiments, an experimental differential profile as compared to a reference differential translational profile of interest has at least a 3 log₂ change in translational rate, translational efficiency, or both for at least 0.05%, at least 0.1%, at least 0.25%, at least 0.5%, at least 1%, at least 5%, at least 10%, at least 15%, at least 20%, at least 30%, at least 40%, at least 50%, at least 60%, at least 70%, at least 80%, or at least 90% or more of a set of selected differentially translated genes or for the entire set of selected differentially translated genes. In some embodiments, an experimental differential profile as compared to a reference differential translational profile of interest has at least a 4 log₂ change in translational levels for at least 0.05%, at least 0.1%, at least 0.25%, at least 0.5%, at least 1%, at least 5%, at least 10%, at least 15%, at least 20%, at least 30%, at least 40%, at least 50%, at least 60%, at least 70%, at least 80%, or at least 90% or more of a set of selected differentially translated genes or for the entire set of selected differentially translated genes.

As described herein, a differential translational profile between a first sample and a control may be “comparable” to a differential translational profile between a second sample and the control (e.g., the differential profile between a disease sample and the disease sample treated with a known active compound may be comparable to the differential profile between the disease sample and the disease sample contacted with a candidate agent; the differential profile between a disease sample and a non-diseased (normal) sample may be comparable to the differential profile between the disease sample and the disease sample contacted with a candidate agent). In certain embodiments, a test differential translational profile is “comparable to” a reference differential translational profile when at least of 99%, 95%, 90%, 80%, 70%, 60%, 50%, 25%, or 10% of a selected portion of differentially translated genes, a majority of differentially translated genes, or all differentially translated genes show a translational profile within 75%, 70%, 65%, 60%, 55%, 50%, 45%, 40%, 35%, 30%, or 25%, respectively, of their corresponding genes in the reference translational profile. In further embodiments, a differential translational profile comprising a selected portion of the differentially translated genes or all the differentially translated genes has a differential translational profile “comparable to” the differential translational profile of the same genes in a reference differential translational profile when the amount of protein translated in the experimental and reference differential translational profiles are within about 3.0 log₂, 2.5 log₂, 2.0 log₂, 1.5 log₂, 1.0 log₂, 0.5 log₂, 0.2 log₂ or closer. In still further embodiments, a differential translational profile comprising a selected portion of the differentially translated genes or all the differentially translated genes has a differential translational profile “comparable to” the differential translational profile of the same genes in a reference differential translational profile when the amount of protein translated in the experimental and reference differential translational profiles differs by no more than about 50%, 45%, 40%, 35%, 30%, 25%, 20%, 15%, 10%, 5%, 1% or less.

In some embodiments, the subject in need thereof is a subject having a pathogenic condition in which protein translation is known or suspected to be aberrant. In some embodiments, the subject has a condition in which aberrant translation is known to be causative for the pathogenic condition. In certain embodiments, the subject has a pathogenic condition in which altering the aberrant translation (e.g., increasing or decreasing) will prevent, ameliorate or treat the pathogenic condition. In certain embodiments, the target is associated with a disease selected from an inflammatory disease, autoimmune disease, fibrotic disorder, neurodegenerative disease, neurodevelopmental disease, metabolic disease, viral infection, cardiomyopathy or cancer.

### VIII. Examples

The following examples are offered to illustrate, but not to limit the claimed invention.

### Example 1

### Generation of a Comprehensive Map of Translationally Controlled mTOR Targets in Cancer Using Ribosome Profiling

Downstream of the phosphatidylinositol-3-OH kinase (PI(3)K)-AKT signalling pathway, mTOR assembles with either raptor or rictor to form two distinct complexes: mTORC1 and mTORC2. The major regulators of protein synthesis downstream of mTORC1 are 4EBP1 (also called EIF4EBP1) and p70S6K1/2. 4EBP1 negatively regulates eIF4E, a key rate-limiting initiation factor for cap-dependent translation. Phosphorylation of 4EBP1 by mTORC1 leads to its dissociation from eIF4E, allowing translation initiation complex formation at the 5′ end of mRNAs. The mTOR-dependent phosphorylation of p70S6K1/2 also promotes translation initiation as well as elongation. In this example, ribosome profiling delineates the translational landscape of the cancer genome at a codon-by-codon resolution upon pharmacological inhibition of mTOR. This method provides a genome-wide characterization of translationally controlled mRNAs downstream of oncogenic mTOR signalling and delineates their functional roles in cancer development.

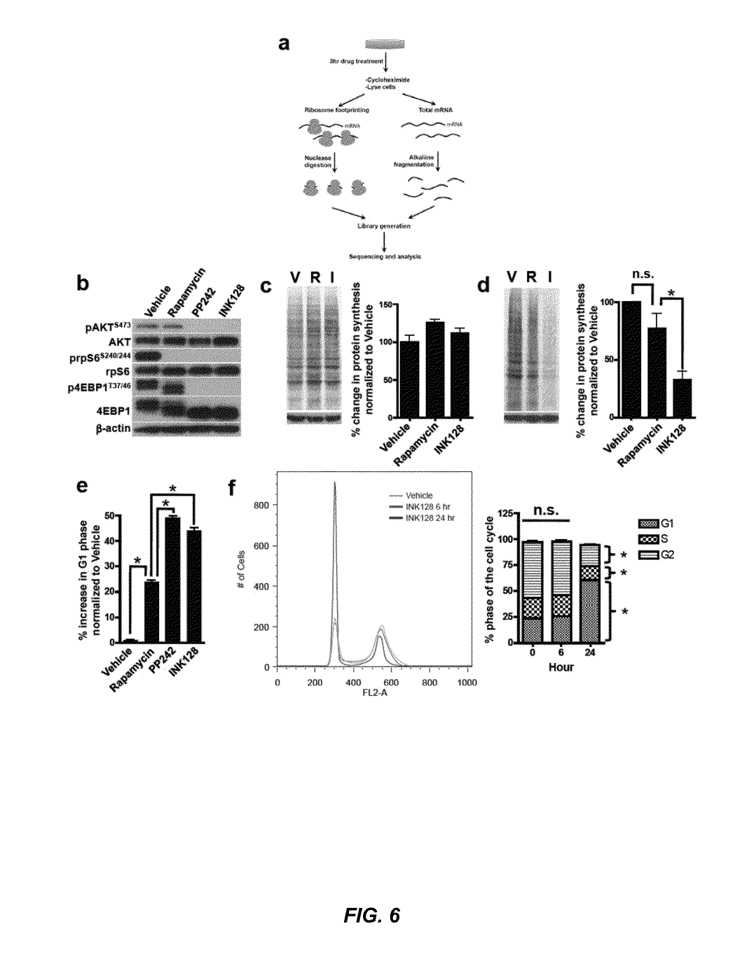

mTOR is deregulated in nearly 100% of advanced human prostate cancers, and genetic findings in mouse models implicate mTOR hyperactivation in prostate cancer initiation. Given the critical role for mTOR in prostate cancer, PC3 human prostate cancer cells, in which mTOR is constitutively hyperactivated, were used to delineate translationally controlled gene expression networks upon complete or partial mTOR inhibition. Ribosome profiling was optimized to assess quantitatively ribosome occupancy genome-wide in cancer cells. In brief, ribosome-protected mRNA fragments were deep-sequenced to determine the number of ribosomes engaged in translating specific mRNAs (see FIG. 6a and Example 6 (“Methods”) below).

Treatment of PC3 cells with an mTOR ATP site inhibitor, PP242 (Feldman et al., PLoS Biol. 7:e38 (2009); Hsieh et al., Cancer Cell 17:249-261 (2010)), significantly inhibited the activity of the three primary downstream mTOR effectors 4EBP1, p70S6K1/2 and AKT. On the contrary, rapamycin, an allosteric mTOR inhibitor, only blocked p70S6K1/2 activity in these cells (FIG. 6b). Short 3-hr drug treatments, which precede alterations in de novo protein synthesis, were used to capture direct changes in mTOR-dependent gene expression by ribosome profiling and to minimize compensatory feedback mechanisms (FIG. 6c-f).

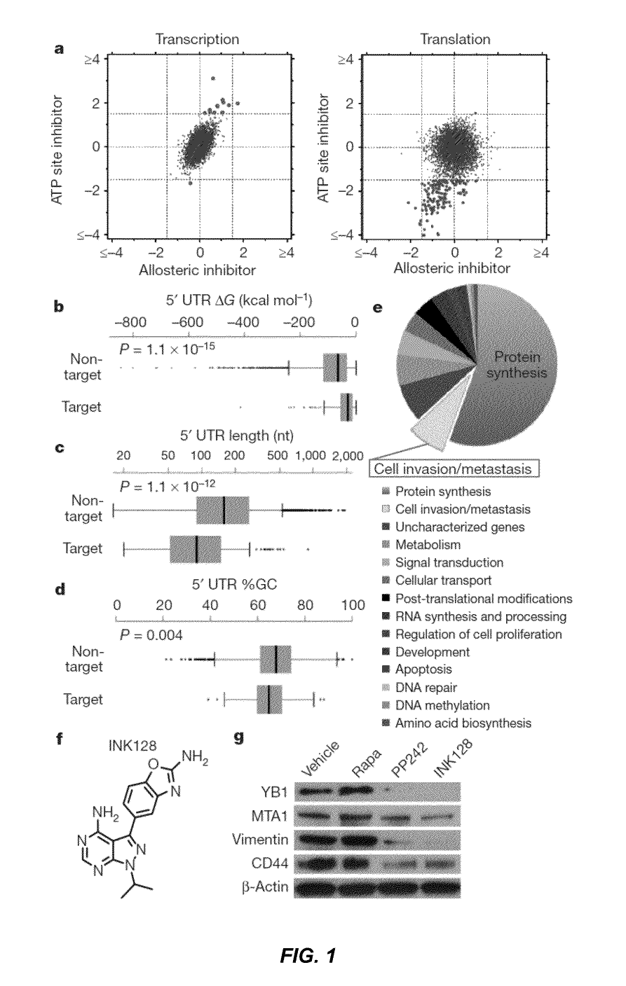

Ribosome profiling revealed 144 target mRNAs were selectively decreased at the translational level upon PP242 treatment (log₂≦−1.5 (false discovery rate <0.05)) as compared to rapamycin treatment, with limited changes in transcription (FIGS. 1a, 7a-b, and 8-10, Table 3, Table 5, Table 6, and Table 7). The fact that at this time point rapamycin treatment did not markedly affect gene expression is consistent with incomplete, allosteric, inhibition of mTOR activity (FIG. 6b). By monitoring footprints of translating 80S ribosomes, these findings showed that the effects of PP242 were largely at the level of translation initiation and not elongation (FIG. 8). It has been proposed that mRNAs translationally regulated by mTOR may contain long 5′ untranslated regions (5′ UTRs) with complex RNA secondary structures. On the contrary, ribosome profiling revealed that mTOR-responsive 5′ UTRs possess less complex features (FIG. 1b-d), providing a unique data set to investigate the nature of regulatory elements that render these mRNAs mTOR-sensitive. It has been previously shown that some mTOR translationally regulated mRNAs, most notably those involved in protein synthesis, possess a 5′ terminal oligopyrimidine tract (5′ TOP) that is regulated by distinct trans-acting factors. Of the 144 mTOR-sensitive target genes, 68% possessed a 5′ TOP (see Table 1). Additionally, another 5′ UTR consensus sequence, termed a pyrimidine-rich translational element (PRTE), was identified within the 5′ UTRs of 63% of mTOR target mRNAs (P=3.2×10⁻¹¹). This PRTE element, unlike the 5′ TOP sequence, consists of an invariant uridine at position 6 flanked by pyrimidines and does not reside at position +1 of the 5′ UTR (FIG. 7c and Table 2). 89% of the mTOR-responsive genes were found to possess a PRTE and/or 5′ TOP, making the presence of one or both sequences a strong predictor for mTOR sensitivity (FIG. 7d and Table 3). Notably, mRNA isoforms arising from distinct transcription start sites may possess both a 5′ TOP and a PRTE. Given the significant number of mRNAs that contain both the PRTE and 5′ TOP, a functional interplay may exist between these regulatory elements. Additionally, these findings show that the PRTE imparts translational control specificity to 4EBP1 activity.

Surprisingly, mTOR-sensitive genes stratified into unique functional categories that may promote cancer development and progression, including cellular invasion (P=0.009), cell proliferation (P=0.04), metabolism (P=0.0002) and regulators of protein modification (P=0.01) (FIG. 1e). The largest fraction of mTOR-responsive mRNAs clustered into a node consisting of key components of the translational apparatus: 70 ribosomal proteins, 6 elongation factors, and 4 translation initiation factors (P=7.5×10⁻⁸²) (FIG. 1e). Therefore, this class of mTOR-responsive mRNAs may represent an important regulon that sustains the elevated protein synthetic capacity of cancer cells.

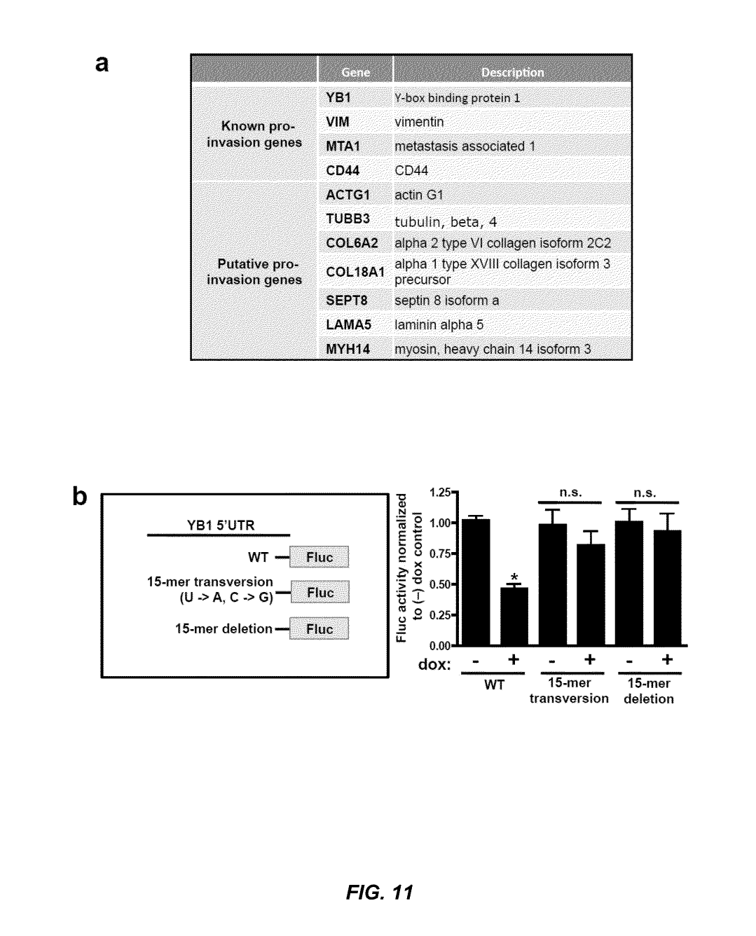

The second largest node of mTOR translationally regulated genes comprised bona fide cell invasion and metastasis mRNAs and putative regulators of this process (FIG. 1e). This group included YB1 (Y-box binding protein 1; also called YBX1), vimentin, MTA1 (metastasis associated 1) and CD44 (FIG. 11a). YB1 regulates the post-transcriptional expression of a network of invasion genes. Vimentin, an intermediate filament protein, is highly upregulated during the epithelial-to-mesenchymal transition associated with cellular invasion. MTA1, a putative chromatin-remodeling protein, is overexpressed in invasive human prostate cancer and has been shown to drive cancer metastasis by promoting neoangiogenesis. CD44 is commonly overexpressed in tumor-initiating cells and is implicated in prostate cancer metastasis. Consistent with their status as mTOR-sensitive genes, YB1, vimentin, MTA1 and CD44 all possess a PRTE (Table 2). Vimentin and CD44 also possess a 5′ TOP (Table 3). To test the functional role of the PRTE in mediating translational control, the PRTE was mutated within the 5′ UTR of YB1, which rendered the YB1 5′ UTR insensitive to inhibition by 4EBP1 (FIG. 11b). These findings highlight a novel cis-regulatory element that may modulate translational control of subsets of mRNAs upon mTOR activation. Moreover, ribosome profiling reveals unexpected transcript-specific translational control, mediated by oncogenic mTOR signaling, including a distinct set of pro-invasion and metastasis genes.

TABLE 5
Mean list of translationally regulated PP242-responsive genes

| | | Rapamycin | | PP242 | |
|---|---|---|---|---|---|
| Gene | Description | mRNA | TrlEff | mRNA | TrlEff |
| EEF2 | eukaryotic translation elongation | 0.39 | −1.12 | 0.76 | −3.60 |
| | factor 2 | | | | |
| EEF1A1 | eukaryotic translation elongation | 0.43 | −1.58 | 0.36 | −3.21 |
| | factor 1 alpha 1 | | | | |
| RPL13A | ribosomal protein L13a | 0.15 | −1.25 | 0.30 | −3.10 |
| RPS12 | ribosomal protein S12 | 0.11 | −1.22 | 0.04 | −3.00 |
| RPL12 | ribosomal protein L12 | 0.07 | −0.94 | 0.12 | −2.95 |
| RPS27 | ribosomal protein S27 | 0.10 | −1.54 | 0.07 | −2.71 |
| RPS28 | ribosomal protein S28 | 0.01 | −0.80 | 0.28 | −2.67 |
| RPL18A | ribosomal protein L18a | 0.17 | −0.82 | 0.23 | −2.63 |
| RPL34 | ribosomal protein L34 | 0.11 | −1.12 | 0.04 | −2.63 |
| RPL28 | ribosomal protein L28 isoform 1 | 0.24 | −1.09 | 0.22 | −2.54 |
| RPL27A | ribosomal protein L27a | 0.06 | −0.96 | 0.07 | −2.53 |
| CRTAP | cartilage associated protein | 0.29 | −1.17 | 0.33 | −2.50 |
| RPL10 | ribosomal protein L10 | 0.09 | −0.79 | 0.25 | −2.46 |
| RPS20 | ribosomal protein S20 isoform 1 | 0.18 | −1.35 | −0.01 | −2.46 |
| RPL21 | ribosomal protein L21 | 0.14 | −1.25 | −0.04 | −2.45 |
| RPL3 | ribosomal protein L3 isoform a | 0.18 | −1.08 | 0.22 | −2.44 |
| RPL39 | ribosomal protein L39 | 0.17 | −1.65 | −0.15 | −2.41 |
| RPL37A | ribosomal protein L37a | 0.08 | −1.02 | 0.01 | −2.38 |
| VIM | vimentin | 0.36 | −0.40 | 0.67 | −2.38 |
| EEF1D | eukaryotic translation elongation | 0.18 | −0.84 | 0.35 | −2.37 |
| | factor 1 delta | | | | |
| GNB2L1 | Guanine nucleotide binding protein | 0.19 | −0.77 | 0.27 | −2.35 |
| | (G protein) | | | | |
| RPS19 | ribosomal protein S19 | 0.15 | −0.74 | 0.23 | −2.34 |
| RPL32 | ribosomal protein L32 | 0.22 | −0.97 | 0.11 | −2.33 |
| RPS15A | ribosomal protein S15a | 0.07 | −0.96 | 0.07 | −2.31 |
| RPL11 | ribosomal protein L11 | 0.09 | −1.08 | 0.14 | −2.31 |
| RPL7A | ribosomal protein L7a | 0.17 | −0.74 | 0.15 | −2.30 |
| YB1 | Y-box binding protein 1 | 0.11 | −0.59 | 0.24 | −2.30 |
| RPS9 | ribosomal protein S9 | 0.10 | −0.60 | 0.34 | −2.27 |
| EIF4B | eukaryotic translation initiation | 0.55 | −1.21 | 0.61 | −2.27 |
| | factor 4B | | | | |
| EEF1G | eukaryotic translation elongation | 0.21 | −1.15 | 0.15 | −2.26 |
| | factor 1, gamma | | | | |
| RPS2 | ribosomal protein S2 | 0.07 | −0.56 | 0.20 | −2.25 |
| RPS5 | ribosomal protein S5 | 0.14 | −0.77 | 0.23 | −2.25 |
| HSPA8 | heat shock 70 kDa protein 8 isoform 1 | −0.21 | −0.46 | −0.40 | −2.25 |
| RPS3A | ribosomal protein S3a | 0.22 | −1.15 | −0.06 | −2.17 |
| RPS3 | ribosomal protein S3 | 0.22 | −0.92 | 0.24 | −2.16 |
| RPL10A | ribosomal protein L10a | 0.16 | −0.94 | 0.14 | −2.16 |
| RPS25 | ribosomal protein S25 | 0.04 | −0.89 | −0.04 | −2.13 |
| GLTSCR2 | glioma tumor suppressor candidate | 0.31 | −0.68 | 0.70 | −2.12 |
| | region gene 2 | | | | |
| HNRNPA1 | heterogeneous nuclear | 0.18 | −0.86 | 0.27 | −2.12 |
| | ribonucleoprotein A1 | | | | |
| RPLP2 | ribosomal protein P2 | 0.26 | −1.18 | 0.14 | −2.10 |
| RPL31 | ribosomal protein L31 isoform 2 | −0.02 | −0.62 | 0.05 | −2.10 |
| PABPC1 | poly(A) binding protein, | 0.35 | −1.44 | 0.16 | −2.09 |
| | cytoplasmic 1 | | | | |
| RPS21 | ribosomal protein S21 | −0.01 | −0.60 | 0.09 | −2.09 |
| RPS4X | ribosomal protein S4, X-linked X | 0.18 | −1.15 | 0.12 | −2.06 |
| | isoform | | | | |
| RPLP1 | ribosomal protein P1 isoform 1 | 0.28 | −1.09 | 0.12 | −2.06 |
| RPL7 | ribosomal protein L7 | 0.15 | −1.06 | 0.01 | −2.02 |
| RPL26 | ribosomal protein L26 | 0.15 | −1.11 | 0.02 | −2.00 |
| PABPC4 | poly A binding protein, cytoplasmic | 0.24 | −0.80 | 0.40 | −1.98 |
| | 4 isoform 1 | | | | |
| RPL36A | ribosomal protein L36a | 0.13 | −1.11 | −0.01 | −1.98 |
| EEF1A2 | eukaryotic translation elongation | 0.03 | −0.03 | 0.40 | −1.94 |
| | factor 1 alpha 2 | | | | |
| TPT1 | tumor protein, translationally- | 0.24 | −1.22 | 0.01 | −1.94 |
| | controlled 1 | | | | |
| AHCY | adenosylhomocysteinase isoform 1 | 0.20 | −0.23 | 0.38 | −1.93 |
| RPL22L1 | ribosomal protein L22-like 1 | 0.15 | −0.68 | 0.39 | −1.90 |
| GAPDH | glyceraldehyde-3-phosphate | 0.17 | −0.27 | 0.28 | −1.90 |
| | dehydrogenase | | | | |
| RPL30 | ribosomal protein L30 | 0.11 | −0.99 | 0.01 | −1.89 |
| RPS11 | ribosomal protein S11 | 0.11 | −0.59 | 0.20 | −1.88 |
| RPL29 | ribosomal protein L29 | 0.10 | −0.50 | 0.20 | −1.88 |
| RPL14 | ribosomal protein L14 | 0.07 | −0.68 | −0.02 | −1.85 |
| RPL36 | ribosomal protein L36 | 0.09 | −0.43 | 0.28 | −1.85 |
| EIF2S3 | eukaryotic translation initiation | 0.33 | −1.04 | 0.15 | −1.85 |
| | factor 2, S3 | | | | |
| RPL23 | ribosomal protein L23 | 0.09 | −0.92 | 0.07 | −1.82 |
| RPS16 | ribosomal protein S16 | 0.13 | −0.38 | 0.19 | −1.81 |
| SLC25A5 | adenine nucleotide translocator 2 | 0.21 | −0.30 | 0.15 | −1.80 |
| RPL17 | ribosomal protein L17 | 0.05 | −0.93 | 0.07 | −1.80 |
| RPL37 | ribosomal protein L37 | 0.11 | −0.68 | 0.10 | −1.79 |
| RPL8 | ribosomal protein L8 | 0.12 | −0.40 | 0.29 | −1.79 |
| NAP1L1 | nucleosome assembly protein 1-like 1 | 0.24 | −0.97 | 0.15 | −1.79 |
| RPS10 | ribosomal protein S10 | 0.16 | −0.69 | 0.19 | −1.78 |
| IPO7 | importin 7 | 0.20 | −0.83 | 0.26 | −1.75 |
| RPS8 | ribosomal protein S8 | 0.09 | −0.44 | 0.14 | −1.74 |
| RPL5 | ribosomal protein L5 | 0.17 | −1.11 | 0.06 | −1.73 |
| RPS24 | ribosomal protein S24 isoform d | 0.11 | −1.16 | −0.01 | −1.73 |
| EEF1B2 | eukaryotic translation elongation | 0.12 | −1.10 | −0.06 | −1.70 |
| | factor 1 beta 2 | | | | |
| RPL6 | ribosomal protein L6 | 0.09 | −0.68 | 0.06 | −1.68 |
| RPS23 | ribosomal protein S23 | 0.15 | −1.19 | −0.03 | −1.68 |
| RPL18 | ribosomal protein L18 | 0.08 | −0.42 | 0.18 | −1.65 |
| RPS29 | ribosomal protein S29 isoform 2 | −0.01 | −0.69 | 0.11 | −1.65 |
| RPS6 | ribosomal protein S6 | 0.14 | −1.06 | −0.02 | −1.65 |
| RPL22 | ribosomal protein L22 | 0.08 | −0.89 | 0.00 | −1.64 |
| UBA52 | ubiquitin and ribosomal protein L40 | 0.12 | −0.22 | 0.18 | −1.62 |
| RPLP0 | ribosomal protein PO | 0.15 | −0.42 | 0.12 | −1.61 |
| RPS27A | ubiquitin and ribosomal protein | 0.16 | −0.89 | −0.04 | −1.61 |
| | S27a | | | | |
| RPL9 | ribosomal protein L9 | 0.16 | −1.00 | −0.08 | −1.59 |
| TKT | transketolase isoform 1 | 0.02 | −0.11 | 0.33 | −1.58 |
| RPL13 | ribosomal protein L13 | 0.14 | −0.38 | 0.26 | −1.56 |
| EIF3H | eukatyotic translation initiation | 0.16 | −0.79 | 0.09 | −1.54 |
| | factor 3, | | | | |
| RPS13 | ribosomal protein S13 | 0.07 | −0.82 | −0.08 | −1.54 |
| RPS7 | ribosomal protein S7 | 0.11 | −0.76 | −0.04 | −1151 |
| RPS14 | ribosomal protein S14 | 0.10 | −0.60 | 0.16 | −1.50 |
| RPL4 | ribosomal protein L4 | 0.22 | −0.85 | 0.10 | −1.50 |
| FAM128B | hypothetical protein LOC80097 | 0.06 | 0.27 | 0.43 | −1.47 |
| EIF3L | eukaryotic translation initiation | 0.28 | −0.85 | 0.21 | −1.47 |
| | factor 3L | | | | |
| RABGGTB | RAB geranylgeranyltransferase, | −0.20 | −0.84 | 0.20 | −1.46 |
| | beta subunit | | | | |
| FASN | fatty acid synthase | −0.37 | 0.47 | 0.30 | −1.42 |
| RPL24 | ribosomal protein L24 | 0.11 | −0.63 | 0.00 | −1.41 |
| ACTG1 | actin, gamma 1 propeptide | 0.02 | −0.07 | 0.28 | −1.40 |
| PFDN5 | prefoldin subunit 5 isoform alpha | 0.11 | −0.51 | 0.04 | −1.38 |
| LMF2 | lipase maturation factor 2 | 0.22 | 0.39 | 0.62 | −1.36 |
| RPL19 | ribosomal protein L19 | 0.14 | −0.66 | 0.11 | −1.35 |
| PGM1 | phosphoglucomutase 1 | 0.40 | −0.55 | 0.23 | −1.35 |
| CCNI | cyclin I | 0.29 | −0.45 | 0.24 | −1.33 |
| IMPDH2 | inosine monophosphate | 0.11 | −0.39 | 0.21 | −1.33 |
| | dehydrogenase 2 | | | | |
| AP2A1 | adaptor-related protein complex 2, | 0.09 | −0.04 | 0.42 | −1.32 |
| | alpha 1 | | | | |
| AGRN | agrin precursor | 0.01 | 0.51 | 0.50 | −1.29 |
| COL6A2 | alpha 2 type VI collagen isoform | −0.08 | 0.43 | 0.57 | −1.29 |
| | 2C2 | | | | |
| CD44 | CD44 antigen isoform 1 | 0.34 | −0.46 | 0.43 | −1.29 |
| RPL41 | ribosomal protein L41 | 0.04 | −1.15 | −0.01 | −1.28 |
| ALKBH7 | spermatogenesis associated 11 | 0.06 | 0.28 | 0.51 | −1.27 |
| | precursor | | | | |
| RPL27 | ribosomal protein L27 | 0.05 | −0.33 | −0.13 | −1.23 |
| RPL15 | ribosomal protein L15 | 0.11 | −0.51 | 0.19 | −1.20 |
| RPS15 | ribosomal protein S15 | −0.01 | 0.03 | 0.21 | −1.19 |
| CLPTM1 | cleft lip and palate associated | 0.07 | 0.26 | 0.41 | −1.13 |
| | transmembrane | | | | |
| FAM83H | FAM83H | −0.17 | 0.71 | 0.33 | −1.11 |
| PGLS | 6-phosphogluconolactonase | 0.03 | 0.20 | 0.21 | −1.11 |
| MTA1 | metastasis associated 1 | 0.00 | −0.05 | 0.21 | −1.09 |
| TSC2 | tuberous sclerosis 2 isoform 1 | −0.15 | 0.34 | 0.21 | −1.09 |
| PACS1 | phosphofurin acidic cluster sorting | 0.07 | 0.04 | 0.45 | −1.09 |
| | protein 1 | | | | |
| CIRBP | cold inducible RNA binding protein | 0.14 | 0.10 | 0.54 | −1.08 |
| SLC19A1 | solute carrier family 19 member 1 | −036 | 0.23 | 0.10 | −1.07 |
| ECSIT | evolutionarily conserved signaling | −0.04 | 0.41 | 0.26 | −1.06 |
| | intermediate | | | | |
| ARD1A | alpha-N-acetyltransferase 1A | −0.04 | 0.01 | 0.03 | −1.05 |
| C21orf66 | GC-rich sequence DNA-binding | −0.30 | −0.09 | −0.31 | −1.03 |
| | factor candidate | | | | |
| ATP5G2 | ATP synthase, H+ transporting, | 0.29 | −0.28 | 0.17 | −1.01 |
| | mitochondrial F0 | | | | |
| LAMA5 | laminin alpha 5 | −0.32 | 0.87 | 0.40 | −0.94 |
| PNKP | polynucleotide kinase 3′ | −0.24 | 0.74 | 0.33 | −0.79 |
| | phosphatase | | | | |
| EVPL | envoplakin | −0.08 | 0.30 | 0.38 | −0.79 |
| NCLN | nicalin | −0.05 | 0.67 | 0.29 | −0.76 |
| PTGES2 | prostaglandin E synthase 2 | −0.19 | 0.52 | 0.17 | −0.65 |
| GAMT | guanidinoacetate N- | n/a | n/a | n/a | n/a |
| | methyltransferase isoform b | | | | |
| CTSH | cathepsin H isoform b | n/a | n/a | n/a | n/a |
| TUBB3 | tubulin, beta, 4 | n/a | n/a | n/a | n/a |
| CSDA | cold shock domain protein A | n/a | n/a | n/a | n/a |
| ETHE1 | ETHE1 protein | n/a | n/a | n/a | n/a |
| LCMT1 | leucine carboxyl methyltransferase | n/a | n/a | n/a | n/a |
| | 1 isoform a | | | | |
| PC | pyruvate carboxylase | n/a | n/a | n/a | n/a |
| SECTM1 | secreted and transmembrane 0 | n/a | n/a | n/a | n/a |
| COL18A1 | alpha 1 type XVIII collagen | n/a | n/a | n/a | n/a |
| | isoform 3 | | | | |
| CHP | calcium binding protein P22 | n/a | n/a | n/a | n/a |
| BRF1 | transcription initiation factor IIIB | n/a | n/a | n/a | n/a |
| C2orf79 | hypothetical protein LOC391356 | n/a | n/a | n/a | n/a |
| SEPT8 | septin 8 isoform a | n/a | n/a | n/a | n/a |
| ABCB7 | ATP-binding cassette, sub-family | n/a | n/a | n/a | n/a |
| | B, member 7 | | | | |
| MYH14 | myosin, heavy chain 14 isoform 3 | n/a | n/a | n/a | n/a |
| SIGMAR1 | sigma non-opioid intracellular | n/a | n/a | n/a | n/a |
| | receptor 1 | | | | |
| C3orf38 | hypothetical protein LOC285237 | n/a | n/a | n/a | n/a |

TABLE 6
List of rapamycin-sensitive translationally regulated genes after 3-hour treatment
with rapamycin (50 nM) or PP242 (2.5 μM) in PC3 cells.

| | | Rapamycin | | PP242 | |
|---|---|---|---|---|---|
| Gene | Description | mRNA | TrlEff | mRNA | TrlEff |
| MAPK6 | mitogen-activated protein kinase 6 | 0.13 | −2.43 | 0.10 | −0.29 |
| RPL39 | ribosomal protein L39 | 0.30 | −2.11 | −0.42 | −2.53 |
| RPS20 | ribosomal protein S20 isoform 1 | 0.14 | −1.79 | −0.10 | −2.78 |
| PRKD3 | protein kinase D3 | −0.22 | −1.72 | −0.46 | 0.68 |
| UBTD2 | dendritic cell-derived ubiquitin- | 0.19 | −1.64 | 0.25 | 0.27 |
| | like protein | | | | |
| RPL28 | ribosomal protein L28 isoform 1 | 0.64 | −1.59 | 0.55 | −3.48 |
| RBPJ | recombining binding protein | 1.09 | −1.58 | 0.17 | −0.03 |
| | suppressor of | | | | |
| EEF1A1 | eukaryotic translation elongation | 0.46 | −1.57 | 0.29 | −3.53 |
| | factor 1 alpha | | | | |
| UCHL5 | ubiquitin carboxyl-terminal | −0.08 | −1.56 | −0.51 | 0.40 |
| | hydrolase L5 | | | | |
| RPS27 | ribosomal protein S27 | 0.07 | −1.55 | 0.06 | −3.35 |
| SDCCAG10 | serologically defined colon cancer | −0.19 | −1.50 | −0.37 | 0.23 |
| | antigen 10 | | | | |
| MAPKAPK2 | mitogen-activated protein kinase- | −0.21 | 1.50 | −0.22 | 0.92 |
| | activated | | | | |
| NFATC21P | nuclear factor of activated T-cells, | −0.16 | 1.54 | 0.08 | 0.35 |
| | 2IP | | | | |
| GTPBP3 | GTP binding protein 3 | −0.73 | 1.56 | 0.15 | −0.83 |
| | (mitochondrial) isoform V | | | | |
| C17orf28 | hypothetical protein LOC283987 | −0.44 | 1.66 | 0.21 | −0.20 |
| VHL | von Hippel-Lindau tumor | −0.23 | 1.67 | 0.43 | 0.52 |
| | suppressor isoform 1 | | | | |
| DDX51 | DEAD (Asp-Glu-Ala-Asp) box | −0.24 | 1.68 | 0.17 | −0.51 |
| | polypeptide 51 | | | | |
| DGCR2 | integral membrane protein | −0.66 | 1.69 | 0.05 | 0.02 |
| | DGCR2 | | | | |
| CCNA1 | cyclin A1 isoform a | −0.51 | 1.81 | −0.33 | 0.66 |
| NR2F1 | nuclear receptor subfamily 2, | 0.05 | 1.94 | 0.87 | −0.09 |
| | group F, member 1 | | | | |
| ACD | adrenocortical dysplasia homolog | −0.96 | 2.06 | 0.20 | −1.02 |
| | isoform 1 | | | | |

TABLE 7
PP242 and rapamycin transcriptional targets.

| | | |
|---|---|---|
| Gene | Description | mRNA |

A. PP242 sensitive transcriptionally regulated genes upon
3-hour treatment with PP242 (2.5 μM) in PC3 cells*

| | | |
|---|---|---|
| FGFBP1 | fibroblast growth factor binding protein 1 | −1.75 |
| BRIX1 | ribosome biogenesis protein BRX1 homolog | −1.51 |
| FOXA1 | forkhead box A1 | 1.45 |
| CYR61 | cysteine-rich, angiogenic inducer, 61 precursor | 1.47 |
| MT2A | metallothionein 2A | 1.47 |
| SOX4 | SRY (sex determining region Y)-box 4 | 1.51 |
| BCL6 | B-cell lymphoma 6 protein isoform 1 | 1.59 |
| KLF6 | Kruppel-like factor 6 isoform A | 1.75 |
| RND3 | ras homolog gene family, member E precursor | 1.78 |
| CTGF | connective tissue growth factor precursor | 1.80 |
| HBP1 | HMG-box transcription factor 1 | 1.88 |
| ARID5B | AT rich interactive domain 5B (MRF1-like) | 1.93 |
| PLAU | plasminogen activator, urokinase isoform 1 | 2.04 |
| GDF15 | growth differentiation factor 15 | 3.02 |

B. Rapamycin sensitive transcriptionally regulated genes
upon 3-hour treatment with rapamycin (50 nM) in PC3 cells*

| | | |
|---|---|---|
| HBP1 | HMG-box transcription factor 1 | −1.75 |

*log₂ fold change

### Example 2

### Translation of Pro-Invasion mRNAs by mTOR

To extend the use of the mTOR pharmacological tools used in ribosome profiling towards functional characterization of the newly identified mTOR-sensitive cell invasion gene signature, a new clinical-grade mTOR ATP site inhibitor was developed that was derived from the PP242 chemical scaffold. In brief, a structure-guided optimization of pyrazolopyrimidine derivatives was performed that improved oral bioavailability while retaining mTOR kinase potency and selectivity. The ATP site inhibitor of mTOR was selected for clinical studies on the basis of its high potency (1.4 nM inhibition constant (Kᵢ)), selectivity for mTOR, low molecular mass, and favorable pharmaceutical properties.

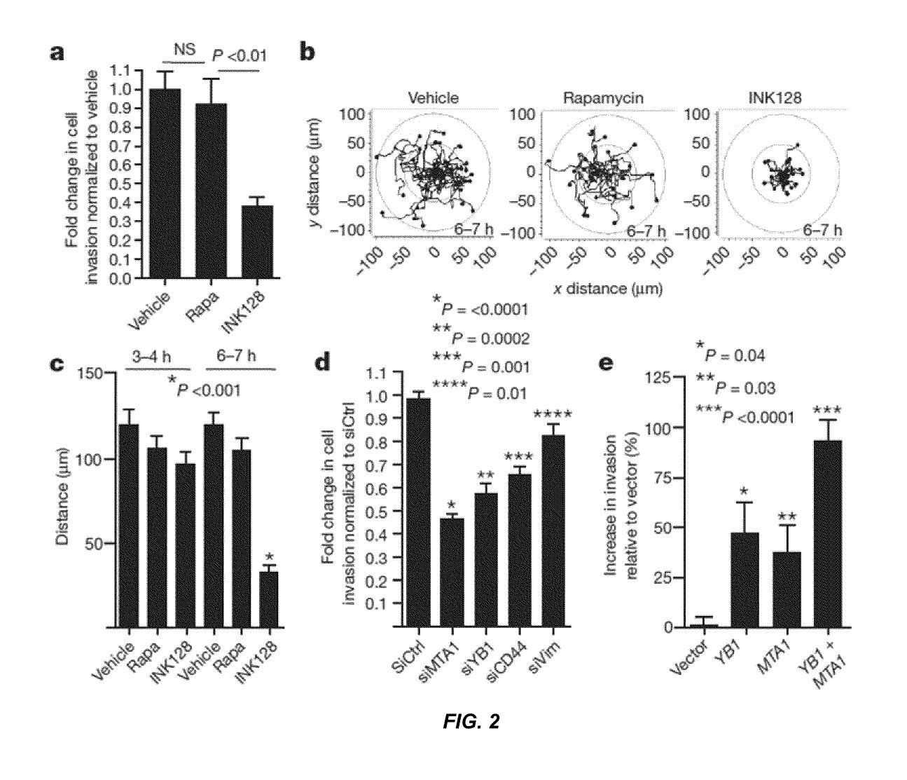

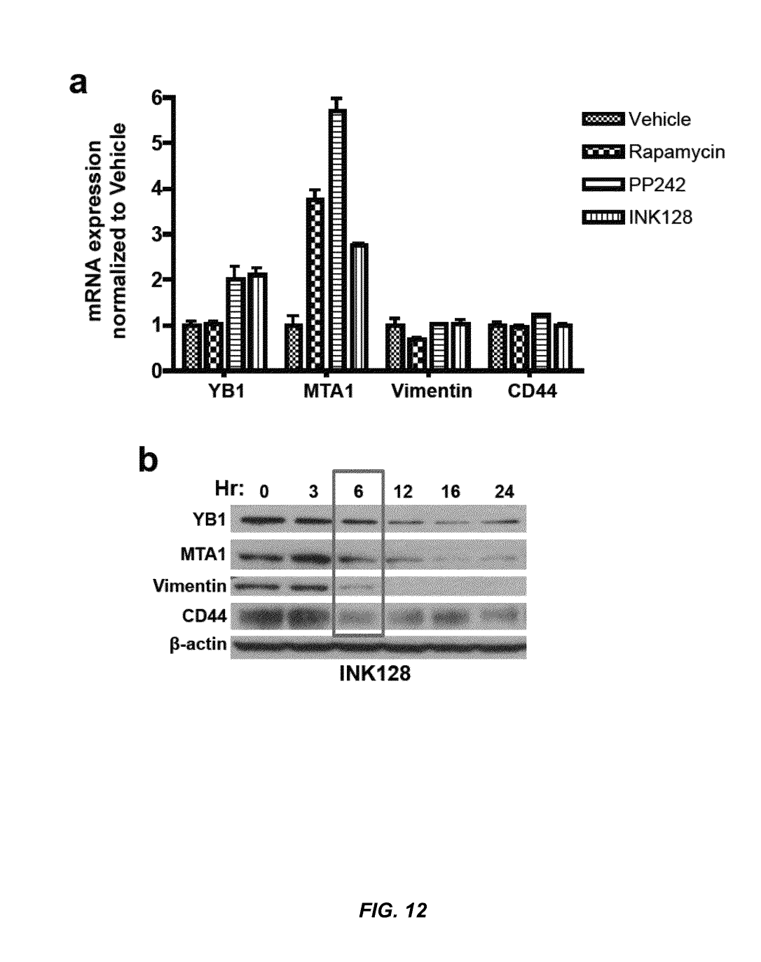

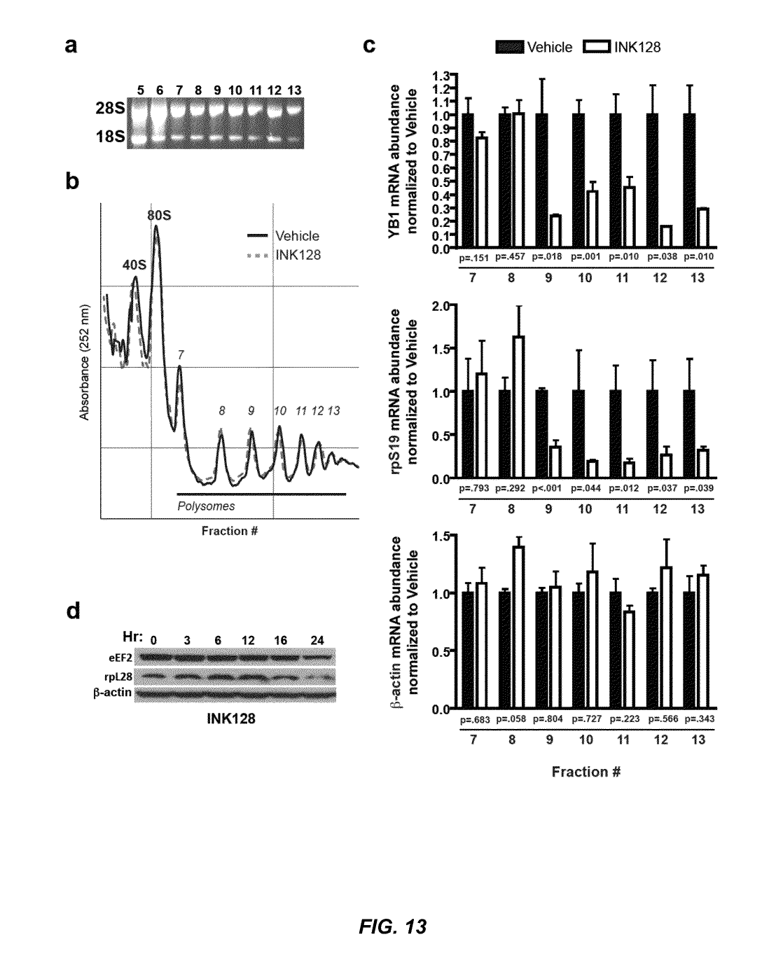

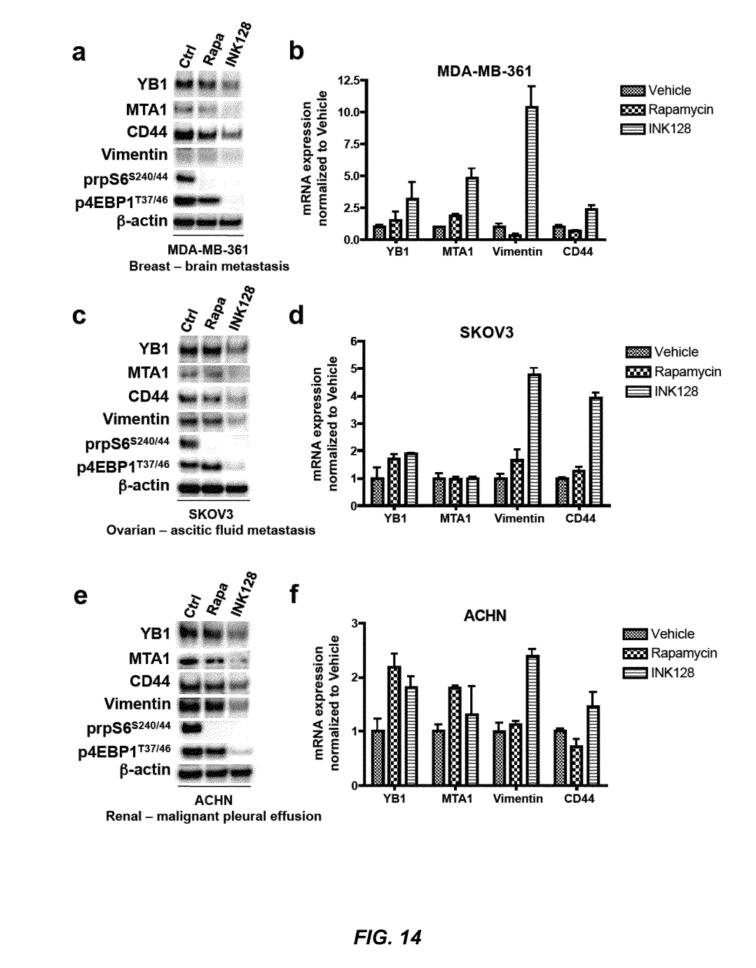

Using either PP242 or the new (or optimized) ATP site inhibitor of mTOR, a selective decrease in the expression of YB1, MTA1, vimentin, and CD44 was observed at the protein but not transcript level in PC3 cells starting at 6 hr of treatment, which preceded any decrease in de novo protein synthesis (FIGS. 1f-1g, 6c-d, 12, and 13). In contrast, rapamycin treatment did not alter their expression (FIGS. 1g and 12a). Similar findings were observed using a broad panel of metastatic cell lines of distinct histological origins (FIG. 14). The four-gene invasion signature (YB1, MTA1, vimentin and CD44) was positively regulated by mTOR hyperactivation, as silencing PTEN expression increased their protein but not mRNA expression levels (FIG. 15). Next, the effects of mTOR ATP site inhibitors on prostate cancer cell migration and invasion were investigated. The ATP site inhibitor of mTOR, but not rapamycin, decreased the invasive potential of PC3 prostate cancer cells (FIG. 2a). Furthermore, the ATP site inhibitor of mTOR inhibited cancer cell migration starting at 6 hr of treatment, precisely correlating with when decreases in the expression of pro-invasion genes were evident, but preceding any changes in the cell cycle or overall global protein synthesis (FIGS. 2b-c, 6c, 6e, 6f, 12b, and 16).

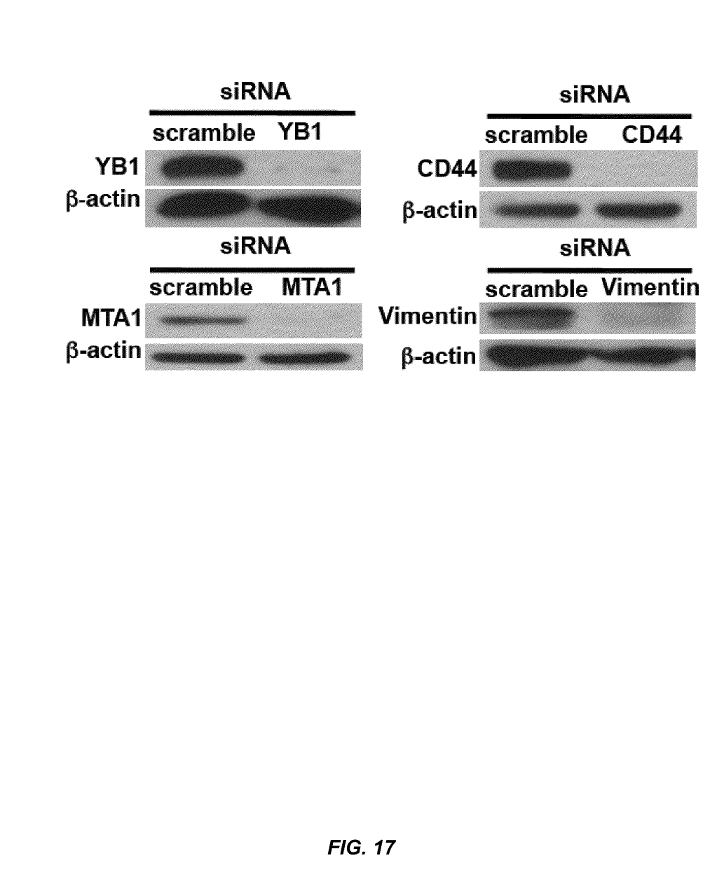

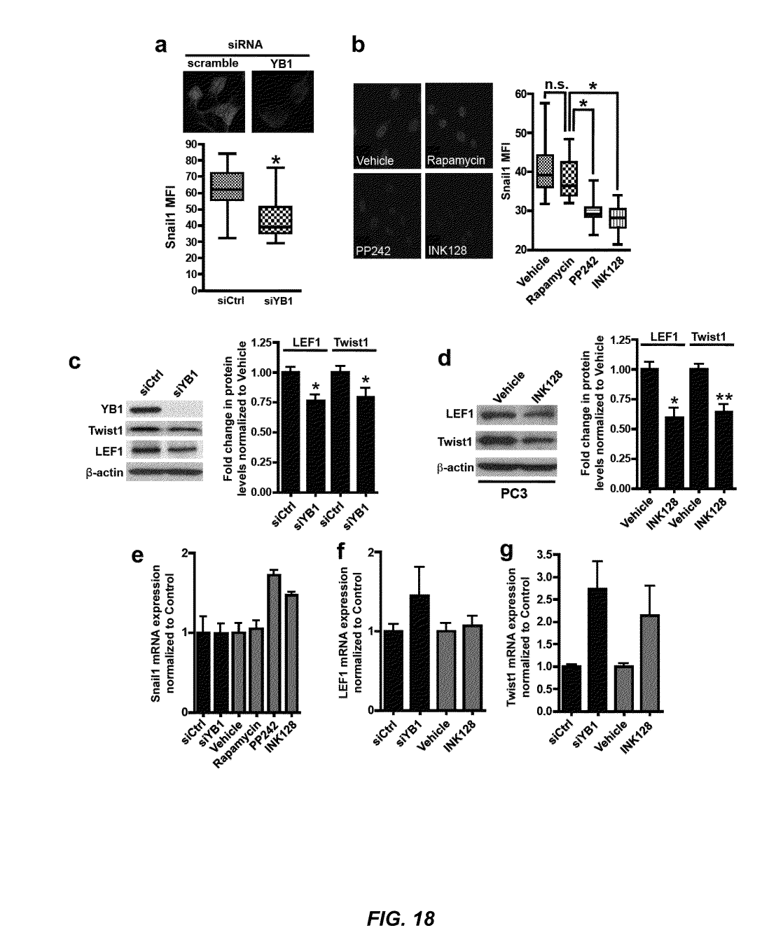

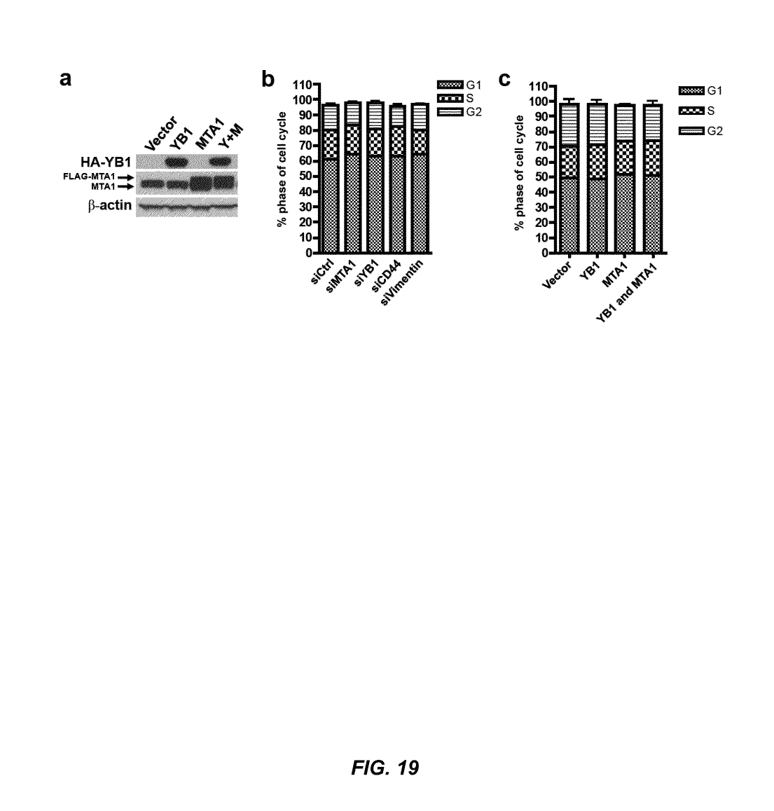

Among the genes comprising the pro-invasion signature, YB1 has been shown to act directly as a translation factor that controls expression of a larger set of genes involved in breast cancer cell invasion. Notably, YB1 translationally-regulated target mRNAs, including SNAIL1 (also called SNAI), LEFT and TWIST1, decreased at the protein but not transcript level upon YB1 knockdown in PC3 cells (FIGS. 17 and 18). To determine the functional role of YB1 in prostate cancer cell invasion, YB1 gene expression was silenced in PC3 cells and a 50% reduction in cell invasion was observed (FIG. 2d). Similarly, knockdown of MTA1, CD44, or vimentin also inhibited prostate cancer cell invasion (FIGS. 2d and 17). These mTOR target mRNAs may be sufficient to endow primary prostate cells with invasive features, as overexpression of YB1 and/or MTA1 (FIG. 19a) in BPH-1 cells, an untransformed prostate epithelial cell line, increased the invasive capacity of these cells in an additive manner (FIG. 2e). Notably, the effects of YB1 and MTA1 on cell invasion were independent from any effect on cell proliferation in both knockdown or overexpression studies (FIG. 19b-c). Therefore, translational control of pro-invasion mRNAs by oncogenic mTOR signaling alters the ability of epithelial cells to migrate and invade, a key feature of cancer metastasis.

### Example 3

### Dissecting mTOR Translational Effectors

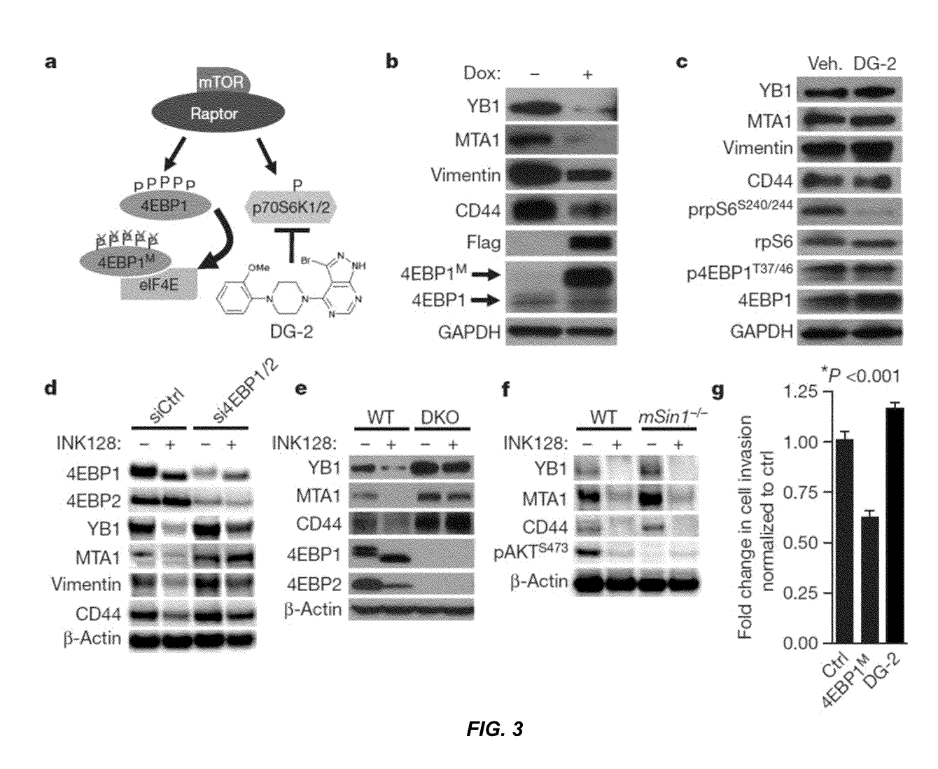

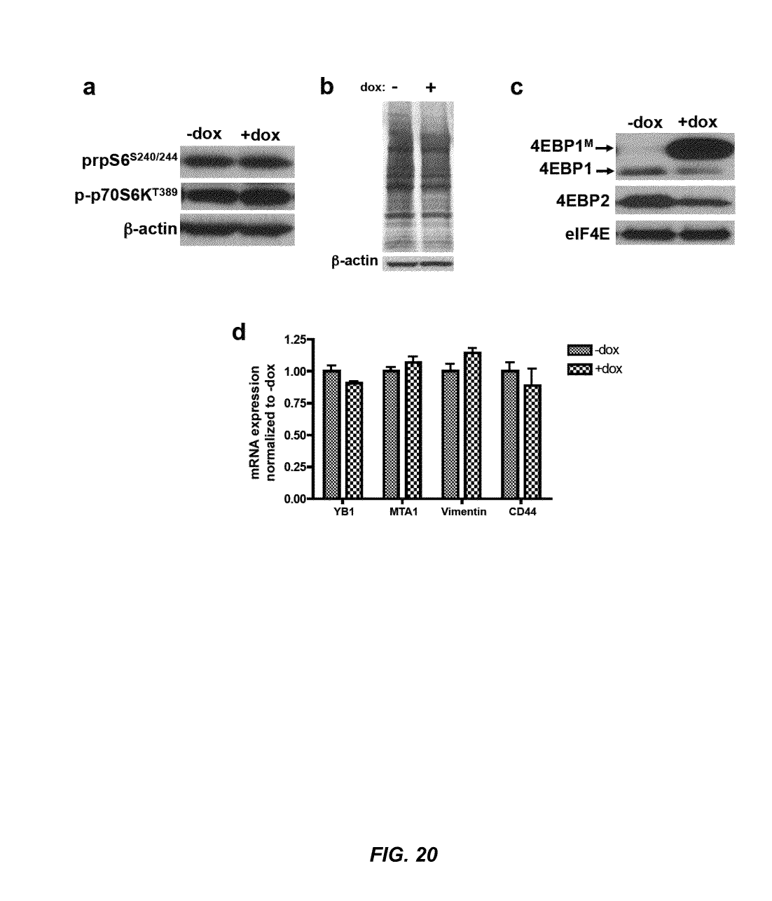

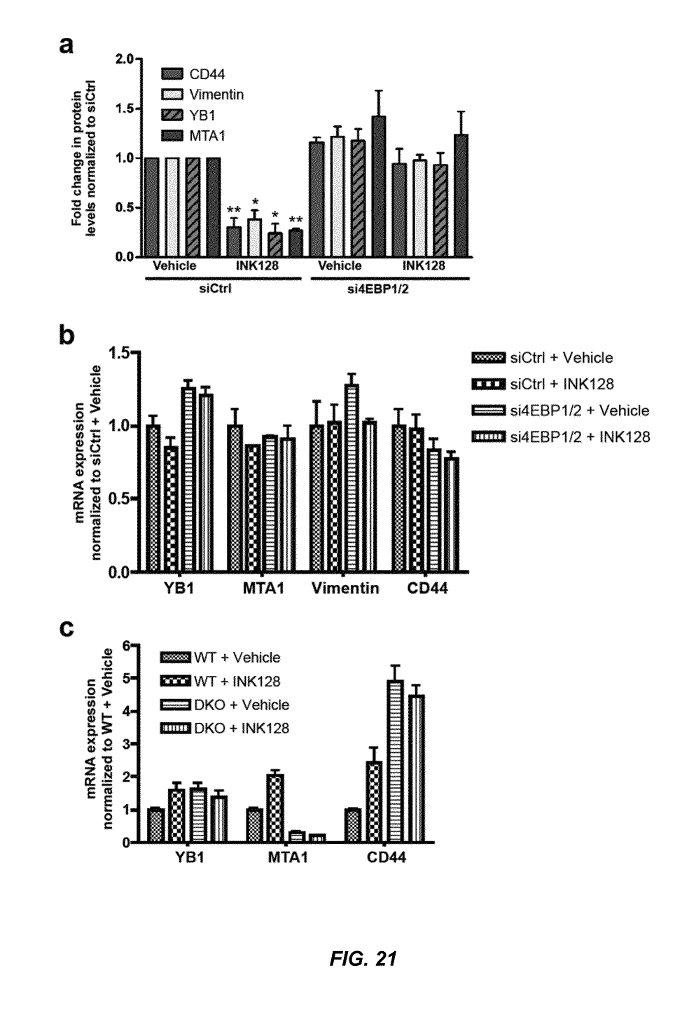

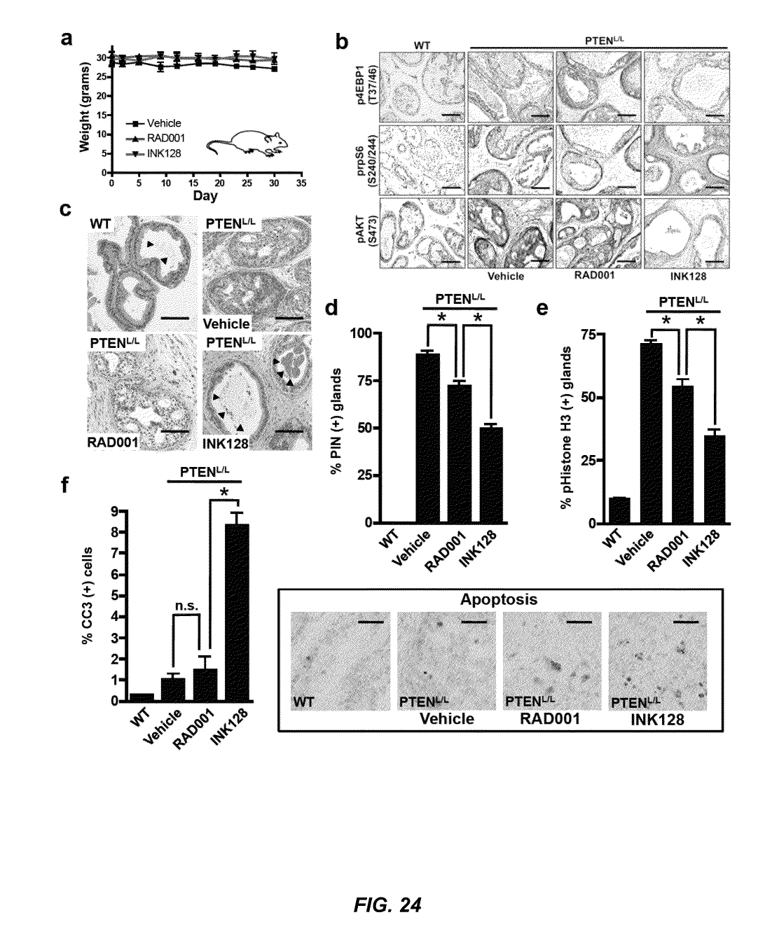

To determine the molecular mechanism by which pro-invasion genes are regulated at the translational level and why these mRNAs are sensitive to an ATP site inhibitor of mTOR but not rapamycin, we investigated whether the downstream translational regulators mTORC1, 4EBP1, and/or p70S6K1/2 controlled the expression of these mTOR-sensitive targets. A human prostate cancer cell line was generated that stably expressed a doxycycline-inducible dominant-negative mutant of 4EBP1 (4EBP1^{M}) (FIG. 3a) (Hsieh et al., Cancer Cell 17:249-261 (2010)). This mutant binds to eIF4E, decreasing its hyperactivation without inhibiting general mTORC1 function (FIG. 20a). Notably, expression of 4EBP1^{M} did not alter global protein synthesis (FIG. 20b), probably because endogenous 4EBP1 and 4EBP2 proteins retain their ability to bind to eIF4E (FIG. 24c). Upon induction of 4EBP1^{M}, YB1, vimentin, CD44 and MTA1 decreased at the protein but not mRNA level (FIGS. 3b-c and 24d).

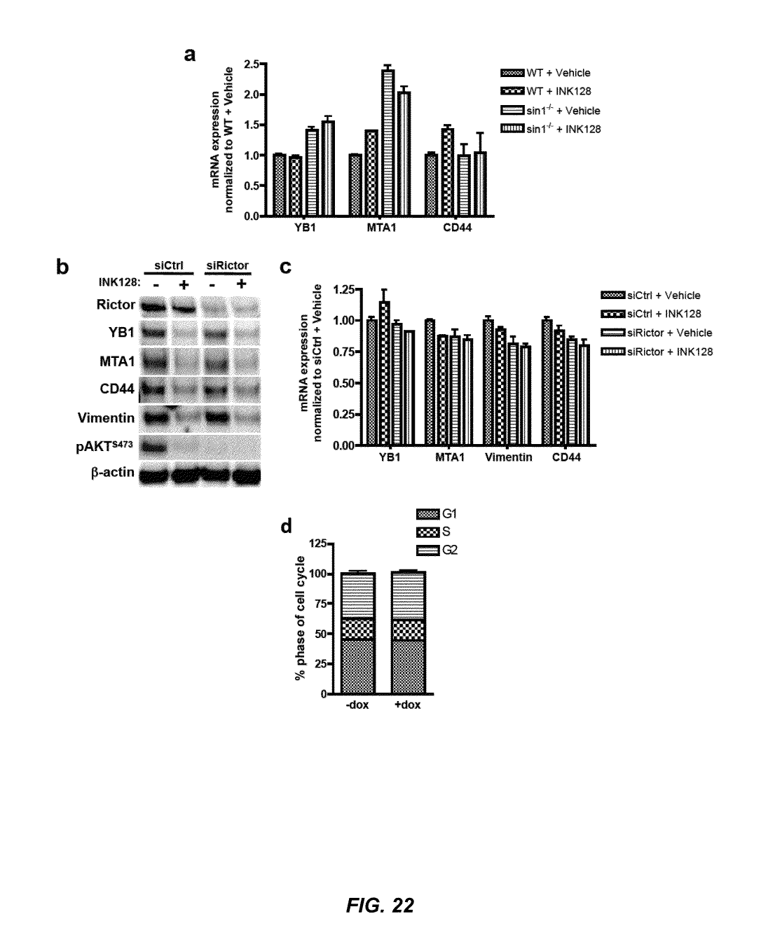

Next, we tested whether an ATP site inhibitor of mTOR decreases expression of the four invasion genes through the 4EBP-eIF4E axis. Notably, knockdown of 4EBP1 and 4EBP2 in PC3 cells or using 4EBP1 and 4EBP2 double knockout mouse embryonic fibroblasts (MEFs) (Dowling et al., Science 328:1172-1176 (2010)) reduced the ability of the ATP site inhibitor of mTOR to decrease expression of these pro-invasion mRNAs (FIGS. 3d-e and 21). Furthermore, ablation of mTORC2 activity had no effect on the expression of these mRNAs or responsiveness to ATP site inhibitor of mTOR (FIGS. 3f and 22a-c). Next, we determined the effect of 4EBP1^{M} on human prostate cancer cell invasion. The expression of 4EBP1^{M} resulted in a significant decrease in prostate cancer cell invasion without affecting the cell cycle, whereas DG-2 had no effect (FIGS. 3g and 22d). These findings demonstrate that eIF4E hyperactivation downstream of oncogenic mTOR regulates translational control of the pro-invasion mRNAs and provides an explanation for the selective targeting of this gene signature by mTOR ATP site inhibitors.

### Example 4

### Examining Cell Invasion Networks In Vivo

Both CK5⁺ and CK8⁺ prostate epithelial cells have been implicated in the initiation of prostate cancer upon loss of PTEN (Wang et al., Nature 461:495-500 (2009); Mulholland et al., Cancer Res. 69:8555-8562 (2009)). Pten^{loxp/loxp}; Pb-cre (Pten^{L/L}) mice are an ideal model of prostate cancer because they display distinct stages of cancer development (prostatic intraepithelial neoplasia, invasive adenocarcinoma, and metastasis) (Wang et al., Cancer Cell 4:209-221 (2003)). However, the expression patterns of YB1, vimentin, CD44 and MTA1 in prostate basal (CK5⁺) and luminal (CK8⁺) epithelial cells have not been characterized.

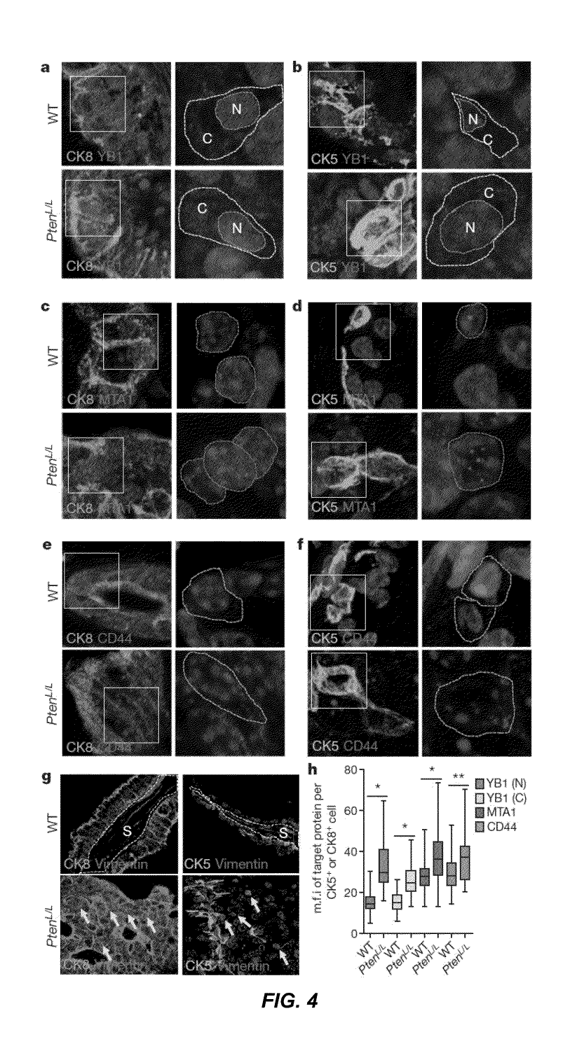

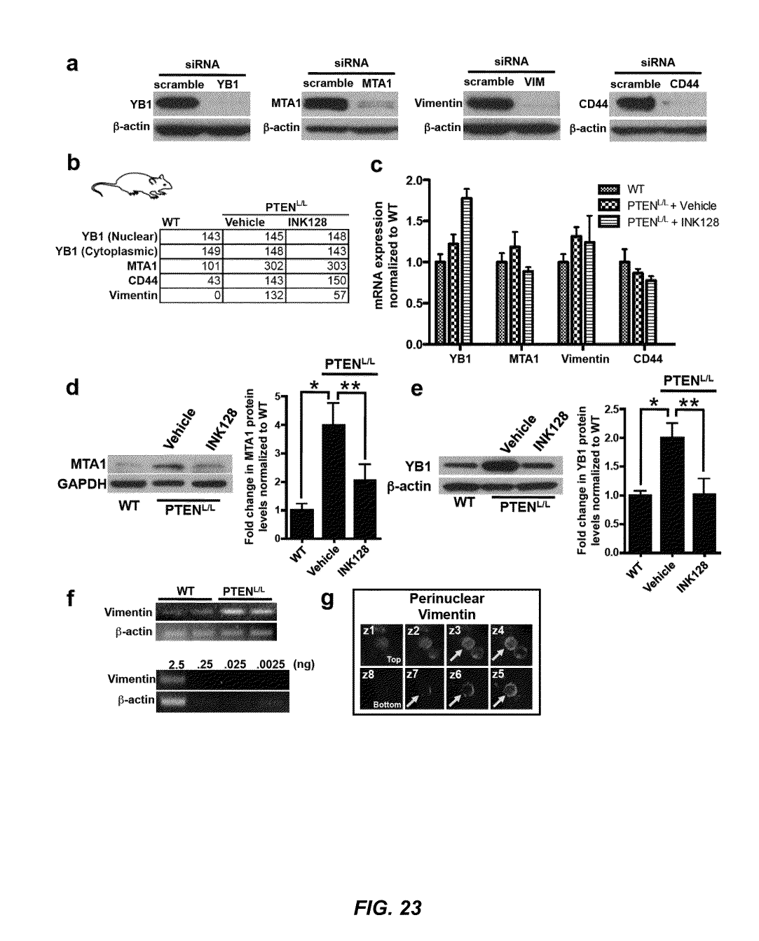

We therefore analyzed their expression patterns in the Pten^{L/L} prostate cancer mouse model, where mTOR is constitutively hyperactivated. YB1 localized to the cytoplasm and nucleus of CK5⁺ and CK8⁺ prostate epithelial cells, consistent with its ability to shuttle between the two cellular compartments (FIGS. 4a-b, 23a-b). MTA1 expression was exclusively nuclear in both cell types (FIG. 4c-d). CD44 expression was observed within a subset of CK5⁺ and CK8⁺ epithelial cells (FIG. 4e-f). CD44, together with other cell-surface markers, has been used to isolate a rare prostate stem-cell population (Leong et al., Nature 456:804-818 (2008)). In contrast, vimentin was not detected in either cell type (FIG. 4g). Next, the impact of mTOR hyperactivation on the expression pattern of the pro-invasion gene signature was determined. YB1, MTA1, and CD44 protein, but not transcript, levels were significantly increased in both Pten^{L/L} luminal and basal epithelial cells compared to wild-type (FIGS. 4h and 23c-e). These studies reveal a unique, translationally controlled signature of gene expression downstream of mTOR hyperactivation in a cancer-initiating subset of pro-state epithelial cells.

### Example 5

### Targeting Prostate Cancer Metastasis

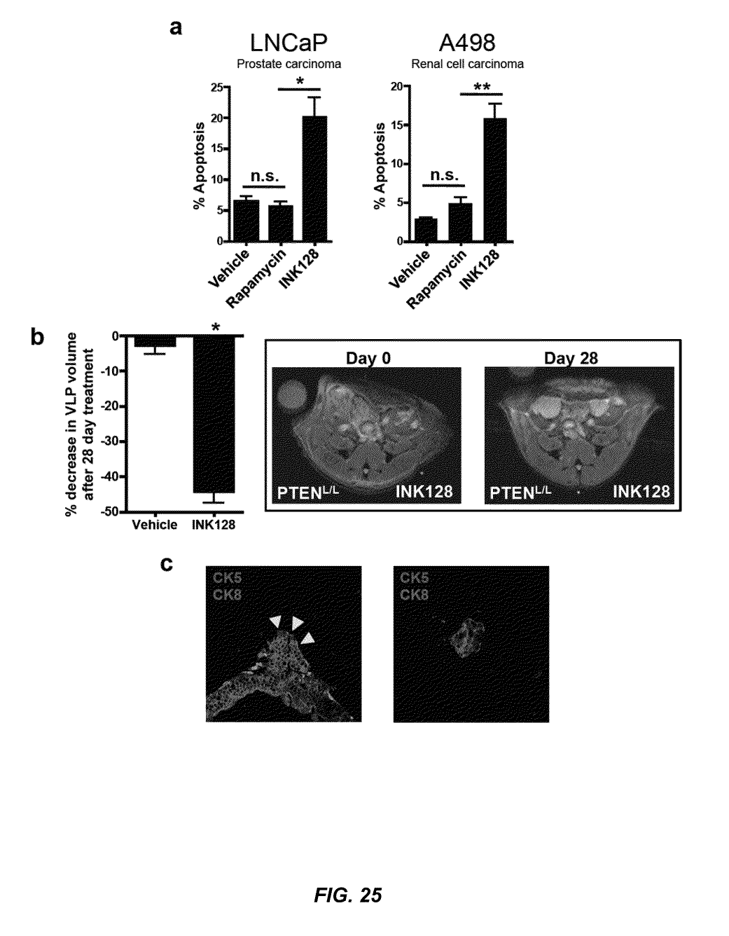

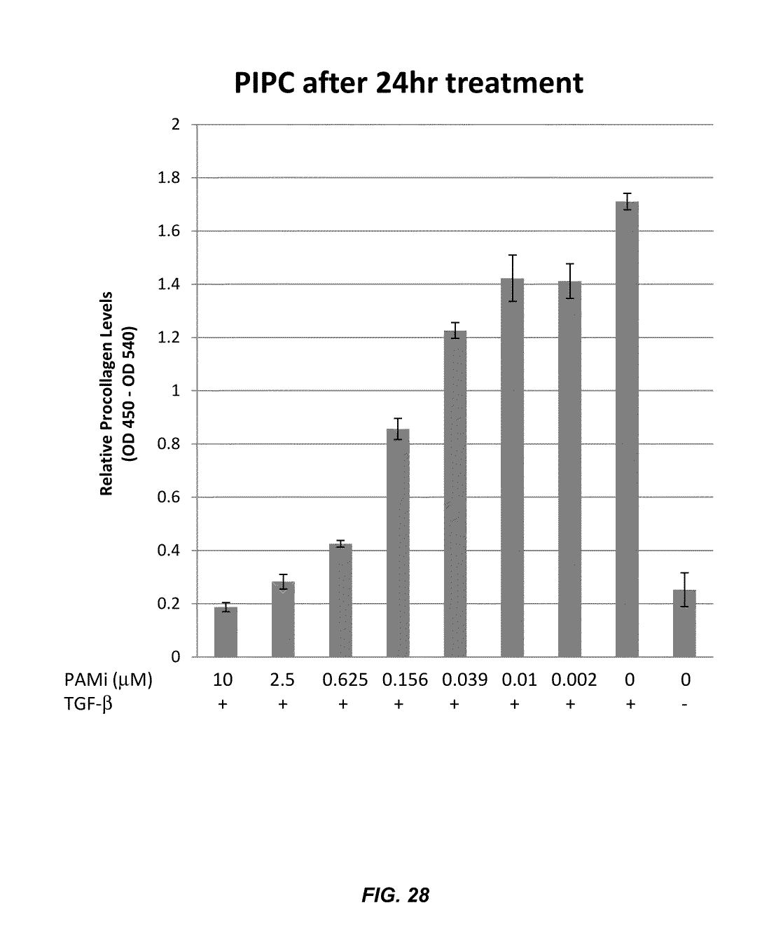

In a preclinical trial of RAD001 (rapalog) versus an ATP site inhibitor of mTOR in Pten^{L/L} mice, 4EBP1 and p70S6K1/2 phosphorylation was completely restored to wild-type levels after treatment with the ATP site inhibitor of mTOR, whereas RAD001 only decreased p70S6K1/2 phosphorylation levels (FIG. 24a-b). Next, the cellular consequences of complete versus partial mTOR inhibition during distinct stages of prostate cancer were determined. Treatment with the ATP site inhibitor of mTOR resulted in a 50% decrease in prostatic intraepithelial neoplasia (PIN) lesions in Pten^{L/L} mice that was associated with decreased proliferation and a tenfold increase in apoptosis (FIG. 24d-f). Notably, the unique cytotoxic properties of ATP site inhibitor of mTOR treatment in Pten^{L/L} mice were evidenced by a marked reduction in prostate cancer volume. In addition, and consistent with these findings, the ATP site inhibitor of mTOR induced programmed cell death in multiple cancer cell lines (FIG. 25a-b). In contrast, RAD001 treatment mainly had cytostatic effects leading to only partial regression of PIN lesions associated with a limited decrease in cell proliferation and no significant effect on apoptosis (FIG. 28c-f).

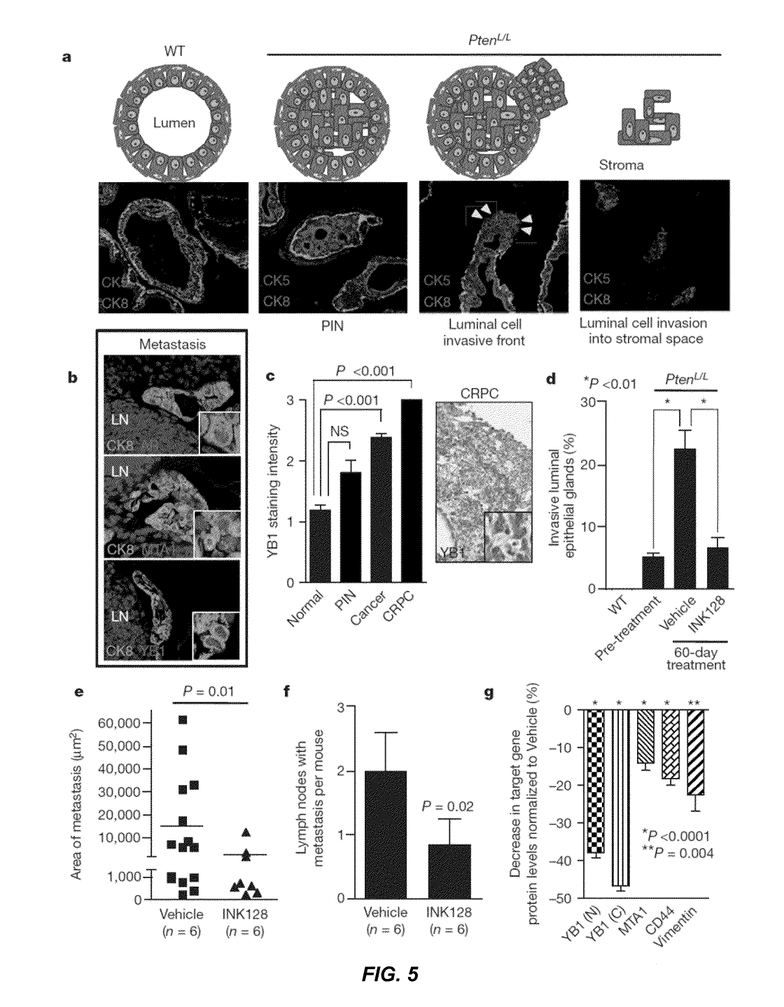

The preclinical trial was extended by examining the effects of the ATP site inhibitor of mTOR treatment on the pro-invasion gene signature and prostate cancer metastasis, which is incurable and the primary cause of patient mortality. Cell invasion is the critical first step in metastasis, required for systemic dissemination. In Pten^{L/L} mice after the onset of PIN, a subset of prostate glands showed characteristics of luminal epithelial cell invasion by 12 months (FIGS. 5a and 25c). After 12 months of age, Pten^{L/L} mice developed lymph-node metastases and these cells maintained strong YB1 and MTA1 expression (FIG. 5b). These findings were extended directly to human prostate cancer patient specimens, in which it was observed that YB1 expression levels increased in a stepwise fashion from normal prostate to castration-resistant prostate cancer (CRPC), an advanced form of the disease associated with increased metastatic potential (FIG. 5c). Similar increases have been observed in MTA1 levels (Hofer et al., Cancer Res. 64:825-829 (2004)).

In human prostate cancer, high-grade primary tumors that display invasive features are more likely to develop systemic metastasis than low-grade non-invasive tumors. Remarkably, treatment with the ATP site inhibitor of mTOR completely blocked the progression of invasive prostate cancer locally in the prostate gland, and profoundly inhibited the total number and size of distant metastases (FIG. 5d-f). This was associated with a marked decrease in the expression of YB1, vimentin, CD44, and MTA1 at the protein, but not transcript, level in specific epithelial cell types within pre-invasive PIN lesions in Pten^{L/L} mice (FIG. 5g and FIG. 23c). Together, these findings reveal an unexpected role for oncogenic mTOR signaling in control of a pro-invasion translational program that, along with the lethal metastatic form of prostate cancer, can be efficiently targeted with clinically relevant mTOR ATP site inhibitors. These findings also demonstrate that translational profiling can be used to identify or validate targets for therapeutic intervention, such as genes that are modulated in cancer.

### Example 6

### Methods

Mice.

Pten^{loxp/loxp} and Pb-cre mice where obtained from Jackson Laboratories and Mouse Models of Human Cancers Consortium (MMHCC), respectively, and maintained in the C57BL/6 background. Mice were maintained under specific pathogen-free conditions, and experiments were performed in compliance with institutional guidelines as approved by the Institutional Animal Care and Use Committee of UCSF.

Cell Culturing and Reagents.

Human cell lines were obtained from the ATCC and maintained in the appropriate medium with supplements as suggested by ATCC. Wild-type, mSin1^{-/-}, and 4EBP1/4EBP2 double knockout MEFs were cultured as previously described (Dowling et al., Science 328:1172-1176 (2010); Jacinto et al., Cell 127:125-137 (2006). SMARTvector 2.0 (Thermo Scientific) lentiviral shRNA constructs were used to knock down PTEN(SH-003023-02-10). For generation of GFP-labeled PC3 cells, SMARTvector 2.0 lentiviral empty vector control particles that contained TurboGFP (S-004000-01) were used. Control (D-001810-01), YB1 (L-010213), MTA1 (L-004127), CD44 (L-009999), vimentin (L-003551), rictor (LL-016984), 4EBP1 (L-003005), and 4EBP2 (L-018671) pooled siRNAs were purchased from Thermo Scientific. The ATP site inhibitors of mTOR INK128 and PP242 were used at 200 nM and 2.5 μM in cell-based assays unless otherwise specified. RAD001 was obtained from LC Laboratories. DG-2 was used at 20 μM in cell-based assays. Rapamycin was purchased from Calbiochem and used at 50 nM in cell-based assays. Doxycyline (Sigma) was used at 1 μg ml⁻¹ in 4EBP1^{M} induction assays. Lipofectamine 2000 (Invitrogen) was used to transfect cancer cell lines with siRNA. Amaxa Cell Line Nucleofector Kit R (Lonza) was used to electroporate BPH-1 cells with overexpression vectors. The 4EBP1^{M} has been previously described (Hsieh et al., Cancer Cell 17:249-261 (2010)).

Plasmids.

pcDNA3-HA-YB1 was provided by V. Evdokimova. pCMV6-Myk-DDK-MTA1 was purchased from Origene. pGL3-Promoter was purchased from Promega. To clone the 5′ UTR of YB1 into pGL3-Promoter, the entire 5′ UTR sequence of YB1 was amplified from PC3 cDNA. PCR fragments were digested with HindIII and NcoI and ligated into the corresponding sites of pGL3-Promoter. The PRTE sequence at position +20-34 in the YB1 5′ UTR (UCSC kgID uc001chs.2) was mutated using the QuikChange Site-Directed Mutagenesis Kit following the manufacturer's protocol (Stratagene).

Ribosome Profiling.

PC3 cells were treated with rapamycin (50 nM) or PP242 (2.5 μM) for 3 hr. Cells were subsequently treated with cycloheximide (100 μg ml⁻¹) and detergent lysis was performed in the dish. The lysate was treated with DNase and clarified, and a sample was taken for RNA-seq analysis. Lysates were subjected to ribosome footprinting by nuclease treatment. Ribosome-protected fragments were purified, and deep sequencing libraries were generated from these fragments, as well as from poly(A) mRNA purified from non-nuclease-treated lysates. These libraries were analyzed by sequencing on an Illumina GAII.

Each sequencing run resulted in approximately 20-25 million raw reads per sample, of which 5-12 million unique reads were used for subsequent analysis. Ribosome footprint and RNA-seq sequencing reads were aligned against a library of transcripts from the UCSC Known Genes database GRCh37/hg19. The first 25 nucleotides of each read were aligned using Bowtie and this initial alignment was then extended to encompass the full fragment-derived portion of the sequencing read while excluding the linker sequence. Read density profiles were then constructed for the canonical transcript of each gene, using only reads with 0 or 1 total mismatches between the read sequence and the reference sequence, comprised of the transcript fragment followed by the linker sequence. Footprint reads were assigned to an A site nucleotide at position +15 to +17 of the alignment, based on the total fragment length; mRNA reads were assigned to the first nucleotide of the alignment. The average read density per codon was then computed for the coding sequence of each transcript, excluding the first 15 and last 5 codons, which can display atypical ribosome accumulation.

Average read density was used as a measure of mRNA abundance (RNA-seq reads) and of protein synthesis (ribosome profiling reads). For most analyses, genes were filtered to require at least 256 reads in the relevant RNA-seq samples. Translational efficiency was computed as the ratio of ribosome footprint read density to RNA-seq read density, scaled to normalize the translational efficiency of the median gene to 1.0 after excluding regulated genes (log₂ fold-change ±1.5 after normalizing for the all-gene median). Changes in protein synthesis, mRNA abundance and translational efficiency were similarly computed as the ratio of read densities between different samples, normalized to give the median gene a ratio of 1.0. This normalization corrects for differences in the absolute number of sequencing reads obtained for different libraries. 3,977 (replicate 1), and 5,333 (replicate 2) unique mRNAs passed a preset read threshold of 256 reads for single-gene quantification for all treatment conditions.

Western Blot Analysis.

Western blot analysis was performed as previously described (Hsieh et al., Cancer Cell 17:249-261 (2010)) with antibodies specific to phospho-AKT^{S473} (Cell Signaling), AKT (Cell Signaling), phospho-p70S6K^{T389} (Cell Signaling), phospho-rpS6^{S240/244} (Cell Signaling), rpS6 (Cell Signaling), phospho-4EBP1^{T37/46} (Cell Signaling), 4EBP1 (Cell Signaling), 4EBP2 (Cell Signaling), YB1 (Cell Signaling), CD44 (Cell Signaling), LEF1 (Cell Signaling), PTEN (Cell Signaling), eEF2 (Cell Signaling), GAPDH (Cell Signaling), vimentin (BD Biosciences), eIF4E (BD Biosciences), Flag (Sigma), β-actin (Sigma), MTA1 (Santa Cruz Biotechnology), Twist (Santa Cruz Biotechnology), rpL28 (Santa Cruz Biotechnology), HA (Covance) and rictor (Bethyl Laboratory).

qPCR Analysis.

RNA was isolated using the manufacturer's protocol for RNA extraction with TRIzol Reagent (Invitrogen) using the Pure Link RNA mini kit (Invitrogen). RNA was DNase-treated with Pure Link Dnase (Invitrogen). DNase-treated RNA was transcribed to cDNA with SuperScript III First-Strand Synthesis System for RT-PCR (Invitrogen), and 1 μl of cDNA was used to run a SYBR green detection qPCR assay (SYBR Green Supermix and MyiQ2, Biorad). Primers were used at 200 nM.

5′ UTR Analysis.

5′ UTRs of the 144 downregulated mTOR target genes were obtained using the known gene ID from the UCSC Genome Browser (GRCh37/hg19). Target versus non-target mRNAs were compared for 5′ UTR length, % G+C content and Gibbs free energy by the Wilcoxon two-sided test. Multiple Eₘ (expectation maximization) for Motif Elicitation (MEME) and Find Individual Motif Occurrences (FIMO) was used to derive the PRTE and determine its enrichment in the 144 mTOR-sensitive genes compared a background list of 3,000 genes. The Database of Transcriptional Start Sites (DBTSS Release 8.0) was used to identify putative 5′ TOP genes and putative transcription start sites in the 144 mTOR target genes.

Luciferase Assay.

PC3 4EBP1^{M} cells were treated with 1 μg ml⁻¹ doxycycline (Sigma) for 24 hr. Cells were transfected with various pGL3-Promoter constructs using lipofectamine 2000 (Invitrogen). After 24 hr, cells were collected. 20% of the cells were aliquoted for RNA isolation. The remaining cells were used for the luciferase assay per the manufacturer's protocol (Promega). Samples were measured for luciferase activity on a Glomax 96-well plate luminometer (Promega). Firefly luciferase activity was normalized to luciferase mRNA expression levels.

Kinase Assays.

mTOR activity was assayed using LanthaScreen Kinase kit reagents (Invitrogen) according to the manufacturer's protocol. PI(3)K α, β, γ, and δ activity were assayed using the PI(3)K HTRF assay kit (Millipore) according to the manufacturer's protocol. The concentration of ATP site inhibitor of mTOR necessary to achieve inhibition of enzyme activity by 50% (IC₅₀) was calculated using concentrations ranging from 20 μM to 0.1 nM (12-point curve). IC₅₀ values were determined using a nonlinear regression model (GraphPad Prism 5).

Cell Proliferation Assay.

PC3 cells were treated with the appropriate drug for 48 hr, and proliferation was measured using Cell Titer-Glo Luminescent reagent (Promega) per the manufacturer's protocol. The concentration of ATP site inhibitor of mTOR necessary to achieve inhibition of cell growth by 50% (IC₅₀) was calculated using concentrations ranging from 20.0 μM to 0.1 nM (12-point curve).

Mouse Xenograft Study.

Nude mice were inoculated subcutaneously in the right subscapular region with 5×10⁶ MDA-MB-361 cells. After tumors reached a size of 150-200 mm³, mice were randomly assigned into vehicle control or treatment groups. The ATP site inhibitor of mTOR was formulated in 5% polyvinylpropyline, 15% NMP, 80% water and administered by oral gavage at 0.3 mg kg⁻¹ and 1 mg kg⁻¹ daily.

Pharmacokinetic Analysis.

The area under the plasma drug concentration versus time curves, AUC₍₀₋ₜₗₐₛₜ₎ and AUC_{(0-inf)}, were calculated from concentration data using the linear trapezoidal rule. The terminal t_{1/2} in plasma was calculated from the elimination rate constant (lz), estimated as the slope of the log-linear terminal portion of the plasma concentration versus time curve, by linear regression analysis. The bioavailability (F) was calculated using F=AUC_{(0-tlast),po}D_{i.v.})/AUC_{(0-last),iv}D_{p.o.})×100%, where D_{i.v.} and D_{p.o.} are intravenous and oral doses, respectively. Cₘₐₓ was a highest drug concentration in plasma after oral administration. Tₘₐₓ was the time at which Cₘₐₓ is observed after extravascular administration of drug. Tₗₐₛₜ was the last time point a quantifiable drug concentration can be measured.

Polysome Analysis.

PC3 cells were treated for 3 hr with either DMSO or the ATP site inhibitor of mTOR (100 nM). Cells were re-suspended in PBS containing 100 μml⁻¹ cycloheximide (Sigma) and incubated on ice for 10 min. Cells were centrifuged at 300 g for 5 min at 4° C. and lysed in 10 mM Tris-HCl pH 8, 140 mM NaCl, 5 mM MgCl₂, 640 U ml⁻¹ Rnasin, 0.05% NP-40, 250 μg ml⁻¹ cycloheximide, 20 mM DTT, and protease inhibitors. Samples were incubated for 20 min on ice, then centrifuged once for 5 min at 3,300 g and once for 5 min at 9,300 g, isolating the supernatant after each centrifugation. Lysates were loaded onto 10-50% sucrose gradients containing 0.1 mg ml⁻¹ heparin and 2 mM DTT and centrifuged at 37,000 r.p.m. for 2.5 hr at 4° C. The sample was subsequently fractionated on a gradient fractionation system (ISCO). RNA was extracted from all fractions and run on a TBE-agarose gel to visualize 18S and 28S rRNA. Fractions 7-13 were found to correspond to the polysome fractions and were used for further qPCR analysis.

[³⁵S] Metabolic Labeling.

PC3 or PC3 4EBP1^{M} cells with or without indicated treatment were incubated with 30 μCi of [³⁵S]-methionine for 1 hr after pre-incubation in methionine-free DMEM (Invitrogen). Cells were prepared using a standard protein lysate protocol, resolved on a 10% SDS polyacrylamide gel and transferred onto a PVDF membrane (Bio-Rad). The membrane was exposed to autoradiography film (Denville) for 24 hr and developed.

Cell Cycle Analysis.

Appropriately treated PC3, BPH-1, or PC3-4EBP1^{M} cells were fixed in 70% ethanol overnight at −20° C. Cells were subsequently washed with PBS and treated with RNase (Roche) for 30 min. After this incubation, the cells were permeabilized and treated with 50 μg ml⁻¹ propidium iodide (Sigma) in a solution of 0.1% Tween, 0.1% sodium citrate. Cell cycle data was acquired using a BD FACS Caliber (BD Biosciences) and analyzed with FlowJo (v.9.1).

Apoptosis Analysis.

Appropriately treated LNCaP and A498 cells were labeled with Annexin V-FITC (BD Biosciences) and propidium iodide (Sigma) following the manufacturer's instructions. PI/Annexin data was acquired using a BD FACS Caliber (BD Biosciences) and analyzed with FlowJo (v.9.1).

Matrigel Invasion Assay.

BioCoat Matrigel Invasion Chambers (modified Boyden Chamber Assay; BD Biosciences) were used according to the manufacturer's instructions.

Real-Time Imaging of Cell Migration.

Real-time imaging of GFP-labeled PC3 cells was performed in poly-D-lysine-coated chamber cover glass slides (Lab-Tek). PC3 GFP cells were plated and allowed to adhere for 24 hr. Wells were wounded with a P200 pipette tip. The chamber slides were imaged with an IX81 Olympus wide-field fluorescence microscope equipped with a CO₂- and temperature-controlled chamber and time-lapse tracking system. Images from DIC and GFP channels were taken every 2 min and processed using ImageJ and analyzed for cell migration with Manual Tracking, using local maximum centering correction to maintain a centroid xy coordinate for each cell per frame over time. Tracking data was subsequently processed with the Chemotaxis and Migration tool from ibidi to create xy coordinate plots, velocity, and distance measurements.

Snail1 Immunocytochemistry.

Appropriately transfected or treated PC3 cells were plated on a poly-L-lysine-coated chamber slide (Lab-Tek) and cultured for 48 hr. Cells were fixed with 4% paraformaldehye (EMS), rinsed with PBS, and permeabilized with 0.1% Triton X-100. The samples were blocked in 5% goat serum and then incubated with anti-Snail1 antibody (Cell Signaling) in 5% goat serum for 2 hr at room temperature. Cells were washed with PBS and incubated with Alexa 594 anti-mouse antibody (Invitrogen) and DAPI (Invitrogen) for 2 hr at room temperature. Specimens were again washed with PBS and subsequently mounted with Aqua Poly/Mount (Polysciences). Image capture and quantification were completed as described below (see “Immunofluorescence”).

Cap-Binding Assay.

PC3 4EBP1^{M} cells were induced with doxycycline (1 μg ml⁻¹, Sigma) for 48 hr, then collected and lysed in buffer A (10 mM Tris-HCl pH 7.6, 150 mM KCl, 4 mM MgCl₂, 1 mM DTT, 1 mM EDTA, and protease inhibitors, supplemented with 1% NP-40). Cell lysates were incubated overnight at 4° C. with 50 ml of the mRNA cap analogue m⁷GTP-sepharose (GE Healthcare) in buffer A. The beads were washed with buffer A supplemented with 0.5% NP-40. Protein complexes were dissociated using 1× sample buffer, and resolved by SDS-PAGE and western blotted with the appropriate antibodies.

Pharmacological Treatment of Pten^{L/L} Mice and MRI Imaging.

Nine- and twelve-month-old Pten^{L/L} mice were gavaged daily with either vehicle (see “Mouse xenograft study”), RAD001 (10 mg kg⁻¹), or an ATP site inhibitor of mTOR (1 mg kg⁻¹) for the indicated times. Weight measurements were taken every 3 days to monitor for toxicity. For the 28-day study, mice were imaged via MRI at day 0 and day 28 in a 14-T GE MR scanner (GE Healthcare).

Prostate Tissue Processing.

Whole mouse prostates were removed from wild-type and Pten^{L/L} mice, microdissected, and frozen in liquid nitrogen. Frozen tissues were subsequently manually disassociated using a biopulverizer (Biospec) and additionally processed for protein and mRNA analysis as described above.

Immunofluorescence.

Prostates and lymph nodes were dissected from mice within 2 hr of the indicated treatment and fixed in 10% formalin overnight at 4° C. Tissues were subsequently dehydrated in ethanol (Sigma) at room temperature, mounted into paraffin blocks, and sectioned at 5 μm. Specimens were de-paraffinized and rehydrated using CitriSolv (Fisher) followed by serial ethanol washes. Antigen unmasking was performed on each section using Citrate pH 6 (Vector Labs) in a pressure cooker at 125° C. for 10-30 min. Sections were washed in distilled water followed by TBS washes. The sections were then incubated in 5% goat serum, 1% BSA in TBS for 1 hr at room temperature. Various primary antibodies were used, including those specific for keratin 5 (Covance), cytokeratin 8 (Abcam and Covance), YB1 (Abcam), vimentin (Abcam), MTA1 (Cell signaling), CD44 (BD Pharmingen), and the androgen receptor (Epitomics), which were diluted 1:50-1:500 in blocking solution and incubated on sections overnight at 4° C. Specimens were then washed in TBS and incubated with the appropriate Alexa 488 and 594 labeled secondary (Invitrogen) at 1:500 for 2 hr at room temperature, with the exception of YB1 which was incubated with biotinylated anti-rabbit secondary (Vector) followed by incubation with Alexa 594 labeled Streptavidin (Invitrogen). A final set of washes in TBS was completed at room temperature followed by mounting with DAPI Hardset Mounting Medium (Vector Lab). A Zeiss Spinning Disc confocal (Zeiss, CSU-X1) was used to image the sections at 40×-100×. Individual prostate cells were quantified for mean fluorescence intensity (m.f.i.) using the Axiovision (Zeiss, Release 4.8) densitometric tool.

Lymph Node Metastasis Measurements.

Mouse lymph nodes were processed as described above and stained for CK8 and androgen receptor. Lymph nodes were imaged using a Zeiss AX10 microscope. Metastases were identified and areas were measured using the Axiovision (Zeiss, Release 4.8) measurement tool.

Semi-Quantitative RT-PCR.

Whole prostates were removed from wild-type and Pten^{L/L} mice, microdissected, dissociated into single-cell suspension, and stained for epithelial cell markers as previously described (Lukacs et al., Nature Protocols 5:702-713 (2010)) using fluorescence-conjugated antibodies for CD49f, Sca-1, CD31, CD45, and Ter119 (BD Biosciences). Luminal epithelial cells were sorted using a FACS Aria (BD Biosciences). Cell pellets were resuspended in 500 μl TRIzol Reagent and RNA was isolated and transcribed into cDNA as described above. Semi-quantitative PCR analysis was performed using oligonucleotides for vimentin and β-actin at 200 nM in a 25 μl reaction with 12.5 μl GoTaq (Promega) for 32 and 33 cycles, respectively, which were within the linear range (FIG. 230.

Immunohistochemistry.

Immunohistochemistry was performed as described above (see “Immunofluorescence”) with the exception that immediately after antigen presentation and TBS washes, specimens were incubated in 3% hydrogen peroxide in TBS followed by TBS washes. The following primary antibodies were used: phospho-AKT^{S473} (Cell Signaling), phospho-rpS6^{S240/244} (Cell Signaling), phospho-4EBP1^{T37/46} (Cell Signaling), phospho-histone H3 (Upstate), and cleaved caspase (Cell Signaling). This was followed by TBS washes and incubation with the appropriate biotinylated secondary antibody (Vector Lab) for 30 min at room temperature. An ABC-HRP Kit (Vector Lab) was used to amplify the signal, followed by a brief incubation in hydrogen peroxide. The protein of interest was detected using DAB (Sigma). Specimens were counterstained with haematoxylin (Thermo Scientific), dehydrated with Citrisolv (Fisher), and mounted with Cytoseal XYL (Vector Lab).

Haematoxylin and Eosin Staining.

Paraffin-embedded prostate specimens were deparaffinized and rehydrated as described above (see “Immunofluorescence”), stained with haematoxylin (Thermo Scientific), and washed with water. This was followed by a brief incubation in differentiation RTU (VWR) and two washes with water followed by two 70% ethanol washes. The samples were then stained with eosin (Thermo Scientific) and dehydrated with ethanol followed by CitriSolv (Fisher). Slides were mounted with Cytoseal XYL (Richard Allan Scientific).

Oligonucleotides.

YB1 5′ UTR cloning and site-directed mutagenesis oligonucleotides are as follows. YB1 5′ UTR cloning: forward 5′-GCTACAAGCTTGGGCTTATCCCGCCT-3′ (SEQ ID NO:146), reverse 5′-TCGATCCATGGGGTTGCGGTGATGGT-3′ (SEQ ID NO:147); deletion (20-34): forward 5′-TGGGCTTATCCCGCCTGTCCTTCGATCGGTAGCGGGAGCG-3′ (SEQ ID NO:148), reverse 5′-CGCTCCCGCTACCGATCGAAGGACAGGCGGGATAAGCCCA-3′ (SEQ ID NO:149); transversion (20-34): forward 5′-TGGGCTTATCCCGCCTGTCCGCGGTAAGAGCGATCTTCGATCGGTAGCGGGAGCG-3′ (SEQ ID NO:150), reverse 5′-CGCTCCCGCTACCGATCGAAGATCGCTCTTACCGCGGACAGGCGGGATAAGCCCA-3′ (SEQ ID NO:151).

Human qPCR oligonucleotides are as follows. β-actin forward 5′-GCAAAGACCTGTACGCCAAC-3′ (SEQ ID NO:152), reverse 5′-AGTACTTGCGCTCAGGAGGA-3′ (SEQ ID NO:153); CD44 forward 5′-CAACAACACAAATGGCTGGT-3′ (SEQ ID NO:154), reverse 5′-CTGAGGTGTCTGTCTCTTTCATCT-3′ (SEQ ID NO:155); vimentin forward 5′-GGCCCAGCTGTAAGTTGGTA-3′ (SEQ ID NO:156), reverse 5′-GGAGCGAGAGTGGCAGAG-3′ (SEQ ID NO:157); Snail1 forward 5′-CACTATGCCGCGCTCTTTC-3′ (SEQ ID NO:158), reverse 5′-GCTGGAAGGTAAACTCTGGATTAGA-3′ (SEQ ID NO:159); YB1 forward 5′-TCGCCAAAGACAGCCTAGAGA-3′ (SEQ ID NO:160), reverse 5′-TCTGCGTCGGTAATTGAAGTTG-3′ (SEQ ID NO:161); MTA1 forward 5′-CAAAGTGGTGTGCTTCTACCG-3′ (SEQ ID NO:162), reverse 5′-CGGCCTTATAGCAGACTGACA-3′ (SEQ ID NO:163); PLAU forward 5′-TTGCTCACCACAACGACATT-3′ (SEQ ID NO:164), reverse 5′-GGCAGGCAGATGGTCTGTAT-3′ (SEQ ID NO:165); FGFBP1 forward 5′-ACTGGATCCGTGTGCTCAG-3′ (SEQ ID NO:166), reverse 5′-GAGCAGGGTGAGGCTACAGA-3′ (SEQ ID NO:167); ARID5B forward 5′-TGGACTCAACTTCAAAGACGTTC-3′ (SEQ ID NO:168), reverse 5′-ACGTTCGTTTCTTCCTCGTC-3′ (SEQ ID NO:169); CTGF forward 5′-CTCCTGCAGGCTAGAGAAGC-3′ (SEQ ID NO:170), reverse 5′-GATGCACTTTTTGCCCTTCTT-3′ (SEQ ID NO:171); RND3 forward 5′-AAAAACTGCGCTGCTCCAT-3′ (SEQ ID NO:172), reverse 5′-TCAAAACTGGCCGTGTAATTC-3′ (SEQ ID NO:173); KLF6 forward 5′-AAAGCTCCCACTTGAAAGCA-3′ (SEQ ID NO:174), reverse 5′-CCTTCCCATGAGCATCTGTAA-3′ (SEQ ID NO:175); BCL6 forward 5′-TTCCGCTACAAGGGCAAC-3′ (SEQ ID NO:176), reverse 5′-TGCAACGATAGGGTTTCTCA-3′ (SEQ ID NO:177); FOXA1 forward 5′-AGGGCTGGATGGTTGTATTG-3′ (SEQ ID NO:178), reverse 5′-ACCGGGACGGAGGAGTAG-3′ (SEQ ID NO:179); GDF15 forward 5′-CCGGATACTCACGCCAGA-3′ (SEQ ID NO:180), reverse 5′-AGAGATACGCAGGTGCAGGT-3′ (SEQ ID NO:181); HBP1 forward 5′-GCTGGTGGTGTTGTCGTG-3′ (SEQ ID NO:182), reverse 5′-CATGTTATGGTGCTCTGACTGC-3′ (SEQ ID NO:183); Twist1 forward 5′-CATCCTCACACCTCTGCATT-3′ (SEQ ID NO:184), reverse 5′-TTCCTTTCAGTGGCTGATTG-3′ (SEQ ID NO:185); LEF1 forward 5′-CCTTGGTGAACGAGTCTGAAATC-3′ (SEQ ID NO:186), reverse 5′-GAGGTTTGTGCTTGTCTGGC-3′ (SEQ ID NO:187); rpS19 forward 5′-GCTGGCCAAACATAAAGAGC-3′ (SEQ ID NO:188), reverse 5′-CTGGGTCTGACACCGTTTCT-3′ (SEQ ID NO:189); 5S rRNA forward 5′-GCCCGATCTCGTCTGATCT-3′ (SEQ ID NO:190), reverse 5′-AGCCTACAGCACCCGGTATT-3′ (SEQ ID NO:191); firefly luciferase forward 5′-AATCAAAGAGGCGAACTGTG-3′ (SEQ ID NO:192), reverse 5′-TTCGTCTTCGTCCCAGTAAG-3′ (SEQ ID NO:193).

Mouse qPCR oligonucleotides are as follows. β-actin forward 5′-CTAAGGCCAACCGTGAAAAG-3′ (SEQ ID NO:194), reverse 5′-ACCAGAGGCATACAGGGACA-3′ (SEQ ID NO:195); Yb1 forward 5′-GGGTTACAGACCACGATTCC-3′ (SEQ ID NO:196), reverse 5′-GGCGATACCGACGTTGAG-3′ (SEQ ID NO:197); vimentin forward 5′-TCCAGCAGCTTCCTGTAGGT-3′ (SEQ ID NO:198), reverse 5′-CCCTCACCTGTGAAGTGGAT-3′ (SEQ ID NO:199); Cd44 forward 5′-ACAGTACCTTACCCACCATG-3′ (SEQ ID NO:200), reverse 5′-GGATGAATCCTCGGAATTAC-3′ (SEQ ID NO:201); Mta1 forward 5′-AGTGCGCCTAATCCGTGGTG-3′ (SEQ ID NO:202), reverse 5′-CTGAGGATGAGAGCAGCTTTCG-3′ (SEQ ID NO:203).

siRNA/shRNA sequences are as follows. Control (D-001810-01) 5′-UGGUUUACAUGUCGACUAA-3′ (SEQ ID NO:204); vimentin (L-003551) 5′-UCACGAUGACCUUGAAUAA-3′ (SEQ ID NO:205), 5′-GGAAAUGGCUCGUCACCUU-3′ (SEQ ID NO:206), 5′-GAGGGAAACUAAUCUGGAU-3′ (SEQ ID NO:207), 5′-UUAAGACGGUUGAAACUAG-3′ (SEQ ID NO:208); YB1 (L-010213) 5′-CUGAGUAAAUGCCGGCUUA-3′ (SEQ ID NO:209), 5′-CGACGCAGACGCCCAGAAA-3′ (SEQ ID NO:210), 5′-GUAAGGAACGGAUAUGGUU-3′ (SEQ ID NO:211), 5′-GCGGAGGCAGCAAAUGUUA-3′ (SEQ ID NO:212); MTA1 (L-004127) 5′-UCACGGACAUUCAGCAAGA-3′ (SEQ ID NO:213), 5′-GGACCAAACCGCAGUAACA-3′ (SEQ ID NO:214), 5′-GCAUCUUGUUGGACAUAUU-3′ (SEQ ID NO:215), 5′-CCAGCAUCAUUGAGUACUA-3′ (SEQ ID NO:216); CD44 (L-009999) 5′-GAAUAUAACCUGCCGCUUU-3′ (SEQ ID NO:217), 5′-CAAGUGGACUCAACGGAGA-3′ (SEQ ID NO:218), 5′-CGAAGAAGGUGUGGGCAGA-3′ (SEQ ID NO:219), 5′-GAUCAACAGUGGCAAUGGA-3′ (SEQ ID NO:220); 4EBP1 (L-003005) 5′-CUGAUGGAGUGUCGGAACU-3′ (SEQ ID NO:221), 5′-CAUCUAUGACCGGAAAUUC-3′ (SEQ ID NO:222), 5′-GCAAUAGCCCAGAAGAUAA-3′ (SEQ ID NO:223), 5′-GAGAUGGACAUUUAAAGCA-3′ (SEQ ID NO:224); 4EBP2 (L-018671) 5′-GCAGCUACCUCAUGACUAU-3′ (SEQ ID NO:225), 5′-GGAGGAACUCGAAUCAUUU-3′ (SEQ ID NO:226), 5′-GCAAUUCUCCCAUGGCUCA-3′ (SEQ ID NO:227), 5′-UUGAACAACUUGAACAAUC-3′ (SEQ ID NO:228); rictor (LL-016984) 5′-GACACAAGCACUUCGAUUA-3′ (SEQ ID NO:229), 5′-GAAGAUUUAUUGAGUCCUA-3′ (SEQ ID NO:230), 5′-GCGAGCUGAUGUAGAAUUA-3′ (SEQ ID NO:231), 5′-GGGAAUACAACUCCAAAUA-3′ (SEQ ID NO:232); PTEN SH-003023-01-10 5′-GCTAAGAGAGGTTTCCGAA-3′ (SEQ ID NO:233), SH-003023-02-10 5′-AGACTGATGTGTATACGTA-3′ (SEQ ID NO:234).

### Example 7

### Effect of mTOR and MEK Inhibitors on Translation Efficiency

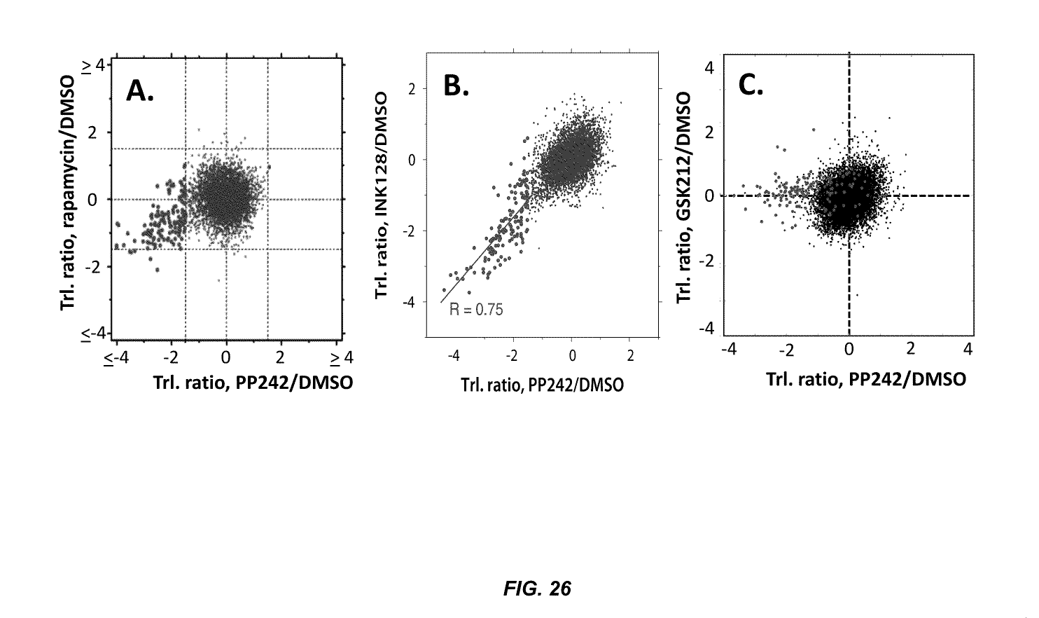

To further examine the effect of mTOR inhibitors on translational efficiency in PC3 prostate cancer cells, the ATP site inhibitor of mTOR PP242 was compared to the allosteric inhibitor of mTOR, rapamycin and to another ATP site inhibitor. FIG. 26 shows a representative comparison of change in translational efficiency versus DMSO control by the allosteric mTOR inhibitor rapamycin and the ATP site inhibitor PP242 (FIG. 26A) and the two ATP site inhibitors INK128 (100 nM) and PP242 (as described in Example 6) (FIG. 26B). Each data point represents a single gene. Data points highlighted in red have statistically significant changes in translational efficiency versus DMSO control as described herein. FIG. 26A shows that most of the genes where translational efficiency decreases due to PP242 also have decreased translational efficiencies caused by rapamycin; however, the magnitude of rapamycin decrease is substantially less than with PP242. In contrast, treatment with INK128 not only impacts the same gene set as PP242, but also has approximately the same magnitude of change on a gene by gene basis (FIG. 26B). This experiment shows that two different drugs that act on a target through the same mechanism (such as PP242 and INK128) will affect translational efficiency in a similar manner—that is, the methods of this disclosure can be used to find active compounds that are pharmacological “mimics” of each other. Even when two compounds have different mechanisms of action on the same target (such as PP242 and rapamycin), effects on translational efficiency can be detected although the degree of the translational effect may be different.

The following experiment was performed to show that translational profiling can be used for a variety of agents and targets. The mTOR inhibitors alter the PI3K/AKT pathway. Here, a MEK/ERK pathway inhibitor (GSK212) was examined.

Cell Culture.

SW620 human colon cancer cells were cultured in DMEM media supplemented with penicillin G (100 U/ml), streptomycin (100 μg/ml), and 10% FBS in a humidified atmosphere of 5% CO₂ maintained at 37° C.

MEK and mTOR Inhibitor Treatment.

SW620 cells (ATCC, passage 12) were seeded at about 75% confluence 24 hrs prior to drug treatment. The following day, cells were treated with either DMSO (vehicle control) or MEK inhibitor GSK-11202012 (referred to herein as “GSK212”) at 250 nM for 8 hrs or with either DMSO or the mTOR inhibitor PP242 at 2.5 μM for 3 hrs. About 6×10⁶ cells/10 cm plate and about 1×10⁶ cells/well of a 6-well plate were harvested for ribosome profiling and Western blot analysis, respectively, following drug treatment.

Western Blot Analysis.

Cells were washed with PBS and lysed in 1× cell lysis buffer (Cell Signaling) for 15 min at 4° C. Lysates were sonicated briefly, clarified by centrifugation for 15 min at 14,000 rpm, and supernatants were then collected. Protein concentration in the soluble fraction was determined by BCA protein assay (Thermo Scientific). A 4-20% Bis-Tris gradient gel (Invitrogen) was used to resolve 20 μg of protein and transferred to nitrocellulose membrane. The resulting membranes were blocked for 1 hr at room temperature with Odyssey blocking solution (LI-COR) and then incubated with primary antibodies at 4° C. overnight. The following day, the blots were washed 3 times, 10 min each in TBST, and incubated with IR-conjugated goat anti-rabbit IgG secondary antibody (IRDye 800 CW at 1:20,000; LI-COR) for 1 hour at room temperature. The blots were then washed, scanned, and specific proteins were detected using the LI-COR Odyssey infrared imager. The following antibodies from Cell Signaling were used at 1:1000 dilution: anti-phospho-eIF4E(Ser209)(#9741), anti-phospho-rpS6(Ser235/236)(#4858), anti-phospho-ERK1/2(Thr202/Tyr204)(#4370), anti-phospho-p70S6K(Thr421/Ser424)(#9204), anti-phospho-p90RSK(Thr359/Ser363)(#9344), anti-phospho-4EBP(Ser65), anti-phospho-pAKT(Ser473), anti-phospho-eIF4E(Ser209), and anti-β-actin (#4970). Actin was used as a loading amount control.

mTOR Inhibitor PP242 and MEK Inhibitor GSK212 are Clearly Distinguishable by Differential Effects on Translational Efficiencies.

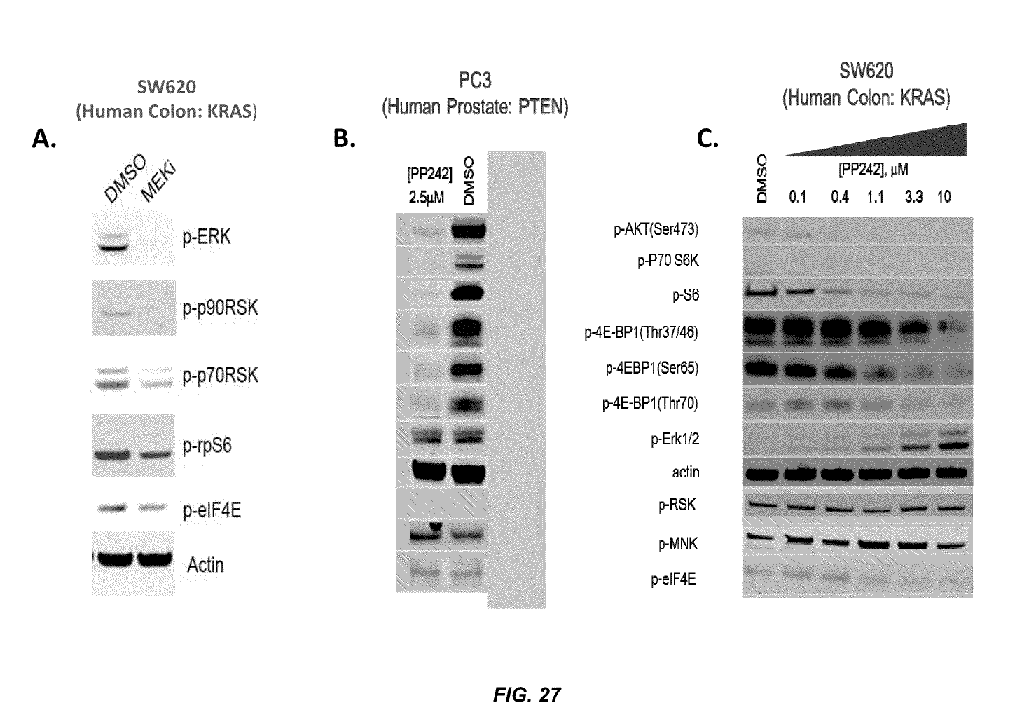

GSK212 is a very potent and selective MEK inhibitor with IC₅₀ values of about 1 nM for both MEK1 and MEK2. The potency of GSK212 in 72 hour proliferation assays on SW620 cells is 20-30 nM (data not shown). In this concentration range, GSK212 has profound effects on the transcriptional program of sensitive cells like SW620. In the experiments described herein, exposure to SW620 cells was at a supra-therapeutic concentration (250 nM) for 8 hours. No evidence of inhibition of proliferation or induction of apoptosis was apparent over this time frame. Phosphorylation of ERK and p90RSK in SW620 cells was completely inhibited (FIG. 27A). At this concentration, only partial inhibition of the phosphorylation of the ribosomal protein S6 (rpS6) and its canonical kinase S6K (p70RSK) was achieved. Similarly, partial inhibition of the phosphorylation of eIF4E was observed (FIG. 27A). When used at concentrations relevant for MEK inhibition (e.g., at 25 nM to 100 nM), little or no effect on phosphorylation of S6, eIF4E and 4EBP1 was detectable (data not shown).

SW620 cells are less sensitive to inhibition by PP242 than are PC3 cells. At 2.5 μM PP242, phosphorylation of S6K, S6 and 4EBP1 was substantially inhibited in PC3 cells (FIG. 27B). The inhibitor is less potent in SW620 cells, such that some phosphorylation of 4EBP was observed even at 10 μM (FIG. 27B). From the dose response shown in this figure, it is nonetheless clear that significant inhibition of phosphorylation could be achieved with 2.5 μM PP242.

As is apparent in FIG. 27, treatment of SW620 cells with the MEK inhibitor and with the mTOR inhibitor have distinctly different impacts on the phosphorylation state of important components of the translational machinery, most notably 4EBP1. A corresponding difference on the translation efficiencies of mRNAs that are strongly dependent on the levels of free eIF4E was confirmed by comparing the effects of 250 nM GSK212 and 2.5 μM PP242 in SW620 cells (FIG. 26C). First, most genes shown to be sensitive to 2.5 μM PP242 in PC3 cells (data points in red) are also sensitive in SW620 cells. Second, with only three exceptions, treatment with the MEK inhibitor has little or no effect on the translational efficiencies of these genes. This further demonstrates the ability of translational efficiency measurements to distinguish between drugs and drug mechanisms of action.

Characteristic Transcriptional Gene Signature of MEK Inhibitor GSK212 can be Observed in Translational Rates as Distinct from Translational Efficiencies.

A signature for MEK inhibition in cells sensitive to these agents as determined by microarray analysis has been described previously (Pratilas et al., Proc. Nat'l Acad. Sci. U.S.A. 105: 4519, 2009). This signature was compared with signatures derived from RNA-seq and transcriptional profiling of GSK212 on SW620 cells, as provided in Table 8. There is general agreement between the published signature and the signatures observed both in transcription (RNA) and in translational rates (RPF). The strong concordance between signatures from transcription and translational rate in this setting corresponds to the MEK signature that was originally identified and is associated with robust transcriptional changes which, for the most part, are reflected in changes in translational rate.

TABLE 8
Transcriptional, translational rate, and translational efficiency signatures of
MEK inhibitor on SW620 cells

| PNAS ID/ | | | | PNAS | Profile | | |
|---|---|---|---|---|---|---|---|
| HGNC ID | SEQ ID NO | ENSEMBL ID | Description | rna | rna | rpf | TE |
| ALF/ | 235 | ENSG00000242441 | general transcription factor Iia, | 2.7 | NA | NA | NA |
| GTF2A1L | | | 1-like | | | | |
| SEMA6A/ | 236 | ENSG00000092421 | semaphorin 6A | 2.1 | 2.3 | 0.7 | −1.5 |
| SEMA6A | | | | | | | |
| HYDIN/ | 237 | ENSG00000157423 | hydrocephalus inducing | 2.1 | mInf | Inf | Inf |
| HYDIN | | | | | | | |
| KIR3DL2/ | 238 | ENSG00000240403 | killer cell immunoglobulin-like | 1.7 | NA | NA | NA |
| KIR3DL2 | | | receptor, three domains, long | | | | |
| | | | cytoplasmic tail, 2 | | | | |
| BYSL/ | 239 | ENSG00000112578 | bystin-like | −1.2 | −0.8 | −0.6 | 0.2 |
| BYSL | | | | | | | |
| ELOVL6/ | 240 | ENSG00000170522 | ELOVL family member 6, | −1.5 | −1.4 | −0.8 | 0.6 |
| ELOVL6 | | | elongation of long chain fatty | | | | |
| | | | acids-like 6 | | | | |
| SLC1A5/ | 241 | ENSG00000105281 | solute carrier family 1 (neutral | −1.2 | −0.1 | −0.5 | −0.3 |
| SLC1A5 | | | amino acid transporter), member | | | | |
| | | | 5 | | | | |
| CHSY1/ | 242 | ENSG00000131873 | carbohydrate (chondroitin) | −1.3 | 0.0 | −0.7 | −0.7 |
| CHSY1 | | | synthase 1 | | | | |
| IL8/ | 243 | ENSG00000169429 | interleukin 8 | −2.5 | −5.6 | −3.2 | 2.4 |
| IL8 | | | | | | | |
| FOS/ | 244 | ENSG00000170345 | v-fos FBJ murine osteosarcoma | −3.4 | −2.9 | −2.7 | 0.2 |
| FOS | | | viral oncogene homolog | | | | |
| B4GALT6/ | 245 | ENSG00000118276 | UDP-Gal:betaGlcNAc beta 1,4- | −1.7 | 0.0 | −0.1 | 0.0 |
| B4GALT6 | | | galactosyltransferase, | | | | |
| | | | polypeptide 6 | | | | |
| CCND1/ | 246 | ENSG00000110092 | cyclin D1 (PRAD1: parathyroid | −2.2 | −1.8 | −2.0 | −0.3 |
| CCND1 | | | adenomatosis 1) | | | | |
| ETV5/ | 247 | ENSG00000244405 | ets variant gene 5 (ets-related | −1.7 | −2.6 | −5.6 | −3.0 |
| ETV5 | | | molecule) | | | | |
| ETV4/ | 248 | ENSG00000175832 | ets variant gene 4 (E1A enhancer | −2.2 | −2.2 | −2.8 | −0.6 |
| ETV4 | | | binding protein, E1AF) | | | | |
| SLC4A7/ | 249 | ENSG00000033867 | solute carrier family 4, sodium | −1.6 | −0.2 | −1.1 | −0.9 |
| SLC4A7 | | | bicarbonate cotransporter, | | | | |
| | | | member 7 | | | | |
| ETV1/ | 250 | ENSG00000006468 | ets variant gene 1 | −2.6 | −3.5 | mInf | mInf |
| ETV1 | | | | | | | |
| MAFF/ | 251 | ENSG00000185022 | v-maf musculoaponeurotic | −2.7 | −1.7 | −1.1 | 0.6 |
| MAFF | | | fibrosarcoma oncogene homolog | | | | |
| | | | F (avian) | | | | |
| IER3/ | 252 | ENSG00000137331 | immediate early response 3 | −3.3 | −2.2 | −2.3 | −0.1 |
| IER3 | | | | | | | |
| LIF/ | 253 | ENSG00000128342 | leukemia inhibitory factor | −3.2 | −1.7 | −0.9 | 0.9 |
| LIF | | | (cholinergic differentiation | | | | |
| | | | factor) | | | | |
| SPRY4/ | 254 | ENSG00000187678 | sprouty homolog 4 (Drosophila) | −2.6 | −5.2 | −7.1 | −2.0 |
| SPRY4 | | | | | | | |
| DUSP4/ | 255 | ENSG00000120875 | dual specificity phosphatase 4 | −2.2 | −2.0 | −2.4 | −0.4 |
| DUSP4 | | | | | | | |
| LNK/ | 256 | ENSG00000111252 | SH2B adaptor protein 3 | −2.0 | 0.2 | −0.7 | −0.9 |
| SH2B3 | | | | | | | |
| GPR3/ | 257 | ENSG00000181773 | G protein-coupled receptor 3 | −2.1 | mInf | −2.8 | Inf |
| GPR3 | | | | | | | |
| TNC/ | 258 | ENSG00000041982 | tenascin C (hexabrachion) | −2.5 | 1.1 | −0.5 | −1.6 |
| TNC | | | | | | | |
| POLR3G/ | 259 | ENSG00000113356 | polymerase (RNA) III (DNA | 1.0 | −0.7 | −0.8 | −0.1 |
| POLR3G | | | directed) polypeptide G (32kD) | | | | |
| WDR3/ | 260 | ENSG00000065183 | WD repeat domain 3 | −1.1 | 0.0 | −1.0 | −1.0 |
| WDR3 | | | | | | | |
| BXDC2/ | 261 | ENSG00000113460 | brix domain containing 2 | −1.2 | −1.1 | −0.8 | 0.2 |
| BRIX1 | | | | | | | |
| CD3EAP/ | 262 | ENSG00000117877 | CD3E antigen, epsilon | −1.4 | −0.6 | −1.1 | −0.5 |
| CD3EAP | | | polypeptide associated protein | | | | |
| EGR1/ | 263 | ENSG00000120738 | early growth response 1 | −2.1 | −1.0 | −4.1 | −3.1 |
| EGR1 | | | | | | | |
| PHLDA2/ | 264 | ENSG00000181649 | pleckstrin homology-like | −2.2 | −2.1 | −1.1 | 1.1 |
| PHLDA2 | | | domain, family A, member 2 | | | | |
| ARID5A/ | 265 | ENSG00000196843 | AT rich interactive domain 5A | −1.7 | −0.5 | −0.9 | −0.4 |
| ARID5A | | | (MRF1-like) | | | | |
| DUSP6/ | 266 | ENSG00000139318 | dual specificity phosphatase 6 | −2.6 | −6.3 | −9.3 | −3.1 |
| DUSP6 | | | | | | | |
| SPRY2/ | 267 | ENSG00000136158 | sprouty homolog 2 (Drosophila) | −4.0 | −1.7 | −1.3 | 0.4 |
| SPRY2 | | | | | | | |
| DDX21/ | 268 | ENSG00000165732 | DEAD (Asp-Glu-Ala-Asp) (SEQ | −1.1 | −0.3 | −1.0 | −0.6 |
| DDX21 | | | ID NO: 287) box polypeptide 21 | | | | |
| GTPBP4/ | 269 | ENSG00000107937 | GTP binding protein 4 | −1.1 | −0.4 | −0.7 | −0.3 |
| GTPBP4 | | | | | | | |
| PPAT/ | 270 | ENSG00000128059 | phosphoribosyl pyrophosphate | −1.1 | −0.4 | −0.7 | −0.3 |
| PPAT | | | amidotransferase | | | | |
| HSPC111/ | 271 | ENSG00000048162 | hypothetical protein HSPC111 | −1.3 | −1.5 | −0.9 | 0.6 |
| NOP16 | | | | | | | |
| MYC/ | 272 | ENSG00000136997 | v-myc myelocytomatosis viral | −2.4 | −1.3 | −1.6 | −0.3 |
| MYC | | | oncogene homolog (avian) | | | | |
| MAP2K3/ | 273 | ENSG00000034152 | mitogen-activated protein kinase | −1.5 | −1.6 | −0.6 | 1.0 |
| MAP2K3 | | | kinase 3 | | | | |
| GNL3/ | 274 | ENSG00000163938 | guanine nucleotide binding | −1.0 | −0.7 | −0.8 | −0.2 |
| GNL3 | | | protein-like 3 (nucleolar) | | | | |
| RRS1/ | 275 | ENSG00000179041 | RRS1 ribosome biogenesis | −1.8 | −0.9 | −0.8 | 0.1 |
| RRS1 | | | regulator homolog (S. cerevisiae) | | | | |
| FOSL1/ | 276 | ENSG00000175592 | FOS-like antigen 1 | −4.2 | −3.6 | −4.1 | −0.5 |
| FOSL1 | | | | | | | |
| FLJ10534/ | 277 | ENSG00000167721 | TSR1 20S rRNA accumulation | −1.1 | −0.3 | −0.8 | −0.6 |
| TSR1 | | | homolog (S. cerevisiae) | | | | |
| SPRED2/ | 278 | ENSG00000198369 | sprouty-related, EVH1 domain | −1.0 | −2.1 | −3.5 | −1.3 |
| SPRED2 | | | containing 2 | | | | |
| HMGA2/ | 279 | ENSG00000149948 | high mobility group AT-hook 2 | −1.6 | −2.9 | −1.7 | 1.2 |
| HMGA2 | | | | | | | |
| PLK3/ | 280 | ENSG00000173846 | polo-like kinase 3 (Drosophila) | −2.3 | −3.1 | −2.4 | 0.7 |
| PLK3 | | | | | | | |
| YRDC/ | 281 | ENSG00000196449 | yrdC domain containing (E. coli) | −1.2 | −0.7 | −0.6 | 0.1 |
| YRDC | | | | | | | |
| POLR1C/ | 282 | ENSG00000171453 | polymerase (RNA) I polypeptide | −1.0 | −0.8 | −0.6 | 0.2 |
| POLR1C | | | C, 30kDa | | | | |
| PPAN/ | 283 | ENSG00000130810 | peter pan homolog (Drosophila) | −1.2 | −0.4 | −1.1 | −0.7 |
| PPAN | | | | | | | |
| PYCRL/ | 284 | ENSG00000104524 | pyrroline-5-carboxylate | −2.6 | −0.2 | 0.0 | 0.1 |
| PYCRL | | | reductase-like | | | | |
| GEMIN4/ | 285 | ENSG00000179409 | gem (nuclear organelle) | −1.2 | −0.1 | −0.6 | −0.5 |
| GEMIN4 | | | associated protein 4 | | | | |
| TNFRSF12A/ | 286 | ENSG00000006327 | tumor necrosis factor receptor | −1.5 | −2.2 | −2.0 | 0.2 |
| TNFRSF12A | | | superfamily, member 12A | | | | |

For Table 8: The transcriptional signature of V600E-BRAF tumor cells treated with MEK inhibitor PD0325901 is compared with the transcriptional, translational, and translational efficiency signatures of SW620 cells treated with the MEK inhibitor GSK212. The depicted gene set and data for V600E-BRAF tumor cells are adapted from Table S2 of V600E-BRAF is associated with disabled feedback inhibition of RAF-MEK signaling and elevated transcriptional output of the pathway (Pratilas et al., 2009). Any gene where the value for the GSK212 treated sample is 0 is shown as “mInf” (log₂(0/x) = −infinity) for the log₂ fold-change value. Any gene where the value for the DMSO sample is 0 is shown as “Inf” (log₂(x/0) = infinity) for the log₂ fold-change value. All values are log₂ MEKi/DMSO. Any gene where data is unavailable in the ribosomal profiling experiment is shown as “NA.”

Unique Insights into MEK Inhibition are Nonetheless Apparent in Translational Efficiencies.

In contrast to solely translation rate, examination of the translational efficiencies of the mRNAs that make up the MEK signature indicates a set of gene products that may have unique importance. Protein synthesis from some mRNAs, such as those from BYSL, DUSP4 and POLR3G, was almost exclusively transcriptionally mediated and accordingly had translational efficiency changes near zero. In contrast, mRNAs from genes like ETV5 and SPRY4, which are transcriptionally down-regulated, had the production of their corresponding proteins further inhibited at the translational level leading to profound control. Conversely, production of the mRNA from the IL8, PHLDA2 and MAP2K3 genes are examples where synthesis is less inhibited (despite transcriptional data) due to an offsetting increase in translational efficiency, such as a counter-regulation. In addition to genes involved in the MEK signature, there were a number of other genes in SW620 cells that had changes in translational efficiency associated with MEK inhibition (data not shown). In any case, such genes having translational efficiency or a combination of translational efficiency and transcription control are of interest as therapeutic targets or for use in examining the action of different therapeutic agents (e.g., such as mimic action).

### Example 8

### Translational Profiling of a Fibrotic Disease Cell Model

TGFβ-mediated transformation of fibroblasts is well-established as an essential step in fibroplasia, a key component of many fibrotic disorders (Blobe et al., N. Engl. J. Med. 342:1350, 2000; Border and Noble, N. Engl. J. Med. 331:1286, 1994). As described in this Example, analysis of changes in translational efficiencies reveals disease-associated cellular changes accompanying this transformation. For example, co-administration of TGF-β with an inhibitor of a PI3K/Akt/mTOR pathway enzyme (“PAMi”) reverses or prevents the changes observed in a fibrotic disorder-related pathway (i.e., normalizes the translational efficiencies of the genes) and inhibits increased production of fibrotic disorder biomarker proteins, type 1 procollagen and α-actin (which are both hallmarks of TGF-β-mediated fibroblast transformation to myofibroblasts). Although these biomarkers are only affected at the transcriptional level and not the translational level, they nonetheless provide a means to monitor the pathogenic state of the cell that is mediated by other fibrosis-related genes that are affected at the translational level.

Cell Culture.

Normal human lung fibroblasts (Lonza #CC-2512) were cultured in DMEM+10% FBS supplemented with Penicillin, Streptomycin and Glutamax (Invitrogen) at 37° C. in a humidified incubator with 5% CO₂. Cell passage numbers 2 through 5 were used for all experiments.

Fibroblast Transformation and Treatment.

On Day 0, fibroblasts were seeded and cultured under normal conditions overnight. On Day 1, media was removed, cells were washed with PBS and then incubated for 48 hrs in serum free media (DMEM supplemented with penicillin, streptomycin, and glutamax). On Day 3, media was removed and cells were cultured for 24 hrs with fresh serum free media ±PAMi and ±10 ng/ml TGF-β. After this 24 hour incubation, about 6×10⁶ cells/10 cm plate and about 1×10⁶ cells/well of a 6-well plate were used for ribosomal profiling and western blot analysis, respectively.

Ribosomal Profiling.

Cells were washed with cold PBS supplemented with cycloheximide and lysed with 1× mammalian lysis buffer for 10 min on ice. Lysates were clarified by centrifugation for 10 min at 14,000 rpm and supernatants were collected. Cell lysates were processed to generate the ribosomal protected fragments and total mRNA according to the instructions included with the ARTseq Ribosome Profiling Kit. Sequencing of total RNA (RNA) and of ribosome-protected fragments of RNA (RPF) was carried out with standard Illumina rna seq methodology.

Bioinformatics Analysis.

RNA-seq reads were processed with tools from the FASTX-Toolkit (fastq_quality_trimmer, fastx_clipper and fastx_trimmer). Unprocessed and processed reads were evaluated for a variety of quality measures using FastQC. Processed reads were mapped to the human genome using Tophat. Gene-by-gene assessment of the number of fragments strictly and uniquely mapping to the coding region of each gene was conducted using HTSeq-count, a component of the HTSeq package. Differential analyses of the transforming effect of TGF-β on fibroblasts and effect of PAMi treatment on this transformation were carried out with the software packages DESeq for transcription (RNA counts) and translational rate (RPF counts) and BABEL for translational efficiency based upon ribosomal occupancy as a function of RNA level (RNA and RPF counts). Genes with low counts in either RPF or RNA were excluded from differential analyses. Pathway and network analyses of differential data was conducted using Ingenuity Pathway Analysis (IPA).

Western Blot Analysis.

Cells were washed with PBS and lysed in 1× cell lysis buffer (Cell Signaling) for 15 min at 4° C. Lysates were sonicated briefly and clarified by centrifugation for 15 min at 14,000 rpm and supernatants were collected. Protein concentration in the soluble fraction was determined by BCA protein assay (Thermo Scientific). Samples of protein (20 μg) were resolved on 4-20% Bis-Tris gradient gel (Invitrogen) and transferred to nitrocellulose membrane. The resulting blots were blocked for 1 hr at room temperature with Odyssey blocking solution (LI-COR) and then incubated with primary antibodies at 4° C. overnight. The following day, the blots were washed 3 times, 10 min each in TBST, and incubated with goat anti-rabbit fluorescent conjugated secondary antibody (IRDye 800 CW at 1:20,000; LI-COR) for 1 hour at room temperature. The blots were then washed and scanned, specific proteins were detected by using the LI-COR Odyssey infrared imager. The following antibodies were used at 1:1000 dilution from Sigma (α-actin #A2547) and Cell Signaling: anti-phospho-4EBP(Ser65), anti-phospho-rpS6(Ser235/236)(#4858), anti-phospho-ERK1/2(Thr202/Tyr204)(#4370), anti-phospho-p70S6K(Thr421/Ser424)(#9204), anti-phospho-pAKT(Ser473), anti-phospho-MNK(Thr197/202), anti-α-actin (#4970).

Procollagen Type 1 Analysis.

Culture Media was collected, centrifuged to pellet cellular debris, and stored at −80° C. Procollagen Type 1 C-Peptide (PIPC) was quantified using the (PIP) EIA kit (Clontech Cat# MK101) according to manufacturer's instructions.

### PI3K/Akt/mTORi Co-Administration Prevents Transformation of Fibroblasts to Myofibroblasts by TGFβ.

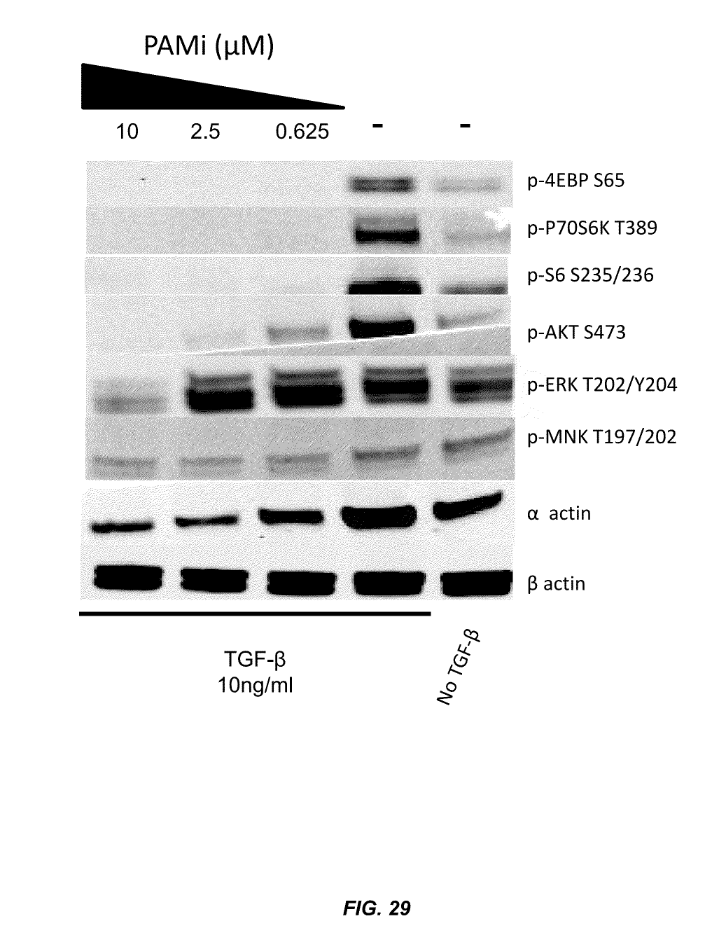

Transformation of fibroblasts to myofibroblasts by treatment with TGF-β for 24 hours was accompanied by an approximately 7-fold increase in procollagen production, while treatment with a PAMi was able to block this increase (EC₅₀ of about 0.2 μM) (FIG. 28). Expression of TGF-β induced myofibroblast differentiation marker, smooth muscle actin (α-SMA), was also analyzed by Western blot analysis (FIG. 29). After 24 hours of TGF-β stimulation, increased α-SMA protein levels were detected, while the level of β-actin did not change. As with procollagen, co-incubation of the cells with a PAMi maintained the α-SMA protein at pretreatment levels. Ribosomal profiling showed that the effect of the PAMi on both procollagen and α-SMA were a consequence of preventing fibroblast transformation and transcriptional regulation (instead of a decrease in translation efficiency of mRNA to protein). Specifically, the translational efficiencies of the procollagen and α-SMA were essentially independent of TGF-β and PAMi treatment.

TGF-β-dependent activation of the PI3K/Akt/mTOR and ERK pathways were also examined by Western blot analysis (FIG. 29). Western analysis also indicates that TGF-β stimulated phosphorylation of AKT, 4EBP, S6K, S6 in the mTOR pathway and modestly increased the phosphorylation of ERK. Co-incubation of cells with the PAMi abolished TGF-β-dependent increases in phosphorylation of AKT, 4EBP, S6K, and S6 at 0.625 μM, as well as decreasing the α-SMA protein to pretreatment levels.

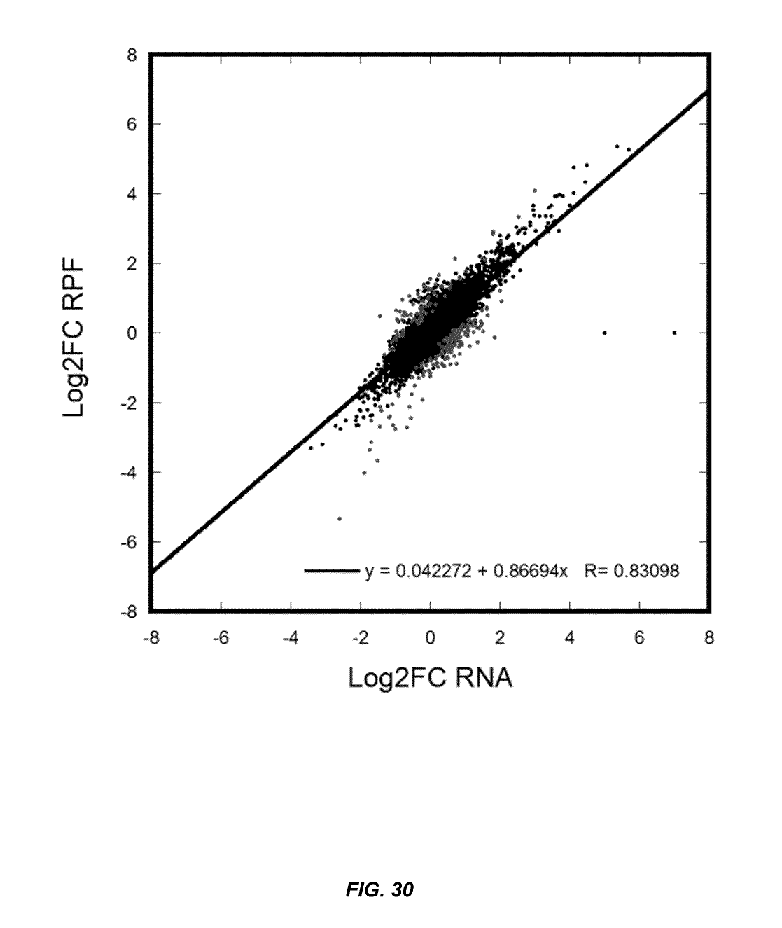

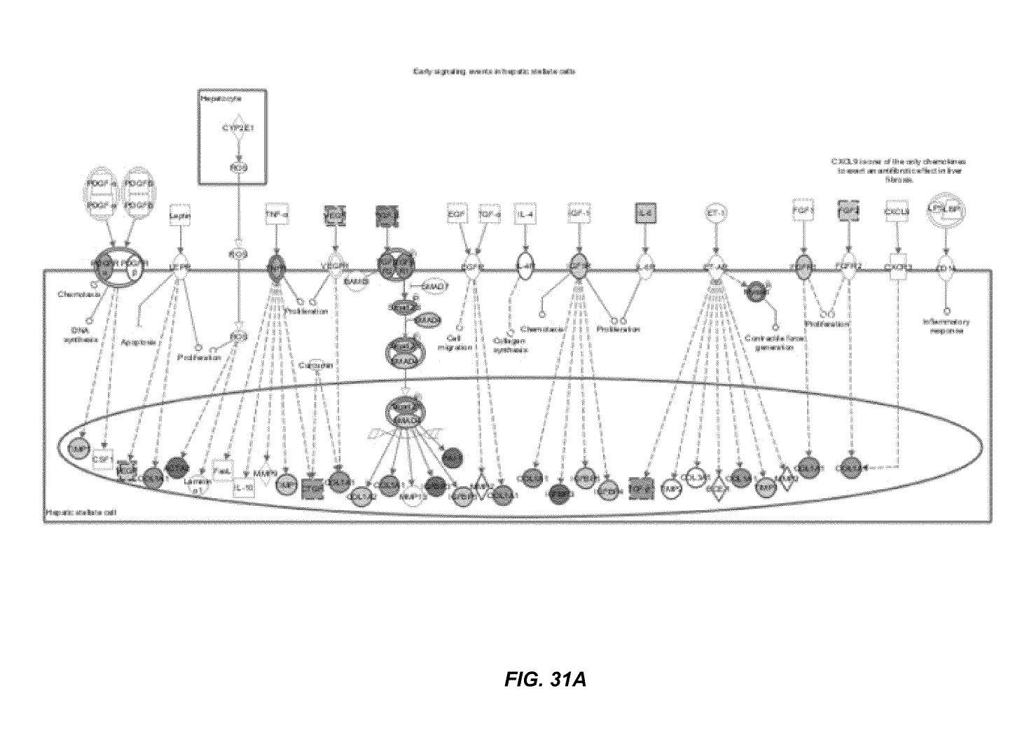

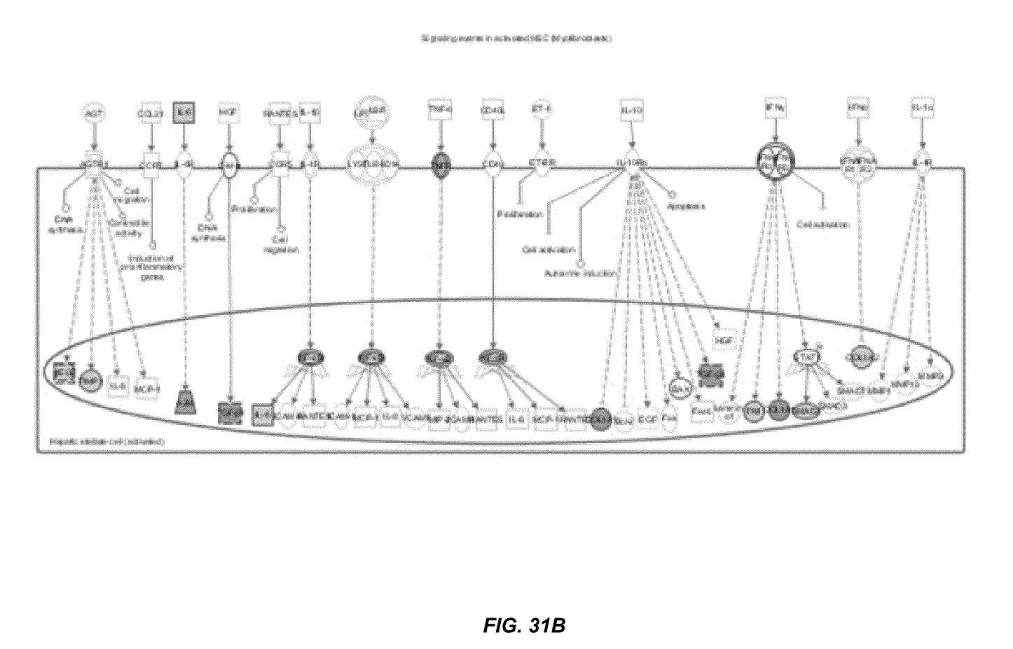

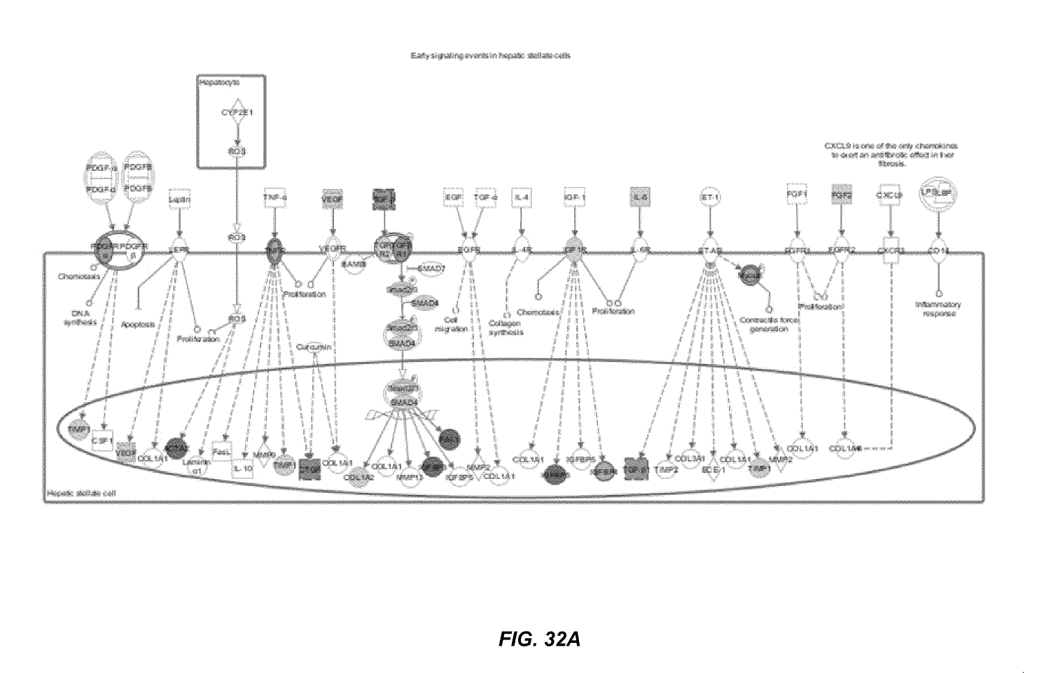

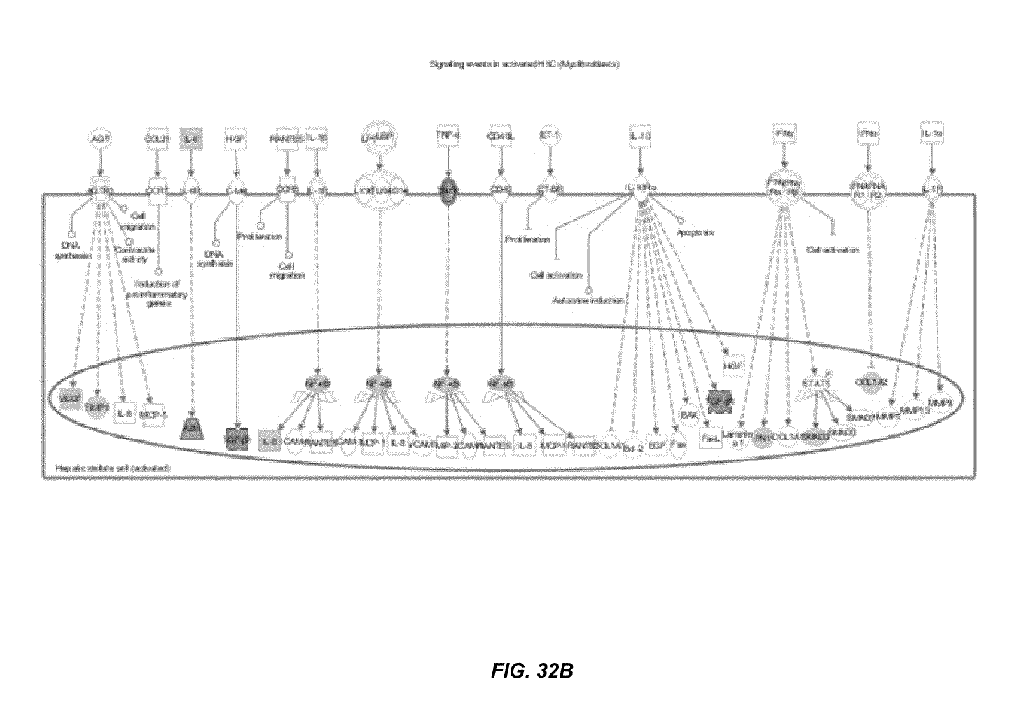

Ribosomal profiling was used to measure changes in transcription and translation on a genome-wide basis accompanying TGF-β-dependent transformation of fibroblasts to myofibroblasts. This system is known to be driven in large part by transcriptional activation, and changes in translational rate and RNA levels on a genome-wide level were highly correlated (see FIG. 30). In contrast, changes in translational efficiency were relatively independent of transcriptional and translational rate changes. Thus, in this case, measurements of translational efficiency provide a unique window into cellular biology of fibrotic disorders. Correspondingly, the outcome of pathway analysis based on gene identification via changes in translational efficiency upon TGF-β treatment is quite distinct from analyses based on transcription or translational rate. These three gene signatures were analyzed for pathway and network connections using Ingenuity Pathway Analysis (IPA). Some characteristics of these gene lists, including the identity of the pathway with the highest statistical association for each signature, are listed in Table 9 (while these are the most significant, it should be noted that significant association of these gene lists with other pathways were observed). Most notably, genes showing changes in RNA levels and translational rates were most strongly associated with Hepatic Fibrosis/Hepatic Stellate Cell Activation (see FIGS. 31 and 32). This action of TGF-β in fibroblasts recapitulates much of the behavior observed in liver fibrosis.

TABLE 9
Properties of Fibrotic Disorder
Gene Signatures from IPA Analysis

| | | RPF | |
|---|---|---|---|
| | RNA | (Translational | Translational |
| | (Transcriptome) | Rate) | Efficiency |
| p-value | 0.1 | 0.05 | 0.05 |
| threshold | | | |
| for | | | |
| differential | | | |
| # genes | 194 | 211 | 238 |
| meeting | | | |
| threshold | | | |
| % of total gene set | 4.20% | 4.50% | 5.10% |
| Most | Hepatic Fibrosis/ | Hepatic Fibrosis/ | Unique |
| significant | Hepatic Stellate | Hepatic Stellate | Fibrosis- |
| pathway/ | Cell Activation | Cell Activation | Associated |
| category | | | Pathway |
| from IPA | | | |
| analysis | | | |

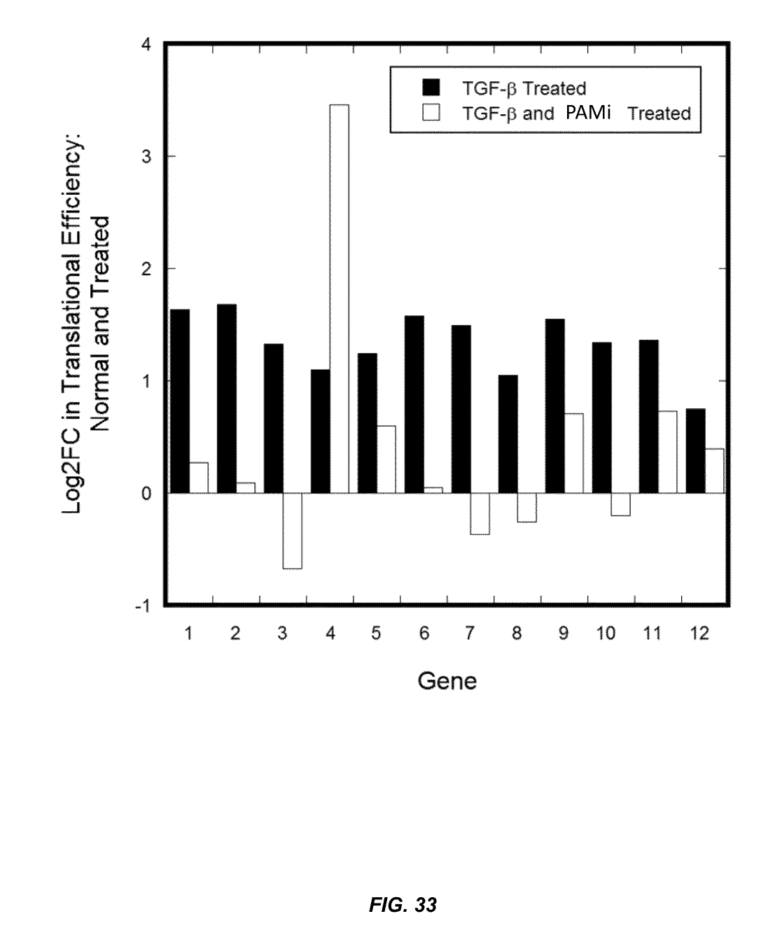

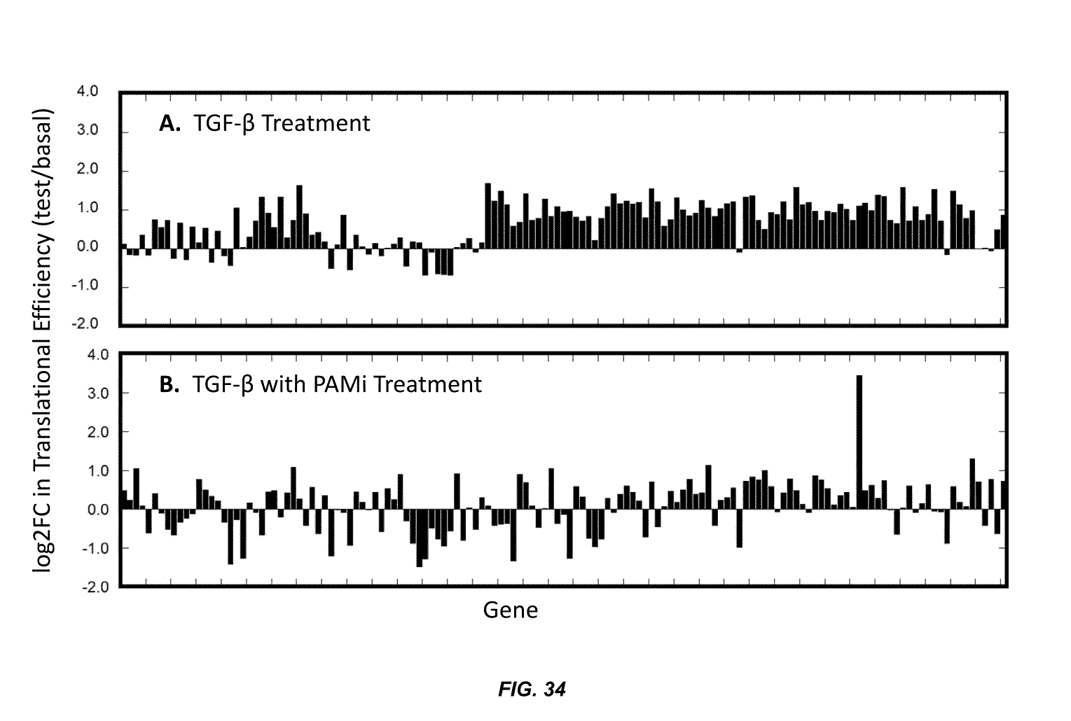

In contrast, the gene signature showing changes in translational efficiency was most strongly associated with regulation of a pathway not previously observed to be associated with fibrotic disorders. All genes identified in this new pathway showed a significant increase in translational efficiency (TE) (FIG. 33), which extends far beyond these few genes. For example, 118 of the 141 genes in the pathway evaluated in this study move in concert, having an increase in translational efficiency (FIG. 34, panel A). The translational efficiencies of the 141 pathway-associated genes in fibroblasts before treatment with TGF-β (which induces a fibrotic-disease type condition) were low (mean value −1.70 log 2 relative to population mean); the impact of TGF-β induced transformation was to increase the translational efficiency of many genes in this signature (mean value of signature upon TGF-β treatment was −1.05). Nonetheless, this was still 2-fold lower than the overall population and indicates this pathway is a bottleneck in cellular transformation. For the subset of 12 genes described previously, 11 of 12 move toward the untransformed, normal state after treatment with PAMi (FIG. 33). The mean increase in translational efficiency by TGF-β in these 12 genes is 1.3 log 2; the presence of PAMi decreases this value to only 0.4 log 2. Similar results are seen for all genes in the pathway (FIG. 34), wherein 104 of 141 genes move toward normal. The mean increase in translational efficiency by TGF-β in these 12 genes was 0.65 log 2; the presence of PAMi decreases this value to only 0.09 log 2. Clearly, the presence of PAMi maintains the translational efficiencies of the genes in fibrotic disorder-associated pathway at their normal state in fibroblasts. Normalization of this pathway by PAMi is due to substantially inhibiting TGF-β induced transformation of fibroblasts to fibrotic myofibroblasts.

Conclusion.

Comparison of translational efficiencies between the normal, healthy state (fibroblasts) and pathogenic state (fibrotic myofibroblasts induced by TGFβ treatment) identified a novel pathway previously not associated with fibrosis, which is a novel insight into a key role of translational efficiency in the pathogenesis of fibrotic disease. Further, a PAMi agent that modulates this fibrotic disorder-associated pathway and prevents TGF-β-mediated fibroblast to myofibroblast transformation confirms the association of this pathway with fibrotic disease and, thus, shows that components and regulators of this pathway are new targets. The methods of the instant disclosure show that new gene signatures having altered translational profiles (e.g., altered translational efficiency) may be identified using such methods. Furthermore, these data show that an agent or therapeutic that normalizes a translational profile may also be identified. Finally, these data show that targets not previously validated for a particular disorder (in this case, fibrosis), can be identified and validated using the methods of this disclosure.

### Example 9

### Translational Profiling of a Neurodevelopmental Disease Model

An exemplary neurodevelopmental disease or disorder is Fragile X syndrome, which is caused by a redundant trinucleotide (CGG) repeat in the 5′ UTR of the fragile X mental retardation 1 gene (FMR1). This causes silencing of the FMR1 gene at the transcriptional level and results in the lack of fragile X mental retardation 1 protein (FMRP) expression. FMRP is a cytoplasmic RNA binding protein that associates with polyribosomes as part of a large ribonucleoprotein complex and acts as a negative regulator of translation. Hence, FMRP is thought to regulate the translation of specific mRNAs that are critical for correct development of neurons and synaptic function. The Fragile X syndrome is directly linked to this lack of FMRP expression or loss of FMRP function (i.e., loss of translational control). Indeed, Fmr1 knockout mice have abnormal dendritic spines, which are thought to be the basis of the disease associated mental retardation (see, e.g., Darnell et al., Cell 146: 247, 2011).

Cell Culture.

SH-SY5Y human neuroblastoma cells were cultured in F12/DMEM media (1:1 ratio) supplemented with penicillin G (100 U/ml), streptomycin (100 μg/ml), and 10% FBS. HEK293 human embryonic fibroblasts were cultured in DMEM media supplemented with penicillin G (100 U/ml), streptomycin (100 μg/ml), and 10% FBS. All cells were cultured in a humidified atmosphere of 5% CO₂ maintained at 37° C.

siRNA Transfection.

SH-SY5Y cells (ATCC, passage 8) and HEK293 (ATCC, passage 12) were reverse transfected with 100 nM siControl (AM4611) or siFRM1 (ID# s5316) for 3 days using Lipofectamine RNAiMax (Invitrogen) according to manufacturer's protocol. All siRNAs were purchased from Invitrogen. About 3×10⁶ cells/10 cm plate and about 5×10⁵ cells/well of a 6-well plate were harvested for ribosome profiling and Western blot analysis following siRNA transfection, respectively.

Western Blot Analysis.

Cells were washed with PBS and lysed in 1× cell lysis buffer (Cell Signaling) for 15 min at 4° C. Lysates were sonicated briefly, clarified by centrifugation for 15 min at 14,000 rpm, and supernatants were then collected. Protein concentration in the soluble fraction was determined by BCA protein assay (Thermo Scientific). A 4-20% Bis-Tris gradient gel (Invitrogen) was used to resolve 20 μg of protein and transferred to nitrocellulose membrane. The resulting membranes were blocked for 1 hr at room temperature with Odyssey blocking solution (LI-COR) and then incubated with primary antibodies at 4° C. overnight. The following day, the blots were washed 3 times, 10 min each in TBST, and incubated with IR-conjugated anti-rabbit IgG and anti-mouse IgG secondary antibody (IRDye 800 CW at 1:20,000; LI-COR) for 1 hour at room temperature. The blots were then washed, scanned, and specific proteins were detected using the LI-COR Odyssey infrared imager. The following antibodies were used at 1:1000 dilution from Cell Signaling: anti-FMRP (#4317), anti-TSC2 (#4308), and anti-β-actin (#4970).

Ribosomal Profiling.

Cells were washed with cold PBS supplemented with cycloheximide and lysed with 1× mammalian lysis buffer for 10 min on ice. Lysates were clarified by centrifugation for 10 min at 14,000 rpm and supernatants were collected. Cell lysates were processed to generate the ribosomal protected fragments and total mRNA according to the instructions included with the ARTseq Ribosome Profiling Kit. Sequencing of total RNA (RNA) and of ribosome-protected fragments of RNA (RPF) was carried out with standard Illumina rna seq methodology.

Bioinformatics Analysis.

RNA-seq reads were processed with tools from the FASTX-Toolkit (fastq_quality_trimmer, fastx_clipper and fastx_trimmer). Unprocessed and processed reads were evaluated for a variety of quality measures using FastQC. Processed reads were mapped to the human genome using Tophat. Gene-by-gene assessment of the number of fragments strictly and uniquely mapping to the coding region of each gene was conducted using HTSeq-count, a component of the HTSeq package. Differential analyses of the knockdown of the FMR1 gene were carried out with the software packages DESeq for transcription (RNA counts) and translational rate (RPF counts) and BABEL for translational efficiency based upon ribosomal occupancy as a function of RNA level (RNA and RPF counts). Genes with low counts in either RPF or RNA were excluded from differential analyses.

### Expression Levels of FMRP and TSC2 Following Transient Knockdown of FMR1.

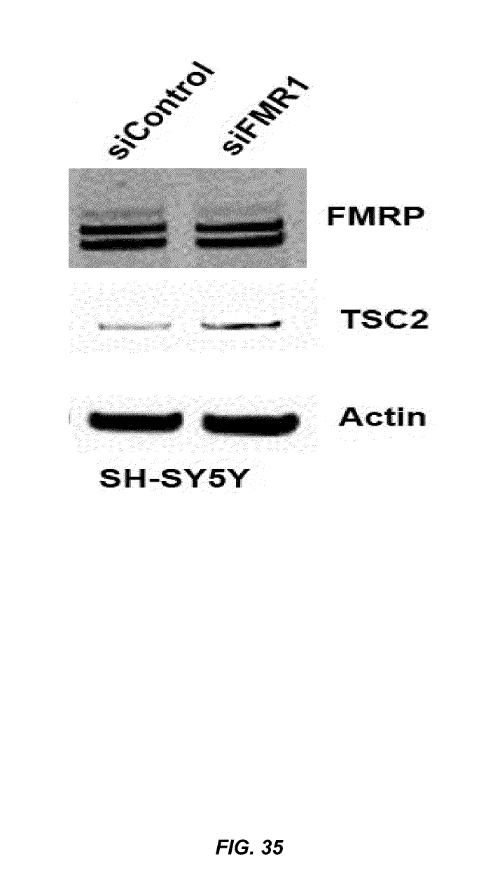

SH-SY5Y cells were transfected with either siControl or siFMR1 at 100 nM for 3 days. Protein levels of FMRP and TSC2 (a known translational target of FMRP) were evaluated by western blot analysis (FIG. 35), and β-actin was used as a loading control. The uppermost band observed in the western blot analysis represents the FMR1 isoform and is sensitive to the siFMR1 knockdown. An approximately 30% knockdown efficiency of FMRP was determined by integrating the band intensities, as well as quantitating by q-PCR analysis (data not shown). The protein expression levels of TSC2 increased after knocking down FMRP, a negative translational regulator.

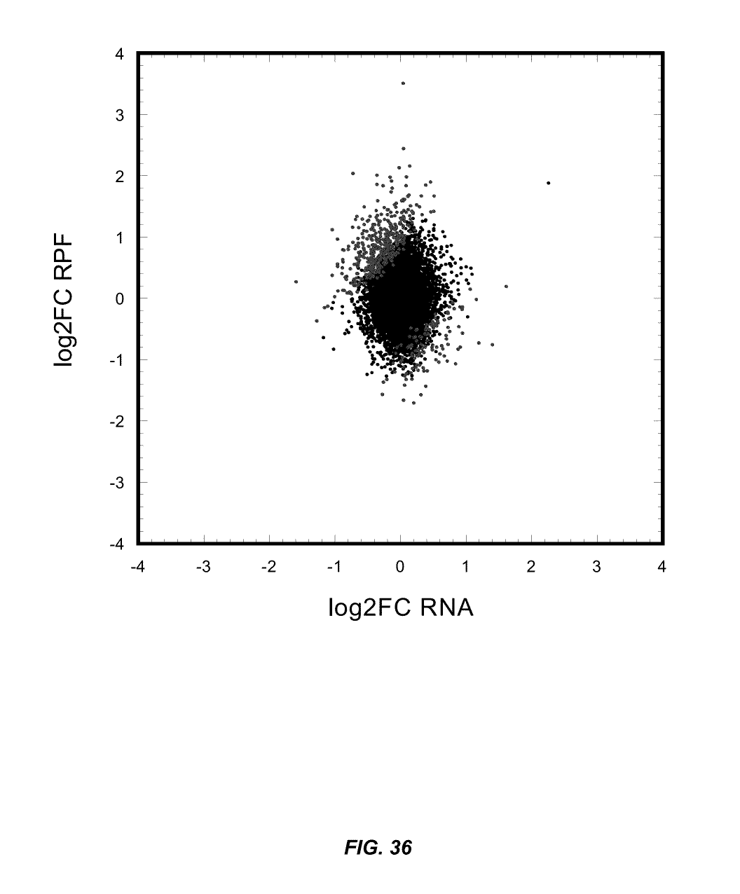

Ribosomal profiling was used to measure changes in transcription and translation on a genome-wide basis after transfecting the cells with either siControl or siFMR1. Analysis of the sequencing results for the FMR1 gene shows that about a 30% reduction was observed, consistent with the western blot and q-PCR analyses. The FMRP specific target, TSC2, showed a corresponding ˜30% increase in the translational rate in the absence of a change in transcriptional levels. On a genome-wide evaluation, knockdown of the FMR1 gene resulted in minimal changes in the transcriptome (see, FIG. 36) with only a log 2 fold change of 2.3 and 1.6 for the top two up-regulated genes (log 2 fold change of −1.6 and −1.2 for the top two down-regulated genes). Changes in the translational rate were identified for a number of genes in the absence of a change in transcriptional levels, corresponding to a change in the translational efficiency. These results indicate that FMRP is responsible for the translational regulation of this specific set of genes.

Known translation targets of FMRP have been reported to include eEF2, eEF1, all three eIF4G isoforms, TSC2 and SYNGAP1. Consistent with these reports, the sequencing data showed that for the knockdown of FMRP, the elongation factors (eEF2 and eEF1) as well as TSC2 and SYNGAP1 had an associated increase in translational rate (increased translation of these targets) by 30-50% in the absence of changes of RNA levels. In contrast, no changes in either RNA levels or translational rates were observed for the three eIF4G isoforms.

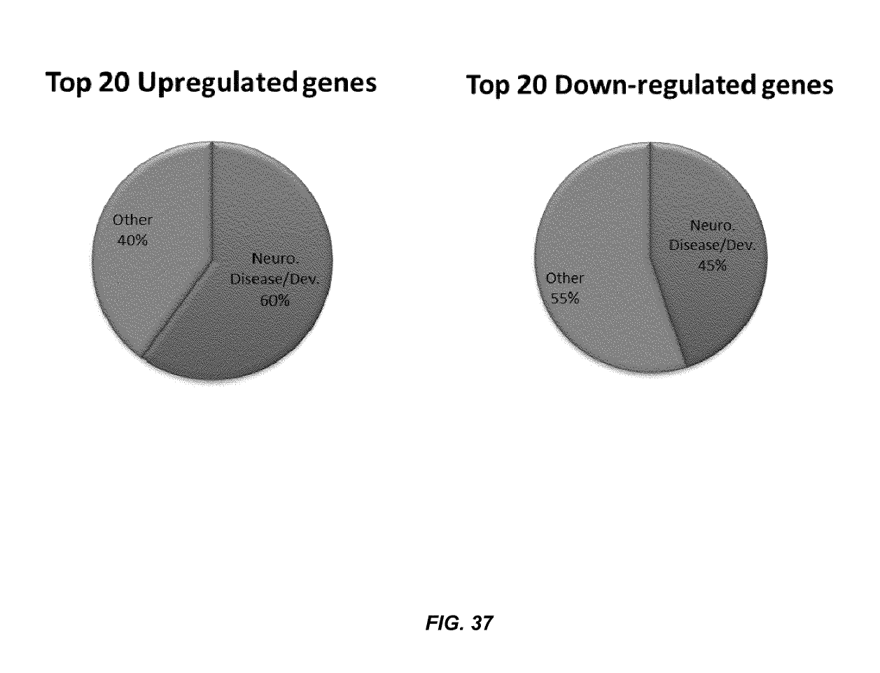

The set of genes identified via changes in translational efficiency or rate upon knockdown of the FMR1 gene is quite distinct from the corresponding set based on transcription. Of particular interest are the top 20 up- or down-regulated genes (log 2 fold increase of 1.9-3.5 (p-value ≦0.001) or decrease of 1.5-2.2 (p-value ≦0.05), respectively) from changes in translational efficiency. Of these 40 genes, only 3 also had significant (p<0.05) movement in mRNA levels. As shown in FIGS. 37, 60 and 45% of these 20 translationally up- and 20 down-regulated genes, respectively, are associated with neurological disease or development. This enrichment for neurological association increased to 70% and 50% for the top 10 up- and down-regulated genes, respectively.

Conclusions.

Fragile X is the most inheritable form of mental retardation. Current concepts of how FMRP regulates the translation of specific mRNAs are still being elucidated. This example shows that ribosome profiling and pathway analysis of genome-wide translational efficiencies after FMRP knockdown translationally regulates genes that are highly associated with neurological disease and development providing a novel insight into the key genes that are translationally regulated. The genes identified represent a new set of validated targets for points of intervention for the treatment of fragile X syndrome.

### Example 10

### Translational Profiling of an Inflammation Cell Model

Macrophages treated with LPS have been shown to stimulate cytokine production as well as activation of both the PI3K and RAS pathways (Weintz et al., Mol. Sys. Biol. 371:1, 2010). In this example, LPS-induced macrophage activation was evaluated by monitoring TNF-α levels along with phosphorylation of components in the PI3K and RAS pathways.

Cell Culture and TNFα Measurements.

RAW264.7 murine macrophages (ATCC) were cultured in DMEM containing 10% FBS supplemented with Penicillin, Streptomycin, Glutamax (Invitrogen) at 37° C. in a humidified incubator with 5% CO2. Cells were treated with inhibitor or DMSO for 2 hrs prior to 1 ng/ml LPS challenge (Sigma) for an additional 1 hr. Media was collected, centrifuged, and supernatants were used for TNF-α ELISA according to manufacturer's instructions (R&D Systems #MTA00B). Approximately 5×10⁶ cells/10 cm dish and 0.5×10⁶ cells/well of a 6-well plate were used for ribosome profiling and Western blot analysis, respectively.

Western Blot Analysis.

Cells were washed with PBS and lysed in 1× cell lysis buffer (Cell Signaling) for 15 min at 4° C. Lysates were sonicated briefly, clarified by centrifugation for 15 min at 14,000 rpm, and supernatants were then collected. Protein concentration in the soluble fraction was determined by BCA protein assay (Thermo Scientific). A 4-20% Bis-Tris gradient gel (Invitrogen) was used to resolve 20 μg of protein and transferred to nitrocellulose membrane. The resulting membranes were blocked for 1 hr at room temperature with Odyssey blocking solution (LI-COR) and then incubated with primary antibodies at 4° C. overnight. The following day, the blots were washed 3 times, 10 min each in TBST, and incubated with IR-conjugated anti-rabbit IgG and anti-mouse IgG secondary antibody (IRDye 800 CW at 1:20,000; LI-COR) for 1 hour at room temperature. The blots were then washed, scanned, and specific proteins were detected using the LI-COR Odyssey infrared imager. The following antibodies were used at 1:1000 dilution from Cell Signaling: anti-phospho-4EBP(Ser65), anti-phospho-rpS6(Ser235/236) (#4858), anti-phospho-ERK1/2(Thr202/Tyr204) (#4370), anti-phospho-p70S6K(Thr421/Ser424) (#9204), anti-phospho-pAKT(Ser473), anti-phospho-eIF4E(Ser209), anti-phospho-RSK(Thr359/Ser363), anti-β-actin (#4970).

Ribosomal Profiling.

Cells were washed with cold PBS supplemented with cycloheximide and lysed with 1× mammalian lysis buffer for 10 min on ice. Lysates were clarified by centrifugation for 10 min at 14,000 rpm and supernatants were collected. Cell lysates were processed to generate the ribosomal protected fragments and total mRNA according to the instructions included with the ARTseq Ribosome Profiling Kit. Sequencing of total RNA (RNA) and of ribosome-protected fragments of RNA (RPF) was carried out with standard Illumina rna seq methodology.

Bioinformatics Analysis.

RNA-seq reads were processed with tools from the FASTX-Toolkit (fastq_quality_trimmer, fastx_clipper and fastx_trimmer). Unprocessed and processed reads were evaluated for a variety of quality measures using FastQC. Processed reads were mapped to the human genome using Tophat. Gene-by-gene assessment of the number of fragments strictly and uniquely mapping to the coding region of each gene was conducted using HTSeq-count, a component of the HTSeq package. Differential analyses of the stimulation of LPS and effect of drug treatment were carried out with the software packages DESeq for transcription (RNA counts) and translational rate (RPF counts) and BABEL for translational efficiency based upon ribosomal occupancy as a function of RNA level (RNA and RPF counts). Genes with low counts in either RPF or RNA were excluded from differential analyses. Pathway and network analyses of differential data was conducted using Ingenuity Pathway Analysis (IPA).

Results.

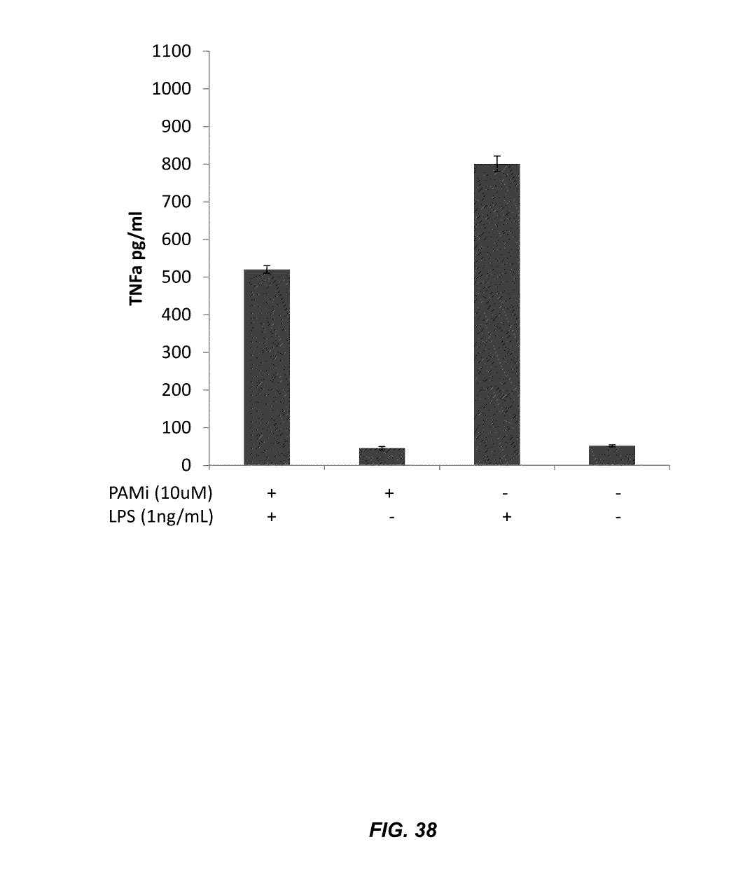

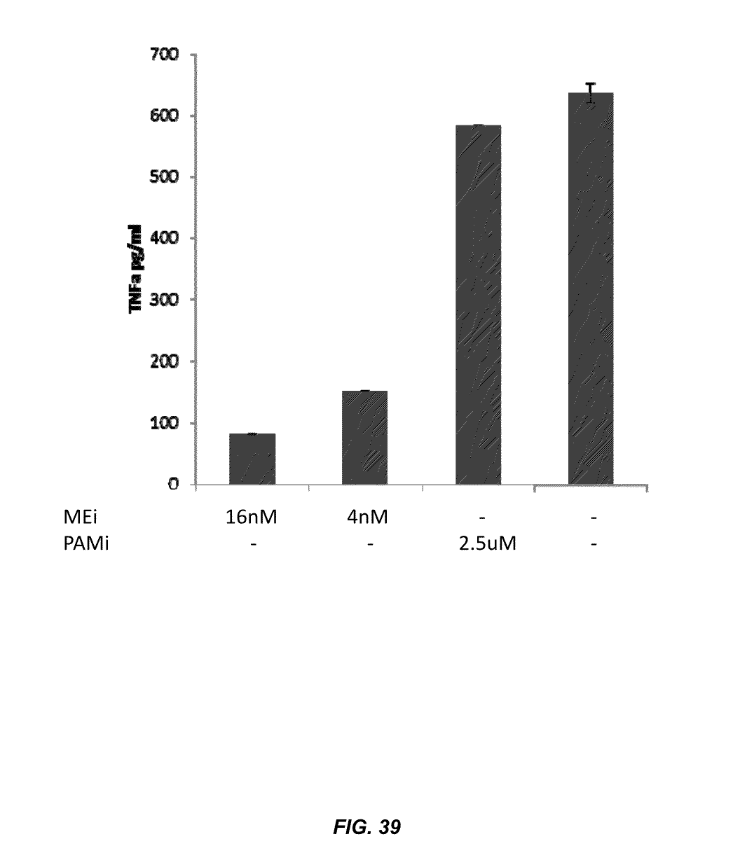

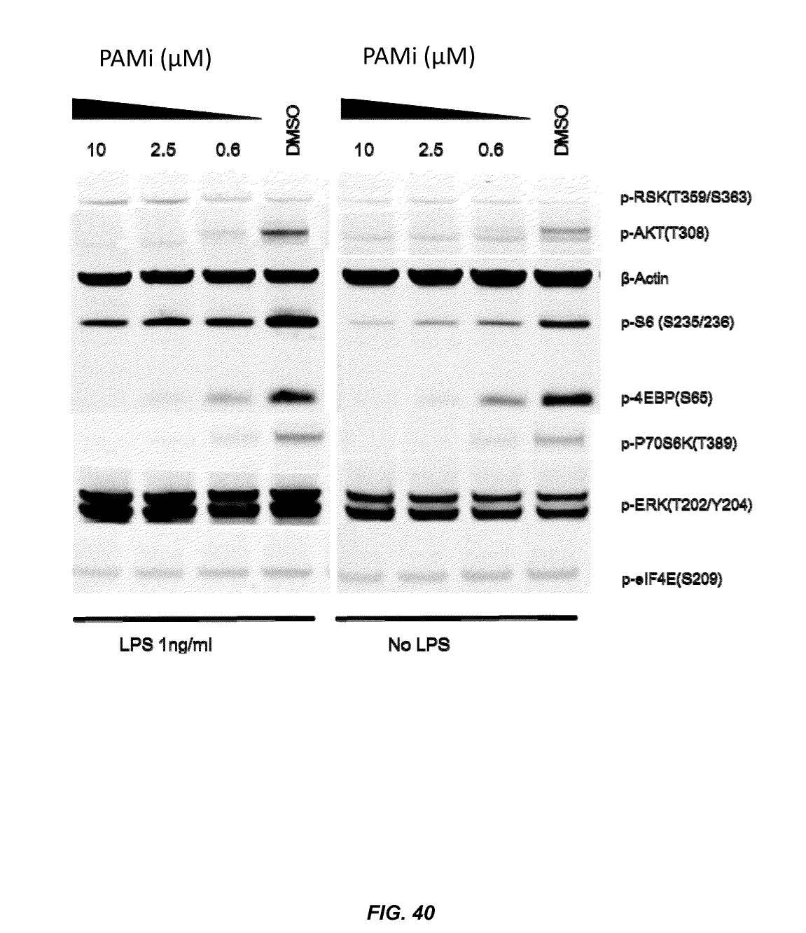

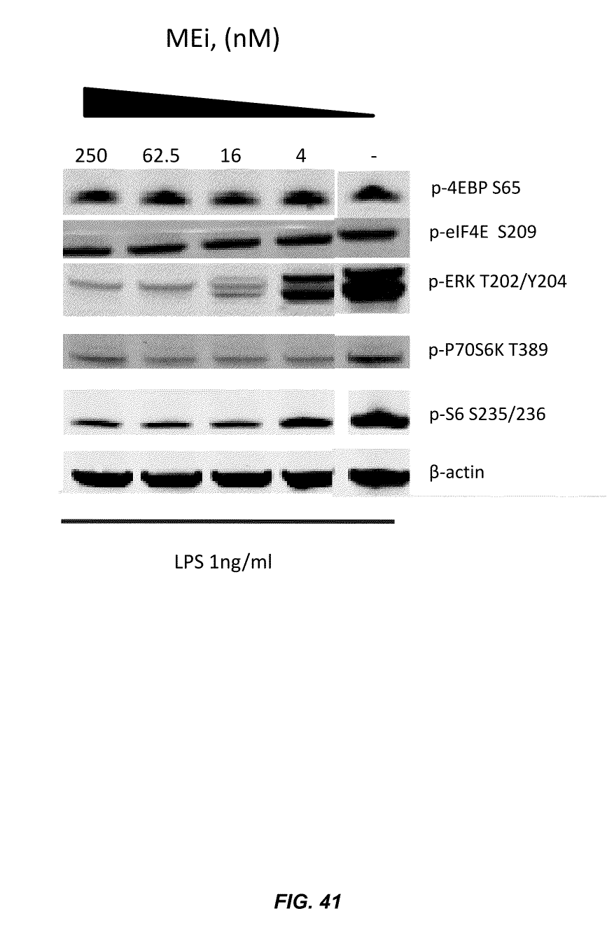

These data show that after 1 hour of 1 ng/mL LPS stimulation, TNF-α levels were seen to rapidly increase (FIG. 38), and increased phosphorylation was observed for RSK and ERK within the RAS pathway, as well as for AKT, S6K (modest) and S6 in the PI3K pathway (see, FIG. 40). No changes in the phosphorylation levels of 4EBP, eIF4E or the housekeeping gene, β-actin, were discernable (FIG. 40). Treatment with an inhibitor of a PI3K/Akt/mTOR pathway enzyme (“PAMi”) or a MEK/ERK pathway enzyme (“MEi”), for 2 hours prior to the LPS stimulation, reduced the levels of TNF-α production (FIGS. 38 and 39). In particular, PAMi substantially inhibited phosphorylation of AKT, 4EBP and S6K at the lowest concentration tested and reduced phosphorylation of S6 and eIF4E, but did not alter the phosphorylation of RSK or ERK at the concentrations used (FIG. 41). The MEi induced a dose dependent inhibition of the phosphorylation of ERK and S6 (with no effects on the phosphorylation of 4EBP, eIF4E, and S6K) (FIG. 41), wherein preincubation with 16 nM MEi essentially prevented LPS stimulated production of TNF-α (FIG. 39).

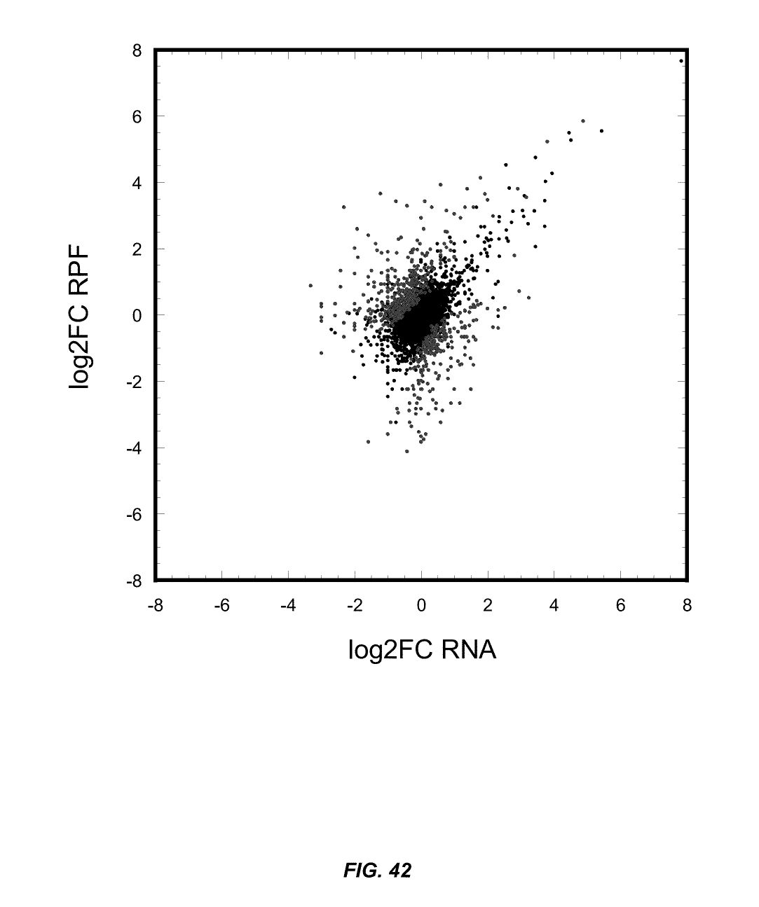

Ribosomal profiling was used to measure changes in transcription and translation on a genome-wide basis after stimulating macrophages with LPS. LPS is known to activate transcription for a number of genes. The majority of transcriptional changes were correlated with a change in translational rate as shown by the data points along the diagonal (see, FIG. 42). Conversely, a significant number of changes in translational rate were independent of transcriptional changes (data points in red along the y-axis where x is zero). The level of TNF-α mRNA increased with LPS stimulation; however, the amount of ribosome protected fragments were in excess to the mRNA increases. These data demonstrate that TNF-α is regulated at the translational level in addition to having transcriptional changes. In addition, a number of genes were seen to be regulated at a translational level in the absence of a change in transcription levels providing a unique window of understanding the mechanism of inflammatory disease.

The gene sets for transcription, translational rate and translational efficiency were analyzed for pathway and network connections using IPA software. The output of the pathway analysis demonstrated that the transcriptome was strongly associated with inflammatory disease (p-value=3.3E-09). The pathway analysis did not highlight pathways for the translational efficiency set of genes that were strongly supported statistically. However, the top 20 genes that were identified as translationally regulated were enriched for association with inflammatory diseases. Specifically, the top 10 translationally up- and down-regulated genes were enriched 70% and 50%, respectively, for association with inflammatory disease (p-value ≦0.05). Only 3 of these 20 translationally regulated genes were statistically significant for changes in RNA levels.

Treatment of macrophages with a PAMi or MEi followed with LPS stimulation showed that drug treatment was able to restore the translational efficiencies back to normal levels for the top 20 regulated genes. Interestingly, PAMi was more effective at renormalizing this gene subset when compared with MEi. Treatment of the cells with these drugs did not correspond with altering the translational efficiency of TNF-α. These results indicate that drug treatment modulates the level of TNF-α by regulating the translational levels of other inflammatory disease related genes.

This example shows that ribosome profiling and pathway analysis of genome-wide translational efficiencies after LPS stimulation translationally regulates genes that are highly associated with inflammatory disease providing a novel insight into the key genes that are translationally regulated.

### Example 11

### Translational Profiling of Primary Cells

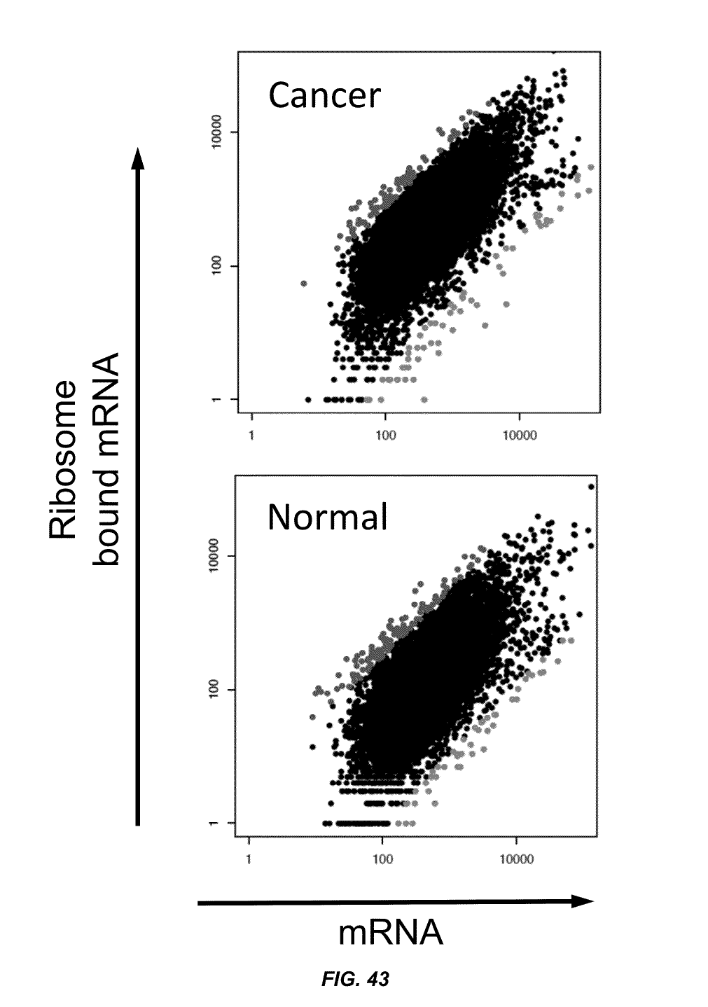

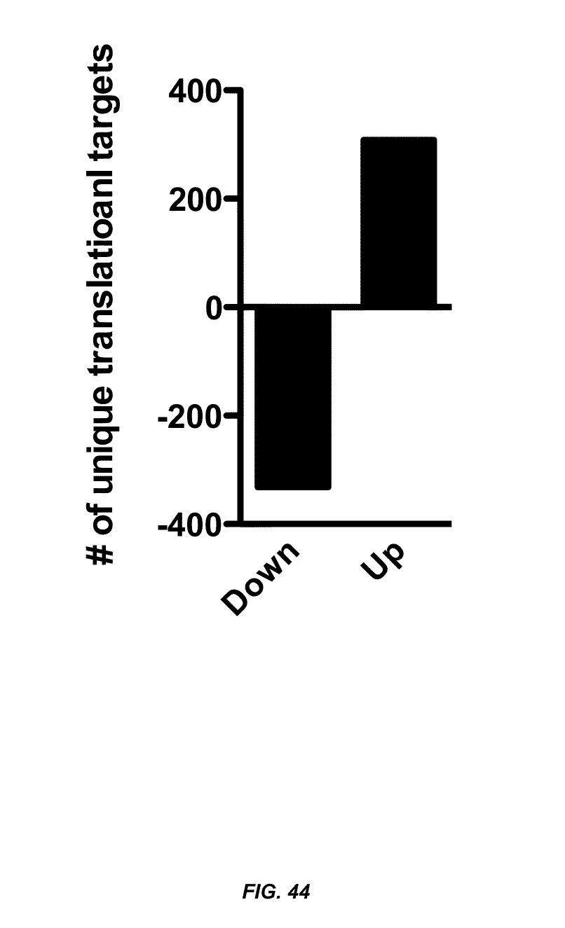

A subject diagnosed with prostate cancer (a Gleason 3+4 tumor) underwent a radical prostatectomy, and the isolated prostate was frozen. Samples removed from frozen pieces of the prostate were reviewed by a pathologist and areas were deemed cancer versus normal. Translational profiles of normal prostate tissue and cancer prostate tissue were generated using ribosomal profiling as described herein. FIG. 43 shows a representative comparison of the change in translational efficiency in normal versus tumor tissue, with each data point representing a single gene. Data points highlighted in red and green have statistically significant changes in translational efficiency versus the population of genes as a whole. For example, the green dots represent genes that have statistically significant lower ribosome occupancy and, therefore, a reduced translational efficiency as compared to the population of genes examined. The differential translational profile between the healthy and cancer tissues shows that there are many genes with significantly greater translational efficiency and significantly reduced translational efficiency (FIG. 44).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method for identifying a candidate therapeutic for treating a disease, the method comprising:
(a) determining a first translational profile for a plurality of genes for a disease sample that has been contacted with a candidate agent;
(b) determining a second translational profile for a plurality of genes for a disease sample that has not been contacted with the agent; and
(c) identifying the agent as a candidate therapeutic for treating the disease when one or more genes are differentially translated in the first translation profile as compared to the second translation profile and when the differential translation results in a biological benefit.

2. A method for identifying a candidate therapeutic for treating a disease, the method comprising:
(a) determining a first translational profile for a plurality of genes from a disease sample that has been contacted with a candidate agent;
(b) determining a second translational profile for a plurality of genes from a disease sample that has been contacted with a known active compound for treating the disease; and
(c) identifying the agent as a candidate therapeutic for use in treating the disease when the first translational profile is comparable to the second translational profile.

3. The method of claim 2, wherein the known active compound is a therapeutic agent for a cancer, an inflammatory disease, an autoimmune disease, a fibrotic disorder, a neurodegenerative disease, a neurodevelopmental disease, a metabolic disease, or a viral infection.

4. The method of claim 2, wherein the translational profiles comprise one or more gene signatures, and wherein the translational profiles of the one or more gene signatures are comparable in the first translational profile and second translational profile.

5. The method of claim 2, wherein the first and second translational profiles are comparable when an amount of protein translated from one or more differentially translated genes in the first and second translational profiles differs by no more than about 25%, 20%, 15%, 10%, 5%, 1% or less.

6. The method of claim 1, wherein the one or more differentially translated genes comprises a plurality of genes.

7. The method of claim 6, wherein the plurality of differentially translated genes comprise one or more gene signatures or are from one or more biological pathways.

8-15. (canceled)

16. The method of claim 1, wherein the disease is a cancer, an inflammatory disease, an autoimmune disease, a fibrotic disorder, a neurodegenerative disease, a neurodevelopmental disease, a metabolic disease, or a viral infection.

17. (canceled)

18. The method of claim 16, wherein the disease is a cancer selected from prostate cancer, breast cancer, bladder cancer, lung cancer, renal cell carcinoma, endometrial cancer, melanoma, ovarian cancer, thyroid cancer, or brain cancer.

19-21. (canceled)

22. The method of claim 1, wherein each translational profile comprises a genome-wide translational profile.

23. The method of claim 22, wherein less than about 20% of the genes in the genome are differentially translated in the first translational profile as compared to the second translational profile.

24. The method of claim 22, wherein less than about 5% of the genes in the genome are differentially translated by at least two-fold in the first translational profile as compared to the second translational profile.

25. (canceled)

26. The method of claim 1, wherein the identified agent inhibits the activity of a downstream effector of an oncogenic signaling pathway, wherein the effector is eIF4E, 4EBP1, p70S6K1/2, or AKT.

27. A method for identifying a candidate therapeutic for treating a disease, the method comprising:
(a) determining three independent translational profiles, each for a plurality of genes from a disease sample, wherein (i) a first translational profile is from a sample not contacted with any compound; (ii) a second translational profile is from a sample that has been contacted with a known active compound for treating the disease; and (iii) a third translational profile is from a sample that has been contacted with a candidate agent;
(b) identifying one or more genes as differentially translated in the first translational profile as compared to the second translational profile; and
(c) identifying the agent as a candidate therapeutic for use in treating the disease when the one or more differentially translated genes from step (b) are in the third translational profile and when the translational profile of the one or more genes in the third translational profile is closer to the translational profile of the one or more genes in the second translational profile than to the translational profile of the one or more genes in the first translational profile.

28. The method of claim 27, wherein the one or more differentially translated genes from the third translational profile have a translational profile closer to the translational profile of the one or more genes in the second translational profile when the amount of protein translated from the one or more differentially translated genes in the third and second translational profiles differs by no more than about 50%, 45%, 40%, 35%, 30%, 25%, 20%, 15%, 10%, 5%, 1% or less.

29. A method for identifying a candidate therapeutic for treating a disease, the method comprising:
(a) determining three independent translational profiles, each for a plurality of genes from a disease sample, wherein (i) a first translational profile is from a sample not contacted with any compound; (ii) a second translational profile is from a sample that has been contacted with a known active compound for treating the disease; and (iii) a third translational profile is from a sample that has been contacted with a candidate agent;
(b) determining a first differential translational profile comprising one or more genes differentially translated in the first translational profile as compared to the second translational profile, and determining a second differential translational profile comprising one or more genes differentially translated in the first translational profile as compared to the third translational profile; and
(c) identifying the agent as a candidate therapeutic for use in treating the disease when the first differential translational profile is comparable to the second differential translational profile.

30. The method of claim 29, wherein the first and second differential translational profiles are comparable when the amount of protein translated from the one or more differentially translated genes in the first and second differential translational profiles differs by no more than about 50%, 45%, 40%, 35%, 30%, 25%, 20%, 15%, 10%, 5%, 1% or less.

31. The method of claim 27, wherein the known active compound is a therapeutic agent for a cancer, an inflammatory disease, an autoimmune disease, a fibrotic disorder, a neurodegenerative disease, a neurodevelopmental disease, a metabolic disease, or a viral infection.

32. The method of claim 27, wherein the one or more differentially translated genes comprises a plurality of genes.

33. The method of claim 32, wherein the plurality of differentially translated genes comprise one or more gene signatures or are from one or more biological pathways.

34-40. (canceled)

41. The method of claim 27, wherein the disease is a cancer, an inflammatory disease, an autoimmune disease, a fibrotic disorder, a neurodegenerative disease, a neurodevelopmental disease, a metabolic disease, or a viral infection.

42. (canceled)

43. The method of claim 41, wherein the disease is a cancer selected from prostate cancer, breast cancer, bladder cancer, lung cancer, renal cell carcinoma, endometrial cancer, melanoma, ovarian cancer, thyroid cancer, or brain cancer.

44-46. (canceled)

47. The method of claim 27, wherein each translational profile comprises a genome-wide translational profile.

48. The method of claim 47, wherein less than about 20% of the genes in the genome are differentially translated in the first translational profile as compared to the second translational profile.

49. The method of claim 47, wherein less than about 5% or less than about 1% of the genes in the genome are differentially translated by at least two-fold in the first translational profile as compared to the second translational profile.

50. The method of claim 1, further comprising chemically synthesizing a structurally related agent derived from the identified candidate therapeutic.

51-53. (canceled)

54. A method for identifying a candidate therapeutic for normalizing a translational profile associated with a disease, the method comprising:
(a) determining a first translational profile for a plurality of genes from a disease sample that has been contacted with a candidate agent;
(b) determining a second translational profile for a plurality of genes from (1) a control non-diseased sample or (2) a control non-diseased sample that has been contacted with the candidate agent; and
(c) identifying the agent as a candidate therapeutic for normalizing a translational profile associated with the disease when the first translational profile is comparable to the second translational profile.

55. A method for identifying a candidate therapeutic for normalizing a translational profile associated with a disease, the method comprising:
(a) determining three independent translational profiles, each for a plurality of genes, wherein (i) a first translational profile is from a disease sample, (ii) a second translational profile is from (1) a control non-diseased sample or (2) a control non-diseased sample that has been contacted with a candidate agent, and (iii) a third translational profile is from a disease sample that has been contacted with the candidate agent;
(b) identifying one or more genes as differentially translated in the first translational profile as compared to the second profile; and
(c) identifying the agent as a candidate therapeutic for normalizing a translational profile associated with the disease when the one or more differentially translated genes from step (b) are in the third translational profile and when the translational profile of the one or more genes in the third translational profile is closer to the translational profile of the one or more genes in the second translational profile than to the translational profile of the one or more genes in the first translational profile.

56. The method of claim 55, wherein the one or more differentially translated genes from the third translational profile have a translational profile closer to the translational profile of the one or more genes in the second translational profile when the amount of protein translated from the one or more differentially translated genes in the third and second translational profiles differs by no more than about 50%, 45%, 40%, 35%, 30%, 25%, 20%, 15%, 10%, 5%, 1% or less.

57. A method for identifying a candidate therapeutic for normalizing a translational profile associated with a disease, the method comprising:
(a) determining three independent translational profiles, each for a plurality of genes, wherein (i) a first translational profile is from a disease sample, (ii) a second translational profile is from (1) a control non-diseased sample or (2) a control non-diseased sample that has been contacted with a candidate agent, and (iii) a third translational profile is from a disease sample that has been contacted with the candidate agent;
(b) determining a first differential translational profile comprising one or more genes differentially translated in the first translational profile as compared to the second translational profile, and determining a second differential translational profile comprising one or more genes differentially translated in the first translational profile as compared to the third translational profile; and
(c) identifying the agent as a candidate therapeutic for normalizing a translational profile associated with the disease when the first differential translational profile is comparable to the second differential translational profile.

58. The method of claim 57, wherein the first and second differential translational profiles are comparable when the amount of protein translated from the one or more differentially translated genes in the first and second differential translational profiles differs by no more than about 50%, 45%, 40%, 35%, 30%, 25%, 20%, 15%, 10%, 5%, 1% or less.

59. The method of claim 54, wherein the disease is a cancer, an inflammatory disease, an autoimmune disease, a fibrotic disorder, a neurodegenerative disease, a neurodevelopmental disease, a metabolic disease, or a viral infection.

60-61. (canceled)

62. The method of claim 54, wherein the one or more differentially translated genes comprises a plurality of genes.

63. The method of claim 62, wherein the plurality of differentially translated genes comprise one or more gene signatures or are from one or more biological pathways.

64-65. (canceled)

66. The method of claim 54, wherein each translational profile comprises a genome-wide translational profile.

67. The method of claim 66, wherein less than about 20% of the genes in the genome are differentially translated in the first translational profile as compared to the second translational profile.

68. The method of claim 66, wherein less than about 5% or less than about 1% of the genes in the genome are differentially translated by at least two-fold in the first translational profile as compared to the second translational profile.

69. A method of validating a target for therapeutic intervention in a disease, the method comprising:
(a) determining a first translational profile for a plurality of genes from a disease sample that has been contacted with an agent that modulates a target;
(b) determining a second translational profile for a plurality of genes from a control disease sample that has not been contacted with the agent; and
(c) validating the target for therapeutic intervention in the disease when one or more genes are differentially translated in the first translational profile as compared to the second translational profile and when the differential translation results in a biological benefit.

70. A method of validating a target for therapeutic intervention in a disease, the method comprising:
(a) determining a first translational profile for a plurality of genes from a disease sample that has been contacted with an agent that modulates a target;
(b) determining a second translational profile for a plurality of genes from a control disease sample that has been contacted with a known active compound for treating the disease; and
(c) validating the target as a target for therapeutic intervention in the disease when the first translational profile is comparable to the second translational profile.

71. The method of claim 70, wherein the known active compound is a therapeutic agent for a cancer, an inflammatory disease, an autoimmune disease, a fibrotic disorder, a neurodegenerative disease, a neurodevelopmental disease, a metabolic disease, or a viral infection.

72. The method of claim 70, wherein the translational profiles comprise one or more gene signatures, and wherein the translational profiles of the one or more gene signatures are comparable in the first translational profile and second translational profile.

73. The method of claim 70, wherein the first and second translational profiles are comparable when an amount of protein translated from one or more differentially translated genes in the first and second translational profiles differs by no more than about 50%, 45%, 40%, 35%, 30%, 25%, 20%, 15%, 10%, 5%, 1% or less.

74. The method of claim 69, wherein the one or more differentially translated genes comprises a plurality of genes.

75. The method of claim 74, wherein the plurality of differentially translated genes comprise one or more gene signatures or are from one or more biological pathways.

76-82. (canceled)

83. The method of claim 69, wherein the disease is a cancer, an inflammatory disease, an autoimmune disease, a fibrotic disorder, a neurodegenerative disease, a neurodevelopmental disease, a metabolic disease, or a viral infection.

84. (canceled)

85. The method of claim 83, wherein the disease is a cancer selected from prostate cancer, breast cancer, bladder cancer, lung cancer, renal cell carcinoma, endometrial cancer, melanoma, ovarian cancer, thyroid cancer, or brain cancer.

86-87. (canceled)

88. The method of claim 69, wherein each translational profile comprises a genome-wide translational profile.

89. The method of claim 88, wherein less than about 20% of the genes in the genome are differentially translated in the first translational profile as compared to the second translational profile.

90. The method of claim 88, wherein less than about 5% or less than about 1% of the genes in the genome are differentially translated by at least two-fold in the first translational profile as compared to the second translational profile.

91. A method for validating a target for therapeutic intervention in a disease, the method comprising:
(a) determining three independent translational profiles, each for a plurality of genes from a disease sample, wherein (i) a first translational profile is from a sample not contacted with any compound, (ii) a second translational profile is from a sample contacted with an agent that modulates a target, and (iii) a third translational profile is from a sample contacted with a known active compound for treating the disease;
(b) identifying one or more genes as differentially translated in the first translational profile as compared to the second translational profile; and
(c) validating the target as a target for therapeutic intervention in the disease when the one or more differentially translated genes from step (b) are in the third translational profile and when the translational profile of the one or more genes in the third translational profile is closer to the translational profile of the one or more genes in the second translational profile than to the translational profile of the one or more genes in the first translational profile.

92. The method of claim 91, wherein the one or more differentially translated genes from the third translational profile have a translational profile closer to the translational profile of the one or more genes in the second translational profile when the amount of protein translated from the one or more differentially translated genes in the third and second translational profiles differs by no more than about 50%, 45%, 40%, 35%, 30%, 25%, 20%, 15%, 10%, 5%, 1% or less.

93. A method for validating a target for therapeutic intervention in a disease, the method comprising:
(a) determining three independent translational profiles, each for a plurality of genes from a disease sample, wherein (i) a first translational profile is from a sample not contacted with any compound, (ii) a second translational profile is from a sample contacted with an agent that modulates a target, and (iii) a third translational profile is from a sample contacted with a known active compound for treating the disease;
(b) determining a first differential translational profile comprising one or more genes differentially translated in the first translational profile as compared to the second translational profile, and determining a second differential translational profile comprising one or more genes differentially translated in the first translational profile as compared to the third translational profile; and
(c) validating the target as a target for therapeutic intervention in the disease when the first differential translational profile is comparable to the second differential translational profile.

94. The method of claim 93, wherein the first and second differential translational profiles are comparable when the amount of protein translated from the one or more differentially translated genes in the first and second differential translational profiles differs by no more than about 50%, 45%, 40%, 35%, 30%, 25%, 20%, 15%, 10%, 5%, 1% or less.

95. The method of claim 91, wherein the known active compound is a therapeutic agent for a cancer, an inflammatory disease, an autoimmune disease, a fibrotic disorder, a neurodegenerative disease, a neurodevelopmental disease, a metabolic disease, or a viral infection.

96. The method of claim 91, wherein the one or more differentially translated genes comprises a plurality of genes.

97. The method of claim 96, wherein the plurality of differentially translated genes comprise one or more gene signatures or are from one or more biological pathways.

98-102. (canceled)

103. The method of claim 91, wherein the disease is a cancer, an inflammatory disease, an autoimmune disease, a fibrotic disorder, a neurodegenerative disease, a neurodevelopmental disease, a metabolic disease, or a viral infection.

104. (canceled)

105. The method of claim 103, wherein the disease is a cancer selected from prostate cancer, breast cancer, bladder cancer, lung cancer, renal cell carcinoma, endometrial cancer, melanoma, ovarian cancer, thyroid cancer, or brain cancer.

106. The method of claim 91, wherein each translational profile comprises a genome-wide translational profile.

107. The method of claim 106, wherein less than about 20% of the genes in the genome are differentially translated in the first translational profile as compared to the second translational profile.

108. The method of claim 106, wherein less than about 5% or less than about 1% of the genes in the genome are differentially translated by at least two-fold in the first translational profile as compared to the second translational profile.

109. A method for validating a target for normalizing a translational profile associated with a disease, the method comprising:
(a) determining a first translational profile for a plurality of genes from a disease sample that has been contacted with an agent that modulates a target;
(b) determining a second translational profile for a plurality of genes from (i) a control non-diseased sample or (ii) a control non-diseased sample that has been contacted with the agent that modulates the target; and
(c) validating the target as a target for normalizing a translational profile associated with the disease when the first translational profile is comparable to the second translational profile.

110. The method of claim 109, wherein the translational profiles comprise one or more gene signatures, and wherein the translational profiles of the one or more gene signatures are comparable in the first and second translational profiles.

111. The method of claim 109, wherein the first and second translational profiles are comparable when an amount of protein translated from one or more differentially translated genes in the first and second translational profiles differs by no more than about 50%, 45%, 40%, 35%, 30%, 25%, 20%, 15%, 10%, 5%, 1% or less.

112. A method for validating a target for normalizing a translational profile associated with a disease, the method comprising:
(a) determining three independent translational profiles, each for a plurality of genes, wherein (i) a first translational profile is from a disease sample, (ii) a second translational profile is from (1) a control non-diseased sample or (2) a control non-diseased sample that has been contacted with an agent that modulates a target, and (iii) a third translational profile is from a disease sample that has been contacted with the agent that modulates the target;
(b) identifying one or more genes as differentially translated in the first translational profile as compared to the second translational profile; and
(c) validating the target as a target for normalizing a translational profile associated with the disease when the one or more differentially translated genes from step (b) are in the third translational profile and when the translational profile of the one or more genes in the third translational profile is closer to the translational profile of the one or more genes in the second translational profile than to the translational profile of the one or more genes in the first translational profile.

113. The method of claim 112, wherein the one or more differentially translated genes from the third translational profile have a translational profile closer to the translational profile of the one or more genes in the second translational profile when the amount of protein translated from the one or more differentially translated genes in the third and second translational profiles differs by no more than about 50%, 45%, 40%, 35%, 30%, 25%, 20%, 15%, 10%, 5%, 1% or less.

114. A method for validating a target for normalizing a translational profile associated with a disease, the method comprising:
(a) determining three independent translational profiles, each for a plurality of genes, wherein (i) a first translational profile is from a disease sample, (ii) a second translational profile is from (1) a control non-diseased sample or (2) a control non-diseased sample that has been contacted with an agent that modulates a target, and (iii) a third translational profile is from a disease sample that has been contacted with the agent that modulates the target;
(b) determining a first differential translational profile comprising one or more genes differentially translated in the first translational profile as compared to the second translational profile, and determining a second differential translational profile comprising one or more genes differentially translated in the first translational profile as compared to the third translational profile; and
(c) validating the target as a target for normalizing a translational profile associated with the disease when the first differential translational profile is comparable to the second differential translational profile.

115. The method of claim 114, wherein the first and second differential translational profiles are comparable when the amount of protein translated from the one or more differentially translated genes in the first and second differential translational profiles differs by no more than about 50%, 45%, 40%, 35%, 30%, 25%, 20%, 15%, 10%, 5%, 1% or less.

116. The method of claim 109, wherein the disease is a cancer, an inflammatory disease, an autoimmune disease, a fibrotic disorder, a neurodegenerative disease, a neurodevelopmental disease, a metabolic disease, or a viral infection.

117. (canceled)

118. The method of claim 116, wherein the disease is a cancer selected from prostate cancer, breast cancer, bladder cancer, lung cancer, renal cell carcinoma, endometrial cancer, melanoma, ovarian cancer, thyroid cancer, or brain cancer.

119-120. (canceled)

121. The method of claim 109, wherein each translational profile comprises a genome-wide translational profile.

122. The method of claim 121, wherein less than about 20% of the genes in the genome are differentially translated in the first translational profile as compared to the second translational profile.

123. The method of claim 121, wherein less than about 5% or less than about 1% of the genes in the genome are differentially translated by at least two-fold in the first translational profile as compared to the second translational profile.

124-127. (canceled)

128. A method of identifying a subject as a candidate for treating a disease with a therapeutic agent, the method comprising:
(a) determining a first translational profile for a plurality of genes in a sample from a subject having or suspected of having a disease selected from a cancer, an inflammatory disease, an autoimmune disease, a neurodegenerative disease, a neurodevelopmental disease, a metabolic disease, and a viral infection;
(b) determining a second translational profile for a plurality of genes in a control sample, wherein the control sample is from a subject known to respond to the therapeutic agent and wherein the sample has not been contacted with the therapeutic agent; and
(c) identifying the subject as a candidate for treating the disease with the therapeutic agent when the first translational profile is comparable to the second translational profile.

129. The method of claim 128, wherein the disease is a cancer selected from prostate cancer, breast cancer, bladder cancer, lung cancer, renal cell carcinoma, endometrial cancer, melanoma, ovarian cancer, thyroid cancer, and brain cancer.

130. (canceled)

131. The method of claim 128, wherein the disease is an inflammatory disease selected from ankylosing spondylitis, atherosclerosis, multiple sclerosis, systemic lupus erythematosus (SLE), psoriasis, psoriatic arthritis, rheumatoid arthritis, ulcerative colitis, inflammatory bowel disease, and Crohn's disease.

132. The method of claim 128, wherein the disease is a fibrotic disorder selected from pulmonary fibrosis, idiopathic pulmonary fibrosis, cystic fibrosis, liver fibrosis, cardiac fibrosis, endomyocardial fibrosis, atrial fibrosis, mediastinal fibrosis, myelofibrosis, retroperitoneal fibrosis, chronic kidney disease, nephrogenic systemic fibrosis, Crohn's disease, hypertrophic scarring, keloid, scleroderma, organ transplant associated fibrosis, and ischemia associated fibrosis.

133. The method of claim 128, wherein the disease is a neurodegenerative disease selected from Parkinson's disease, Alzheimer's disease, Amyotrophic Lateral Sclerosis, Creutzfeldt-Jakob disease, Huntington's disease, Lewy body dementia, frontotemporal dementia, corticobasal degeneration, primary progressive aphasia, and progressive supranuclear palsy.

134. The method of claim 128, wherein the disease is a neurodevelopmental disease selected from autism, autism spectrum disorders, Fragile X Syndrome, attention deficit disorder, and pervasive development disorder.

135. The method of claim 128, wherein the disease is a viral infection selected from adenovirus, bunyavirus, herpesvirus, papovavirus, paramyxovirus, picornavirus, rhabdovirus, orthomyxovirus, poxvirus, reovirus, retrovirus, lentivirus, and flavivirus.

136. The method of claim 128, wherein the translational profiles comprise one or more gene signatures, and wherein the translational profiles of the one or more gene signatures are comparable in the first and second translational profiles.

137-138. (canceled)

139. The method of claim 128, wherein the first and second translational profiles are comparable when an amount of protein translated from one or more differentially translated genes in the first and second translational profiles differs by no more than about 50%, 45%, 40%, 35%, 30%, 25%, 20%, 15%, 10%, 5%, 1% or less.

140. The method of claim 128, wherein each translational profile comprises a genome-wide translational profile.

141. The method of claim 140, wherein less than about 20% of the genes in the genome are differentially translated in the first translational profile as compared to the second translational profile.

142. The method of claim 140, wherein less than about 5% or less than about 1% of the genes in the genome are differentially translated by at least two-fold in the first translational profile as compared to the second translational profile.

143. (canceled)

144. A method for treating a disease selected from a cancer, an inflammatory disease, an autoimmune disease, a neurodegenerative disease, a neurodevelopmental disease, a metabolic disease, and a viral infection, comprising administering a therapeutic agent to a subject identified according to the method of claim 128, thereby treating the subject.

145. A method for treating a disease selected from a cancer, an inflammatory disease, an autoimmune disease, a neurodegenerative disease, a neurodevelopmental disease, a metabolic disease, and a viral infection, the method comprising administering to a subject having the disease a therapeutic agent identified according to the method of claim 1, thereby treating the subject.

146. A method for treating a disease selected from a cancer, an inflammatory disease, an autoimmune disease, a neurodegenerative disease, a neurodevelopmental disease, a metabolic disease, and a viral infection, the method comprising administering to a subject having the disease an agent that modulates a target, wherein the target was validated according to the method of claim 69, thereby treating the subject.

147. A method for treating a disease selected from a cancer, an inflammatory disease, an autoimmune disease, a neurodegenerative disease, a neurodevelopmental disease, a metabolic disease, and a viral infection by normalizing the disease translational profile, the method comprising administering to a subject having the disease a therapeutic agent identified according to the method of claim 54, thereby treating the subject.

148. The method of claim 147, wherein the cancer is prostate cancer, breast cancer, bladder cancer, lung cancer, renal cell carcinoma, endometrial cancer, melanoma, ovarian cancer, thyroid cancer, or brain cancer.

149. The method of claim 147, wherein the inflammatory disease is ankylosing spondylitis, atherosclerosis, multiple sclerosis, systemic lupus erythematosus (SLE), psoriasis, psoriatic arthritis, rheumatoid arthritis, ulcerative colitis, inflammatory bowel disease, or Crohn's disease.

150. The method of claim 147, wherein the fibrotic disease is pulmonary fibrosis, idiopathic pulmonary fibrosis, cystic fibrosis, liver fibrosis, cardiac fibrosis, endomyocardial fibrosis, atrial fibrosis, mediastinal fibrosis, myelofibrosis, retroperitoneal fibrosis, chronic kidney disease, nephrogenic systemic fibrosis, Crohn's disease, hypertrophic scarring, keloid, scleroderma, organ transplant associated fibrosis, or ischemia associated fibrosis.

151. The method of claim 147, wherein the neurodegenerative disease is Parkinson's disease, Alzheimer's disease, Amyotrophic Lateral Sclerosis, Creutzfeldt-Jakob disease, Huntington's disease, Lewy body dementia, frontotemporal dementia, corticobasal degeneration, primary progressive aphasia, or progressive supranuclear palsy.

152. The method of claim 147, wherein the neurodevelopmental disease is autism, autism spectrum disorders, Fragile X Syndrome, attention deficit disorder, or a pervasive development disorder.

153. The method of claim 147, wherein the viral infection is adenovirus, bunyavirus, herpesvirus, papovavirus, paramyxovirus, picornavirus, rhabdovirus, orthomyxovirus, poxvirus, reovirus, retrovirus, lentivirus, or flavivirus.
